(19) United States
(12) Patent Application Publication
(10) Pub. No.: US 2024/0108751 A1
(43) Pub. Date: Apr. 4, 2024

(54) RNA-TARGETING COMPOSITIONS AND METHODS FOR TREATING MYOTONIC DYSTROPHY TYPE 1

(71) Applicant: Locanabio, Inc., San Diego, CA (US)

(72) Inventors: David A. NELLES, San Diego, CA (US); Ranjan BATRA, San Diego, CA (US); Daniela ROTH, San Diego, CA (US); Dimitrios ZISOULIS, San Diego, CA (US); Angeline TA, San Diego, CA (US)

(21) Appl. No.: 18/039,812

(22) PCT Filed: Dec. 1, 2021
(86) PCT No.: PCT/US2021/061490

§ 371 Date: Jun. 1, 2023

Related U.S. Application Data
(60) Provisional application No. 63/278,746, filed on Nov. 12, 2021.
(60) Provisional application No. 63/130,092, filed on Dec. 23, 2020.
(60) Provisional application No. 63/119,977, filed on Dec. 1, 2020.

(51) Int. Cl. A61K 48/00; C07K 14/47; A61P 21/00; C12N 15/11; C12N 9/22; C12N 15/86

(52) U.S. Cl. CPC A61K 48/0041; C07K 14/4705; A61P 21/00; C12N 15/11; C12N 9/22; C12Y 301/01; C12N 15/86; A61K 38/00

(57) ABSTRACT

Disclosed are RNA-targeting gene therapy compositions and methods for destroying or blocking toxic target CUG repeat RNA and treating DM1.

### RELATED APPLICATIONS

This application claims benefit of, and priority to, U.S. Ser. No. 63/119,977 filed on Dec. 1, 2020, U.S. Ser. No. 63/130,092 filed on Dec. 23, 2020, and U.S. Ser. No. 63/278,746 filed on Nov. 12, 2021; the contents of each are hereby incorporated by reference in their entireties.

### FIELD OF THE DISCLOSURE

The disclosure is directed to molecular biology, gene therapy, and compositions and methods for modifying expression and activity of RNA molecules.

### INCORPORATION BY REFERENCE OF SEQUENCE LISTING

The contents of the text file named “LOCN_007_001WO_SeqList_ST25”, which was created on Dec. 1, 2021 and is 1.81 MB in size, are hereby incorporated by reference in their entirety.

### BACKGROUND

There are long-felt but unmet needs in the art for providing effective RNA-targeting systems which provide effective gene therapies. In particular, the disclosure provides compositions and methods for specifically targeting and destroying toxic RNAs expressed from repetitive tracts in the microsatellite repeat expansion (MRE) disease known as myotonic dystrophy type 1 (DM1). DM1 is a multisystemic, autosomal-dominant inherited disorder caused by CTG MREs in the 3′ untranslated region of the DMPK gene. As for all MRE diseases, available treatments address symptoms of DM1 but do not target its underlying etiology. Elimination of MREs in DNA with genome editing could eliminate the pathogenic MREs causing DM1 but generation of DNA breaks near repeats activates the repair machinery whose activity is linked to expansion growth and may cause further mutation of the repeats and/or may fail to distinguish the pathogenic repeats from the normal repeats which possess regulatory roles in transcription. Other potential DM1 therapeutics have been evaluated, such as antisense oligonucleotides, shRNAs and small molecules, but these suffer from issues related to frequent redosing, poor penetration of affected tissues, lack of direct engagement with repeats, toxicity and off-target effects. In an effort to overcome these issues, Cas9-based RNA-targeting systems (RCas9) have been shown to be capable of specifically targeting toxic CUG repeat RNA and providing long-term repair of the disease phenotypes associated with DM1 in adult onset myotonic dystrophy in mice. However, other non-Cas9 RNA binding systems need to be delineated and developed for providing effective, sustained, and scalable gene therapy for the treatment of DM1. Such non-Cas9 RNA-binding systems targeting CUG MREs are important for manufacturing scale in that the system components are small enough to rely on a unitary vector. These non-RCas9 systems are also important for avoiding any deleterious immunological responses triggered by immunogenic Cas9 components. Accordingly, new and improved RNA-targeting, non-RCas9 gene therapy compositions and systems capable of eliminating toxic CUG repeats, and methods using the same for treating DM1, are provided herein.

### SUMMARY

The disclosure provides compositions and methods for treating myotonic dystrophy type 1 (DM1). The compositions and methods disclosed herein result in dose-dependent reduction in CUG^{exp} (CUG-repeat expansion) RNA via either destruction or blocking, reduced DMPK, and subsequent correction in alternative splicing and myotonia.

Disclosed herein is a composition comprising a nucleic acid encoding a non-guided RNA-binding protein comprising a PUF or PUMBY protein capable of binding a toxic target CUG repeat RNA sequence, wherein the RNA-binding protein is not capable of cleaving the toxic target CUG repeat RNA sequence.

Disclosed herein is a composition comprising a nucleic acid sequence encoding a non-guided RNA-binding fusion protein comprising a) a PUF or PUMBY protein capable of binding a toxic target CUG repeat RNA sequence and b) an endonuclease capable of cleaving the toxic target RNA sequence, wherein the endonuclease is a nuclease domain of a ZC3H12A zinc-finger endonuclease.

The disclosure provides a composition comprising a nucleic acid sequence encoding an RNA-binding polypeptide comprising a non-guided RNA binding polypeptide or a guided RNA-binding polypeptide capable of binding a toxic target CUG repeat RNA sequence.

In some embodiments, the RNA-binding polypeptide is a fusion protein.

In some embodiments, the fusion protein comprises the RNA binding polypeptide fused to an endonuclease capable of cleaving the toxic CUG repeat RNA sequence.

In some embodiments, the non-guided RNA binding polypeptide is a PUF or PUMBY protein.

In some embodiments, the guided RNA-binding polypeptide is a Cas13d protein. In some embodiments, the cas13d protein is catalytically dead. In some embodiments, the cas13d protein comprises an amino acid sequence set forth in any one of SEQ ID NOs 583 or 586-589.

In some embodiments, the endonuclease is a nuclease domain of a ZC3H12A zinc-finger endonuclease.

In some embodiments, the PUF RNA binding protein comprises an amino acid sequence set forth in any one of SEQ ID NOs 444-451, 461, 570, or 638-649. In some embodiments, the PUF RNA binding protein comprises an amino acid sequence set forth in SEQ ID NO: 444.

In some embodiments, the toxic target CUG RNA repeat sequence comprises any one of the nucleic acid sequences set forth in SEQ ID NOs 453-456.

In some embodiments, the toxic target CUG RNA repeat sequence comprises the nucleic acid sequence set forth in SEQ ID NO: 454.

In some embodiments, the CUG-targeting PUF protein is encoded by a nucleic acid sequence as set forth in SEQ ID NO: 452. In some embodiments, the PUF or PUMBY protein is a human PUF or PUMBY protein.

In some embodiments, the PUF or PUMBY protein is linked to the ZC3H12A endonuclease by a linker sequence.

In some embodiments, the linker comprises the amino acid sequence set forth in SEQ ID NO: 411.

In some embodiments, the fusion protein comprises one or more signal sequences selected from the group consisting of a nuclear localization sequence (NLS), and a nuclear export sequence (NES).

In some embodiments, the ZC3H12A zinc finger nuclease comprises the amino acid sequence set forth in SEQ ID NO: 358 or SEQ ID NO: 359.

In some embodiments, the fusion protein comprises the amino acid sequence set forth in any one of SEQ ID NOs 559-567. In some embodiments, the fusion protein is encoded by a nucleic acid sequence comprising SEQ ID NO: 460, SEQ ID NO: 516 or SEQ ID NO: 517.

In some embodiments, the nucleic acid molecule encoding the fusion protein comprises a promoter. In some embodiments, the promoter is a tCAG promoter, an EFS/UBB promoter, a desmin promoter, a CK8e promoter, or an EFS promoter.

The disclosure provides a vector comprising a composition of any embodiment of the disclosure.

In some embodiments, the vector is selected from the group consisting of: adeno-associated virus (AAV), retrovirus, lentivirus, adenovirus, nanoparticle, micelle, liposome, lipoplex, polymersome, polyplex, and dendrimer. In some embodiments, is an AAV vector. The disclosure provides an AAV vector of any embodiment of the disclosure, wherein the AAV vector comprises: a first AAV ITR sequence; a first promoter sequence; a polynucleotide sequence encoding for at least one CUG-repeat RNA binding polypeptide; and a second AAV ITR sequence.

In some embodiments, the CUG-repeat RNA binding polypeptide comprises a PUF or PUMBY protein. In some embodiments, the polynucleotide sequence encoding the PUF or PUMBY sequence comprises a nucleic acid sequence set forth in SEQ ID NO: 452.

In some embodiments, the CUG-repeat RNA binding polypeptide comprises a Cas13d protein. In some embodiments, the polynucleotide sequence encoding the Cas13d sequence comprises a nucleic acid sequence set forth in SEQ ID NO: 601, 612, or 615-618.

In some embodiments, the first promoter sequence comprises a nucleic acid sequence set forth in SEQ ID NO:568, 569, 608, 609, 634-637.

In some embodiments, the first AAV ITR sequence comprises a nucleic acid sequence set forth in SEQ ID NO: 599 or 600. In some embodiments, the second AAV ITR sequence comprises a nucleic acid sequence set forth in SEQ ID NO: 599 or 600.

In some embodiments, the vector further comprises a second promoter sequence. In some embodiments, the second promoter controls expression of a guide RNA (gRNA) wherein the gRNA comprises (i) a DR sequence and (ii) a spacer sequence. In some embodiments, the second promoter comprises a nucleic acid sequences set forth in SEQ ID NO: 519.

In some embodiments, the vector further comprises a polyA sequence. In some embodiments, the vector comprises at least one linker sequence. In some embodiments, the vector comprises at least one nuclear localization sequence.

In some embodiments, the vector is encoded be a nucleic set forth in any of one of SEQ ID NO: 574-582, 584-585, 590-597.

The disclosure provides a pharmaceutical composition comprising: a) the AAV viral vector of any embodiment of the disclosure; and b) at least one pharmaceutically acceptable excipient and/or additive.

The disclosure provides an AAV viral vector comprising: a) an AAV vector of any embodiment of the disclosure; and b) an AAV capsid protein. In some embodiments, the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein. In some embodiments, the AAV capsid protein is an AAV9 capsid protein

A cell comprising the vector of any one of the embodiments of the disclosure.

The disclosure provides a method of treating myotonic dystrophy type 1 (DM1) in a mammal comprising administering a composition or AAV vector according to any embodiment of the disclosure to a toxic target CUG microsatellite repeat expansion (MRE) RNA sequence in tissues of the mammal whereby the level of expression of the toxic target RNA is reduced.

In some embodiments, the composition or AAV vector is administered to the subject intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracistemally, intranervally, intrapleurally, topically, intralymphatically, intracisternally or intranerve.

In some embodiments, the composition or AAV vector is administered to the subject intravenously.

In some embodiments, the reduced level of expression of the toxic target RNA thereby ameliorates symptoms of DM1 in the mammal. In some embodiments, the level of expression of the toxic target RNA is reduced compared to the reduction in the level of expression of untreated toxic target CUG RNA. In some embodiments, the level of reduction is between 1-fold and 20-fold.

In some embodiments, the PUF RNA binding protein comprises an amino acid sequence set forth in any one of SEQ ID NOs 444-451, 461, 570, or 638-649.

In some embodiments, the PUF RNA binding protein comprises an amino acid sequence set forth in SEQ ID NO: 444.

In some embodiments, the toxic target CUG RNA repeat sequence comprises any one of SEQ ID NOs 453-456.

In some embodiments, the toxic target CUG RNA repeat sequence comprises SEQ ID NO: 454.

In some embodiments, the CUG-targeting PUF protein is encoded by a nucleic acid sequence as set forth in SEQ ID NO: 452.

In some embodiments, the PUF or PUMBY protein is a human PUF or PUMBY protein.

In some embodiments, the PUF or PUMBY protein is linked to the ZC3H12A by a linker sequence.

In some embodiments, the linker comprises the amino acid sequence set forth in SEQ ID NO: 411.

In some embodiments, the fusion protein comprises one or more signal sequences selected from the group consisting of a nuclear localization sequence (NLS), and a nuclear export sequence (NES).

In some embodiments, the ZC3H12A zinc finger nuclease comprises the amino acid sequence set forth in SEQ ID NO: 358 or SEQ ID NO: 359.

In some embodiments, the fusion protein comprises the amino acid sequence set forth in any one of SEQ ID NOs 559-567.

In some embodiments, the fusion protein is encoded by a nucleic acid sequence comprising SEQ ID NO: 460, SEQ ID NO: 516 or SEQ ID NO: 517.

In some embodiments, the nucleic acid molecule encoding the fusion protein comprises a promoter.

In some embodiments, the promoter is a tCAG promoter.

The disclosure provides a vector comprising any of the preceding compositions.

In some embodiments, the vector is selected from the group consisting of: adeno-associated virus (AAV), retrovirus, lentivirus, adenovirus, nanoparticle, micelle, liposome, lipoplex, polymersome, polyplex, and dendrimer. In some embodiments, the vector is an AAV vector. In some embodiments, the AAV vector is AAV9 or AAVrh74.

The disclosure provides a cell comprising the vector of the disclosure.

Disclosed herein is a method of treating myotonic dystrophy type 1 (DM1) in a mammal comprising administering a composition to a toxic target CUG microsatellite repeat expansion (MRE) RNA sequence in tissues of the mammal, wherein the composition comprises a nucleic acid sequence encoding a non-guided RNA-binding fusion protein comprising a) a PUF RNA-binding protein capable of binding a toxic target CUG RNA repeat sequence, and b) an endonuclease capable of cleaving the toxic target CUG RNA repeat sequence, whereby the level of expression of the toxic target RNA is reduced.

In some embodiments, the PUF RNA binding protein comprises any one of SEQ ID NOs 444-451, 461, 570, or 638-649.

In some embodiments, the PUF RNA binding protein comprises SEQ ID NO: 444.

In some embodiments, the toxic target CUG RNA repeat sequence comprises any one of SEQ ID NOs 453-456.

In some embodiments, the toxic target CUG RNA repeat sequence comprises SEQ ID NO: 453.

In some embodiments, the composition is administered to the tissue of the mammal by intravenous administration.

In some embodiments, the reduced level of expression of the toxic target RNA thereby ameliorates symptoms of DM1 in the mammal.

In some embodiments, the level of expression of the toxic target RNA is reduced compared to the reduction in the level of expression of untreated toxic target CUG RNA.

In some embodiments, the level of reduction is between 1-fold and 20-fold.

In some embodiments, the endonuclease is a ZC3H12A zinc-finger endonuclease.

In some embodiments, the ZC3H12A zinc finger nuclease comprises the amino acid sequence set forth in SEQ ID NO: 358 or SEQ ID NO: 359.

In some embodiments, the nucleic acid sequence encoding the fusion protein comprises a promoter.

In some embodiments, the promoter is a tCAG promoter.

In some embodiments, the promoter is a muscle-specific promoter.

In some embodiments, the muscle-specific promoter is a desmin promoter (full-length or truncated).

In some embodiments, the fusion protein comprises the amino acid sequences set forth in any one of SEQ ID NOs 559-567.

In some embodiments, the fusion protein is encoded by a nucleic acid sequence comprising SEQ ID NO: 460, SEQ ID NO: 516 or SEQ ID NO: 517.

A composition comprising a nucleic acid sequence encoding a non-naturally occurring or engineered clustered regularly interspaced short palindromic repeats (CRISPR)-associated (Cas) system comprising: (a) at least one RNA-guided RNase Cas protein; and b) at least one cognate CRISPR-Cas system guide RNA (gRNA) capable of forming a complex with one of the at least one Cas proteins, wherein the gRNA comprises (i) a DR sequence and (ii) a spacer sequence, wherein the spacer sequence hybridizes with the target CUG MRE molecule, and wherein the spacer sequence comprises a spacer sequence selected from the group consisting of: agcagcagcagcagcagcagcagcag (SEQ ID NO: 457), gcagcagcagcagcagcagcagcagc (SEQ ID NO: 458), and cagcagcagcagcagcagcagcagca (SEQ ID NO: 459), or a portion thereof, wherein the CRISPR-Cas system is capable of binding and cleaving the target CUG MRE.

A composition comprising a nucleic acid sequence encoding a non-naturally occurring or engineered clustered regularly interspaced short palindromic repeats (CRISPR)-associated (Cas) system comprising: (a) at least one RNA-guided RNase Cas protein; and b) at least one cognate CRISPR-Cas system guide RNA (gRNA) capable of forming a complex with one of the at least one Cas proteins, wherein the gRNA comprises (i) a DR sequence and (ii) a spacer sequence, wherein the spacer sequence hybridizes with the target CUG MRE molecule, and wherein the spacer sequence comprises a spacer sequence selected from the group consisting of: agcagcagcagcagcagcagcagcag (SEQ ID NO: 457), gcagcagcagcagcagcagcagcagc (SEQ ID NO: 458), and cagcagcagcagcagcagcagcagca (SEQ ID NO: 459), or a portion thereof, wherein the CRISPR-Cas system is capable of binding and cleaving the target CUG MRE, wherein the CRISPR-Cas system is catalytically inactive, and wherein the CRISPR-Cas is capable of binding but not cleaving the target CUG MRE.

In some embodiments, the Cas protein is Cas13a, Cas13b, Cas13c, or Cas13d. In some embodiments, the Cas protein is Cas13d.

In some embodiments, the RNA-guided RNase Cas protein or the non-guided RNA-binding polypeptide is a first RNA-binding polypeptide which is fused with a second RNA-binding polypeptide. In one embodiment, the second RNA-binding polypeptide is capable of binding RNA in a manner in which it associates with RNA. In some embodiments, the second RNA-binding polypeptide is capable of associating with RNA in a manner in which it cleaves RNA. In one embodiment, the second RNA-binding polypeptide is a nuclease domain of a ZC3H12A zinc-finger endonuclease.

In some embodiments, nucleic acid encoding the Cas or dCas system comprises a promoter. In some embodiments, the promoter is an EFS promoter. In some embodiments, the promoter is a muscle-specific promoter. In some embodiments, the muscle-specific promoter is a desmin promoter (full-length or truncated)

Disclosed herein is a vector comprising any of the preceding compositions.

In another embodiment, the vector is selected from the group consisting of: adeno-associated virus (AAV), retrovirus, lentivirus, adenovirus, nanoparticle, micelle, liposome, lipoplex, polymersome, polyplex, and dendrimer.

In another embodiment, the vector is an AAV vector.

In another embodiment, the AAV vector is AAV9 or AAVrh74.

Disclosed herein is a cell comprising the vector.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows results of a CUG⁹⁶⁰ qPCR assay which demonstrate exemplary embodiments of the CUG-targeting Cas13d compositions and PUF compositions disclosed herein destroy DM1 toxic CUG repeats. Reduction of the toxic repeats in a Cas13d-based system (labeled Cas13d-L1) is shown using three different guides CUG-g1, CUG-g2, and CUG-g3. Reduction of the toxic repeats in a PUF-based system is shown using an exemplary nucleic acid molecule encoding a PUF(CUG)-E17 fusion protein (labeled CUG-f1) and an E17-PUF(CUG) fusion protein (labeled CUG-f2). E17 is a domain of the ZC3H12A endonuclease. Results are normalized to non-targeting controls and shown as mean +/−s.d. of biological replicates (n=2).

FIG. 2 shows the results of an RNA Fluorescence In Situ Hybridization (FISH) assay with the exemplary CUG-targeting Cas13d and PUF compositions disclosed herein as compared to non-targeting controls.

FIG. 3 shows the results of a CUG⁹⁶⁰ qPCR assay which demonstrates destruction of the DM1 toxic CUG repeat RNA using the Cas13d compositions and PUF compositions disclosed herein as compared to the RCas9 system. Results were normalized to non-targeting controls and are shown as mean +/−s.d. of biological replicates (n=2).

FIG. 4A-C shows exemplary vector configurations of the DM1 gene therapy compositions disclosed herein. FIG. 4A illustrates a DM1 gene therapy construct configuration comprising CUG-targeting PUF-E17 operably linked to truncated CAG promoter (ICAG). FIG. 4B illustrates a DM1 gene therapy construct configuration comprising a CUG-targeting catalytically dead Cas13d fused to E17 and corresponding guide operably linked to EFS promoter. FIG. 4C illustrates a DM1 gene therapy construct configuration comprising a CUG-targeting Cas13d and corresponding guide operably linked to EFS promoter.

FIG. 5 depicts an alignment of a CUG-targeting PUF with human PUM1 with mismatches highlighted.

FIG. 6A-B shows results of a CUG⁹⁶⁰ assay. FIG. 6A shows knockdown of DMPK-CUG⁹⁶⁰ reporter mRNA. Specifically, Cas13d-CUG (A01215) decreases CUG⁹⁶⁰ repeat mRNA expression in CosM6 cells. Levels of CUG⁹⁶⁰ repeat mRNA (in context of human DMPK exons 11-15) are normalized to the reference gene GAPDH and transfection control GFP (expressed from the same plasmid as CUG⁹⁶⁰). Data are expressed as fold change in cells transfected with Cas13d-CUG relative to cells transfected with Cas13d non-targeting (NT) negative controls (n=3 different experiments). FIG. 6B shows that endogenous DMPK mRNA is preserved. Specifically, Cas13d-CUG did not decrease normal DMPK mRNA levels in HEK293 cells whereas a Cas13d-DMPK positive control targeting a repeat flanking region knocked down total DMPK mRNA by 58%.

FIG. 7 depicts two mechanisms of action for DM1 gene therapy: 1) Repeat destruction, and 2) Repeat blocking.

FIG. 8 shows three different embodiments of the DM1 AAV-based gene therapy compositions disclosed herein packaged in AAV9 vectors. Specifically, FIG. 8 depicts 1) therapeutic construct A01215 for destruction of the repetitive CUG based on a CRISPR/Cas13d system with cognate CUG targeting gRNA, 2) therapeutic construct A01344 for destruction of the repetitive CUG based on a PUF (Human PUM1-derived) protein fused with a human endonuclease domain (E17) engineered to target and cleave repetitive CUG RNA, and 3) therapeutic construct A01686 based on a PUF (Human PUM1-derived) protein engineered to target and bind (but not cleave) repetitive CUG RNA.

FIG. 9A-B show reduction of nuclear CUG^{exp} foci in patient muscle cells. FIG. 9A shows RNA-FISH to evaluate nuclear CUG^{exp} RNA foci in DM1 patient myocytes (2600 CUG repeats) treated with modified AAVrh74 (eAAV) encoding GFP (A01475—Control), Cas13d-CUG (A01215), or PUF(CUG)-E17 (A01344). FIG. 9B shows quantification of RNA FISH for number of nuclear CUG^{exp} RNA foci normalized to eAAV-GFP (A01475-control) demonstrating dose dependent reduction of toxic CUG RNA foci with A01215 (Cas13d-CUG) and A01344 (PUF(CUG)-E17).

FIG. 10A-G shows destruction of CUG^{exp} RNA and correction of DM1-associated mis-splicing and myotonia in HSA_{LR} DM1 mice. FIG. 10A depicts the mechanism of action of AAV-based A01215(Cas13d-CUG) and A01344 (PUF(CUG)-E17) mediated destruction of CUG^{exp} and consequent correction of alternative splicing and myotonia. FIG. 10B depicts the injection scheme showing injections of the vehicle and treatment in contralateral tibialis anterior (TA). FIG. 10C shows reduction of nuclear CUG^{exp} RNA foci in treated TA muscle using RNA-FISH with CAG10 probe. FIG. 10D shows a dose dependent decrease in HSA-CUG^{exp} RNA with AAV9-based A01344 (PUF(CUG)-E17) using RT-ddPCR. FIG. 10E-F shows correction of alternative splicing of DM1-associated Atp2a1 Exon 22 and ClCn1 Exon 7a, respectively, using semi quantitative RT-PCR followed by capillary electrophoresis. FIG. 10G shows reduction in myotonia represented as % of needle insertions resulting in myotonic runs using needle electromyography (EMG).

FIG. 11A-G shows blocking of CUG^{exp} RNA and correction of DM1-associated mis-splicing and myotonia in HSA_{LR} DM1 mice. FIG. 11A depicts the mechanism of action of AAV-based A01686 (PUF(CUG)) mediated blocking of CUG^{exp} and consequent correction of alternative splicing and myotonia. FIG. 11B depicts the injection scheme showing injections of the vehicle and treatment in contralateral tibialis anterior (TA). FIG. 11C shows reduction of nuclear CUG^{exp} RNA foci in treated TA muscle using RNA-FISH with CAG10 probe. FIG. 11D shows a dose dependent decrease in HSA-CUG^{exp} RNA with AAV9-based A01686 (PUF(CUG)) using RT-ddPCR. FIG. 11E-F shows correction of alternative splicing of DM1-associated Atp2a1 Exon 22 and ClCn1 Exon 7a, respectively, using semi quantitative RT-PCR followed by capillary electrophoresis. FIG. 11G shows reduction in myotonia represented as % of needle insertions resulting in myotonic runs using needle electromyography (EMG).

FIG. 12A-B depicts exemplary vector configurations of the DM1 blocking (without cleavage) gene therapy compositions disclosed herein. FIG. 12A shows several PUF(CUG) embodiments and FIG. 12B shows several dCas13d(CUG) embodiments.

### DETAILED DESCRIPTION

The disclosure provides RNA-targeting gene therapy compositions and methods for treating myotonic dystrophy type 1 (DM1).

DM1 is a multisystemic, autosomal-dominant inherited disorder caused by CTG microsatellite repeat expansions (MREs) in the 3′ untranslated region of the DMPK gene. RNA transcripts containing the CUG repeat expansions sequester muscleblind-like (MBNL) proteins which are the regulators of the alternative splicing switch from fetal to adult isoforms. Adult DM1 patients experience debilitating myotonia and progressive weakness in skeletal muscle while infants born with DM1 (congenital DM or CDM) are hypotonic and display respiratory insufficiency. The DMPK gene encodes a protein called myotonic dystrophy protein kinase which is believed to play a role in muscle, heart, and brain cells. The protein may be involved in communication within cells. It also appears to regulate the production and function of important structures inside muscle cells by interacting with other proteins. For example, myotonic dystrophy protein kinase has been shown to inhibit part of a muscle protein called myosin phosphatase. Myosin phosphatase is an enzyme that plays a role in muscle tensing (contraction) and relaxation. One region of the DMPK gene contains a segment of three DNA building blocks (nucleotides) that is repeated multiple times. This sequence, which is written as CTG, is called a triplet or trinucleotide repeat. In most unaffected people, the number of CTG repeats in this gene ranges from 5 to 34. In DM1 patients, there is a CTG repeat expansion which increases the size of the CTG repeat in the DMPK gene. DM1 is classified as either adult-onset or as congenital forms that are distinguished by the size of the expanded CTG tract. Repeats in such CTG repeat expansions can range from about 50 to about 1,000 CTG repeats in most cells and in certain cell types, such as muscle cells, the number of repeats are typically greater. Indeed, the size of the trinucleotide repeat expansion is associated with the severity of signs and symptoms of DM1. Classic features such as muscle weakness and wasting beginning in adulthood and correlate with about 100 to about 1,000 CTG repeats per cell. The more severe congenital form of DM1 tends to correlate with over 1,000 CTG repeats per cell. The mild form of DM1 typically ranges from about 50 to about 150 CTG repeats per cell. DM1 is classified as either adult-onset or as congenital forms that are distinguished by the size of the expanded CTG tract. The repetitive RNAs produced by DMPK locus form nuclear RNA foci that sequester RNA binding proteins such as MBNL1 (Muscleblind Like Splicing Regulator 1) and divert them from their homeostatic RNA processing activities. Loss of MBNL1 function is linked to hundreds of alternative splicing defects and respiratory insufficiency which contribute in varying degrees to patient mortality. Targeting and eliminating (or blocking) CUG repeats is a therapeutic strategy for DM1.

The gene therapy compositions disclosed herein provide efficacious cleavage or blocking of toxic CUG repeats in methods of treating DM1. Building on prior work using RCas9 systems, disclosed herein are multiple RNA-binding systems which do not rely on RCas9 system components. While Cas9-based RNA-targeting systems (RCas9) are capable of specifically targeting toxic CUG repeat RNA and providing long-term repair of the disease phenotypes associated with DM1 in adult onset myotonic dystrophy in mice, other non-Cas9 RNA binding systems disclosed herein provide efficient cleavage or blocking of toxic CUG repeat RNA. Such non-Cas9 RNA-binding systems targeting CUG MREs are important for scaling of therapeutic systems in manufacturing. In particular, the non-Cas9 system components are a small enough size to rely on a unitary (single) vector. The non-RCas9 systems disclosed herein are capable of achieving effective knockdown or blocking of the toxic CUG repeats compared to RCas9 systems and non-RCas9 systems are also important for avoiding any deleterious immunological responses triggered by immunogenic and unwieldy Cas9 components.

Disclosed herein are compositions comprising nucleic acid molecules, and vectors comprising the same, encoding non-Cas9 RNA-binding systems capable of binding toxic CUG repeat RNA for treating DM1. Such compositions are capable of targeting and binding for either knockdown/destruction or blocking the toxic CUG repeats, both mechanisms (destruction and blocking) causing a correction of MBNL sequestration, alternative splicing, and myotonia. Indeed, in one embodiment, a gene therapy blocking composition comprising PUF(CUG) will bind CUG^{exp} RNA directly and block MBNL sequestration to preserve near normal free MBNL levels and function that will reverse DM1 disease phenotypes such as splicing dysfunction, myotonia and others. In some aspects, compositions suitable for blocking CAG-repeat RNA bind a CUG-repeat containing RNA and prevent translation of the CUG-repeat RNA. In some aspects, this prevented translation results in reduced protein expression from CUG-repeat containing RNA sequences. These systems disclosed herein comprise either RNA-guided RNase Cas or non-guided PUF, PUMBY or PPR protein configurations.

In some embodiments, the guided or non-guided CUG-repeat targeting system targets expanded CUG repeats (CUG^{exp}), wherein the CUG repeats are CUG⁵⁰ or more. In some embodiments, the CUG repeats are CUG¹⁰⁰ or more. In some aspects, the CUG repeats are CUG⁵⁰⁰ or more. In some aspects, the CUG repeats are CUG⁹⁶⁰. In some aspects, the CUG¹⁰⁰⁰ repeats are 1000 CUG repeats or more. To be clear, CUG⁵⁰ or CUG¹⁰⁰ or CUG⁹⁶⁰ or CUG¹⁰⁰⁰ refers to 50 CUG repeats or 100 CUG repeats or 960 CUG repeats or 1000 CUG repeats, respectively, in a CUG repeat containing gene. Any other number or range of CUG repeats are possible, including 50, 55, 60, 65, 70, 75, 76, 77, 78, 79, 80, 81, 82, 83, 84, 85, 90, 95, 100, 105, 110, 115, 120, 150, 180, 200, 250, 300, 350, 400, 450, 500, 550, 600, 650, 700, 750, 800, 850, 900, 950, 960, 1000 CUG repeats, or any other number of CUG repeats in between.

In any of the preceding or subsequent RNA-targeting compositions for treating DM1, any particular construct element (e.g., linker, promoter, signal sequence, etc.) described in the context of a specific RNA-targeting composition, can be substituted for another of the same element type (e.g., linker, promoter, signal sequence, etc.). In some embodiments, any particular construct element can be omitted or removed (such as a tag sequence). In other words, the exemplary combinations of elements in any particular gene therapy composition described herein is not intended to be limiting.

### RNA-Guided CUG-Repeat RNA Binding Systems

In some embodiments, the non-Cas9-binding system is comprised of an RNA-guided RNA-binding polypeptide. In some embodiments, a nucleic acid sequence encodes an RNA-guided RNA-binding polypeptide which is an RNase Cas protein (or a deactivated RNase Cas protein). In one embodiment, the nucleic acid sequence further comprises a gRNA sequence comprising a spacer sequence which binds to a toxic target CUG repeat RNA and a direct repeat (DR) sequence which binds to the RNase Cas protein. In one embodiment, a Cas13d(CUG) system is catalytically active, in which case, the Cas13d nucleoprotein complex cleaves and destroys toxic RNA CUG repeats. In another embodiment, a Cas13d(CUG system is catalytically inactive, in which case, the Cas13d nucleoprotein complex binds and blocks (but does not cleave) the RNA CUG repeats. In yet another embodiment, a Cas13d(CUG) comprises a catalytically inactive Cas13d(CUG) fused to an endonuclease which is capable of cleaving the toxic RNA CUG repeats. In such an embodiment, the endonuclease is an active RNase. Exemplary endonucleases with RNase activity can be found herein and these include, for example, a domain from a ZC3H12A zinc-finger (also referred herein as E17) or a PIN endonuclease. In some embodiments, a nucleic acid sequence encoding a CUG-repeat targeting composition comprises a first promoter sequence that controls expression of a Cas13d protein or Cas13d fusion protein and a second promoter sequences that controls expression of the at least one guide RNA sequence.

TABLE 1
Exemplary spacer sequences used in
sgRNAs for CUG targeting with
RNase Cas systems for treating DM1:

| Spacer | Spacer Sequences |
|---|---|
| 1 | agcagcagcagcagcagcagcagcag |
| | (SEQ ID NO: 457) |
| 2 | gcagcagcagcagcagcagcagcagc |
| | (SEQ ID NO: 458) |
| 3 | cagcagcagcagcagcagcagcagca |
| | (SEQ ID NO: 459) |

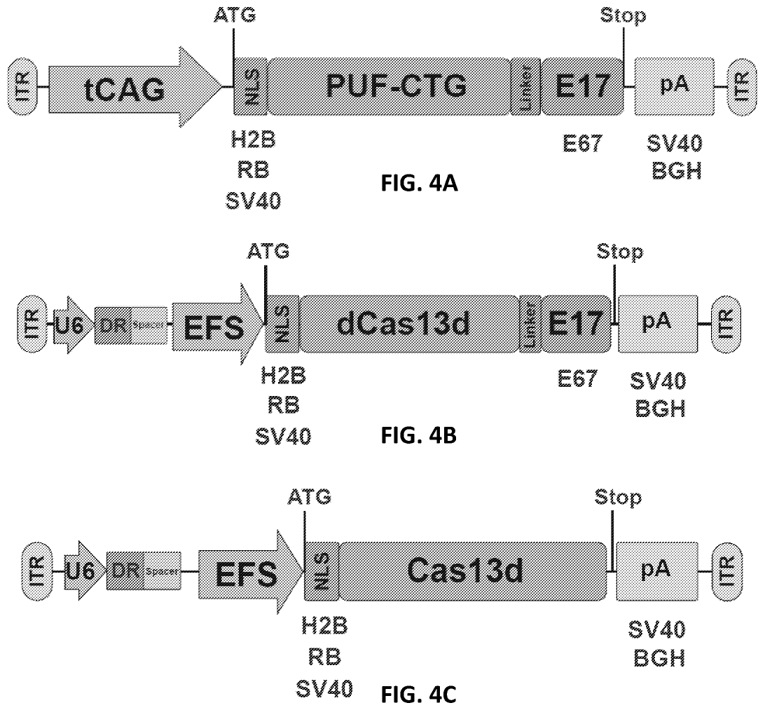

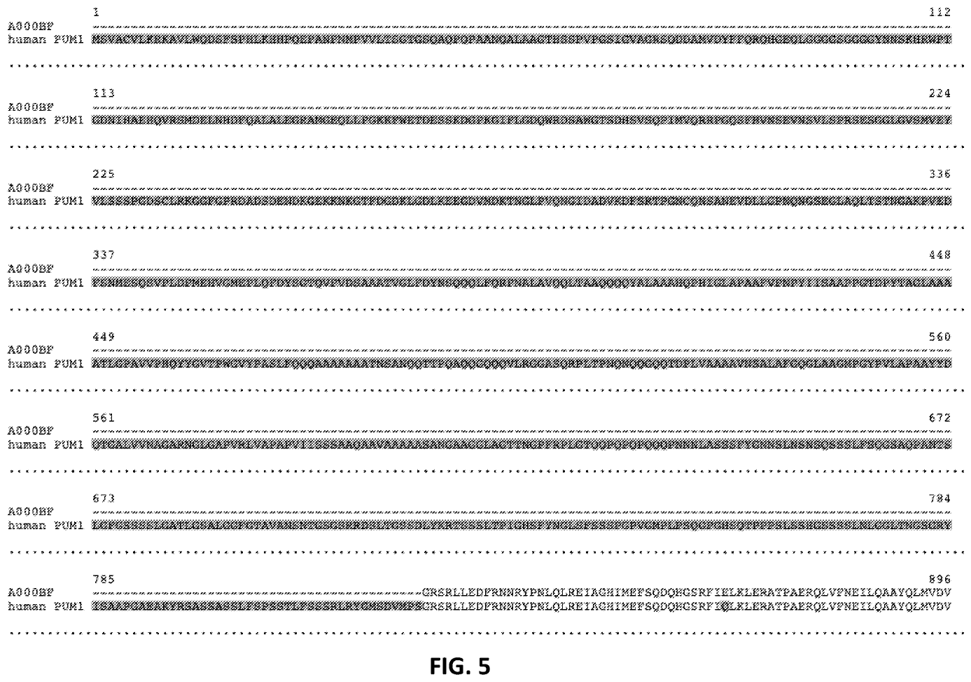

In one embodiment, the RNase Cas protein is a Cas13 protein. In another embodiment, the Cas13 protein is a Cas13d protein. In another embodiment, the Cas13d protein is a deactivated RNase Cas13d protein (dCas13d). In another embodiment, the dCas13d protein is a fusion protein comprising 1) dCas13d and 2) a polypeptide encoding a protein or fragment thereof having nuclease activity. In another embodiment, the dCas13d protein is a fusion protein comprising 1) dCas13d and 2) ZC3H12A, a zinc-finger endonuclease or a truncated version thereof (referred to as E17 or SEQ ID NO: 358 herein). In some embodiments, the Cas configuration comprises a signal sequence(s) such as NLS(s) and/or NES(s). In some embodiments, the dCas13d is linked to the E17 endonuclease via a linker sequence. In one embodiment, the linker sequence is VDTANGS (SEQ ID NO: 411). In some embodiments, the Cas13d or dCas13d fusion proteins are operably linked to a promoter sequence. In some embodiments, the promoter sequence comprises an enhancer and/or an intron. In some embodiments, the promoter sequence is an EFS promoter sequence (FIGS. 4B and 4C).

In some embodiments, a CUG-repeat targeting cas13d or dCas13d protein of the disclosure comprises from N-terminal to C-terminal: Cas13d (Seq212), a linker, and an SV-40 NLS. In some aspects, the CUG-repeat targeting dCas13d protein is used for methods of blocking CUG-repeat RNA sequence expression.

Active Cas13d (Seq212) with truncated
Desmin promoter (A02207)

| Plasmid Element | Amino Acid Sequences |
|---|---|
| Cas13d Seq212 | KKKHQSAAEKRQVKKLKNQEKAQKY |
| | ASEPSPLQSDTAGVECSQKKTVVSH |
| | IASSKTLAKAMGLKSTLVMGDKLVI |
| | TSFAASKAVGGAGYKSANIEKITDL |
| | QGRVIEEHERMFSADVGEKNIELSK |
| | NDCHTNVNNPVVTNIGKDYIGLKSR |
| | LEQEFFGKTFENDNLHVQLAYNILD |
| | IKKILGTYVNNIIYIFYNLNRAGTG |
| | RDERMYDDLIGTLYAYKPMEAQQTY |
| | LLKGDKDMRRFEEVKQLLQNTSAYY |
| | VYYGTLFEKVKAKSKKEQRAKEAEI |
| | DACTAHNYDVLRLLSLMRQLCMHSV |
| | AGTAFKLAESALFNIEDVLSADLKE |
| | ILDEAFSGAVNKLNDGFVQHSGNNL |
| | YVLQQLYPNETIERIAEKYYRLTVR |
| | KEDLNMGVNIKKLRELIVGQYFPEV |
| | LDKEYDLSKNGDSVVTYRSKIYTVM |
| | NYILLYYLEDHDSSRESMVEALRQN |
| | REGDEGKEEIYRQFAKKVWNGVSGL |
| | FGVCLNLFKTEKRNKFRSKVALPDV |
| | SGAAYMLSSENIDYFVKMLFFVCKF |
| | LDGKEINELLCALINKFDNIADILD |
| | AAAQCGSSVWFVDSYRFFERSRRIS |
| | AQIRIVKNIASKDFKKSKKDSDESY |
| | PEQLYLDALALLGDVISKYKQNRDG |
| | SVVIDDQGNAVLTEQYKRFRYEFFE |
| | EIKRDESGGIKYKKSGKPEYNHQRR |
| | NFILNNVLKSKWFFYVVKYNRPSSC |
| | RELMKNKEILRFVLRDIPDSQVRRY |
| | FKAVQGEEAYASAEAMRTRLVDALS |
| | QFSVTACLDEVGGMTDKEFASQRAV |
| | DSKEKLRAIIRLYLTVAYLITKSMV |
| | KVNTRFSIAFSVLERDYYLLIDGKK |
| | KSSDYTGEDMLALTRKFVGEDAGLY |
| | REWKEKNAEAKDKYFDKAERKKVLR |
| | QNDKMIRKMHFTPHSLNYVQKNLES |
| | VQSNGLAAVIKEYRNAVAHLNIINR |
| | LDEYIGSARADSYYSLYCYCLQMYL |
| | SKNFSVGYLINVQKQLEEHHTYMKD |
| | LMWLLNIPFAYNLARYKNLSNEKLF |
| | YDEEAAAEKADKAENERGE |
| | (SEQ ID NO: 572) |
| Linker | GS |
| SV-40 NLS | PKKKRKV (SEQ ID NO: 571) |

### Non-Guided CUG-Repeat RNA Binding Systems

In some embodiments, the non-Cas9 RNA-binding system does not comprise an RNA-guided RNA-binding polypeptide. Instead, the non-Cas9 RNA-binding system is comprised of a non-RNA-guided RNA-binding polypeptide such as a PUF protein or a PUMBY protein, or RNA-binding portion thereof. In one embodiment, a non-guided RNA-binding fusion protein disclosed herein comprises a) a PUF or PUMBY RNA-binding sequence capable of binding a toxic target CUG repeat sequence comprising UGCUGCUG (SEQ ID NO: 453) and b) an endonuclease capable of cleaving the toxic target CUG repeat sequence.

In one embodiment, the target RNA sequence is selected from the group consisting of UGCUGCUGCUGCUG (SEQ ID NO: 454), UGCUGCUGCUGCUGC (SEQ ID NO: 455), and UGCUGCUGCUGCUGCU (SEQ ID NO: 456).

In one embodiment, the target RNA sequence is selected from the group consisting of CUGCUGCU (SEQ ID NO: 472), CUGCUGCUGCUGCU (SEQ ID NO: 473), CUGCUGCUGCUGCUG (SEQ ID NO: 474), and CUGCUGCUGCUGCUGC (SEQ ID NO: 475).

In one embodiment, the target RNA sequence is selected from the group consisting of GCUGCUGC (SEQ ID NO: 476), GCUGCUGCUGCUGC (SEQ ID NO: 477), GCUGCUGCUGCUGCU (SEQ ID NO: 478), and GCUGCUGCUGCUGCUG (SEQ ID NO: 479).

In one embodiment, the PUF or PUMBY RNA-binding fusion protein comprises a) PUF or PUMBY CUG-targeting protein and b) ZC3H12A, a zinc-finger endonuclease or a truncated version thereof (referred to as E17 or SEQ ID NO: 358 herein). In some embodiments, the CUG-targeting PUF or PUMBY fusion protein is configured N-terminal to C-terminal as follows:
PUF(CUG)-E17
E17-PUF(CUG)
PUMBY(CUG)-E17, or
E17-PUMBY(CUG).

In some embodiments, the PUF or PUMBY fusion configurations include a linker between the PUF(CUG) or PUMBY(CUG) and the E17. In one embodiment, the linker sequence is VDTANGS (SEQ ID NO: 411).

In some embodiments, the CUG-targeting PUF or PUMBY fusion protein comprising a linker is configured N-terminal to C-terminal as follows:
PUF(CUG)-linker-E17
E17-linker-PUF(CUG)
PUMBY(CUG)-linker-E17; or
E17-linker-PUMBY(CUG).

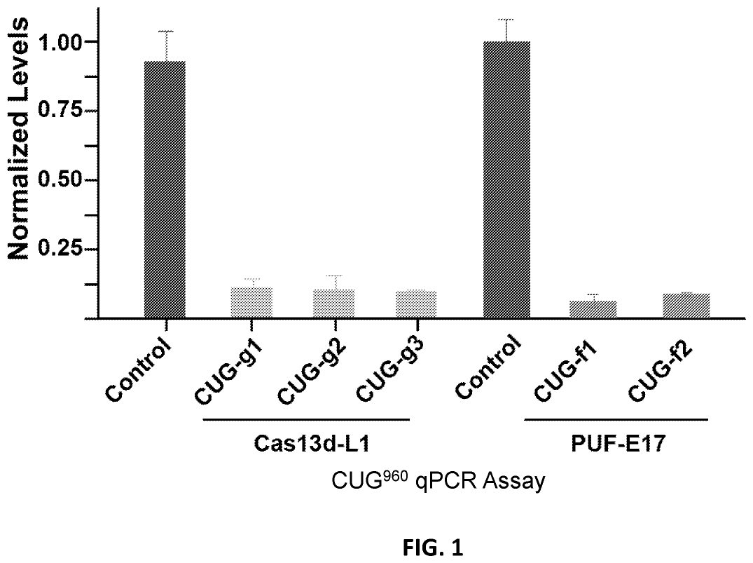

An exemplary embodiment of the N- to C-terminal orientation of a PUF(CUG)-linker-E17 is the first orientation CUG frame (CUG-f1) of FIG. 1 which is orientated N- to C-terminal as PUF(CUG)-E17. An exemplary embodiment of the N- to C-terminal orientation of a E17-linker-PUF(CUG) is the second orientation CUG frame (CUG-f2) of FIG. 1 which is orientated N- to C-terminal as E17-linker-PUF(CUG).

In one embodiment, the CUG-targeting PUF or PUMBY fusion protein configuration from N-terminal to C-terminal is PUF(CUG)-VDTANGS-E17 or PUMBY(CUG)-VDTANGS-E17. In another embodiment, the CUG-targeting PUF or PUMBY fusion protein configuration from N-terminal to C-terminal is E17-VDTANGS-PUF(CUG) or E17-VDTANGS-PUMBY(CUG).

In some embodiments, the PUF or PUMBY configurations include one or more tags or signal sequences such as FLAG, NLS, NES or a combination thereof. In one embodiment, the FLAG tag sequence is DYKDDDDK (SEQ ID NO: 436). In one embodiment, the NLS signal sequence is a human NLS. In one embodiment, the NES is a human NES. In one embodiment, the NLS is a SV40 NLS. In another embodiment, the SV40 NLS sequence is PKKKRKV (SEQ ID NO: 437). In one embodiment, the configuration comprises two different tags and/or signal sequences. In another embodiment, the configuration comprises two or more signal sequences. In some embodiments, the tag(s) and/or signal(s) is/are located at the N-terminal. In some embodiments, the tag(s) and/or signal(s) is/are located at the C-terminal. In some embodiments, a tag(s) and/or signal(s) is/are located at the N-terminal and a tag(s) and/or signal(s) is/are located at the C-terminal. In one embodiment, the CUG-targeting PUF or PUMBY fusion protein comprising one or more tags and/or signals is/are configured N-terminal to C-terminal as follows:
FLAG-NLS-PUF(CUG)-linker-E17; or
FLAG-NLS-PUMBY(CUG)-linker-E17;
NLS-PUF(CUG)-linker-E17; or
NLS-PUMBY(CUG)-linker-E17.

In one embodiment, the CUG-targeting PUF or PUMBY fusion protein comprising one or more tags and/or signal(s) is/are configured N-terminal to C-terminal as follows:
FLAG-NLS-PUF(CUG)-VDTANGS-E17; or
FLAG-NLS-PUMBY(CUG)-VDTANGS-E17;
NLS-PUF(CUG)-VDTANGS-E17; or
NLS-PUMBY(CUG)-VDTANGS-E17.

Table 2A-2B: Exemplary PUF configurations for targeting CUG MRE:

PUF targeting CUG (DM1) with endonuclease for destruction:

| Protein | | Target | Amino Acid |
|---|---|---|---|
| Type | Elements | Sequence | Sequence of PUF |
| 8PUF | N-terminal | UGCUGCUG | GRSRLLEDFRNNRYP |
| | SV40 NLS; | (SEQ ID | NLQLREIAGHIMEFS |
| | Linker | NO: | QDQHGSRFIELKLER |
| | between | 453) | ATPAERQLVFNEILQ |
| | PUF and E17 | | AAYQLMVDVFGNYVI |
| | endonuclease | | QKFFEFGSLEQKLAL |
| | (VDTANGS); | | AERIRGHVLSLALQM |
| | C-terminal | | YGSYVIRKALEFIPS |
| | E17 | | DQQNEMVRELDGHVL |
| | | | KCVKDQNGSYVVEKC |
| | | | IECVQPQSLQFIIDA |
| | | | FKGQVFALSTHPYGN |
| | | | RVIQRILEHCLPDQT |
| | | | LPILEELHQHTEQLV |
| | | | QDQYGSYVIRHVLEH |
| | | | GRPEDKSKIVAEIRG |
| | | | NVLVLSQHKFASNVV |
| | | | EKCVTHASRTERAVL |
| | | | IDEVCTMNDGPHSAL |
| | | | YTMMKDQYANYVVQK |
| | | | MIDVAEPGQRKIVMH |
| | | | KIRPHIATLRKYTYG |
| | | | KHILAKLEKYYMKNG |
| | | | VDLG |
| | | | (SEQ ID NO: 444) |

PUF targeting CUG (DM1) with no endonuclease for blocking:

| | | | Target | |
|---|---|---|---|---|
| | Protein | | Se- | Amino Acid |
| Construct | Type | Elements | quence | Sequence |
| A01685 | 8PUF | N- | UGCUGC | GRSRLLEDFRNNRYPN |
| | | terminal | UG (SEQ | LQLREIAGHIMEFSQ |
| | | Rb NLS, | ID NO: | DQHGSRFIELKLERA |
| | | Linker | 453) | TPAERQLVFNEILQA |
| | | between | | AYQLMVDVFGNYVIQ |
| | | NLS | | KFFEFGSLEQKLALA |
| | | and | | ERIRGHVLSLALQMY |
| | | 8PUF | | GSYVIRKALEFIPSD |
| | | | | QQNEMVRELDGHVLK |
| | | | | CVKDQNGSYVVEKCI |
| | | | | ECVQPQSLQFIIDAF |
| | | | | KGQVFALSTHPYGNR |
| | | | | VIQRILEHCLPDQTL |
| | | | | PILEELHQHTEQLVQ |
| | | | | DQYGSYVIRHVLEHG |
| | | | | RPEDKSKIVAEIRGN |
| | | | | VLVLSQHKFASNVVE |
| | | | | KCVTHASRTERAVLI |
| | | | | DEVCTMNDGPHSALY |
| | | | | TMMKDQYANYVVQKM |
| | | | | IDVAEPGQRKIVMHK |
| | | | | IRPHIATLRKYTYGK |
| | | | | HILAKLEKYYMKNGV |
| | | | | DLG (SEQ ID NO: |
| | | | | 444) |

In some embodiments, an AAV vector of the disclosure comprising a CUG-targeting PUF protein comprises from 5′ to 3′ as set forth in Table A. In some aspects, the AAV vector comprises the nucleic acid sequence set forth in SEQ ID NO: 573 or 574.

TABLE A
PUF-CUG-E17 with Desmin (FL) promoter (A02205)

| Plasmid | |
|---|---|
| Element | Amino Acid Sequences |
| Rb-NLS | DRVLKRSAEGSNPPKPLKKLR |
| | (SEQ ID NO: 543) |
| Linker | GGS |
| 8PUF | GRSRLLEDFRNNRYPNLQLREIAGH |
| | IMEFSQDQHGSRFIELKLERATPAE |
| | RQLVFNEILQAAYQLMVDVFGNYVI |
| | QKFFEFGSLEQKLALAERIRGHVLS |
| | LALOMYGSYVIRKALEFIPSDQQNE |
| | MVRELDGHVLKCVKDQNGSYVVEKC |
| | IECVQPQSLQFIIDAFKGQVFALST |
| | HPYGNRVIQRILEHCLPDQTLPILE |
| | ELHQHTEQLVQDQYGSYVIRHVLEH |
| | GRPEDKSKIVAEIRGNVLVLSQHKF |
| | ASNVVEKCVTHASRTERAVLIDEVC |
| | TMNDGPHSALYTMMKDQYANYVVQK |
| | MIDVAEPGQRKIVMHKIRPHIATLR |
| | KYTYGKHILAKLEKYYMKNGVDLG |
| | (SEQ ID NO: 444) |
| Linker | VDTANGS (SEQ ID NO: 411) |
| E17 | GGGTPKAPNLEPPLPEEEKEGSDLR |
| | PVVIDGSNVAMSHGNKEVFSCRGIL |
| | LAVNWFLERGHTDITVFVPSWRKEQ |
| | PRPDVPITDQHILRELEKKKILVFT |
| | PSRRVGGKRVVCYDDRFIVKLAYES |
| | DGIVVSNDTYRDLQGERQEWKRFIE |
| | ERLLMYSFVNDKFMPPDDPLGRHGP |
| | SLDNFLRKKPLTLE |
| | (SEQ ID NO: 358) |

In one embodiment, the nucleic acid encoding the PUF(CUG) or PUMBY(CUG) fusion construct or PUF(CUG) or PUMBY(CUG) without an endonuclease is operably linked to a promoter sequence for expression in a cell. In one embodiment, the promoter sequence is a truncated CAG (tCAG) promoter (FIG. 4A). In some embodiments, the promoter sequence comprises an enhancer sequence and/or an intron sequence. In one embodiment, the promoter is a EFS/UBB promoter. In some embodiments, the promoter sequence is a muscle-specific promoter.

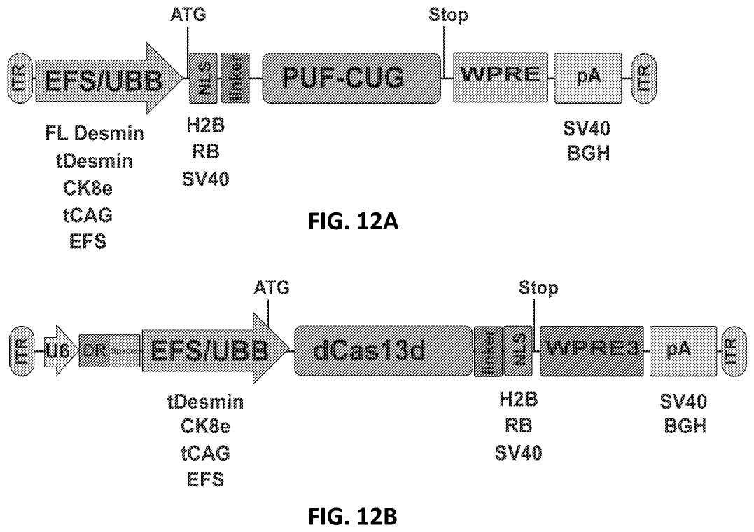

In one embodiment, the nucleic acid encoding the PUF(CUG) (with or without an endonuclease), Cas13d(CUG) or dCas13d(CUG) (dCas13d(CUG) with or without an endonuclease) is operably linked to a promoter sequence for expression in a cell (FIG. 4B-4C and FIG. 12B). In one embodiment, the promoter sequence is an EFS promoter (FIG. 4B-4C). In one embodiment, the promoter is a EFS/UBB promoter (FIG. 12B). In some embodiments, the promoter sequence comprises an enhancer sequence and/or an intron sequence. In some embodiments, the promoter sequence is a muscle-specific promoter (FIG. 12B).

In some embodiments, the muscle-specific promoter is a desmin promoter as follows:

Sequences for Muscle specific Desmin promoters:

| | |
|---|---|
| Human Desmin (FL) | taccccctgccccccacagctcctc |
| | tcctgtgccttgtttcccagccatg |
| | cgttctcctctataaatacccgctc |
| | tggtatttggggttggcagctgttg |
| | ctgccagggagatggttgggttgac |
| | atgcggctcctgacaaaacacaaac |
| | ccctggtgtgtgtgggcgtgggtgg |
| | tgtgagtagggggatgaatcaggga |
| | gggggcAggggacccagggggcagg |
| | agccacacaaagtctgtgcgggggg |
| | ggagcgcacatagcaattggaaact |
| | gaaagcttatcagaccctttctgga |
| | aatcagcccactgtttataaacttg |
| | aggccccaccctcgacagtaccggg |
| | gaggaagagggcctgcactagtcca |
| | gagggaaactgaggctcagggcCag |
| | ctcgcccatagacatacatggcagg |
| | caggctttggccaggatccctccgc |
| | ctgccaggcgtctccctgccctccc |
| | ttcctgcctagagacccccaccctc |
| | aagcctggctggtctttgcctgaga |
| | cccaaacctcttcgacttcaagaga |
| | atatttaggaacaaggtggtttagg |
| | gcctttcctgggaacaggccttgac |
| | cctttaagaaatgacccaaagtctc |
| | tccttgaccaaaaaggggaccctca |
| | aactaaagggaagcctctcttctgc |
| | tgtctcccctgaccccactcccccc |
| | caccccaggacgaggagataaccag |
| | ggctgaaagaggcccgcctgggggc |
| | tgcagacatgcttgctgcctgccct |
| | ggcgaaggattggcaggcttgcccg |
| | tcacaggacccccgctggctgactc |
| | aggggcgcaggcctTttgcggggga |
| | gctggcctccccgcccccacggcca |
| | cgggccgccctttcctggcaggaca |
| | gcgggatcttgcagctgtcagggga |
| | ggggaggcgggggctgatgtcagga |
| | gggatacaaatagtgccgacggctg |
| | ggggccctGTCTCCCCTCGCCGCAT |
| | CCACTCTCCGGCCGGCCGCCTGCCC |
| | GCCGCCTCCTCCGTGCGCCCGCCAG |
| | CCTCGCCCG |
| | (SEQ ID NO: 568) |
| Human Truncated | ACCCATGCCTCCTCAGGTACCCCCT |
| Desmin (tDesmin) | GCCCCCCACAGCTCCTCTCCTGTGC |
| | CTTGTTTCCCAGCCATGCGTTCTCC |
| | TCTATAAATACCCGCTCTGGTATTT |
| | GGGGTTGGCAGCTGTTGCTGCCAGG |
| | GAGATGGTTGGGTTGACATGCGGCT |
| | CCTGACAAAACACAAACCCCTGGTG |
| | TGTGTGGGCGTGGGTGGTGTGAGTA |
| | GGGGGATGAATCAGGGAGGGGGCaG |
| | GGGACCCAGGGGGCAGGAGCCACAC |
| | AAAGTCTGTGCGGGGGTGGGAGCGC |
| | ACATAGCAATTGGAAACTGAAAGCT |
| | TATCAGACCCTTTCTGGAAATCAGC |
| | CCACTGTTTATAAACTTGAGGCCCC |
| | ACCCTCGAGATAACCAGGGCTGAAA |
| | GAGGCCCGCCTGGGGGCTGCAGACA |
| | TGCTTGCTGCCTGCCCTGGCGAAGG |
| | ATTGGCAGGCTTGCCCGTCACAGGA |
| | CCCCCGCTGGCTGACTCAGGGGCGC |
| | AGGCCTtTTGCGGGGGAGCTGGCCT |
| | CCCCGCCCCCACGGCCACGGGCCGC |
| | CCTTTCCTGGCAGGACAGCGGGATC |
| | TTGCAGCTGTCAGGGGAGGGGAGGC |
| | GGGGGCTGATGTCAGGAGGGATAtA |
| | AATAGTGCCGACGGCTGGGGGCCCT |
| | GTCTCCCCTCGCCGCATCCACTCTC |
| | CGGCCGGCCGCCTGCCCGCCGCCTC |
| | CTCCGTGCGCCCGCCAGCCTCGCCC |
| | GCGCCGTC |
| | (SEQ ID NO: 569) |

In another embodiment, the PUF(CUG) or PUMBY(CUG) or Cas13d(CUG) or dCas13d(CUG) configurations are packaged in an AAV vector. In one embodiment, the AAV vector is an AAV9 vector. In another embodiment, the AAV vector is an AAVrh74 vector.

In another embodiment, the PUF(CUG) or PUMBY(CUG) or Cas13d(CUG) or dCas13d(CUG) configurations are packaged in an AAV vector. In one embodiment, the AAV vector is an AAV9 vector. In another embodiment, the AAV vector is an AAVrh74 vector.

In some embodiments, an AAV vector of the disclosure comprising a CUG-targeting active Cas13d protein comprises from 5′ to 3′ is set forth in Table B. In some aspects, the AAV vector comprises the nucleic acid sequence set forth in SEQ ID NO: 573 or 574.

TABLE B
CUG-Targeting Cas13d AAV vector

| Plasmid | |
|---|---|
| Element | Amino Acid Sequences |
| 5′ ITR | Cctgcaggcagctgcgcgctcgctc |
| | gctcactgaggccgcccgggcgtcg |
| | ggcgacctttggtcgcccggcctca |
| | gtgagcgagcgagcgcgcagagagg |
| | gagtggccaactccatcactagggg |
| | ttcct (SEQ ID NO: 599) |
| Human U6 | GAGGGCCTATTTCCCATGATTCCTT |
| promoter | CATATTTGCATATACGATACAAGGC |
| | TGTTAGAGAGATAATTAGAATTAAT |
| | TTGACTGTAAACACAAAGATATTAG |
| | TACAAAATACGTGACGTAGAAAGTA |
| | ATAATTTCTTGGGTAGTTTGCAGTT |
| | TTAAAATTATGTTTTAAAATGGACT |
| | ATCATATGCTTACCGTAACTTGAAA |
| | GTATTTCGATTTCTTGGCTTTATAT |
| | ATCTTGTGGAAAGGACGAAACACC |
| | (SEQ ID NO: 519) |
| Seq212 direct | gtacaatagccctgcagtaaggcag |
| repeat (DR) | ggttctaAGAC |
| | (SEQ ID NO: 213) |
| Spacer (CTG | CAGCAGCAGCAGCAGCAGCAGCAGC |
| guide 3) | A (SEQ ID NO: 459) |
| human tdesmin | ACCCATGCCTCCTCAGGTACCCCCT |
| promoter | GCCCCCCACAGCTCCTCTCCTGTGC |
| | CTTGTTTCCCAGCCATGCGTTCTCC |
| | TCTATAAATACCCGCTCTGGTATTT |
| | GGGGTTGGCAGCTGTTGCTGCCAGG |
| | GAGATGGTTGGGTTGACATGCGGCT |
| | CCTGACAAAACACAAACCCCTGGTG |
| | TGTGTGGGCGTGGGTGGTGTGAGTA |
| | GGGGGATGAATCAGGGAGGGGGCaG |
| | GGGACCCAGGGGGCAGGAGCCACAC |
| | AAAGTCTGTGCGGGGGTGGGAGCGC |
| | ACATAGCAATTGGAAACTGAAAGCT |
| | TATCAGACCCTTTCTGGAAATCAGC |
| | CCACTGTTTATAAACTTGAGGCCCC |
| | ACCCTCGAGATAACCAGGGCTGAAA |
| | GAGGCCCGCCTGGGGGCTGCAGACA |
| | TGCTTGCTGCCTGCCCTGGCGAAGG |
| | ATTGGCAGGCTTGCCCGTCACAGGA |
| | CCCCCGCTGGCTGACTCAGGGGCGC |
| | AGGCCTtTTGCGGGGGAGCTGGCCT |
| | CCCCGCCCCCACGGCCACGGGCCGC |
| | CCTTTCCTGGCAGGACAGCGGGATC |
| | TTGCAGCTGTCAGGGGAGGGGAGGC |
| | GGGGGCTGATGTCAGGAGGGATAtA |
| | AATAGTGCCGACGGCTGGGGGCCCT |
| | GTCTCCCCTCGCCGCATCCACTCTC |
| | CGGCCGGCCGCCTGCCCGCCGCCTC |
| | CTCCGTGCGCCCGCCAGCCTCGCCC |
| | GCGCCGTC (SEQ ID NO: 569) |
| Kozak Sequence | GCCGCCACCATG |
| | (SEQ ID NO: 529) |
| Cas13d Seq212 | AAGAAGAAGCACCAGAGCGCC |
| | GCCGAGAAGAGGCAAGTGAAGAAGC |
| | TCAAGAATCAAGAGAAGGCCCAGAA |
| | GTACGCTAGCGAGCCTTCCCCCCTC |
| | CAGAGCGATACAGCTGGCGTGGAAT |
| | GCTCCCAGAAAAAGACAGTCGTCAG |
| | CCACATTGCCAGCTCCAAGACACTG |
| | GCCAAGGCTATGGGACTCAAATCCA |
| | CACTGGTCATGGGCGACAAGCTGGT |
| | CATCACCAGCTTTGCTGCTAGCAAG |
| | GCTGTCGGAGGCGCTGGCTACAAAA |
| | GCGCTAACATTGAAAAAATCACAGA |
| | TCTGCAAGGAAGGGTCATTGAGGAG |
| | CACGAAAGGATGTTTAGCGCCGATG |
| | TCGGAGAGAAAAATATCGAACTGAG |
| | CAAGAATGACTGCCACACCAACGTC |
| | AACAACCCCGTGGTGACCAACATCG |
| | GAAAGGATTACATCGGACTGAAATC |
| | TAGGCTGGAGCAAGAGTTTTTCGGC |
| | AAGACATTCGAGAATGACAATCTGC |
| | ATGTGCAGCTGGCCTACAATATCCT |
| | CGACATCAAGAAAATTCTGGGAACC |
| | TATGTGAACAATATCATTTATATCT |
| | TCTACAATCTGAATAGGGCTGGCAC |
| | CGGCAGAGATGAGAGGATGTATGAC |
| | GACCTCATCGGCACACTGTACGCTT |
| | ACAAACCCATGGAGGCTCAACAGAC |
| | CTATCTGCTCAAAGGCGACAAGGAT |
| | ATGAGGAGGTTTGAGGAGGTGAAAC |
| | AGCTGCTGCAAAACACCTCCGCTTA |
| | CTATGTGTATTACGGCACACTGTTC |
| | GAGAAGGTGAAGGCTAAGAGCAAGA |
| | AGGAACAGAGGGCTAAGGAGGCCGA |
| | AATCGACGCTTGTACCGCCCATAAC |
| | TACGATGTGCTGAGACTGCTGTCCC |
| | TCATGAGGCAGCTGTGCATGCACTC |
| | CGTCGCTGGAACAGCCTTTAAGCTG |
| | GCTGAGTCCGCTCTGTTCAACATTG |
| | AGGATGTGCTCAGCGCCGATCTGAA |
| | GGAAATCCTCGATGAAGCCTTCTCC |
| | GGCGCCGTGAACAAGCTCAATGACG |
| | GATTCGTGCAGCACTCCGGCAACAA |
| | TCTGTACGTGCTCCAGCAGCTGTAC |
| | CCTAATGAGACCATCGAGAGAATCG |
| | CCGAGAAGTACTACAGACTCACCGT |
| | GAGGAAGGAGGATCTGAACATGGGA |
| | GTCAACATTAAAAAGCTGAGGGAGC |
| | TGATCGTGGGCCAATACTTTCCCGA |
| | GGTCCTCGACAAAGAATACGACCTC |
| | TCCAAGAATGGAGACAGCGTGGTGA |
| | CATACAGAAGCAAGATTTATACCGT |
| | GATGAATTACATTCTGCTGTATTAC |
| | CTCGAGGACCACGACTCCAGCAGAG |
| | AAAGCATGGTCGAAGCTCTGAGACA |
| | AAACAGAGAGGGCGATGAAGGCAAG |
| | GAGGAGATCTATAGACAGTTTGCCA |
| | AGAAGGTGTGGAACGGCGTGTCCGG |
| | ACTGTTTGGCGTGTGTCTGAACCTC |
| | TTCAAGACCGAAAAGAGAAACAAGT |
| | TTAGGAGCAAAGTCGCCCTCCCCGA |
| | TGTGTCCGGCGCTGCCTATATGCTC |
| | TCCTCCGAGAACATCGACTACTTTG |
| | TCAAGATGCTCTTCTTTGTGTGTAA |
| | GTTTCTGGATGGCAAAGAAATCAAC |
| | GAGCTGCTGTGCGCTCTGATCAACA |
| | AATTTGATAATATTGCCGATATTCT |
| | GGATGCTGCCGCTCAATGTGGCTCC |
| | TCCGTCTGGTTCGTGGACAGCTATA |
| | GGTTCTTCGAGAGATCTAGGAGGAT |
| | TAGCGCCCAGATTAGAATCGTGAAG |
| | AACATCGCTTCCAAGGATTTTAAGA |
| | AATCCAAGAAGGATTCCGATGAGAG |
| | CTACCCCGAGCAGCTGTATCTGGAT |
| | GCTCTGGCTCTGCTCGGAGACGTCA |
| | TCTCCAAGTACAAGCAGAATAGAGA |
| | TGGCAGCGTCGTCATCGATGACCAA |
| | GGCAATGCCGTGCTGACAGAGCAAT |
| | ACAAGAGGTTTAGATATGAATTTTT |
| | CGAGGAGATCAAGAGGGACGAAAGC |
| | GGCGGCATCAAGTACAAGAAGTCCG |
| | GAAAACCCGAGTACAACCATCAGAG |
| | AAGGAATTTTATTCTGAATAATGTG |
| | CTGAAAAGCAAATGGTTTTTCTATG |
| | TGGTGAAGTACAATAGGCCCAGCAG |
| | CTGCAGAGAACTGATGAAGAATAAG |
| | GAAATTCTGAGGTTCGTGCTGAGAG |
| | ACATCCCCGACTCCCAAGTGAGAAG |
| | ATACTTTAAGGCCGTCCAAGGAGAG |
| | GAAGCTTACGCTAGCGCCGAAGCTA |
| | TGAGGACAAGACTGGTCGACGCTCT |
| | GTCCCAATTTAGCGTCACAGCTTGT |
| | CTGGATGAAGTGGGCGGCATGACAG |
| | ACAAGGAATTCGCCTCCCAGAGGGC |
| | CGTCGATAGCAAAGAAAAACTGAGA |
| | GCCATCATCAGACTGTATCTGACAG |
| | TCGCCTATCTGATTACCAAGAGCAT |
| | GGTGAAGGTGAATACAAGGTTTAGC |
| | ATTGCCTTTAGCGTGCTGGAGAGGG |
| | ACTACTATCTGCTCATTGACGGCAA |
| | GAAGAAATCCAGCGACTACACCGGA |
| | GAGGATATGCTGGCTCTGACCAGAA |
| | AATTTGTGGGCGAAGATGCTGGACT |
| | GTATAGAGAGTGGAAAGAGAAGAAC |
| | GCTGAAGCCAAGGACAAATATTTTG |
| | ACAAGGCCGAAAGGAAGAAGGTGCT |
| | GAGACAGAACGATAAGATGATCAGA |
| | AAGATGCACTTCACACCCCACTCCC |
| | TCAATTACGTCCAAAAGAATCTCGA |
| | AAGCGTCCAGAGCAACGGACTGGCC |
| | GCCGTCATCAAGGAATATAGAAATG |
| | CCGTCGCTCACCTCAATATCATCAA |
| | TAGACTGGACGAGTACATTGGCTCC |
| | GCTAGGGCTGATAGCTACTACTCTC |
| | TGTACTGTTACTGCCTCCAAATGTA |
| | TCTGAGCAAGAACTTCAGCGTGGGC |
| | TACCTCATCAACGTGCAAAAGCAGC |
| | TGGAGGAGCACCACACCTACATGAA |
| | GGATCTCATGTGGCTGCTCAACATC |
| | CCCTTCGCTTACAACCTCGCCAGAT |
| | ACAAAAATCTGTCCAACGAAAAACT |
| | CTTTTACGACGAGGAAGCCGCCGCC |
| | GAAAAGGCTGACAAGGCTGAGAACG |
| | AGAGAGGCGAA |
| | (SEQ ID NO: 601) |
| Linker | GGAAGC |
| SV-40 NLS | CCCAAGAAGAAAAGGAAGGTC |
| | (SEQ ID NO: 532) |
| Linker | GGCGGAtct |
| Myc Tag | GAGCAgAAACTGATTAGcGAAGAgG |
| | ATCTC (SEQ ID NO: 602) |
| WPRE3 | GATAATCAACCTCTGGATTACAAAA |
| | TTTGTGAAAGATTGACTGGTATTCT |
| | TAACTATGTTGCTCCTTTTACGCTA |
| | TGTGGATACGCTGCTTTAATGCCTT |
| | TGTATCATGCTATTGCTTCCCGTAT |
| | GGCTTTCATTTTCTCCTCCTTGTAT |
| | AAATCCTGGTTAGTTCTTGCCACGG |
| | CGGAACTCATCGCCGCCTGCCTTGC |
| | CCGCTGCTGGACAGGGGCTCGGCTG |
| | TTGGGCACTGACAATTCCGTGG |
| | (SEQ ID NO: 603) |
| SV-40 poly A | AACTTGTTTATTGCAGCTTATAATG |
| | GTTACAAATAAAGCAATAGCATCAC |
| | AAATTTCACAAATAAAGCATTTTTT |
| | TCACTGCATTCTAGTTGTGGTTTGT |
| | CCAAACTCATCAATGTATCTTA |
| | (SEQ ID NO: 533) |
| 3′ITR | Aggaacccctagtgatggagttggc |
| | cactccctctctgcgcgctcgctcg |
| | ctcactgaggccgggcgaccaaagg |
| | tcgcccgacgcccgggctttgcccg |
| | ggcggcctcagtgagcgagcgagcg |
| | cgcagctgcctgcagg |
| | (SEQ ID NO: 600) |

In some embodiments, an AAV vector of the disclosure, referred to as A02205, comprising a CUG-targeting PUF protein fused to an endonuclease comprises from 5′ to 3′ the elements as set forth in Table C. In some aspects, the AAV vector comprises the nucleic acid sequence set forth in SEQ ID NO: 574 or 575.

TABLE C
Exemplary CUG-Targeting PUF-endonuclease AAV vector A02205

| Plasmid | |
|---|---|
| Element | Amino Acid Sequences |
| 5′ITR | CCTGCAGGCAGCTGCGCGCTCGCTCGCTCACTGAGGCCGCCCGGG |
| | CGTCGGGCGACCTTTGGTCGCCCGGCCTCAGTGAGCGAGCGAGCG |
| | CGCAGAGAGGGAGTGGCCAACTCCATCACTAGGGGTTCCT (SEQ ID |
| | NO: 599) |
| Human | taccccctgccccccacagctcctctcctgtgccttgtttcccagccatgcgttctcctctataaatacccgctctg |
| Desmin | gtatttggggttggcagctgttgctgccagggagatggttgggttgacatgcggctcctgacaaaacacaaacc |
| Promoter | cctggtgtgtgtgggcgtgggtggtgtgagtagggggatgaatcagggagggggcAggggacccagggg |
| (FL) | gcaggagccacacaaagtctgtgcggggggggagcgcacatagcaattggaaactgaaagcttatcagac |
| | cctttctggaaatcagcccactgtttataaacttgaggccccaccctcgacagtaccggggaggaagagggcc |
| | tgcactagtccagagggaaactgaggctcagggcCagctcgcccatagacatacatggcaggcaggctttg |
| | gccaggatccctccgcctgccaggcgtctccctgccctcccttcctgcctagagacccccaccctcaagcctg |
| | gctggtctttgcctgagacccaaacctcttcgacttcaagagaatatttaggaacaaggtggtttagggcctttcc |
| | tgggaacaggccttgaccctttaagaaatgacccaaagtctctccttgaccaaaaaggggaccctcaaactaaa |
| | gggaagcctctcttctgctgtctcccctgaccccactcccccccaccccaggacgaggagataaccagggct |
| | gaaagaggcccgcctgggggctgcagacatgcttgctgcctgccctggcgaaggattggcaggcttgcccg |
| | tcacaggacccccgctggctgactcaggggcgcaggcctTttgcgggggagctggcctccccgcccccac |
| | ggccacgggccgccctttcctggcaggacagcgggatcttgcagctgtcaggggaggggaggcgggggct |
| | gatgtcaggagggatacaaatagtgccgacggctgggggccctGTCTCCCCTCGCCGCATC |
| | CACTCTCCGGCCGGCCGCCTGCCCGCCGCCTCCTCCGTGCGCCCGC |
| | CAGCCTCGCCCG (SEQ ID NO: 608) |
| Kozak | gccgccACCatg (SEQ ID NO: 529) |
| Rb-NLS | Gaccgtgtgctcaaaagaagtgctgaaggaagcaaccctcctaaaccactgaaaaaactacgc (SEQ ID NO: |
| | 604) |
| Linker | ggaGGATCG |
| 8PUF | GGACGAAGCCGACTCTTGGAAGACTTCAGAAACAATCGGTATCCG |
| | AACCTTCAGCTGAGAGAAATTGCTGGTCACATCATGGAATTTTCTC |
| | AAGATCAACATGGAAGCCGGTTTATTGAACTTAAACTCGAACGAG |
| | CCACCCCGGCCGAAAGGCAATTGGTGTTCAATGAAATTCTTCAGG |
| | CCGCATACCAACTCATGGTTGATGTTTTTGGGAACTATGTTATTCA |
| | AAAGTTTTTTGAGTTCGGGTCACTGGAGCAAAAGTTGGCATTGGC |
| | AGAGCGAATCCGGGGCCATGTTCTGAGCCTCGCTCTCCAAATGTA |
| | CGGTAGTTATGTCATTCGCAAAGCACTCGAGTTCATACCATCAGAT |
| | CAACAGAATGAGATGGTGCGGGAGCTGGATGGGCATGTTTTGAAA |
| | TGCGTGAAAGACCAAAACGGTAGCTACGTAGTTGAGAAATGCATC |
| | GAATGCGTCCAACCACAGTCTCTCCAATTTATTATAGATGCATTTA |
| | AGGGTCAGGTTTTCGCGCTTTCTACGCACCCGTATGGGAACCGAGT |
| | GATTCAGAGAATCTTGGAGCACTGCCTGCCGGATCAGACACTCCC |
| | TATCTTGGAGGAATTGCACCAGCATACCGAACAATTGGTGCAAGA |
| | TCAATACGGTTCATATGTTATTCGGCACGTTCTTGAGCATGGAAGG |
| | CCAGAGGACAAGTCAAAGATCGTCGCTGAGATTAGAGGTAACGTA |
| | TTGGTGCTCTCACAACACAAATTTGCATCTAATGTGGTGGAGAAAT |
| | GTGTTACTCATGCTTCTAGAACGGAAAGGGCAGTTCTCATAGACG |
| | AAGTTTGCACAATGAATGATGGTCCTCATAGCGCACTTTATACCAT |
| | GATGAAGGACCAGTATGCAAACTATGTCGTCCAGAAAATGATCGA |
| | TGTGGCGGAGCCCGGTCAACGGAAAATCGTGATGCACAAAATCCG |
| | ACCTCACATTGCTACACTCAGAAAATACACGTATGGAAAACATAT |
| | TCTGGCTAAGCTGGAGAAATATTACATGAAGAATGGAGTGGATCT |
| | GGGG (SEQ ID NO: 452) |
| Linker | GTGGATACTGCCAATGGCAGC (SEQ ID NO: 605) |
| E17 | Ggtggtggcacccctaaggctcccaacctggagcctccactcccagaagaggaaaaggagggcagcgac |
| | ctgagaccagtggtcatcgatgggagcaacgtggccatgagccatgggaacaaggaggtcttctcctgccgg |
| | ggcatcctgctggcagtgaactggtttctggagcggggccacacagacatcacagtgtttgtgccatcctgga |
| | ggaaggagcagcctcggcccgacgtgcccatcacagaccagcacatcctgcgggaactggagaagaaga |
| | agatcctggtgttcacaccatcacgacgcgtgggtggcaagcgggtggtgtgctatgacgacagattcattgtg |
| | aagctggcctacgagtctgacgggatcgtggtttccaacgacacataccgtgacctccaaggcgagcggcag |
| | gagtggaagcgcttcatcgaggagcggctgctcatgtactccttcgtcaatgacaagtttatgccccctgatgac |
| | ccactgggccggcacgggcccagcctggacaacttcctgcgtaagaagccactcactttggag (SEQ ID |
| | NO: 606) |
| WPRE | Aatcaacctctggattacaaaatttgtgaaagattgactggtattcttaactatgttgctccttttacgctatgtggat |
| | acgctgctttaatgcctttgtatcatgctattgcttcccgtatggctttcattttctcctccttgtataaatcctggtt |
| | gctgtctctttatgaggagttgtggcccgttgtcaggcaacgtggcgtggtgtgcactgtgtttgctgacgcaacccc |
| | cactggttggggcattgccaccacctgtcagctcctttccgggactttcgctttccccctccctattgccacggcg |
| | gaactcatcgccgcctgccttgcccgctgctggacaggggctcggctgttgggcactgacaattccgtggtgtt |
| | gtcggggaaatcatcgtcctttccttggctgctcgcctgtgttgccacctggattctgcgcgggacgtccttctgc |
| | tacgtcccttcggccctcaatccagcggaccttccttcccgcggcctgctgccggctctgcggcctcttccgcg |
| | tcttcgccttcgccctcagacgagtcggatctccctttgggccgcctccccgc (SEQ ID NO: 607) |
| SV-40 | AACTTGTTTATTGCAGCTTATAATGGTTACAAATAAAGCAATAGCA |
| poly A | TCACAAATTTCACAAATAAAGCATTTTTTTCACTGCATTCTAGTTG |
| | TGGTTTGTCCAAACTCATCAATGTATCTTA (SEQ ID NO: 533) |
| 3′ITR | AGGAACCCCTAGTGATGGAGTTGGCCACTCCCTCTCTGCGCGCTCG |
| | CTCGCTCACTGAGGCCGGGCGACCAAAGGTCGCCCGACGCCCGGG |
| | CTTTGCCCGGGCGGCCTCAGTGAGCGAGCGAGCGCGCAGCTGCCT |
| | GCAGG (SEQ ID NO: 600) |

PUF-CUG No Endonuclease with Desmin (FL) Promoter (A02239)

In some embodiments, the disclosure provides a CUG-targeting PUF protein fused to an RB NLS as set forth in Table D.

TABLE D
Exemplary CUG-Targeting 8PUF

| Plasmid | |
|---|---|
| Element | Amino Acid Sequences |
| Rb-NLS | DRVLKRSAEGSNPPKPLKKLR (SEQ ID NO: 543) |
| Linker | GGS |
| 8PUF | GRSRLLEDFRNNRYPNLQLREIAGHIMEFSQDQHGSRFIELKLERATPAERQL |
| | VFNEILQAAYQLMVDVFGNYVIQKFFEFGSLEQKLALAERIRGHVLSLALOM |
| | YGSYVIRKALEFIPSDQQNEMVRELDGHVLKCVKDQNGSYVVEKCIECVQP |
| | QSLQFIIDAFKGQVFALSTHPYGNRVIQRILEHCLPDQTLPILEELHQHTEQLV |
| | QDQYGSYVIRHVLEHGRPEDKSKIVAEIRGNVLVLSQHKFASNVVEKCVTH |
| | ASRTERAVLIDEVCTMNDGPHSALYTMMKDQYANYVVQKMIDVAEPGQR |
| | KIVMHKIRPHIATLRKYTYGKHILAKLEKYYMKNGVDLG (SEQ ID NO: 444) |

In some embodiments, an AAV vector of the disclosure, referred to as A02239, comprising a CUG-targeting PUF protein comprises from 5′ to 3′ the elements as set forth in Table E. In some aspects, the AAV vector comprises the nucleic acid sequence set forth in SEQ ID NO: 576 or 577.

TABLE E
Exemplary CUG-Targeting PUF AAV vector A02239

| Plasmid | |
|---|---|
| Element | Amino Acid Sequences |
| 5′ITR | CCTGCAGGCAGCTGCGCGCTCGCTCGCTCACTGAGGCCGCCCGGG |
| | CGTCGGGCGACCTTTGGTCGCCCGGCCTCAGTGAGCGAGCGAGCG |
| | CGCAGAGAGGGAGTGGCCAACTCCATCACTAGGGGTTCCT (SEQ ID |
| | NO: 599) |
| Human | taccccctgccccccacagctcctctcctgtgccttgtttcccagccatgogttctcctctataaatacccgctctg |
| Desmin | gtatttggggttggcagctgttgctgccagggagatggttgggttgacatgcggctcctgacaaaacacaaacc |
| Promoter | cctggtgtgtgtgggcgtgggtggtgtgagtagggggatgaatcagggagggggcAggggacccagggg |
| (FL) | gcaggagccacacaaagtctgtgcggggggggagcgcacatagcaattggaaactgaaagcttatcagac |
| | cctttctggaaatcagcccactgtttataaacttgaggccccaccctcgacagtaccggggaggaagagggcc |
| | tgcactagtccagagggaaactgaggctcagggcCagctcgcccatagacatacatggcaggcaggctttg |
| | gccaggatccctccgcctgccaggcgtctccctgccctcccttcctgcctagagacccccaccctcaagcctg |
| | gctggtctttgcctgagacccaaacctcttcgacttcaagagaatatttaggaacaaggtggtttagggcctttcc |
| | tgggaacaggccttgaccctttaagaaatgacccaaagtctctccttgaccaaaaaggggaccctcaaactaaa |
| | gggaagcctctcttctgctgtctcccctgaccccactcccccccaccccaggacgaggagataaccagggct |
| | gaaagaggcccgcctgggggctgcagacatgcttgctgcctgccctggcgaaggattggcaggcttgcccg |
| | tcacaggacccccgctggctgactcaggggcgcaggcctTttgcgggggagctggcctccccgcccccac |
| | ggccacgggccgccctttcctggcaggacagcgggatcttgcagctgtcaggggggggaggcgggggct |
| | gatgtcaggagggatacaaatagtgccgacggctgggggccctGTCTCCCCTCGCCGCATC |
| | CACTCTCCGGCCGGCCGCCTGCCCGCCGCCTCCTCCGTGCGCCCGC |
| | CAGCCTCGCCCG (SEQ ID NO: 608) |
| Kozak | gccgccACCatg (SEQ ID NO: 529) |
| Rb-NLS | Gaccgtgtgctcaaaagaagtgctgaaggaagcaaccctcctaaaccactgaaaaaactacgc (SEQ ID NO: |
| | 604) |
| Linker | ggaGGATCG |
| 8PUF | GGACGAAGCCGACTCTTGGAAGACTTCAGAAACAATCGGTATCCG |
| | AACCTTCAGCTGAGAGAAATTGCTGGTCACATCATGGAATTTTCTC |
| | AAGATCAACATGGAAGCCGGTTTATTGAACTTAAACTCGAACGAG |
| | CCACCCCGGCCGAAAGGCAATTGGTGTTCAATGAAATTCTTCAGG |
| | CCGCATACCAACTCATGGTTGATGTTTTTGGGAACTATGTTATTCA |
| | AAAGTTTTTTGAGTTCGGGTCACTGGAGCAAAAGTTGGCATTGGC |
| | AGAGCGAATCCGGGGCCATGTTCTGAGCCTCGCTCTCCAAATGTA |
| | CGGTAGTTATGTCATTCGCAAAGCACTCGAGTTCATACCATCAGAT |
| | CAACAGAATGAGATGGTGCGGGAGCTGGATGGGCATGTTTTGAAA |
| | TGCGTGAAAGACCAAAACGGTAGCTACGTAGTTGAGAAATGCATC |
| | GAATGCGTCCAACCACAGTCTCTCCAATTTATTATAGATGCATTTA |
| | AGGGTCAGGTTTTCGCGCTTTCTACGCACCCGTATGGGAACCGAGT |
| | GATTCAGAGAATCTTGGAGCACTGCCTGCCGGATCAGACACTCCC |
| | TATCTTGGAGGAATTGCACCAGCATACCGAACAATTGGTGCAAGA |
| | TCAATACGGTTCATATGTTATTCGGCACGTTCTTGAGCATGGAAGG |
| | CCAGAGGACAAGTCAAAGATCGTCGCTGAGATTAGAGGTAACGTA |
| | TTGGTGCTCTCACAACACAAATTTGCATCTAATGTGGTGGAGAAAT |
| | GTGTTACTCATGCTTCTAGAACGGAAAGGGCAGTTCTCATAGACG |
| | AAGTTTGCACAATGAATGATGGTCCTCATAGCGCACTTTATACCAT |
| | GATGAAGGACCAGTATGCAAACTATGTCGTCCAGAAAATGATCGA |
| | TGTGGCGGAGCCCGGTCAACGGAAAATCGTGATGCACAAAATCCG |
| | ACCTCACATTGCTACACTCAGAAAATACACGTATGGAAAACATAT |
| | TCTGGCTAAGCTGGAGAAATATTACATGAAGAATGGAGTGGATCT |
| | GGGG (SEQ ID NO: 452) |
| WPRE | Aatcaacctctggattacaaaatttgtgaaagattgactggtattcttaactatgttgctccttttacgctatgtggat |
| | acgctgctttaatgcctttgtatcatgctattgcttcccgtatggctttcattttctcctccttgtataaatcctggtt |
| | gctgtctctttatgaggagttgtggcccgttgtcaggcaacgtggcgtggtgtgcactgtgtttgctgacgcaacccc |
| | cactggttggggcattgccaccacctgtcagctcctttccgggactttcgctttccccctccctattgccacggcg |
| | gaactcatcgccgcctgccttgcccgctgctggacaggggctcggctgttgggcactgacaattccgtggtgtt |
| | gtcggggaaatcatcgtcctttccttggctgctcgcctgtgttgccacctggattctgcgcgggacgtccttctgc |
| | tacgtcccttcggccctcaatccagcggaccttccttcccgcggcctgctgccggctctgcggcctcttccgcg |
| | tcttcgccttcgccctcagacgagtcggatctccctttgggccgcctccccgc (SEQ ID NO: 607) |
| SV-40 | AACTTGTTTATTGCAGCTTATAATGGTTACAAATAAAGCAATAGCA |
| poly A | TCACAAATTTCACAAATAAAGCATTTTTTTCACTGCATTCTAGTTG |
| | TGGTTTGTCCAAACTCATCAATGTATCTTA (SEQ ID NO: 533) |
| 3′ITR | AGGAACCCCTAGTGATGGAGTTGGCCACTCCCTCTCTGCGCGCTCG |
| | CTCGCTCACTGAGGCCGGGCGACCAAAGGTCGCCCGACGCCCGGG |
| | CTTTGCCCGGGCGGCCTCAGTGAGCGAGCGAGCGCGCAGCTGCCT |
| | GCAGG (SEQ ID NO: 600) |

### Exemplary Blocking RNA-Targeting Compositions

CTG microsatellite expansions in the non-coding 3′ untranslated region of DMPK cause DM1. Expanded CUG (CUG^{exp}) repeats in DMPK mRNA directly sequester MBNL proteins causing loss of their function. MBNL loss of function is directly responsible for alternative splicing defects and clinical manifestations observed in DM1. PUF(CUG) or dCas13d(CUG) will bind CUG^{exp} RNA directly and block MBNL sequestration to preserve near normal free MBNL levels and function that will reverse DM1 disease phenotypes such as splicing dysfunction, myotonia and others. The following PUF(CUG) and dCas13d(CUG) RNA-targeting constructs are exemplary embodiments for blocking.

In some embodiments, the disclosure provides a CUG-targeting PUF protein fused to an RB NLS as set forth in Table F.

TABLE F
Exemplary CUG-Targeting 8PUF for blocking CUG RNA

| Plasmid Element | Amino Acid Sequences |
|---|---|
| Rb-NLS | DRVLKRSAEGSNPPKPLKKLR (SEQ ID NO: 543) |
| Linker | GGS |
| 8PUF | GRSRLLEDFRNNRYPNLQLREIAGHIMEFSQDQHGSRFIELKL |
| | ERATPAERQLVFNEILQAAYQLMVDVFGNYVIQKFFEFGSLE |
| | QKLALAERIRGHVLSLALQMYGSYVIRKALEFIPSDQQNEMV |
| | RELDGHVLKCVKDQNGSYVVEKCIECVQPQSLQFIIDAFKGQ |
| | VFALSTHPYGNRVIQRILEHCLPDQTLPILEELHQHTEQLVQD |
| | QYGSYVIRHVLEHGRPEDKSKIVAEIRGNVLVLSQHKFASNV |
| | VEKCVTHASRTERAVLIDEVCTMNDGPHSALYTMMKDQYA |
| | NYVVQKMIDVAEPGQRKIVMHKIRPHIATLRKYTYGKHILA |
| | KLEKYYMKNGVDLG (SEQ ID NO: 444) |

In some embodiments, an AAV vector of the disclosure comprising a CUG-targeting PUF protein suitable for blocking comprises from 5′ to 3′ the elements as set forth in Table G. In some aspects, the AAV vector comprises the nucleic acid sequence set forth in SEQ ID NO: 578 or 579.

TABLE G
Exemplary CUG-Targeting blocking PUF
Nucleotide sequences of elements in order N-terminal to C-terminal

| Plasmid Element | Amino Acid Sequences |
|---|---|
| 5′ITR | CCTGCAGGCAGCTGCGCGCTCGCTCGCTCACTGAGG |
| | CCGCCCGGGCGTCGGGCGACCTTTGGTCGCCCGGCCT |
| | CAGTGAGCGAGCGAGCGCGCAGAGAGGGAGTGGCC |
| | AACTCCATCACTAGGGGTTCCT (SEQ ID NO: 599) |
| EFS/UBB Promoter | GGGCAGAGCGCACATCGCCCACAGTCCCCGAGAAGT |
| | TGGGGGGAGGGGTCGGCAATTGAaCCGGTGCCTAGA |
| | GAAGGTGGCGCGGGGTAAACTGGGAAAGTGATGTCG |
| | TGTACTGGCTCCGCCTTTTTCCCGAGGGTGGGGGAGA |
| | ACCGTATATAAGTGCAGTAGTCGCCGTGAACGTTCTT |
| | TTTCGCAACGGGTTTGCCGCCAGAACACAGaattccagGT |
| | AAGTCCCGCAGCCGTAACGACCTTGGGGGGGTGTGA |
| | GATTCTCATTCTAATTTTGAAGAATATTAGGTGTAAA |
| | AGCAAGAAATACAATGATCCTGAGGTGACACGCTTA |
| | TGTTTTACTTTTAAACTAGGT (SEQ ID NO: 609) |
| Kozak | gccgccACCatg (SEQ ID NO: 529) |
| Rb-NLS | Gaccgtgtgctcaaaagaagtgctgaaggaagcaaccctcctaaaccactgaaaaaactacgc |
| | (SEQ ID NO: 604) |
| Linker | ggaGGATCG |
| 8PUF | GGACGAAGCCGACTCTTGGAAGACTTCAGAAACAAT |
| | CGGTATCCGAACCTTCAGCTGAGAGAAATTGCTGGT |
| | CACATCATGGAATTTTCTCAAGATCAACATGGAAGC |
| | CGGTTTATTGAACTTAAACTCGAACGAGCCACCCCG |
| | GCCGAAAGGCAATTGGTGTTCAATGAAATTCTTCAG |
| | GCCGCATACCAACTCATGGTTGATGTTTTTGGGAACT |
| | ATGTTATTCAAAAGTTTTTTGAGTTCGGGTCACTGGA |
| | GCAAAAGTTGGCATTGGCAGAGCGAATCCGGGGCCA |
| | TGTTCTGAGCCTCGCTCTCCAAATGTACGGTAGTTAT |
| | GTCATTCGCAAAGCACTCGAGTTCATACCATCAGATC |
| | AACAGAATGAGATGGTGCGGGAGCTGGATGGGCATG |
| | TTTTGAAATGCGTGAAAGACCAAAACGGTAGCTACG |
| | TAGTTGAGAAATGCATCGAATGCGTCCAACCACAGT |
| | CTCTCCAATTTATTATAGATGCATTTAAGGGTCAGGT |
| | TTTCGCGCTTTCTACGCACCCGTATGGGAACCGAGTG |
| | ATTCAGAGAATCTTGGAGCACTGCCTGCCGGATCAG |
| | ACACTCCCTATCTTGGAGGAATTGCACCAGCATACCG |
| | AACAATTGGTGCAAGATCAATACGGTTCATATGTTAT |
| | TCGGCACGTTCTTGAGCATGGAAGGCCAGAGGACAA |
| | GTCAAAGATCGTCGCTGAGATTAGAGGTAACGTATT |
| | GGTGCTCTCACAACACAAATTTGCATCTAATGTGGTG |
| | GAGAAATGTGTTACTCATGCTTCTAGAACGGAAAGG |
| | GCAGTTCTCATAGACGAAGTTTGCACAATGAATGAT |
| | GGTCCTCATAGCGCACTTTATACCATGATGAAGGACC |
| | AGTATGCAAACTATGTCGTCCAGAAAATGATCGATG |
| | TGGCGGAGCCCGGTCAACGGAAAATCGTGATGCACA |
| | AAATCCGACCTCACATTGCTACACTCAGAAAATACA |
| | CGTATGGAAAACATATTCTGGCTAAGCTGGAGAAAT |
| | ATTACATGAAGAATGGAGTGGATCTGGGG (SEQ ID NO: |
| | 452) |
| WPRE | Aatcaacctctggattacaaaatttgtgaaagattgactggtattcttaactatgttgctcct |
| | tttacgctatgtggatacgctgctttaatgcctttgtatcatgctattgcttcccgtatggcttt |
| | cattttctcctccttgtataaatcctggttgctgtctctttatgaggagttgtggcccgttgtca |
| | ggcaacgtggcgtggtgtgcactgtgtttgctgacgcaacccccactggttggggcatt |
| | gccaccacctgtcagctcctttccgggactttcgctttccccctccctattgccacggcgg |
| | aactcatcgccgcctgccttgcccgctgctggacaggggctcggctgttgggcactga |
| | caattccgtggtgttgtcggggaaatcatcgtcctttccttggctgctcgcctgtgttgcca |
| | cctggattctgcgcgggacgtccttctgctacgtcccttcggccctcaatccagcggacc |
| | ttccttcccgcggcctgctgccggctctgcggcctcttccgcgtcttcgccttcgccctca |
| | gacgagtcggatctccctttgggccgcctccccgc (SEQ ID NO: 607) |
| SV-40 poly A | AACTTGTTTATTGCAGCTTATAATGGTTACAAATAAA |
| | GCAATAGCATCACAAATTTCACAAATAAAGCATTTTT |
| | TTCACTGCATTCTAGTTGTGGTTTGTCCAAACTCATC |
| | AATGTATCTTA (SEQ ID NO: 533) |
| 3′ITR | AGGAACCCCTAGTGATGGAGTTGGCCACTCCCTCTCT |
| | GCGCGCTCGCTCGCTCACTGAGGCCGGGCGACCAAA |
| | GGTCGCCCGACGCCCGGGCTTTGCCCGGGCGGCCTC |
| | AGTGAGCGAGCGAGCGCGCAGCTGCCTGCAGG (SEQ ID |
| | NO: 600) |

PUF Targeting CUG (DM1) No Endonuclease with Myc Tag

In some embodiments, the disclosure provides a CUG-targeting PUF protein fused to an RB NLS as set forth in Table H.

TABLE H
Exemplary CUG-Targeting 8PUF for blocking CUG RNA

| Plasmid Element | Amino Acid Sequences |
|---|---|
| Rb-NLS | DRVLKRSAEGSNPPKPLKKLR (SEQ ID NO: 543) |
| Linker | GGS |
| 8PUF | GRSRLLEDFRNNRYPNLQLREIAGHIMEFSQDQHGSRFIELKL |
| | ERATPAERQLVFNEILQAAYQLMVDVFGNYVIQKFFEFGSLE |
| | QKLALAERIRGHVLSLALQMYGSYVIRKALEFIPSDQQNEMV |
| | RELDGHVLKCVKDQNGSYVVEKCIECVQPQSLQFIIDAFKGQ |
| | VFALSTHPYGNRVIQRILEHCLPDQTLPILEELHQHTEQLVQD |
| | QYGSYVIRHVLEHGRPEDKSKIVAEIRGNVLVLSQHKFASNV |
| | VEKCVTHASRTERAVLIDEVCTMNDGPHSALYTMMKDQYA |
| | NYVVQKMIDVAEPGQRKIVMHKIRPHIATLRKYTYGKHILA |
| | KLEKYYMKNGVDLG (SEQ ID NO: 444) |
| Linker | GGS |
| Myc tag | EQKLISEEDL (SEQ ID NO: 610) |

In some embodiments, an AAV vector of the disclosure comprising a CUG-targeting PUF protein suitable for blocking comprises from 5′ to 3′ the elements as set forth in Table I. In some aspects, the AAV vector comprises the nucleic acid sequence set forth in SEQ ID NO: 581 or 582.

TABLE I
Exemplary AAV vector comprising a CUG-Targeting blocking PUF

| Plasmid Element | Amino Acid Sequences |
|---|---|
| 5′ITR | CCTGCAGGCAGCTGCGCGCTCGCTCGCTCACTGAGG |
| | CCGCCCGGGCGTCGGGCGACCTTTGGTCGCCCGGCCT |
| | CAGTGAGCGAGCGAGCGCGCAGAGAGGGAGTGGCC |
| | AACTCCATCACTAGGGGTTCCT (SEQ ID NO: 599) |
| EFS/UBB Promoter | GGGCAGAGCGCACATCGCCCACAGTCCCCGAGAAGT |
| | TGGGGGGAGGGGTCGGCAATTGAaCCGGTGCCTAGA |
| | GAAGGTGGCGCGGGGTAAACTGGGAAAGTGATGTCG |
| | TGTACTGGCTCCGCCTTTTTCCCGAGGGTGGGGGAGA |
| | ACCGTATATAAGTGCAGTAGTCGCCGTGAACGTTCTT |
| | TTTCGCAACGGGTTTGCCGCCAGAACACAGaattccagGT |
| | AAGTCCCGCAGCCGTAACGACCTTGGGGGGGTGTGA |
| | GATTCTCATTCTAATTTTGAAGAATATTAGGTGTAAA |
| | AGCAAGAAATACAATGATCCTGAGGTGACACGCTTA |
| | TGTTTTACTTTTAAACTAGGT (SEQ ID NO: 609) |
| Kozak | gccgccACCatg (SEQ ID NO: 529) |
| Rb-NLS | Gaccgtgtgctcaaaagaagtgctgaaggaagcaaccctcctaaaccactgaaaaaactacgc |
| | (SEQ ID NO: 604) |
| Linker | ggaGGATCG |
| 8PUF | GGACGAAGCCGACTCTTGGAAGACTTCAGAAACAAT |
| | CGGTATCCGAACCTTCAGCTGAGAGAAATTGCTGGT |
| | CACATCATGGAATTTTCTCAAGATCAACATGGAAGC |
| | CGGTTTATTGAACTTAAACTCGAACGAGCCACCCCG |
| | GCCGAAAGGCAATTGGTGTTCAATGAAATTCTTCAG |
| | GCCGCATACCAACTCATGGTTGATGTTTTTGGGAACT |
| | ATGTTATTCAAAAGTTTTTTGAGTTCGGGTCACTGGA |
| | GCAAAAGTTGGCATTGGCAGAGCGAATCCGGGGCCA |
| | TGTTCTGAGCCTCGCTCTCCAAATGTACGGTAGTTAT |
| | GTCATTCGCAAAGCACTCGAGTTCATACCATCAGATC |
| | AACAGAATGAGATGGTGCGGGAGCTGGATGGGCATG |
| | TTTTGAAATGCGTGAAAGACCAAAACGGTAGCTACG |
| | TAGTTGAGAAATGCATCGAATGCGTCCAACCACAGT |
| | CTCTCCAATTTATTATAGATGCATTTAAGGGTCAGGT |
| | TTTCGCGCTTTCTACGCACCCGTATGGGAACCGAGTG |
| | ATTCAGAGAATCTTGGAGCACTGCCTGCCGGATCAG |
| | ACACTCCCTATCTTGGAGGAATTGCACCAGCATACCG |
| | AACAATTGGTGCAAGATCAATACGGTTCATATGTTAT |
| | TCGGCACGTTCTTGAGCATGGAAGGCCAGAGGACAA |
| | GTCAAAGATCGTCGCTGAGATTAGAGGTAACGTATT |
| | GGTGCTCTCACAACACAAATTTGCATCTAATGTGGTG |
| | GAGAAATGTGTTACTCATGCTTCTAGAACGGAAAGG |
| | GCAGTTCTCATAGACGAAGTTTGCACAATGAATGAT |
| | GGTCCTCATAGCGCACTTTATACCATGATGAAGGACC |
| | AGTATGCAAACTATGTCGTCCAGAAAATGATCGATG |
| | TGGCGGAGCCCGGTCAACGGAAAATCGTGATGCACA |
| | AAATCCGACCTCACATTGCTACACTCAGAAAATACA |
| | CGTATGGAAAACATATTCTGGCTAAGCTGGAGAAAT |
| | ATTACATGAAGAATGGAGTGGATCTGGGG (SEQ ID |
| | NO: 452) |
| Linker | GGCGGAAGT (SEQ ID NO: 611) |
| Myc tag | GAGCAAAAACTGATTAGTGAAGAAGATCTC |
| WPRE | Aatcaacctctggattacaaaatttgtgaaagattgactggtattcttaactatgttgctcct |
| | tttacgctatgtggatacgctgctttaatgcctttgtatcatgctattgcttcccgtatggcttt |
| | cattttctcctccttgtataaatcctggttgctgtctctttatgaggagttgtggcccgttgtca |
| | ggcaacgtggcgtggtgtgcactgtgtttgctgacgcaacccccactggttggggcatt |
| | gccaccacctgtcagctcctttccgggactttcgctttccccctccctattgccacggcgg |
| | aactcatcgccgcctgccttgcccgctgctggacaggggctcggctgttgggcactga |
| | caattccgtggtgttgtcggggaaatcatcgtcctttccttggctgctcgcctgtgttgcca |
| | cctggattctgcgcgggacgtccttctgctacgtcccttcggccctcaatccagcggacc |
| | ttccttcccgcggcctgctgccggctctgcggcctcttccgcgtcttcgccttcgccctca |
| | gacgagtcggatctccctttgggccgcctccccgc (SEQ ID NO: 607) |
| SV-40 poly A | AACTTGTTTATTGCAGCTTATAATGGTTACAAATAAA |
| | GCAATAGCATCACAAATTTCACAAATAAAGCATTTTT |
| | TTCACTGCATTCTAGTTGTGGTTTGTCCAAACTCATC |
| | AATGTATCTTA (SEQ ID NO: 533) |
| 3′ITR | AGGAACCCCTAGTGATGGAGTTGGCCACTCCCTCTCT |
| | GCGCGCTCGCTCGCTCACTGAGGCCGGGCGACCAAA |
| | GGTCGCCCGACGCCCGGGCTTTGCCCGGGCGGCCTC |
| | AGTGAGCGAGCGAGCGCGCAGCTGCCTGCAGG (SEQ ID |
| | NO: 600) |

A01560: dCas13d dSeq212 Targeting CUG No Endonuclease (4 Point Mutations on Catalytic Domain)

In some embodiments, the disclosure provides a CUG-targeting catalytically inactive Cas (dCas13d) having 4 point mutations as set forth in Table J.

TABLE J
Exemplary CUG-Targeting dCas13d for blocking CUG RNA

| Plasmid Element | Amino Acid Sequences |
|---|---|
| dCas13d Seq212 | KKKHQSAAEKRQVKKLKNQEKAQKYASEPSPLQSDTAGVECSQKKTVVS |
| | HIASSKTLAKAMGLKSTLVMGDKLVITSFAASKAVGGAGYKSANIEKITDL |
| | QGRVIEEHERMFSADVGEKNIELSKNDCHTNVNNPVVTNIGKDYIGLKSRL |
| | EQEFFGKTFENDNLHVQLAYNILDIKKILGTYVNNIIYIFYNLNRAGTGRDE |
| | RMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMAQLCMASVA |
| | GTAFKLAESALFNIEDVLSADLKEILDEAFSGAVNKLNDGFVQHSGNNLYV |
| | LQQLYPNETIERIAEKYYRLTVRKEDLNMGVNIKKLRELIVGQYFPEVLDK |
| | EYDLSKNGDSVVTYRSKIYTVMNYILLYYLEDHDSSRESMVEALRQNREG |
| | DEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQC |
| | GSSVWFVDSYRFFERSRRISAQIRIVKNIASKDFKKSKKDSDESYPEQLYLD |
| | ALALLGDVISKYKQNRDGSVVIDDQGNAVLTEQYKRFRYEFFEEIKRDESG |
| | GIKYKKSGKPEYNHQRRNFILNNVLKSKWFFYVVKYNRPSSCRELMKNKEI |
| | LRFVLRDIPDSQVRRYFKAVQGEEAYASAEAMRTRLVDALSQFSVTACLD |
| | EVGGMTDKEFASQRAVDSKEKLRAIIRLYLTVAYLITKSMVKVNTRFSIAFS |
| | VLERDYYLLIDGKKKSSDYTGEDMLALTRKFVGEDAGLYREWKEKNAEA |
| | KDKYFDKAERKKVLRQNDKMIRKMHFTPHSLNYVQKNLESVQSNGLAAV |
| | IKEYANAVAALNIINRLDEYIGSARADSYYSLYCYCLQMYLSKNFSVGYLI |
| | NVQKQLEEHHTYMKDLMWLLNIPFAYNLARYKNLSNEKLFYDEEAAAEK |
| | ADKAENERGE (SEQ ID NO: 583) |
| Linker | GS |
| SV-40 NLS | PKKKRKV (SEQ ID NO: 437) |
| Linker | GGS |
| Myc Tag | EQKLISEEDL (SEQ ID NO: 610) |

In some embodiments, an AAV vector of the disclosure, referred to as A01560, comprising a CUG-targeting dCas13d protein comprises from 5′ to 3′ the elements as set forth in Table K. In some aspects, the AAV vector comprises the nucleic acid sequence set forth in SEQ ID NO: 584 or 585.

TABLE K
Exemplary AAV vector A01560 comprising a CUG-Targeting dCas13d

| Plasmid Element | Amino Acid Sequences |
|---|---|
| 5′ ITR | Cctgcaggcagctgcgcgctcgctcgctcactgaggccgcccgggcgtcgggcgacctttggtcgcccggc |
| | ctcagtgagcgagcgagcgcgcagagagggagtggccaactccatcactaggggttcct (SEQ ID |
| | NO: 599) |
| Human U6 promoter | Gagggcctatttcccatgattccttcatatttgcatatacgatacaaggctgttagagagataattggaatt |
| | aatttgactgtaaacacaaagatattagtacaaaatacgtgacgtagaaagtaataatttcttgggtagttt |
| | gcagttttaaaattatgttttaaaatggactatcatatgcttaccgtaacttgaaagtatttcgatttcttg |
| | gctttatatatcttgtggaaaggacgaaacacc (SEQ ID NO: 519) |
| Seq212 direct | Tagccctgcagtaaggcagggttctaagac (SEQ ID NO: 614) |
| repeat (DR) | |
| Spacer (CTG guide 3) | CAGCAGCAGCAGCAGCAGCAGCAGCA (SEQ ID NO: 459) |
| EFS/UBB promoter | GGGCAGAGCGCACATCGCCCACAGTCCCCGAGAAGTTGGGGGGAGGGG |
| | TCGGCAATTGAaCCGGTGCCTAGAGAAGGTGGCGCGGGGTAAACTGGG |
| | AAAGTGATGTCGTGTACTGGCTCCGCCTTTTTCCCGAGGGTGGGGGAGA |
| | ACCGTATATAAGTGCAGTAGTCGCCGTGAACGTTCTTTTTCGCAACGGG |
| | TTTGCCGCCAGAACACAGaattccagGTAAGTCCCGCAGCCGTAACGACCTT |
| | GGGGGGGTGTGAGATTCTCATTCTAATTTTGAAGAATATTAGGTGTAAA |
| | AGCAAGAAATACAATGATCCTGAGGTGACACGCTTATGTTTTACTTTTA |
| | AACTAGGT (SEQ ID NO: 609) |
| Kozak Sequence | GCCGCCACCATG (SEQ ID NO: 529) |
| Dead Cas13d Seq212 | AAGAAGAAGCACCAGAGCGCCGCCGAGAAGAGGCAAGTGAAGAAGCT |
| | CAAGAATCAAGAGAAGGCCCAGAAGTACGCTAGCGAGCCTTCCCCCCT |
| | CCAGAGCGATACAGCTGGCGTGGAATGCTCCCAGAAAAAGACAGTCGT |
| | CAGCCACATTGCCAGCTCCAAGACACTGGCCAAGGCTATGGGACTCAA |
| | ATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAA |
| | ATCACAGATCTGCAAGGAAGGGTCATTGAGGAGCACGAAAGGATGTTT |
| | AGCGCCGATGTCGGAGAGAAAAATATCGAACTGAGCAAGAATGACTGC |
| | CACACCAACGTCAACAACCCCGTGGTGACCAACATCGGAAAGGATTAC |
| | ATCGGACTGAAATCTAGGCTGGAGCAAGAGTTTTTCGGCAAGACATTCG |
| | AGAATGACAATCTGCATGTGCAGCTGGCCTACAATATCCTCGACATCAA |
| | GAAAATTCTGGGAACCTATGTGAACAATATCATTTATATCTTCTACAAT |
| | CTGAATAGGGCTGGCACCGGCAGAGATGAGAGGATGTATGACGACCTC |
| | ATCGGCACACTGTACGCTTACAAACCCATGGAGGCTCAACAGACCTATC |
| | TGCTCAAAGGCGACAAGGATATGAGGAGGTTTGAGGAGGTGAAACAGC |
| | TGCTGCAAAACACCTCCGCTTACTATGTGTATTACGGCACACTGTTCGA |
| | GAAGGTGAAGGCTAAGAGCAAGAAGGAACAGAGGGCTAAGGAGGCCG |
| | AAATCGACGCTTGTACCGCCCATAACTACGATGTGCTGAGACTGCTGTC |
| | CCTCATGgcGCAGCTGTGCATGgcCTCCGTCGCTGGAACAGCCTTTAAGC |
| | TGGCTGAGTCCGCTCTGTTCAACATTGAGGATGTGCTCAGCGCCGATCT |
| | GAAGGAAATCCTCGATGAAGCCTTCTCCGGCGCCGTGAACAAGCTCAAT |
| | GACGGATTCGTGCAGCACTCCGGCAACAATCTGTACGTGCTCCAGCAGC |
| | TGTACCCTAATGAGACCATCGAGAGAATCGCCGAGAAGTACTACAGAC |
| | TCACCGTGAGGAAGGAGGATCTGAACATGGGAGTCAACATTAAAAAGC |
| | TGAGGGAGCTGATCGTGGGCCAATACTTTCCCGAGGTCCTCGACAAAGA |
| | ATACGACCTCTCCAAGAATGGAGACAGCGTGGTGACATACAGAAGCAA |
| | GATTTATACCGTGATGAATTACATTCTGCTGTATTACCTCGAGGACCAC |
| | GACTCCAGCAGAGAAAGCATGGTCGAAGCTCTGAGACAAAACAGAGAG |
| | GGCGATGAAGGCAAGGAGGAGATCTATAGACAGTTTGCCAAGAAGGTG |
| | TGGAACGGCGTGTCCGGACTGTTTGGCGTGTGTCTGAACCTCTTCAAGA |
| | CCGAAAAGAGAAACAAGTTTAGGAGCAAAGTCGCCCTCCCCGATGTGT |
| | CCGGCGCTGCCTATATGCTCTCCTCCGAGAACATCGACTACTTTGTCAA |
| | GATGCTCTTCTTTGTGTGTAAGTTTCTGGATGGCAAAGAAATCAACGAG |
| | CTGCTGTGCGCTCTGATCAACAAATTTGATAATATTGCCGATATTCTGG |
| | ATGCTGCCGCTCAATGTGGCTCCTCCGTCTGGTTCGTGGACAGCTATAG |
| | GTTCTTCGAGAGATCTAGGAGGATTAGCGCCCAGATTAGAATCGTGAAG |
| | AACATCGCTTCCAAGGATTTTAAGAAATCCAAGAAGGATTCCGATGAG |
| | AGCTACCCCGAGCAGCTGTATCTGGATGCTCTGGCTCTGCTCGGAGACG |
| | TCATCTCCAAGTACAAGCAGAATAGAGATGGCAGCGTCGTCATCGATG |
| | ACCAAGGCAATGCCGTGCTGACAGAGCAATACAAGAGGTTTAGATATG |
| | AATTTTTCGAGGAGATCAAGAGGGACGAAAGCGGCGGCATCAAGTACA |
| | AGAAGTCCGGAAAACCCGAGTACAACCATCAGAGAAGGAATTTTATTC |
| | TGAATAATGTGCTGAAAAGCAAATGGTTTTTCTATGTGGTGAAGTACAA |
| | TAGGCCCAGCAGCTGCAGAGAACTGATGAAGAATAAGGAAATTCTGAG |
| | GTTCGTGCTGAGAGACATCCCCGACTCCCAAGTGAGAAGATACTTTAAG |
| | GCCGTCCAAGGAGAGGAAGCTTACGCTAGCGCCGAAGCTATGAGGACA |
| | AGACTGGTCGACGCTCTGTCCCAATTTAGCGTCACAGCTTGTCTGGATG |
| | AAGTGGGCGGCATGACAGACAAGGAATTCGCCTCCCAGAGGGCCGTCG |
| | ATAGCAAAGAAAAACTGAGAGCCATCATCAGACTGTATCTGACAGTCG |
| | CCTATCTGATTACCAAGAGCATGGTGAAGGTGAATACAAGGTTTAGCAT |
| | TGCCTTTAGCGTGCTGGAGAGGGACTACTATCTGCTCATTGACGGCAAG |
| | AAGAAATCCAGCGACTACACCGGAGAGGATATGCTGGCTCTGACCAGA |
| | AAATTTGTGGGCGAAGATGCTGGACTGTATAGAGAGTGGAAAGAGAAG |
| | AACGCTGAAGCCAAGGACAAATATTTTGACAAGGCCGAAAGGAAGAAG |
| | GTGCTGAGACAGAACGATAAGATGATCAGAAAGATGCACTTCACACCC |
| | CACTCCCTCAATTACGTCCAAAAGAATCTCGAAAGCGTCCAGAGCAACG |
| | GACTGGCCGCCGTCATCAAGGAATATgcAAATGCCGTCGCTgcCCTCAAT |
| | ATCATCAATAGACTGGACGAGTACATTGGCTCCGCTAGGGCTGATAGCT |
| | ACTACTCTCTGTACTGTTACTGCCTCCAAATGTATCTGAGCAAGAACTTC |
| | AGCGTGGGCTACCTCATCAACGTGCAAAAGCAGCTGGAGGAGCACCAC |
| | ACCTACATGAAGGATCTCATGTGGCTGCTCAACATCCCCTTCGCTTACA |
| | ACCTCGCCAGATACAAAAATCTGTCCAACGAAAAACTCTTTTACGACGA |
| | GGAAGCCGCCGCCGAAAAGGCTGACAAGGCTGAGAACGAGAGAGGCG |
| | AA (SEQ ID NO: 612) |
| Linker | GGAAGC |
| SV-40 NLS | CCCAAGAAGAAAAGGAAGGTC (SEQ ID NO: 532) |
| Linker | GGCGGAtct (SEQ ID NO: 613) |
| Myc Tag | GAGCAgAAACTGATTAGcGAAGAgGATCTC (SEQ ID NO: 602) |
| WPRE3 | GATAATCAACCTCTGGATTACAAAATTTGTGAAAGATTGACTGGTATTC |
| | TTAACTATGTTGCTCCTTTTACGCTATGTGGATACGCTGCTTTAATGCCT |
| | TTGTATCATGCTATTGCTTCCCGTATGGCTTTCATTTTCTCCTCCTTGTAT |
| | AAATCCTGGTTAGTTCTTGCCACGGCGGAACTCATCGCCGCCTGCCTTG |
| | CCCGCTGCTGGACAGGGGCTCGGCTGTTGGGCACTGACAATTCCGTGG |
| | (SEQ ID NO: 603) |
| SV-40 poly A | AACTTGTTTATTGCAGCTTATAATGGTTACAAATAAAGCAATAGCATCA |
| | CAAATTTCACAAATAAAGCATTTTTTTCACTGCATTCTAGTTGTGGTTTG |
| | TCCAAACTCATCAATGTATCTTA (SEQ ID NO: 533) |
| 3′ITR | Aggaacccctagtgatggagttggccactccctctctgcgcgctcgctcgctcactgaggccgggcgacca |
| | aaggtcgcccgacgcccgggctttgcccgggcggcctcagtgagcgagcgagcgcgcagctgcctgcagg |
| | (SEQ ID NO: 600) |

Cas13d: dSeq212 Targeting CUG No Endonuclease (with 1 Point Mutation H919A at HEPN2)

In some embodiments, the disclosure provides a CUG-targeting catalytically inactive Cas (dCas13d) having an H919A mutation at HEPN2 as set forth in Table L.

TABLE L
Exemplary CUG-Targeting dcas13d for blocking CUG RNA

| Plasmid | |
|---|---|
| Element | Amino Acid Sequences |
| Dead Seq212 | KKKHQSAAEKRQVKKLKNQEKAQKYASEPSPLQSDTAGVECSQKKTVVSHIASSKTLAKAMG |
| H919A | LKSTLVMGDKLVITSFAASKAVGGAGYKSANIEKITDLQGRVIEEHERMFSADVGEKNIELSKN |
| | DCHTNVNNPVVTNIGKDYIGLKSRLEQEFFGKTFENDNLHVQLAYNILDIKKILGTYVNNIIYIF |
| | YNLNRAGTGRDERMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMRQLCMHSVAGTAFKLAESALFN |
| | IEDVLSADLKEILDEAFSGAVNKLNDGFVQHSGNNLYVLQQLYPNETIERIAEKYYRLTVRKED |
| | LNMGVNIKKLRELIVGQYFPEVLDKEYDLSKNGDSVVTYRSKIYTVMNYILLYYLEDHDSSRE |
| | SMVEALRQNREGDEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQCGSSVWFVDSYRF |
| | FERSRRISAQIRIVKNIASKDFKKSKKDSDESYPEQLYLDALALLGDVISKYKQNRDGSVVIDDQ |
| | GNAVLTEQYKRFRYEFFEEIKRDESGGIKYKKSGKPEYNHQRRNFILNNVLKSKWFFYVVKYN |
| | RPSSCRELMKNKEILRFVLRDIPDSQVRRYFKAVQGEEAYASAEAMRTRLVDALSQFSVTACL |
| | DEVGGMTDKEFASQRAVDSKEKLRAIIRLYLTVAYLITKSMVKVNTRFSIAFSVLERDYYLLID |
| | GKKKSSDYTGEDMLALTRKFVGEDAGLYREWKEKNAEAKDKYFDKAERKKVLRQNDKMIR |
| | KMHFTPHSLNYVQKNLESVOSNGLAAVIKEYRNAVAALNIINRLDEYIGSARADSYYSLYCYC |
| | LQMYLSKNFSVGYLINVQKQLEEHHTYMKDLMWLLNIPFAYNLARYKNLSNEKLFYDEEAAA |
| | EKADKAENERGE (SEQ ID NO: 586) |
| Linker | GS |
| SV-40 NLS | PKKKRKV (SEQ ID NO: 437) |
| Linker | GGS |
| Myc Tag | EQKLISEEDL (SEQ ID NO: 610) |

In some embodiments, an AAV vector of the disclosure, comprising a CUG-targeting dCas13d protein comprises from 5′ to 3′ the elements as set forth in Table M. In some aspects, the AAV vector comprises the nucleic acid sequence set forth in SEQ ID NO: 590 or 591.

TABLE M
Exemplary AAV vector comprising a CUG-Targeting dCas13d

| Plasmid | |
|---|---|
| Element | Amino Acid Sequences |
| 5′ ITR | Cctgcaggcagctgcgcgctcgctcgctcactgaggccgcccgggcgtcgggcgacctttggtcgcccggcctcagtg |
| | agcgagcgagcgcgcagagagggagtggccaactccatcactaggggttcct (SEQ ID NO: 599) |
| Human U6 | Gagggcctatttcccatgattccttcatatttgcatatacgatacaaggctgttagagagataattggaattaatttg |
| promoter | actgtaaacacaaagatattagtacaaaatacgtgacgtagaaagtaataatttcttgggtagtttgcagttttaaaa |
| | ttatgttttaaaatggactatcatatgcttaccgtaacttgaaagtatttcgatttcttggctttatatatcttgtgga |
| | aaggacgaaacacc (SEQ ID NO: 519) |
| Seq212 direct | Tagccctgcagtaaggcagggttctaagac (SEQ ID NO: 614) |
| repeat (DR) | |
| Spacer (CTG | CAGCAGCAGCAGCAGCAGCAGCAGCA (SEQ ID NO: 459) |
| guide 3) | |
| EFS/UBB | GGGCAGAGCGCACATCGCCCACAGTCCCCGAGAAGTTGGGGGGAGGGGTCGGCAATTGAa |
| promoter | CCGGTGCCTAGAGAAGGTGGCGCGGGGTAAACTGGGAAAGTGATGTCGTGTACTGGCTCC |
| | GCCTTTTTCCCGAGGGTGGGGGAGAACCGTATATAAGTGCAGTAGTCGCCGTGAACGTTCT |
| | TTTTCGCAACGGGTTTGCCGCCAGAACACAGaattccagGTAAGTCCCGCAGCCGTAACGACCT |
| | TGGGGGGGTGTGAGATTCTCATTCTAATTTTGAAGAATATTAGGTGTAAAAGCAAGAAATA |
| | CAATGATCCTGAGGTGACACGCTTATGTTTTACTTTTAAACTAGGT (SEQ ID NO: 609) |
| Kozak Sequence | GCCGCCACCATG (SEQ ID NO: 529) |
| dCas13dSeq212 | AAGAAGAAGCACCAGAGCGCCGCCGAGAAGAGGCAAGTGAAGAAGCTCAAGAATCAAGA |
| H919A | GAAGGCCCAGAAGTACGCTAGCGAGCCTTCCCCCCTCCAGAGCGATACAGCTGGCGTGGA |
| | ATGCTCCCAGAAAAAGACAGTCGTCAGCCACATTGCCAGCTCCAAGACACTGGCCAAGGC |
| | TATGGGACTCAAATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAAATCACAGATCTG |
| | CAAGGAAGGGTCATTGAGGAGCACGAAAGGATGTTTAGCGCCGATGTCGGAGAGAAAAA |
| | TATCGAACTGAGCAAGAATGACTGCCACACCAACGTCAACAACCCCGTGGTGACCAACAT |
| | CGGAAAGGATTACATCGGACTGAAATCTAGGCTGGAGCAAGAGTTTTTCGGCAAGACATT |
| | CGAGAATGACAATCTGCATGTGCAGCTGGCCTACAATATCCTCGACATCAAGAAAATTCTG |
| | GGAACCTATGTGAACAATATCATTTATATCTTCTACAATCTGAATAGGGCTGGCACCGGCA |
| | GAGATGAGAGGATGTATGACGACCTCATCGGCACACTGTACGCTTACAAACCCATGGAGG |
| | CTCAACAGACCTATCTGCTCAAAGGCGACAAGGATATGAGGAGGTTTGAGGAGGTGAAAC |
| | AGCTGCTGCAAAACACCTCCGCTTACTATGTGTATTACGGCACACTGTTCGAGAAGGTGAA |
| | GGCTAAGAGCAAGAAGGAACAGAGGGCTAAGGAGGCCGAAATCGACGCTTGTACCGCCC |
| | ATAACTACGATGTGCTGAGACTGCTGTCCCTCATGagGCAGCTGTGCATGcaCTCCGTCGCTG |
| | GAACAGCCTTTAAGCTGGCTGAGTCCGCTCTGTTCAACATTGAGGATGTGCTCAGCGCCGA |
| | TCTGAAGGAAATCCTCGATGAAGCCTTCTCCGGCGCCGTGAACAAGCTCAATGACGGATTC |
| | GTGCAGCACTCCGGCAACAATCTGTACGTGCTCCAGCAGCTGTACCCTAATGAGACCATCG |
| | AGAGAATCGCCGAGAAGTACTACAGACTCACCGTGAGGAAGGAGGATCTGAACATGGGA |
| | GTCAACATTAAAAAGCTGAGGGAGCTGATCGTGGGCCAATACTTTCCCGAGGTCCTCGAC |
| | AAAGAATACGACCTCTCCAAGAATGGAGACAGCGTGGTGACATACAGAAGCAAGATTTAT |
| | ACCGTGATGAATTACATTCTGCTGTATTACCTCGAGGACCACGACTCCAGCAGAGAAAGCA |
| | TGGTCGAAGCTCTGAGACAAAACAGAGAGGGCGATGAAGGCAAGGAGGAGATCTATAGA |
| | CAGTTTGCCAAGAAGGTGTGGAACGGCGTGTCCGGACTGTTTGGCGTGTGTCTGAACCTCT |
| | TCAAGACCGAAAAGAGAAACAAGTTTAGGAGCAAAGTCGCCCTCCCCGATGTGTCCGGCG |
| | CTGCCTATATGCTCTCCTCCGAGAACATCGACTACTTTGTCAAGATGCTCTTCTTTGTGTGT |
| | AAGTTTCTGGATGGCAAAGAAATCAACGAGCTGCTGTGCGCTCTGATCAACAAATTTGATA |
| | ATATTGCCGATATTCTGGATGCTGCCGCTCAATGTGGCTCCTCCGTCTGGTTCGTGGACAGC |
| | TATAGGTTCTTCGAGAGATCTAGGAGGATTAGCGCCCAGATTAGAATCGTGAAGAACATC |
| | GCTTCCAAGGATTTTAAGAAATCCAAGAAGGATTCCGATGAGAGCTACCCCGAGCAGCTG |
| | TATCTGGATGCTCTGGCTCTGCTCGGAGACGTCATCTCCAAGTACAAGCAGAATAGAGATG |
| | GCAGCGTCGTCATCGATGACCAAGGCAATGCCGTGCTGACAGAGCAATACAAGAGGTTTA |
| | GATATGAATTTTTCGAGGAGATCAAGAGGGACGAAAGCGGCGGCATCAAGTACAAGAAGT |
| | CCGGAAAACCCGAGTACAACCATCAGAGAAGGAATTTTATTCTGAATAATGTGCTGAAAA |
| | GCAAATGGTTTTTCTATGTGGTGAAGTACAATAGGCCCAGCAGCTGCAGAGAACTGATGA |
| | AGAATAAGGAAATTCTGAGGTTCGTGCTGAGAGACATCCCCGACTCCCAAGTGAGAAGAT |
| | ACTTTAAGGCCGTCCAAGGAGAGGAAGCTTACGCTAGCGCCGAAGCTATGAGGACAAGAC |
| | TGGTCGACGCTCTGTCCCAATTTAGCGTCACAGCTTGTCTGGATGAAGTGGGCGGCATGAC |
| | AGACAAGGAATTCGCCTCCCAGAGGGCCGTCGATAGCAAAGAAAAACTGAGAGCCATCAT |
| | CAGACTGTATCTGACAGTCGCCTATCTGATTACCAAGAGCATGGTGAAGGTGAATACAAG |
| | GTTTAGCATTGCCTTTAGCGTGCTGGAGAGGGACTACTATCTGCTCATTGACGGCAAGAAG |
| | AAATCCAGCGACTACACCGGAGAGGATATGCTGGCTCTGACCAGAAAATTTGTGGGCGAA |
| | GATGCTGGACTGTATAGAGAGTGGAAAGAGAAGAACGCTGAAGCCAAGGACAAATATTTT |
| | GACAAGGCCGAAAGGAAGAAGGTGCTGAGACAGAACGATAAGATGATCAGAAAGATGCA |
| | CTTCACACCCCACTCCCTCAATTACGTCCAAAAGAATCTCGAAAGCGTCCAGAGCAACGGA |
| | CTGGCCGCCGTCATCAAGGAATATagAAATGCCGTCGCTgcCCTCAATATCATCAATAGACT |
| | GGACGAGTACATTGGCTCCGCTAGGGCTGATAGCTACTACTCTCTGTACTGTTACTGCCTC |
| | CAAATGTATCTGAGCAAGAACTTCAGCGTGGGCTACCTCATCAACGTGCAAAAGCAGCTG |
| | GAGGAGCACCACACCTACATGAAGGATCTCATGTGGCTGCTCAACATCCCCTTCGCTTACA |
| | ACCTCGCCAGATACAAAAATCTGTCCAACGAAAAACTCTTTTACGACGAGGAAGCCGCCG |
| | CCGAAAAGGCTGACAAGGCTGAGAACGAGAGAGGCGAA (SEQ ID NO: 615) |
| Linker | GGAAGC |
| SV-40 NLS | CCCAAGAAGAAAAGGAAGGTC (SEQ ID NO: 532) |
| Linker | GGCGGAtct (SEQ ID NO: 613) |
| Myc Tag | GAGCAgAAACTGATTAGcGAAGAgGATCTC (SEQ ID NO: 602) |
| WPRE3 | GATAATCAACCTCTGGATTACAAAATTTGTGAAAGATTGACTGGTATTCTTAACTATGTTG |
| | CTCCTTTTACGCTATGTGGATACGCTGCTTTAATGCCTTTGTATCATGCTATTGCTTCCCGTA |
| | TGGCTTTCATTTTCTCCTCCTTGTATAAATCCTGGTTAGTTCTTGCCACGGCGGAACTCATC |
| | GCCGCCTGCCTTGCCCGCTGCTGGACAGGGGCTCGGCTGTTGGGCACTGACAATTCCGTGG |
| | (SEQ ID NO: 603) |
| SV-40 poly A | AACTTGTTTATTGCAGCTTATAATGGTTACAAATAAAGCAATAGCATCACAAATTTCACAA |
| | ATAAAGCATTTTTTTCACTGCATTCTAGTTGTGGTTTGTCCAAACTCATCAATGTATCTTA |
| | (SEQ ID NO: 533) |
| 3′ITR | Aggaacccctagtgatggagttggccactccctctctgcgcgctcgctcgctcactgaggccgggcgaccaaaggtcg |
| | cccgacgcccgggctttgcccgggcggcctcagtgagcgagcgagcgcgcagctgcctgcagg (SEQ ID NO: 600) |

Cas13d: dSeq212 Targeting CUG No Endonuclease (with 1 Point Mutation R914A at HEPN2)

In some embodiments, the disclosure provides a CUG-targeting catalytically inactive Cas (dCas13d) having an H914A mutation as set forth in Table N.

TABLE N
Exemplary CUG-Targeting dcas 13d for blocking CUG RNA

| Plasmid | |
|---|---|
| Element | Amino Acid Sequences |
| Dead dCas13d | KKKHQSAAEKRQVKKLKNQEKAQKYASEPSPLQSDTAGVECSQKKTVVSHIASSKTLAKAMG |
| Seq212 R914A | LKSTLVMGDKLVITSFAASKAVGGAGYKSANIEKITDLQGRVIEEHERMFSADVGEKNIELSKN |
| | DCHTNVNNPVVTNIGKDYIGLKSRLEQEFFGKTFENDNLHVQLAYNILDIKKILGTYVNNIIYIF |
| | YNLNRAGTGRDERMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMRQLCMHSVAGTAFKLAESALFN |
| | IEDVLSADLKEILDEAFSGAVNKLNDGFVQHSGNNLYVLQQLYPNETIERIAEKYYRLTVRKED |
| | LNMGVNIKKLRELIVGQYFPEVLDKEYDLSKNGDSVVTYRSKIYTVMNYILLYYLEDHDSSRE |
| | SMVEALRQNREGDEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQCGSSVWFVDSYRF |
| | FERSRRISAQIRIVKNIASKDFKKSKKDSDESYPEQLYLDALALLGDVISKYKQNRDGSVVIDDQ |
| | GNAVLTEQYKRFRYEFFEEIKRDESGGIKYKKSGKPEYNHQRRNFILNNVLKSKWFFYVVKYN |
| | RPSSCRELMKNKEILRFVLRDIPDSQVRRYFKAVQGEEAYASAEAMRTRLVDALSQFSVTACL |
| | DEVGGMTDKEFASQRAVDSKEKLRAIIRLYLTVAYLITKSMVKVNTRFSIAFSVLERDYYLLID |
| | GKKKSSDYTGEDMLALTRKFVGEDAGLYREWKEKNAEAKDKYFDKAERKKVLRQNDKMIR |
| | KMHFTPHSLNYVQKNLESVQSNGLAAVIKEYANAVAHLNIINRLDEYIGSARADSYYSLYCYC |
| | LQMYLSKNFSVGYLINVQKQLEEHHTYMKDLMWLLNIPFAYNLARYKNLSNEKLFYDEEAAA |
| | EKADKAENERGE (SEQ ID NO: 587) |
| Linker | GS |
| SV-40 NLS | PKKKRKV (SEQ ID NO: 437) |
| Linker | GGS |
| Myc Tag | EQKLISEEDL (SEQ ID NO: 610) |

In some embodiments, an AAV vector of the disclosure, comprising a CUG-targeting dCas13d protein comprises from 5′ to 3′ the elements as set forth in Table O. In some aspects, the AAV vector comprises the nucleic acid sequence set forth in SEQ ID NO. 592 or 593.

TABLE O
Exemplary AAV vector comprising a CUG-Targeting dCas13d

| Plasmid | |
|---|---|
| Element | Amino Acid Sequences |
| 5′ ITR | Cctgcaggcagctgcgcgctcgctcgctcactgaggccgcccgggcgtcgggcgacctttggtcgcccggcctcagtgag |
| | cgagcgagcgcgcagagagggagtggccaactccatcactaggggttcct (SEQ ID NO: 599) |
| Human U6 | Gagggcctatttcccatgattccttcatatttgcatatacgatacaaggctgttagagagataattggaattaatttgac |
| promoter | tgtaaacacaaagatattagtacaaaatacgtgacgtagaaagtaataatttcttgggtagtttgcagttttaaaattat |
| | gttttaaaatggactatcatatgcttaccgtaacttgaaagtatttcgatttcttggctttatatatcttgtggaaagga |
| | cgaaacacc (SEQ ID NO: 519) |
| Seq212 direct | Tagccctgcagtaaggcagggttctaagac (SEQ ID NO: 614) |
| repeat (DR) | |
| Spacer (CTG | CAGCAGCAGCAGCAGCAGCAGCAGCA (SEQ ID NO: 459) |
| guide 3) | |
| EFS/UBB | GGGCAGAGCGCACATCGCCCACAGTCCCCGAGAAGTTGGGGGGAGGGGTCGGCAATTGAa |
| promoter | CCGGTGCCTAGAGAAGGTGGCGCGGGGTAAACTGGGAAAGTGATGTCGTGTACTGGCTCC |
| | GCCTTTTTCCCGAGGGTGGGGGAGAACCGTATATAAGTGCAGTAGTCGCCGTGAACGTTCT |
| | TTTTCGCAACGGGTTTGCCGCCAGAACACAGaattccagGTAAGTCCCGCAGCCGTAACGACCT |
| | TGGGGGGGTGTGAGATTCTCATTCTAATTTTGAAGAATATTAGGTGTAAAAGCAAGAAATA |
| | CAATGATCCTGAGGTGACACGCTTATGTTTTACTTTTAAACTAGGT (SEQ ID NO: 609) |
| Kozak Sequence | GCCGCCACCATG (SEQ ID NO: 529) |
| Dead Seq212 | AAGAAGAAGCACCAGAGCGCCGCCGAGAAGAGGCAAGTGAAGAAGCTCAAGAATCAAGA |
| R914A | GAAGGCCCAGAAGTACGCTAGCGAGCCTTCCCCCCTCCAGAGCGATACAGCTGGCGTGGA |
| | ATGCTCCCAGAAAAAGACAGTCGTCAGCCACATTGCCAGCTCCAAGACACTGGCCAAGGC |
| | TATGGGACTCAAATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAAATCACAGATCTG |
| | CAAGGAAGGGTCATTGAGGAGCACGAAAGGATGTTTAGCGCCGATGTCGGAGAGAAAAA |
| | TATCGAACTGAGCAAGAATGACTGCCACACCAACGTCAACAACCCCGTGGTGACCAACAT |
| | CGGAAAGGATTACATCGGACTGAAATCTAGGCTGGAGCAAGAGTTTTTCGGCAAGACATT |
| | CGAGAATGACAATCTGCATGTGCAGCTGGCCTACAATATCCTCGACATCAAGAAAATTCTG |
| | GGAACCTATGTGAACAATATCATTTATATCTTCTACAATCTGAATAGGGCTGGCACCGGCA |
| | GAGATGAGAGGATGTATGACGACCTCATCGGCACACTGTACGCTTACAAACCCATGGAGG |
| | CTCAACAGACCTATCTGCTCAAAGGCGACAAGGATATGAGGAGGTTTGAGGAGGTGAAAC |
| | AGCTGCTGCAAAACACCTCCGCTTACTATGTGTATTACGGCACACTGTTCGAGAAGGTGAA |
| | GGCTAAGAGCAAGAAGGAACAGAGGGCTAAGGAGGCCGAAATCGACGCTTGTACCGCCC |
| | ATAACTACGATGTGCTGAGACTGCTGTCCCTCATGagGCAGCTGTGCATGcaCTCCGTCGCTG |
| | GAACAGCCTTTAAGCTGGCTGAGTCCGCTCTGTTCAACATTGAGGATGTGCTCAGCGCCGA |
| | TCTGAAGGAAATCCTCGATGAAGCCTTCTCCGGCGCCGTGAACAAGCTCAATGACGGATTC |
| | GTGCAGCACTCCGGCAACAATCTGTACGTGCTCCAGCAGCTGTACCCTAATGAGACCATCG |
| | AGAGAATCGCCGAGAAGTACTACAGACTCACCGTGAGGAAGGAGGATCTGAACATGGGA |
| | GTCAACATTAAAAAGCTGAGGGAGCTGATCGTGGGCCAATACTTTCCCGAGGTCCTCGAC |
| | AAAGAATACGACCTCTCCAAGAATGGAGACAGCGTGGTGACATACAGAAGCAAGATTTAT |
| | ACCGTGATGAATTACATTCTGCTGTATTACCTCGAGGACCACGACTCCAGCAGAGAAAGCA |
| | TGGTCGAAGCTCTGAGACAAAACAGAGAGGGCGATGAAGGCAAGGAGGAGATCTATAGA |
| | CAGTTTGCCAAGAAGGTGTGGAACGGCGTGTCCGGACTGTTTGGCGTGTGTCTGAACCTCT |
| | TCAAGACCGAAAAGAGAAACAAGTTTAGGAGCAAAGTCGCCCTCCCCGATGTGTCCGGCG |
| | CTGCCTATATGCTCTCCTCCGAGAACATCGACTACTTTGTCAAGATGCTCTTCTTTGTGTGT |
| | AAGTTTCTGGATGGCAAAGAAATCAACGAGCTGCTGTGCGCTCTGATCAACAAATTTGATA |
| | ATATTGCCGATATTCTGGATGCTGCCGCTCAATGTGGCTCCTCCGTCTGGTTCGTGGACAGC |
| | TATAGGTTCTTCGAGAGATCTAGGAGGATTAGCGCCCAGATTAGAATCGTGAAGAACATC |
| | GCTTCCAAGGATTTTAAGAAATCCAAGAAGGATTCCGATGAGAGCTACCCCGAGCAGCTG |
| | TATCTGGATGCTCTGGCTCTGCTCGGAGACGTCATCTCCAAGTACAAGCAGAATAGAGATG |
| | GCAGCGTCGTCATCGATGACCAAGGCAATGCCGTGCTGACAGAGCAATACAAGAGGTTTA |
| | GATATGAATTTTTCGAGGAGATCAAGAGGGACGAAAGCGGCGGCATCAAGTACAAGAAGT |
| | CCGGAAAACCCGAGTACAACCATCAGAGAAGGAATTTTATTCTGAATAATGTGCTGAAAA |
| | GCAAATGGTTTTTCTATGTGGTGAAGTACAATAGGCCCAGCAGCTGCAGAGAACTGATGA |
| | AGAATAAGGAAATTCTGAGGTTCGTGCTGAGAGACATCCCCGACTCCCAAGTGAGAAGAT |
| | ACTTTAAGGCCGTCCAAGGAGAGGAAGCTTACGCTAGCGCCGAAGCTATGAGGACAAGAC |
| | TGGTCGACGCTCTGTCCCAATTTAGCGTCACAGCTTGTCTGGATGAAGTGGGCGGCATGAC |
| | AGACAAGGAATTCGCCTCCCAGAGGGCCGTCGATAGCAAAGAAAAACTGAGAGCCATCAT |
| | CAGACTGTATCTGACAGTCGCCTATCTGATTACCAAGAGCATGGTGAAGGTGAATACAAG |
| | GTTTAGCATTGCCTTTAGCGTGCTGGAGAGGGACTACTATCTGCTCATTGACGGCAAGAAG |
| | AAATCCAGCGACTACACCGGAGAGGATATGCTGGCTCTGACCAGAAAATTTGTGGGCGAA |
| | GATGCTGGACTGTATAGAGAGTGGAAAGAGAAGAACGCTGAAGCCAAGGACAAATATTTT |
| | GACAAGGCCGAAAGGAAGAAGGTGCTGAGACAGAACGATAAGATGATCAGAAAGATGCA |
| | CTTCACACCCCACTCCCTCAATTACGTCCAAAAGAATCTCGAAAGCGTCCAGAGCAACGGA |
| | CTGGCCGCCGTCATCAAGGAATATgcAAATGCCGTCGCTcaCCTCAATATCATCAATAGACT |
| | GGACGAGTACATTGGCTCCGCTAGGGCTGATAGCTACTACTCTCTGTACTGTTACTGCCTC |
| | CAAATGTATCTGAGCAAGAACTTCAGCGTGGGCTACCTCATCAACGTGCAAAAGCAGCTG |
| | GAGGAGCACCACACCTACATGAAGGATCTCATGTGGCTGCTCAACATCCCCTTCGCTTACA |
| | ACCTCGCCAGATACAAAAATCTGTCCAACGAAAAACTCTTTTACGACGAGGAAGCCGCCG |
| | CCGAAAAGGCTGACAAGGCTGAGAACGAGAGAGGCGAA (SEQ ID NO: 616) |
| Linker | GGAAGC |
| SV-40 NLS | CCCAAGAAGAAAAGGAAGGTC (SEQ ID NO: 532) |
| Linker | GGCGGAtct (SEQ ID NO: 613) |
| Myc Tag | GAGCAgAAACTGATTAGcGAAGAgGATCTC (SEQ ID NO: 602) |
| WPRE3 | GATAATCAACCTCTGGATTACAAAATTTGTGAAAGATTGACTGGTATTCTTAACTATGTTG |
| | CTCCTTTTACGCTATGTGGATACGCTGCTTTAATGCCTTTGTATCATGCTATTGCTTCCCGTA |
| | TGGCTTTCATTTTCTCCTCCTTGTATAAATCCTGGTTAGTTCTTGCCACGGCGGAACTCATC |
| | GCCGCCTGCCTTGCCCGCTGCTGGACAGGGGCTCGGCTGTTGGGCACTGACAATTCCGTGG |
| | (SEQ ID NO: 603) |
| SV-40 poly A | AACTTGTTTATTGCAGCTTATAATGGTTACAAATAAAGCAATAGCATCACAAATTTCACAA |
| | ATAAAGCATTTTTTTCACTGCATTCTAGTTGTGGTTTGTCCAAACTCATCAATGTATCTTA |
| | (SEQ ID NO: 533) |
| 3′ITR | Aggaacccctagtgatggagttggccactccctctctgcgcgctcgctcgctcactgaggccgggcgaccaaaggtcgcc |
| | cgacgcccgggctttgcccgggcggcctcagtgagcgagcgagcgcgcagctgcctgcagg (SEQ ID NO: 600) |

Cas13d: dSeq212 Targeting CUG No Endonuclease (with 1 Point Mutation R293A at HEPN1)

In some embodiments, the disclosure provides a CUG-targeting catalytically inactive Cas (dCas13d) having an R293A mutation at HEPN1 as set forth in Table P.

TABLE P
Exemplary CUG-Targeting dcas13d for blocking CUG RNA

| Plasmid | |
|---|---|
| Element | Amino Acid Sequences |
| dCas13d Seq212 | KKKHQSAAEKRQVKKLKNQEKAQKYASEPSPLQSDTAGVECSQKKTVVSHIASSKTLAKAMG |
| R293A | LKSTLVMGDKLVITSFAASKAVGGAGYKSANIEKITDLQGRVIEEHERMFSADVGEKNIELSKN |
| | DCHTNVNNPVVTNIGKDYIGLKSRLEQEFFGKTFENDNLHVQLAYNILDIKKILGTYVNNIIYIF |
| | YNLNRAGTGRDERMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMAQLCMHSVAGTAFKLAESALFN |
| | IEDVLSADLKEILDEAFSGAVNKLNDGFVQHSGNNLYVLQQLYPNETIERIAEKYYRLTVRKED |
| | LNMGVNIKKLRELIVGQYFPEVLDKEYDLSKNGDSVVTYRSKIYTVMNYILLYYLEDHDSSRE |
| | SMVEALRQNREGDEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQCGSSVWFVDSYRF |
| | FERSRRISAQIRIVKNIASKDFKKSKKDSDESYPEQLYLDALALLGDVISKYKQNRDGSVVIDDQ |
| | GNAVLTEQYKRFRYEFFEEIKRDESGGIKYKKSGKPEYNHQRRNFILNNVLKSKWFFYVVKYN |
| | RPSSCRELMKNKEILRFVLRDIPDSQVRRYFKAVQGEEAYASAEAMRTRLVDALSQFSVTACL |
| | DEVGGMTDKEFASQRAVDSKEKLRAIIRLYLTVAYLITKSMVKVNTRFSIAFSVLERDYYLLID |
| | GKKKSSDYTGEDMLALTRKFVGEDAGLYREWKEKNAEAKDKYFDKAERKKVLRQNDKMIR |
| | KMHFTPHSLNYVQKNLESVQSNGLAAVIKEYRNAVAHLNIINRLDEYIGSARADSYYSLYCYC |
| | LQMYLSKNFSVGYLINVQKQLEEHHTYMKDLMWLLNIPFAYNLARYKNLSNEKLFYDEEAAA |
| | EKADKAENERGE (SEQ ID NO: 588) |
| Linker | GS |
| SV-40 NLS | PKKKRKV (SEQ ID NO: 437) |
| Linker | GGS |
| Myc Tag | EQKLISEEDL (SEQ ID NO: 610) |

In some embodiments, an AAV vector of the disclosure, comprising a CUG-targeting dCas13d protein comprises from 5′ to 3′ the elements as set forth in Table Q. In some aspects, the AAV vector comprises the nucleic acid sequence set forth in SEQ ID NO. 594 or 595.

TABLE Q
Exemplary AAV vector comprising a CUG-Targeting dCas13d

| Plasmid | |
|---|---|
| Element | Amino Acid Sequences |
| 5′ ITR | Cctgcaggcagctgcgcgctcgctcgctcactgaggccgcccgggcgtcgggcgacctttggtcgcccggcctcagtgagc |
| | gagcgagcgcgcagagagggagtggccaactccatcactaggggttcct (SEQ ID NO: 599) |
| Human U6 | Gagggcctatttcccatgattccttcatatttgcatatacgatacaaggctgttagagagataattggaattaatttgact |
| promoter | gtaaacacaaagatattagtacaaaatacgtgacgtagaaagtaataatttcttgggtagtttgcagttttaaaattatgt |
| | tttaaaatggactatcatatgcttaccgtaacttgaaagtatttcgatttcttggctttatatatcttgtggaaaggacga |
| | aacacc (SEQ ID NO: 519) |
| Seq212 direct | Tagccctgcagtaaggcagggttctaagac (SEQ ID NO: 614) |
| repeat (DR) | |
| Spacer (CTG | CAGCAGCAGCAGCAGCAGCAGCAGCA (SEQ ID NO: 459) |
| guide 3) | |
| EFS/UBB | GGGCAGAGCGCACATCGCCCACAGTCCCCGAGAAGTTGGGGGGAGGGGTCGGCAATTGAa |
| promoter | CCGGTGCCTAGAGAAGGTGGCGCGGGGTAAACTGGGAAAGTGATGTCGTGTACTGGCTCC |
| | GCCTTTTTCCCGAGGGTGGGGGAGAACCGTATATAAGTGCAGTAGTCGCCGTGAACGTTCT |
| | TTTTCGCAACGGGTTTGCCGCCAGAACACAGaattccagGTAAGTCCCGCAGCCGTAACGACCT |
| | TGGGGGGGTGTGAGATTCTCATTCTAATTTTGAAGAATATTAGGTGTAAAAGCAAGAAATA |
| | CAATGATCCTGAGGTGACACGCTTATGTTTTACTTTTAAACTAGGT (SEQ ID NO: 609) |
| Kozak Sequence | GCCGCCACCATG (SEQ ID NO: 529) |
| Dead Seq212 | AAGAAGAAGCACCAGAGCGCCGCCGAGAAGAGGCAAGTGAAGAAGCTCAAGAATCAAGA |
| R293A | GAAGGCCCAGAAGTACGCTAGCGAGCCTTCCCCCCTCCAGAGCGATACAGCTGGCGTGGA |
| | ATGCTCCCAGAAAAAGACAGTCGTCAGCCACATTGCCAGCTCCAAGACACTGGCCAAGGC |
| | TATGGGACTCAAATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAAATCACAGATCTG |
| | CAAGGAAGGGTCATTGAGGAGCACGAAAGGATGTTTAGCGCCGATGTCGGAGAGAAAAA |
| | TATCGAACTGAGCAAGAATGACTGCCACACCAACGTCAACAACCCCGTGGTGACCAACAT |
| | CGGAAAGGATTACATCGGACTGAAATCTAGGCTGGAGCAAGAGTTTTTCGGCAAGACATT |
| | CGAGAATGACAATCTGCATGTGCAGCTGGCCTACAATATCCTCGACATCAAGAAAATTCTG |
| | GGAACCTATGTGAACAATATCATTTATATCTTCTACAATCTGAATAGGGCTGGCACCGGCA |
| | GAGATGAGAGGATGTATGACGACCTCATCGGCACACTGTACGCTTACAAACCCATGGAGG |
| | CTCAACAGACCTATCTGCTCAAAGGCGACAAGGATATGAGGAGGTTTGAGGAGGTGAAAC |
| | AGCTGCTGCAAAACACCTCCGCTTACTATGTGTATTACGGCACACTGTTCGAGAAGGTGAA |
| | GGCTAAGAGCAAGAAGGAACAGAGGGCTAAGGAGGCCGAAATCGACGCTTGTACCGCCC |
| | ATAACTACGATGTGCTGAGACTGCTGTCCCTCATGgcGCAGCTGTGCATGcaCTCCGTCGCTG |
| | GAACAGCCTTTAAGCTGGCTGAGTCCGCTCTGTTCAACATTGAGGATGTGCTCAGCGCCGA |
| | TCTGAAGGAAATCCTCGATGAAGCCTTCTCCGGCGCCGTGAACAAGCTCAATGACGGATTC |
| | GTGCAGCACTCCGGCAACAATCTGTACGTGCTCCAGCAGCTGTACCCTAATGAGACCATCG |
| | AGAGAATCGCCGAGAAGTACTACAGACTCACCGTGAGGAAGGAGGATCTGAACATGGGA |
| | GTCAACATTAAAAAGCTGAGGGAGCTGATCGTGGGCCAATACTTTCCCGAGGTCCTCGAC |
| | AAAGAATACGACCTCTCCAAGAATGGAGACAGCGTGGTGACATACAGAAGCAAGATTTAT |
| | ACCGTGATGAATTACATTCTGCTGTATTACCTCGAGGACCACGACTCCAGCAGAGAAAGCA |
| | TGGTCGAAGCTCTGAGACAAAACAGAGAGGGCGATGAAGGCAAGGAGGAGATCTATAGA |
| | CAGTTTGCCAAGAAGGTGTGGAACGGCGTGTCCGGACTGTTTGGCGTGTGTCTGAACCTCT |
| | TCAAGACCGAAAAGAGAAACAAGTTTAGGAGCAAAGTCGCCCTCCCCGATGTGTCCGGCG |
| | CTGCCTATATGCTCTCCTCCGAGAACATCGACTACTTTGTCAAGATGCTCTTCTTTGTGTGT |
| | AAGTTTCTGGATGGCAAAGAAATCAACGAGCTGCTGTGCGCTCTGATCAACAAATTTGATA |
| | ATATTGCCGATATTCTGGATGCTGCCGCTCAATGTGGCTCCTCCGTCTGGTTCGTGGACAGC |
| | TATAGGTTCTTCGAGAGATCTAGGAGGATTAGCGCCCAGATTAGAATCGTGAAGAACATC |
| | GCTTCCAAGGATTTTAAGAAATCCAAGAAGGATTCCGATGAGAGCTACCCCGAGCAGCTG |
| | TATCTGGATGCTCTGGCTCTGCTCGGAGACGTCATCTCCAAGTACAAGCAGAATAGAGATG |
| | GCAGCGTCGTCATCGATGACCAAGGCAATGCCGTGCTGACAGAGCAATACAAGAGGTTTA |
| | GATATGAATTTTTCGAGGAGATCAAGAGGGACGAAAGCGGCGGCATCAAGTACAAGAAGT |
| | CCGGAAAACCCGAGTACAACCATCAGAGAAGGAATTTTATTCTGAATAATGTGCTGAAAA |
| | GCAAATGGTTTTTCTATGTGGTGAAGTACAATAGGCCCAGCAGCTGCAGAGAACTGATGA |
| | AGAATAAGGAAATTCTGAGGTTCGTGCTGAGAGACATCCCCGACTCCCAAGTGAGAAGAT |
| | ACTTTAAGGCCGTCCAAGGAGAGGAAGCTTACGCTAGCGCCGAAGCTATGAGGACAAGAC |
| | TGGTCGACGCTCTGTCCCAATTTAGCGTCACAGCTTGTCTGGATGAAGTGGGCGGCATGAC |
| | AGACAAGGAATTCGCCTCCCAGAGGGCCGTCGATAGCAAAGAAAAACTGAGAGCCATCAT |
| | CAGACTGTATCTGACAGTCGCCTATCTGATTACCAAGAGCATGGTGAAGGTGAATACAAG |
| | GTTTAGCATTGCCTTTAGCGTGCTGGAGAGGGACTACTATCTGCTCATTGACGGCAAGAAG |
| | AAATCCAGCGACTACACCGGAGAGGATATGCTGGCTCTGACCAGAAAATTTGTGGGCGAA |
| | GATGCTGGACTGTATAGAGAGTGGAAAGAGAAGAACGCTGAAGCCAAGGACAAATATTTT |
| | GACAAGGCCGAAAGGAAGAAGGTGCTGAGACAGAACGATAAGATGATCAGAAAGATGCA |
| | CTTCACACCCCACTCCCTCAATTACGTCCAAAAGAATCTCGAAAGCGTCCAGAGCAACGGA |
| | CTGGCCGCCGTCATCAAGGAATATagAAATGCCGTCGCTcaCCTCAATATCATCAATAGACT |
| | GGACGAGTACATTGGCTCCGCTAGGGCTGATAGCTACTACTCTCTGTACTGTTACTGCCTC |
| | CAAATGTATCTGAGCAAGAACTTCAGCGTGGGCTACCTCATCAACGTGCAAAAGCAGCTG |
| | GAGGAGCACCACACCTACATGAAGGATCTCATGTGGCTGCTCAACATCCCCTTCGCTTACA |
| | ACCTCGCCAGATACAAAAATCTGTCCAACGAAAAACTCTTTTACGACGAGGAAGCCGCCG |
| | CCGAAAAGGCTGACAAGGCTGAGAACGAGAGAGGCGAA (SEQ ID NO: 617) |
| Linker | GGAAGC |
| SV-40 NLS | CCCAAGAAGAAAAGGAAGGTC (SEQ ID NO: 532) |
| Linker | GGCGGAtct (SEQ ID NO: 613) |
| Myc Tag | GAGCAgAAACTGATTAGcGAAGAgGATCTC (SEQ ID NO: 602) |
| WPRE3 | GATAATCAACCTCTGGATTACAAAATTTGTGAAAGATTGACTGGTATTCTTAACTATGTTG |
| | CTCCTTTTACGCTATGTGGATACGCTGCTTTAATGCCTTTGTATCATGCTATTGCTTCCCGTA |
| | TGGCTTTCATTTTCTCCTCCTTGTATAAATCCTGGTTAGTTCTTGCCACGGCGGAACTCATC |
| | GCCGCCTGCCTTGCCCGCTGCTGGACAGGGGCTCGGCTGTTGGGCACTGACAATTCCGTGG |
| | (SEQ ID NO: 603) |
| SV-40 poly A | AACTTGTTTATTGCAGCTTATAATGGTTACAAATAAAGCAATAGCATCACAAATTTCACAA |
| | ATAAAGCATTTTTTTCACTGCATTCTAGTTGTGGTTTGTCCAAACTCATCAATGTATCTTA |
| | (SEQ ID NO: 533) |
| 3′ITR | Aggaacccctagtgatggagttggccactccctctctgcgcgctcgctcgctcactgaggccgggcgaccaaaggtcgccc |
| | gacgcccgggctttgcccgggcggcctcagtgagcgagcgagcgcgcagctgcctgcagg (SEQ ID NO: 600) |

Cas13d: dSeq212 Targeting CUG No Endonuclease (with 1 Point Mutation H298A at HEPN1)

In some embodiments, the disclosure provides a CUG-targeting catalytically inactive Cas (dCas13d) having an H298A mutation at HEPN1 as set forth in Table R.

TABLE R
Exemplary CUG-Targeting dcas 13d for blocking CUG RNA

| Plasmid | |
|---|---|
| Element | Amino Acid Sequences |
| dCas13d Seq212 | KKKHQSAAEKRQVKKLKNQEKAQKYASEPSPLQSDTAGVECSQKKTVVSHIASSKTLAKAMG |
| H298A | LKSTLVMGDKLVITSFAASKAVGGAGYKSANIEKITDLQGRVIEEHERMFSADVGEKNIELSKN |
| | DCHTNVNNPVVTNIGKDYIGLKSRLEQEFFGKTFENDNLHVQLAYNILDIKKILGTYVNNIIYIF |
| | YNLNRAGTGRDERMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMRQLCMASVAGTAFKLAESALFN |
| | IEDVLSADLKEILDEAFSGAVNKLNDGFVQHSGNNLYVLQQLYPNETIERIAEKYYRLTVRKED |
| | LNMGVNIKKLRELIVGQYFPEVLDKEYDLSKNGDSVVTYRSKIYTVMNYILLYYLEDHDSSRE |
| | SMVEALRQNREGDEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQCGSSVWFVDSYRF |
| | FERSRRISAQIRIVKNIASKDFKKSKKDSDESYPEQLYLDALALLGDVISKYKQNRDGSVVIDDQ |
| | GNAVLTEQYKRFRYEFFEEIKRDESGGIKYKKSGKPEYNHQRRNFILNNVLKSKWFFYVVKYN |
| | RPSSCRELMKNKEILRFVLRDIPDSQVRRYFKAVQGEEAYASAEAMRTRLVDALSQFSVTACL |
| | DEVGGMTDKEFASQRAVDSKEKLRAIIRLYLTVAYLITKSMVKVNTRFSIAFSVLERDYYLLID |
| | GKKKSSDYTGEDMLALTRKFVGEDAGLYREWKEKNAEAKDKYFDKAERKKVLRQNDKMIR |
| | KMHFTPHSLNYVQKNLESVQSNGLAAVIKEYRNAVAHLNIINRLDEYIGSARADSYYSLYCYC |
| | LQMYLSKNFSVGYLINVQKQLEEHHTYMKDLMWLLNIPFAYNLARYKNLSNEKLFYDEEAAA |
| | EKADKAENERGE (SEQ ID NO: 589) |
| Linker | GS |
| SV-40 NLS | PKKKRKV (SEQ ID NO: 437) |
| Linker | GGS |
| Myc Tag | EQKLISEEDL (SEQ ID NO: 610) |

In some embodiments, an AAV vector of the disclosure, comprising a CUG-targeting dCas13d protein comprises from 5′ to 3′ the elements as set forth in Table S. In some aspects, the AAV vector comprises the nucleic acid sequence set forth in SEQ ID NO. 596 or 597.

TABLE S
Exemplary AAV vector comprising a CUG-Targeting dCas13d

| Plasmid | |
|---|---|
| Element | Amino Acid Sequences |
| 5′ ITR | Cctgcaggcagctgcgcgctcgctcgctcactgaggccgcccgggcgtcgggcgacctttggtcgcccggcctca |
| | gtgagcgagcgagcgcgcagagagggagtggccaactccatcactaggggttcct (SEQ ID NO: 599) |
| Human U6 | Gagggcctatttcccatgattccttcatatttgcatatacgatacaaggctgttagagagataattggaattaa |
| promoter | tttgactgtaaacacaaagatattagtacaaaatacgtgacgtagaaagtaataatttcttgggtagtttgcagt |
| | tttaaaattatgttttaaaatggactatcatatgcttaccgtaacttgaaagtatttcgatttcttggctttata |
| | tatcttgtggaaaggacgaaacacc (SEQ ID NO: 519) |
| Seq212 direct | Tagccctgcagtaaggcagggttctaagac (SEQ ID NO: 614) |
| repeat (DR) | |
| Spacer (CTG | CAGCAGCAGCAGCAGCAGCAGCAGCA (SEQ ID NO: 459) |
| guide 3) | |
| EFS/UBB | GGGCAGAGCGCACATCGCCCACAGTCCCCGAGAAGTTGGGGGGAGGGGTCGGCAATTGAa |
| promoter | CCGGTGCCTAGAGAAGGTGGCGCGGGGTAAACTGGGAAAGTGATGTCGTGTACTGGCTCC |
| | GCCTTTTTCCCGAGGGTGGGGGAGAACCGTATATAAGTGCAGTAGTCGCCGTGAACGTTCT |
| | TTTTCGCAACGGGTTTGCCGCCAGAACACAGaattccagGTAAGTCCCGCAGCCGTAACGACCT |
| | TGGGGGGGTGTGAGATTCTCATTCTAATTTTGAAGAATATTAGGTGTAAAAGCAAGAAATA |
| | CAATGATCCTGAGGTGACACGCTTATGTTTTACTTTTAAACTAGGT (SEQ ID NO: 609) |
| Kozak Sequence | GCCGCCACCATG (SEQ ID NO: 529) |
| Dead Seq212 | AAGAAGAAGCACCAGAGCGCCGCCGAGAAGAGGCAAGTGAAGAAGCTCAAGAATCAAGA |
| H298A | GAAGGCCCAGAAGTACGCTAGCGAGCCTTCCCCCCTCCAGAGCGATACAGCTGGCGTGGA |
| | ATGCTCCCAGAAAAAGACAGTCGTCAGCCACATTGCCAGCTCCAAGACACTGGCCAAGGC |
| | TATGGGACTCAAATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAAATCACAGATCTG |
| | CAAGGAAGGGTCATTGAGGAGCACGAAAGGATGTTTAGCGCCGATGTCGGAGAGAAAAA |
| | TATCGAACTGAGCAAGAATGACTGCCACACCAACGTCAACAACCCCGTGGTGACCAACAT |
| | CGGAAAGGATTACATCGGACTGAAATCTAGGCTGGAGCAAGAGTTTTTCGGCAAGACATT |
| | CGAGAATGACAATCTGCATGTGCAGCTGGCCTACAATATCCTCGACATCAAGAAAATTCTG |
| | GGAACCTATGTGAACAATATCATTTATATCTTCTACAATCTGAATAGGGCTGGCACCGGCA |
| | GAGATGAGAGGATGTATGACGACCTCATCGGCACACTGTACGCTTACAAACCCATGGAGG |
| | CTCAACAGACCTATCTGCTCAAAGGCGACAAGGATATGAGGAGGTTTGAGGAGGTGAAAC |
| | AGCTGCTGCAAAACACCTCCGCTTACTATGTGTATTACGGCACACTGTTCGAGAAGGTGAA |
| | GGCTAAGAGCAAGAAGGAACAGAGGGCTAAGGAGGCCGAAATCGACGCTTGTACCGCCC |
| | ATAACTACGATGTGCTGAGACTGCTGTCCCTCATGagGCAGCTGTGCATGgcCTCCGTCGCT |
| | GGAACAGCCTTTAAGCTGGCTGAGTCCGCTCTGTTCAACATTGAGGATGTGCTCAGCGCCG |
| | ATCTGAAGGAAATCCTCGATGAAGCCTTCTCCGGCGCCGTGAACAAGCTCAATGACGGATT |
| | CGTGCAGCACTCCGGCAACAATCTGTACGTGCTCCAGCAGCTGTACCCTAATGAGACCATC |
| | GAGAGAATCGCCGAGAAGTACTACAGACTCACCGTGAGGAAGGAGGATCTGAACATGGG |
| | AGTCAACATTAAAAAGCTGAGGGAGCTGATCGTGGGCCAATACTTTCCCGAGGTCCTCGA |
| | CAAAGAATACGACCTCTCCAAGAATGGAGACAGCGTGGTGACATACAGAAGCAAGATTTA |
| | TACCGTGATGAATTACATTCTGCTGTATTACCTCGAGGACCACGACTCCAGCAGAGAAAGC |
| | ATGGTCGAAGCTCTGAGACAAAACAGAGAGGGCGATGAAGGCAAGGAGGAGATCTATAG |
| | ACAGTTTGCCAAGAAGGTGTGGAACGGCGTGTCCGGACTGTTTGGCGTGTGTCTGAACCTC |
| | TTCAAGACCGAAAAGAGAAACAAGTTTAGGAGCAAAGTCGCCCTCCCCGATGTGTCCGGC |
| | GCTGCCTATATGCTCTCCTCCGAGAACATCGACTACTTTGTCAAGATGCTCTTCTTTGTGTG |
| | TAAGTTTCTGGATGGCAAAGAAATCAACGAGCTGCTGTGCGCTCTGATCAACAAATTTGAT |
| | AATATTGCCGATATTCTGGATGCTGCCGCTCAATGTGGCTCCTCCGTCTGGTTCGTGGACA |
| | GCTATAGGTTCTTCGAGAGATCTAGGAGGATTAGCGCCCAGATTAGAATCGTGAAGAACA |
| | TCGCTTCCAAGGATTTTAAGAAATCCAAGAAGGATTCCGATGAGAGCTACCCCGAGCAGC |
| | TGTATCTGGATGCTCTGGCTCTGCTCGGAGACGTCATCTCCAAGTACAAGCAGAATAGAGA |
| | TGGCAGCGTCGTCATCGATGACCAAGGCAATGCCGTGCTGACAGAGCAATACAAGAGGTT |
| | TAGATATGAATTTTTCGAGGAGATCAAGAGGGACGAAAGCGGCGGCATCAAGTACAAGAA |
| | GTCCGGAAAACCCGAGTACAACCATCAGAGAAGGAATTTTATTCTGAATAATGTGCTGAA |
| | AAGCAAATGGTTTTTCTATGTGGTGAAGTACAATAGGCCCAGCAGCTGCAGAGAACTGAT |
| | GAAGAATAAGGAAATTCTGAGGTTCGTGCTGAGAGACATCCCCGACTCCCAAGTGAGAAG |
| | ATACTTTAAGGCCGTCCAAGGAGAGGAAGCTTACGCTAGCGCCGAAGCTATGAGGACAAG |
| | ACTGGTCGACGCTCTGTCCCAATTTAGCGTCACAGCTTGTCTGGATGAAGTGGGCGGCATG |
| | ACAGACAAGGAATTCGCCTCCCAGAGGGCCGTCGATAGCAAAGAAAAACTGAGAGCCATC |
| | ATCAGACTGTATCTGACAGTCGCCTATCTGATTACCAAGAGCATGGTGAAGGTGAATACAA |
| | GGTTTAGCATTGCCTTTAGCGTGCTGGAGAGGGACTACTATCTGCTCATTGACGGCAAGAA |
| | GAAATCCAGCGACTACACCGGAGAGGATATGCTGGCTCTGACCAGAAAATTTGTGGGCGA |
| | AGATGCTGGACTGTATAGAGAGTGGAAAGAGAAGAACGCTGAAGCCAAGGACAAATATTT |
| | TGACAAGGCCGAAAGGAAGAAGGTGCTGAGACAGAACGATAAGATGATCAGAAAGATGC |
| | ACTTCACACCCCACTCCCTCAATTACGTCCAAAAGAATCTCGAAAGCGTCCAGAGCAACGG |
| | ACTGGCCGCCGTCATCAAGGAATATagAAATGCCGTCGCTcaCCTCAATATCATCAATAGAC |
| | TGGACGAGTACATTGGCTCCGCTAGGGCTGATAGCTACTACTCTCTGTACTGTTACTGCCTC |
| | CAAATGTATCTGAGCAAGAACTTCAGCGTGGGCTACCTCATCAACGTGCAAAAGCAGCTG |
| | GAGGAGCACCACACCTACATGAAGGATCTCATGTGGCTGCTCAACATCCCCTTCGCTTACA |
| | ACCTCGCCAGATACAAAAATCTGTCCAACGAAAAACTCTTTTACGACGAGGAAGCCGCCG |
| | CCGAAAAGGCTGACAAGGCTGAGAACGAGAGAGGCGAA (SEQ ID NO: 618) |
| Linker | GGAAGC |
| SV-40 NLS | CCCAAGAAGAAAAGGAAGGTC (SEQ ID NO: 532) |
| Linker | GGCGGAtct |
| Myc Tag | GAGCAgAAACTGATTAGcGAAGAgGATCTC (SEQ ID NO: 602) |
| WPRE3 | GATAATCAACCTCTGGATTACAAAATTTGTGAAAGATTGACTGGTATTCTTAACTATGTTG |
| | CTCCTTTTACGCTATGTGGATACGCTGCTTTAATGCCTTTGTATCATGCTATTGCTTCCCGTA |
| | TGGCTTTCATTTTCTCCTCCTTGTATAAATCCTGGTTAGTTCTTGCCACGGCGGAACTCATC |
| | GCCGCCTGCCTTGCCCGCTGCTGGACAGGGGCTCGGCTGTTGGGCACTGACAATTCCGTGG |
| | (SEQ ID NO: 603) |
| SV-40 poly A | AACTTGTTTATTGCAGCTTATAATGGTTACAAATAAAGCAATAGCATCACAAATTTCACAA |
| | ATAAAGCATTTTTTTCACTGCATTCTAGTTGTGGTTTGTCCAAACTCATCAATGTATCTTA |
| | (SEQ ID NO: 533) |
| 3′ITR | Aggaacccctagtgatggagttggccactccctctctgcgcgctcgctcgctcactgaggccgggcgaccaaag |
| | gtcgcccgacgcccgggctttgcccgggcggcctcagtgagcgagcgagcgcgcagctgcctgcagg (SEQ |
| | ID NO: 600) |

### Guide RNAs for RNA-Guided RNA-Binding Proteins

The terms guide RNA (gRNA) and single guide RNA (sgRNA) are used interchangeably throughout the disclosure.

Guide RNAs (gRNAs) of the disclosure may comprise of a spacer sequence and a “direct repeat” (DR) sequence. In some embodiments, a guide RNA is a single guide RNA (sgRNA) comprising a contiguous spacer sequence and DR sequence. In some embodiments, the spacer sequence and the DR sequence are not contiguous. In some embodiments, the gRNA comprises a DR sequence. DR sequences refer to the repetitive sequences in the CRISPR locus (naturally-occurring in a bacterial genome or plasmid) that are interspersed with the spacer sequences. It is well known that one would be able to infer the DR sequence of a corresponding (or cognate) Cas protein if the sequence of the associated CRISPR locus is known. In some embodiments, a guide RNA comprises a direct repeat (DR) sequence and a spacer sequence. In some embodiments, a sequence encoding a guide RNA or single guide RNA of the disclosure comprises or consists of a spacer sequence and a DR sequence, that are separated by a linker sequence. In some embodiments, the linker sequence may comprise or consist of 1, 2, 3, 4, 5, 6, 7, 8, 9, 10, 15, 20, 25, 30, 35, 40, 45, 50 or any number of nucleotides (nt) in between. In some embodiments, the linker sequence may comprise at least 1, 2, 3, 4, 5, 6, 7, 8, 9, 10, 15, 20, 25, 30, 35, 40, 45, 50 or any number of nucleotides in between. In some embodiments, the DR sequence is a Cas13d DR sequence.

In one embodiment, the gRNA that hybridizes with the one or more target RNA molecules in a Cas13d-mediated manner includes one or more direct repeat (DR) sequences, one or more spacer sequences, such as, e.g., one or more sequences comprising an array of DR-spacer-DR-spacer. In one embodiment, a plurality of gRNAs are generated from a single array, wherein each gRNA can be different, for example target different RNAs or target multiple regions of a single RNA, or combinations thereof. In some embodiments, an isolated gRNA includes one or more direct repeat sequences, such as an unprocessed (e.g., about 36 nt) or processed DR (e.g., about 30 nt). In some embodiments, a gRNA can further include one or more spacer sequences specific for (e.g., is complementary to) the target RNA. In certain such embodiments, multiple polIII promoters can be used to drive multiple gRNAs, spacers and/or DRs. In one embodiment, a guide array comprises a DR (about 36nt)-spacer (about 30nt)-DR (about 36nt)-spacer (about 30nt).

Guide RNAs (gRNAs) of the disclosure may comprise non-naturally occurring nucleotides. In some embodiments, a guide RNA of the disclosure or a sequence encoding the guide RNA comprises or consists of modified or synthetic RNA nucleotides. Exemplary modified RNA nucleotides include, but are not limited to, pseudouridine (Ψ), dihydrouridine (D), inosine (I), and 7-methylguanosine (m7G), hypoxanthine, xanthine, xanthosine, 7-methylguanine, 5,6-Dihydrouracil, 5-methylcytosine, 5-methylcytidine, 5-hydropxymethylcytosine, isoguanine, and isocytosine.

Guide RNAs (gRNAs) of the disclosure may bind modified RNA within a target sequence. Within a target sequence, guide RNAs (gRNAs) of the disclosure may bind modified or mutated (e.g., pathogenic) RNA. Exemplary epigenetically or post-transcriptionally modified RNA include, but are not limited to, 2′-O-Methylation (2′-OMe) (2′-O-methylation occurs on the oxygen of the free 2′-OH of the ribose moiety), N6-methyladenosine (m6A), and 5-methylcytosine (m5C).

In some embodiments of the compositions of the disclosure, a guide RNA of the disclosure comprises at least one sequence encoding a non-coding C/D box small nucleolar RNA (snoRNA) sequence. In some embodiments, the snoRNA sequence comprises at least one sequence that is complementary to the target RNA, wherein the target sequence of the RNA molecule comprises at least one 2′-OMe. In some embodiments, the snoRNA sequence comprises at least one sequence that is complementary to the target RNA, wherein the at least one sequence that is complementary to the target RNA comprises a box C motif (RUGAUGA) (SEQ ID NO: 523) and a box D motif (CUGA) (SEQ ID NO: 524).

Spacer sequences of the disclosure bind to the target sequence of an RNA molecule. In some embodiments, spacer sequences of the disclosure bind to pathogenic target RNA.

In some embodiments of the compositions of the disclosure, the sequence comprising the gRNA further comprises a spacer sequence that specifically binds to the target RNA sequence. In some embodiments, the spacer sequence has at least 50%, 55%, 60%, 65%, 70%, 75%, 80%, 87%, 90%, 95%, 97%, 99% or any percentage in between of complementarity to the target RNA sequence. In some embodiments, the spacer sequence has 100% complementarity to the target RNA sequence. In some embodiments, the spacer sequence comprises or consists of 20 nucleotides. In some embodiments, the spacer sequence comprises or consists of 21 nucleotides, 22 nucleotides, 23 nucleotides, 24 nucleotides, 25 nucleotides, 26 nucleotides, 27 nucleotides, 28 nucleotides, or 29 nucleotides. In some embodiments, the spacer sequence comprises or consists of 26 nucleotides. In some embodiments, the spacer sequence is non-processed and comprises or consists of 30 nucleotides. In some embodiments the non-processed spacer sequence comprises or consists of 30-36 nucleotides.

DR sequences of the disclosure bind the Cas polypeptide of the disclosure. Upon binding of the spacer sequence of the gRNA to the target RNA sequence, the Cas protein bound to the DR sequence of the gRNA is positioned at the target RNA sequence. DR sequence having sufficient complementarity to its cognate Cas protein, or nucleic acid thereof, binds selectively to the target nucleic acid sequence of the Cas protein and has at least 50%, 55%, 60%, 65%, 70%, 75%, 80%, 85%, 90%, 95%, 96, 97%, 98%, 99%, or any percentage identity in between to the sequence. In some embodiments, a sequence having sufficient complementarity has 100% identity. In some embodiments, DR sequences of the disclosure comprise a secondary structure or a tertiary structure. Exemplary secondary structures include, but are not limited to, a helix, a stem loop, a bulge, a tetraloop and a pseudoknot. Exemplary tertiary structures include, but are not limited to, an A-form of a helix, a B-form of a helix, and a Z-form of a helix. Exemplary tertiary structures include, but are not limited to, a twisted or helicized stem loop. Exemplary tertiary structures include, but are not limited to, a twisted or helicized pseudoknot. In some embodiments, DR sequences of the disclosure comprise at least one secondary structure or at least one tertiary structure. In some embodiments, DR sequences of the disclosure comprise one or more secondary structure(s) or one or more tertiary structure(s).

In some embodiments of the compositions of the disclosure, a guide RNA or a portion thereof selectively binds to a tetraloop motif in an RNA molecule of the disclosure. In some embodiments, a target sequence of an RNA molecule comprises a tetraloop motif. In some embodiments, the tetraloop motif is a “GRNA” motif comprising or consisting of one or more of the sequences of GAAA, GUGA, GCAA or GAGA.

In some embodiments of the compositions of the disclosure, a guide RNA or a portion thereof that binds to a target sequence of an RNA molecule hybridizes to the target sequence of the RNA molecule. In some embodiments, a guide RNA or a portion thereof that binds to a first RNA binding protein or to a second RNA binding protein covalently binds to the first RNA binding protein or to the second RNA binding protein. In some embodiments, a guide RNA or a portion thereof that binds to a first RNA binding protein or to a second RNA binding protein non-covalently binds to the first RNA binding protein or to the second RNA binding protein.

In some embodiments of the compositions of the disclosure, a guide RNA or a portion thereof comprises or consists of between 10 and 100 nucleotides, inclusive of the endpoints. In some embodiments, a spacer sequence of the disclosure comprises or consists of between 10 and 30 nucleotides, inclusive of the endpoints. In some embodiments, a spacer sequence of the disclosure comprises or consists of 15, 16, 17, 18, 19, 20, 21, 22, 23, 24, 25, 26, 27, 28, 29 or 30 nucleotides. In some embodiments, the spacer sequence of the disclosure comprises or consists of 20 nucleotides. In some embodiments, the spacer sequence of the disclosure comprises or consists of 21 nucleotides. In some embodiments, the spacer sequence of the disclosure comprises or consists of 26 nucleotides.

Guide molecules generally exist in various states of processing. In one example, an unprocessed guide RNA is 36nt of DR followed by 30-32 nt of spacer. The guide RNA is processed (truncated/modified) by Cas13d itself or other RNases into the shorter “mature” form. In some embodiments, an unprocessed guide sequence is about, or at least about 30, 35, 40, 45, 50, 55, 60, 61, 62, 63, 64, 65, 66, 67, 68, 69, 70, 71, 72, 73, 74, 75, or more nucleotides (nt) in length. In some embodiments, a processed guide sequence is about 44 to 60 nt (such as 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nt). In some embodiments, an unprocessed spacer is about 28-32 nt long (such as 25, 26, 27, 28, 29, 30, 31, 32, 33, 34, or 35 nt) while the mature (processed) spacer can be about 10 to 30 nt, 10 to 25 nt, 14 to 25 nt, 20 to 22 nt, or 14-30 nt (such as 10, 11, 12, 13, 14, 15, 16, 17, 18, 19, 20, 21, 22, 23, 24, 25, 26, 27, 28, 29, 30, 31, 32, 33, 34, or 35 nt). In some embodiments, an unprocessed DR is about 36 nt (such as 30, 31, 32, 33, 34, 35, 36, 37, 38, 39, 40 or 41 nt), while the processed DR is about 30 nt (such as 25, 26, 27, 28, 29, 30, 31, 32, 33, 34, or 35 nt). In some embodiments, a DR sequence is truncated by 1-10 nucleotides (such as 1, 2, 3, 4, 5, 6, 7, 8, 9, to 10 nucleotides at e.g., the 5′ end in order to be expressed as mature pre-processed guide RNAs.

In some embodiments of the compositions of the disclosure, a guide RNA or a portion thereof does not comprise a nuclear localization sequence (NLS).

In some embodiments of the compositions of the disclosure, a guide RNA or a portion thereof comprises a sequence complementary to a protospacer flanking sequence (PFS). In some embodiments, including those wherein a guide RNA or a portion thereof comprises a sequence complementary to a PFS, the first RNA binding protein may comprise a sequence isolated or derived from a Cas13 protein. In some embodiments, including those wherein a guide RNA or a portion thereof comprises a sequence complementary to a PFS, the first RNA binding protein may comprise a sequence encoding a Cas13 protein or an RNA-binding portion thereof In some embodiments, the guide RNA or a portion thereof does not comprise a sequence complementary to a PFS.

In some embodiments of the compositions of the disclosure, guide RNA sequence of the disclosure comprises a promoter sequence to drive expression of the guide RNA. In some embodiments, a vector comprising a guide RNA sequence of the disclosure comprises a promoter sequence to drive expression of the guide RNA. In some embodiments, the promoter to drive expression of the guide RNA is a constitutive promoter. In some embodiments, the promoter sequence is an inducible promoter. In some embodiments, the promoter is a sequence which is a tissue-specific and/or cell-type specific promoter. In some embodiments, the promoter is a hybrid or a recombinant promoter. In some embodiments, the promoter is a promoter capable of expressing the guide RNA in a mammalian cell. In some embodiments, the promoter is a promoter capable of expressing the guide RNA in a human cell. In some embodiments, the promoter is a promoter capable of expressing the guide RNA and restricting the guide RNA to the nucleus of the cell. In some embodiments, the promoter is a human RNA polymerase promoter or a sequence isolated or derived from a sequence encoding a human RNA polymerase promoter. In some embodiments, the promoter is a U6 promoter or a sequence isolated or derived from a sequence encoding a U6 promoter. In some embodiments, the U6 promoter is a human U6 promoter. In some embodiments, the promoter is a human tRNA promoter or a sequence isolated or derived from a sequence encoding a human tRNA promoter. In some embodiments, the promoter is a human valine tRNA promoter or a sequence isolated or derived from a sequence encoding a human valine tRNA promoter.

In some embodiments of the compositions of the disclosure, a promoter to drive expression of the guide RNA further comprises a regulatory element. In some embodiments, a vector comprising a promoter sequence to drive expression of the guide RNA further comprises a regulatory element. In some embodiments, a regulatory element enhances expression of the guide RNA. Exemplary regulatory elements include, but are not limited to, an enhancer element, an intron, an exon, or a combination thereof.

In some embodiments of the compositions of the disclosure, a vector of the disclosure comprises one or more of a sequence encoding a guide RNA, a promoter sequence to drive expression of the guide RNA and a sequence encoding a regulatory element. In some embodiments of the compositions of the disclosure, the vector further comprises a sequence encoding a fusion protein of the disclosure.

### RNA-Guided RNA-Binding Proteins

In some embodiments of the compositions of the disclosure, gRNAs correspond to target RNA molecules and an RNA-guided RNA binding protein. In some embodiments, the gRNAs correspond to an RNA-guided RNA binding fusion protein, wherein the fusion protein comprises first and second RNA binding proteins. In some embodiments, the first RNA-binding protein in the fusion protein is a deactivated RNA-binding protein, e.g., a deactivated Cas or catalytically dead Cas protein. In some embodiments, along a sequence encoding the RNA-binding fusion protein, the sequence encoding the first RNA binding protein is positioned 5′ of the sequence encoding the second RNA binding protein. In some embodiments, along a sequence encoding the fusion protein, the sequence encoding the first RNA binding protein is positioned 3′ of the sequence encoding the second RNA binding protein.

In some embodiments of the compositions of the disclosure, the sequence encoding the first RNA binding protein comprises a sequence isolated or derived from a protein capable of binding an RNA molecule. In some embodiments, the sequence encoding the first RNA binding protein comprises a sequence isolated or derived from a protein capable of selectively binding an RNA molecule and not binding a DNA molecule, a mammalian DNA molecule or any DNA molecule. In some embodiments, the sequence encoding the first RNA binding protein comprises a sequence isolated or derived from a protein capable of binding an RNA molecule and inducing a break in the RNA molecule. In some embodiments, the sequence encoding the first RNA binding protein comprises a sequence isolated or derived from a protein capable of binding an RNA molecule, inducing a break in the RNA molecule, and not binding a DNA molecule, a mammalian DNA molecule or any DNA molecule. In some embodiments, the sequence encoding the first RNA binding protein comprises a sequence isolated or derived from a protein capable of binding an RNA molecule, inducing a break in the RNA molecule, and neither binding nor inducing a break in a DNA molecule, a mammalian DNA molecule or any DNA molecule.

In some embodiments of the compositions of the disclosure, the sequence encoding the first RNA-guided RNA binding protein comprises a sequence isolated or derived from a protein with no DNA nuclease activity.

In some embodiments of the compositions of the disclosure, the sequence encoding the RNA-guided RNA binding protein disclosed herein comprises a sequence isolated or derived from a CRISPR Cas protein. In some embodiments, the CRISPR Cas protein is not a Type II CRISPR Cas protein. In some embodiments, the CRISPR Cas protein is not a Cas9 protein.

In some embodiments of the compositions of the disclosure, the sequence encoding the RNA-guided RNA binding protein comprises a Type VI CRISPR Cas protein or portion thereof In some embodiments, the Type VI CRISPR Cas protein comprises a Cas13 protein or portion thereof. Exemplary Cas13 proteins of the disclosure may be isolated or derived from any species, including, but not limited to, bacteria or archaea. Exemplary Cas13 proteins of the disclosure may be isolated or derived from any species, including, but not limited to, Leptotrichia wadei, Listeria seeligeri serovar 1/2b (strain ATCC 35967/DSM 20751/CIP 100100/SLCC 3954), Lachnospiraceae bacterium, Clostridium aminophilum DSM 10710, Carnobacterium gallinarum DSM 4847, Paludibacter propionicigenes WB4, Listeria weihenstephanensis FSL R9-0317, Listeria weihenstephanensis FSL R9-0317, bacterium FSL M6-0635 (Listeria newyorkensis), Leptotrichia wadei F0279, Rhodobacter capsulatus SB 1003, Rhodobacter capsulatus R121, Rhodobacter capsulatus DE442 and Corynebacterium ulcerans. Exemplary Cas13 proteins of the disclosure may be DNA nuclease inactivated. Exemplary Cas13 proteins of the disclosure include, but are not limited to, Cas13a, Cas13b, Cas13c, Cas13d and orthologs thereof Exemplary Cas13b proteins of the disclosure include, but are not limited to, subtypes 1 and 2 referred to herein as Csx27 and Csx28, respectively.

Exemplary Cas13a proteins include, but are not limited to:

| Cas13a | Cas13a | | | |
|---|---|---|---|---|
| number | abbreviation | Organism name | Accession number | Direct Repeat sequence |
| Cas13a1 | LshCas13a | Leptotrichia | WP_018451595.1 | CCACCCCAATATCGAAGGGGACTAA |
| | | shahii | | AAC (SEQ ID NO: 393) |
| Cas13a2 | LwaCas13a | Leptotrichia | WP_021746774.1 | GATTTAGACTACCCCAAAAACGAAG |
| | | wadei | | GGGACTAAAAC (SEQ ID NO: |
| | | | | 394) |
| Cas13a3 | LseCas13a | Listeria | WP_012985477.1 | GTAAGAGACTACCTCTATATGAAAG |
| | | seeligeri | | AGGACTAAAAC (SEQ ID NO: |
| | | | | 395) |
| Cas13a4 | LbmCas13a | Lachnospiraceae | WP_044921188.1 | GTATTGAGAAAAGCCAGATATAGTT |
| | | bacterium | | GGCAATAGAC (SEQ ID NO: 396) |
| | | MA2020 | | |
| Cas13a5 | LbnCas13a | Lachnospiraceae | WP_022785443.1 | GTTGATGAGAAGAGCCCAAGATAG |
| | | bacterium | | AGGGCAATAAC (SEQ ID NO: |
| | | NK4A179 | | 397) |
| Cas13a6 | CamCas13a | [Clostridium] | WP_031473346.1 | GTCTATTGCCCTCTATATCGGGCTGT |
| | | aminophilum | | TCTCCAAAC (SEQ ID NO: 398) |
| | | DSM 10710 | | |
| Cas13a7 | CgaCas13a | Carnobacterium | WP_034560163.1 | ATTAAAGACTACCTCTAAATGTAAG |
| | | gallinarum DSM | | AGGACTATAAC (SEQ ID NO: |
| | | 4847 | | 399) |
| Cas13a8 | Cga2Cas13a | Carnobacterium | WP_034563842.1 | AATATAAACTACCTCTAAATGTAAG |
| | | gallinarum DSM | | AGGACTATAAC (SEQ ID NO: |
| | | 4847 | | 400) |
| Cas13a9 | Pprcas13a | Paludibacter | WP_013443710.1 | CTTGTGGATTATCCCAAAATTGAAG |
| | | propionicigenes | | GGAACTACAAC (SEQ ID NO: |
| | | WB4 | | 401) |
| Cas13a10 | LweCas13a | Listeria | WP_036059185.1 | GATTTAGAGTACCTCAAAATAGAAG |
| | | weihenstephanensis | | AGGTCTAAAAC (SEQ ID NO: |
| | | FSL R9-0317 | | 402) |
| Cas13a11 | LbfCas13a | Listeriaceae | WP_036091002.1 | GATTTAGAGTACCTCAAAACAAAAG |
| | | bacterium FSL | | AGGACTAAAAC (SEQ ID NO: |
| | | M6-0635 | | 403) |
| | | (Listeria | | |
| | | newyorkensis) | | |
| Cas13a12 | Lwa2cas13a | Leptotrichia | WP_021746774.1 | GATATAGATAACCCCAAAAACGAA |
| | | wadei F0279 | | GGGATCTAAAAC (SEQ ID NO: |
| | | | | 404) |
| Cas13a13 | RcsCas13a | Rhodobacter | WP_013067728.1 | GCCTCACATCACCGCCAAGACGACG |
| | | capsulatus SB | | GCGGACTGAAC (SEQ ID NO: 405) |
| | | 1003 | | |
| Cas13a14 | RcrCas13a | Rhodobacter | WP_023911507.1 | GCCTCACATCACCGCCAAGACGACG |
| | | capsulatus R121 | | GCGGACTGAAC (SEQ ID NO: |
| | | | | 406) |
| Cas13a15 | RcdCas13a | Rhodobacter | WP_023911507.1 | GCCTCACATCACCGCCAAGACGACG |
| | | capsulatus | | GCGGACTGAAC (SEQ ID NO: |
| | | DE442 | | 407) |

Exemplary wild type Cas13a proteins of the disclosure may comprise or consist of the amino acid sequence of SEQ ID NO: 408.

Exemplary Cas13b proteins include, but are not limited to:

| Species | Cas13b Accession | Cas13b Size (aa) |
|---|---|---|
| Paludibacter propionicigenes WB4 | WP_013446107.1 | 1155 |
| Prevotella sp. P5-60 | WP_044074780.1 | 1091 |
| Prevotella sp. P4-76 | WP_044072147.1 | 1091 |
| Prevotella sp. P5-125 | WP_044065294.1 | 1091 |
| Prevotella sp. P5-119 | WP_042518169.1 | 1091 |
| Capnocytophaga canimorsus Cc5 | WP_013997271.1 | 1200 |
| Phaeodactylibacter xiamenensis | WP_044218239.1 | 1132 |
| Porphyromonas gingivalis W83 | WP_005873511.1 | 1136 |
| Porphyromonas gingivalis F0570 | WP_021665475.1 | 1136 |
| Porphyromonas gingivalis ATCC 33277 | WP_012458151.1 | 1136 |
| Porphyromonas gingivalis F0185 | ERJ81987.1 | 1136 |
| Porphyromonas gingivalis F0185 | WP_021677657.1 | 1136 |
| Porphyromonas gingivalis SJD2 | WP_023846767.1 | 1136 |
| Porphyromonas gingivalis F0568 | ERJ65637.1 | 1136 |
| Porphyromonas gingivalis W4087 | ERJ87335.1 | 1136 |
| Porphyromonas gingivalis W4087 | WP_021680012.1 | 1136 |
| Porphyromonas gingivalis F0568 | WP_021663197.1 | 1136 |
| Porphyromonas gingivalis | WP_061156637.1 | 1136 |
| Porphyromonas gulae | WP_039445055.1 | 1136 |
| Bacteroides pyogenes F0041 | ERI81700.1 | 1116 |
| Bacteroides pyogenes JCM 10003 | WP_034542281.1 | 1116 |
| Alistipes sp. ZOR0009 | WP_047447901.1 | 954 |
| Flavobacterium branchiophilum FL-15 | WP_014084666.1 | 1151 |
| Prevotella sp. MA2016 | WP_036929175.1 | 1323 |
| Myroides odoratimimus CCUG 10230 | EHO06562.1 | 1160 |
| Myroides odoratimimus CCUG 3837 | EKB06014.1 | 1158 |
| Myroides odoratimimus CCUG 3837 | WP_006265509.1 | 1158 |
| Myroides odoratimimus CCUG 12901 | WP_006261414.1 | 1158 |
| Myroides odoratimimus CCUG 12901 | EHO08761.1 | 1158 |
| Myroides odoratimimus (NZ_CP013690.1) | WP_058700060.1 | 1160 |
| Bergeyella zoohelcum ATCC 43767 | EKB54193.1 | 1225 |
| Capnocytophaga cynodegmi | WP_041989581.1 | 1219 |
| Bergeyella zoohelcum ATCC 43767 | WP_002664492.1 | 1225 |
| Flavobacterium sp. 316 | WP_045968377.1 | 1156 |
| Psychroflexus torquis ATCC 700755 | WP_015024765.1 | 1146 |
| Flavobacterium columnare ATCC 49512 | WP_014165541.1 | 1180 |
| Flavobacterium columnare | WP_060381855.1 | 1214 |
| Flavobacterium columnare | WP_063744070.1 | 1214 |
| Flavobacterium columnare | WP_065213424.1 | 1215 |
| Chryseobacterium sp. YR477 | WP_047431796.1 | 1146 |
| Riemerella anatipestifer ATCC 11845 = DSM | WP_004919755.1 | 1096 |
| 15868 | | |
| Riemerella anatipestifer RA-CH-2 | WP_015345620.1 | 949 |
| Riemerella anatipestifer | WP_049354263.1 | 949 |
| Riemerella anatipestifer | WP_061710138.1 | 951 |
| Riemerella anatipestifer | WP_064970887.1 | 1096 |
| Prevotella saccharolytica F0055 | EKY00089.1 | 1151 |
| Prevotella saccharolytica JCM 17484 | WP_051522484.1 | 1152 |
| Prevotella buccae ATCC 33574 | EFU31981.1 | 1128 |
| Prevotella buccae ATCC 33574 | WP_004343973.1 | 1128 |
| Prevotella buccae D17 | WP_004343581.1 | 1128 |
| Prevotella sp. MSX73 | WP_007412163.1 | 1128 |
| Prevotella pallens ATCC 700821 | EGQ18444.1 | 1126 |
| Prevotella pallens ATCC 700821 | WP_006044833.1 | 1126 |
| Prevotella intermedia ATCC 25611 = DSM 20706 | WP_036860899.1 | 1127 |
| Prevotella intermedia | WP_061868553.1 | 1121 |
| Prevotella intermedia 17 | AFJ07523.1 | 1135 |
| Prevotella intermedia | WP_050955369.1 | 1133 |
| Prevotella intermedia | BAU18623.1 | 1134 |
| Prevotella intermedia ZT | KJJ86756.1 | 1126 |
| Prevotella aurantiaca JCM 15754 | WP_025000926.1 | 1125 |
| Prevotella pleuritidis F0068 | WP_021584635.1 | 1140 |
| Prevotella pleuritidis JCM 14110 | WP_036931485.1 | 1117 |
| Prevotella falsenii DSM 22864 = JCM 15124 | WP_036884929.1 | 1134 |
| Porphyromonas gulae | WP_039418912.1 | 1176 |
| Porphyromonas sp. COT-052 OH4946 | WP_039428968.1 | 1176 |
| Porphyromonas gulae | WP_039442171.1 | 1175 |
| Porphyromonas gulae | WP_039431778.1 | 1176 |
| Porphyromonas gulae | WP_046201018.1 | 1176 |
| Porphyromonas gulae | WP_039434803.1 | 1176 |
| Porphyromonas gulae | WP_039419792.1 | 1120 |
| Porphyromonas gulae | WP_039426176.1 | 1120 |
| Porphyromonas gulae | WP_039437199.1 | 1120 |
| Porphyromonas gingivalis TDC60 | WP_013816155.1 | 1120 |
| Porphyromonas gingivalis ATCC 33277 | WP_012458414.1 | 1120 |
| Porphyromonas gingivalis A7A1-28 | WP_058019250.1 | 1176 |
| Porphyromonas gingivalis JCVI SC001 | EOA10535.1 | 1176 |
| Porphyromonas gingivalis W50 | WP_005874195.1 | 1176 |
| Porphyromonas gingivalis | WP_052912312.1 | 1176 |
| Porphyromonas gingivalis AJW4 | WP_053444417.1 | 1120 |
| Porphyromonas gingivalis | WP_039417390.1 | 1120 |
| Porphyromonas gingivalis | WP_061156470.1 | 1120 |

Exemplary wild type Bergeyella zoohelcum ATCC 43767 Cas13b (BzCas13b) proteins of the disclosure may comprise or consist of the amino acid sequence of SEQ ID NO: 409.

In some embodiments of the compositions of the disclosure, the sequence encoding the RNA binding protein comprises a sequence isolated or derived from a Cas13d protein. Cas13d is an effector of the type VI-D CRISPR-Cas systems. In some embodiments, the Cas13d protein is an RNA-guided RNA endonuclease enzyme that can cut or bind RNA. In some embodiments, the Cas13d protein can include one or more higher eukaryotes and prokaryotes nucleotide-binding (HEPN) domains. In some embodiments, the Cas13d protein can include either a wild-type or mutated HEPN domain. In some embodiments, the Cas13d protein includes a mutated HEPN domain that cannot cut RNA but can process guide RNA. In some embodiments, the Cas13d protein does not require a protospacer flanking sequence. Also see WO Publication No. WO2019/040664 & US2019/0062724, which is incorporated herein by reference in its entirety, for further examples and sequences of Cas13d protein, without limitation.

In some embodiments, Cas13d sequences of the disclosure include without limitation SEQ ID NOS: 1-296 of WO 2019/040664, so numbered herein and included herewith.

SEQ ID NO: 1 is an exemplary Cas13d sequence from Eubacterium siraeum containing a HEPN site.

SEQ ID NO: 2 is an exemplary Cas13d sequence from Eubacterium siraeum containing a mutated HEPN site.

SEQ ID NO: 3 is an exemplary Cas13d sequence from uncultured Ruminococcus sp. containing a HEPN site.

SEQ ID NO: 4 is an exemplary Cas13d sequence from uncultured Ruminococcus sp. containing a mutated HEPN site.

SEQ ID NO: 5 is an exemplary Cas13d sequence from Gut_metagenome_contig2791000549.

SEQ ID NO: 6 is an exemplary Cas13d sequence from Gut_metagenome_contig855000317

SEQ ID NO: 7 is an exemplary Cas13d sequence from Gut_metagenome_contig3389000027.

SEQ ID NO: 8 is an exemplary Cas13d sequence from Gut_metagenome_contig8061000170.

SEQ ID NO: 9 is an exemplary Cas13d sequence from Gut_metagenome_contig1509000299.

SEQ ID NO: 10 is an exemplary Cas13d sequence from Gut_metagenome_contig9549000591.

SEQ ID NO: 11 is an exemplary Cas13d sequence from Gut_metagenome_contig71000500.

SEQ ID NO: 12 is an exemplary Cas13d sequence from human gut metagenome.

SEQ ID NO: 13 is an exemplary Cas13d sequence from Gut_metagenome_contig3915000357.

SEQ ID NO: 14 is an exemplary Cas13d sequence from Gut_metagenome_contig4719000173.

SEQ ID NO: 15 is an exemplary Cas13d sequence from Gut_metagenome_contig6929000468.

SEQ ID NO: 16 is an exemplary Cas13d sequence from Gut_metagenome_contig7367000486.

SEQ ID NO: 17 is an exemplary Cas13d sequence from Gut_metagenome_contig7930000403.

SEQ ID NO: 18 is an exemplary Cas13d sequence from Gut_metagenome_contig993000527.

SEQ ID NO: 19 is an exemplary Cas13d sequence from Gut_metagenome_contig6552000639.

SEQ ID NO: 20 is an exemplary Cas13d sequence from Gut_metagenome_contig11932000246.

SEQ ID NO: 21 is an exemplary Cas13d sequence from Gut_metagenome_contig12963000286.

SEQ ID NO: 22 is an exemplary Cas13d sequence from Gut_metagenome_contig2952000470.

SEQ ID NO: 23 is an exemplary Cas13d sequence from Gut_metagenome_contig451000394.

SEQ ID NO: 24 is an exemplary Cas13d sequence from Eubacterium_siraeum_DSM_15702.

SEQ ID NO: 25 is an exemplary Cas13d sequence from gut_metagenome_P19E0k2120140920,_c369000003.

SEQ ID NO: 26 is an exemplary Cas13d sequence from Gut_metagenome_contig7593000362.

SEQ ID NO: 27 is an exemplary Cas13d sequence from Gut_metagenome_contig12619000055.

SEQ ID NO: 28 is an exemplary Cas13d sequence from Gut_metagenome_contig1405000151.

SEQ ID NO: 29 is an exemplary Cas13d sequence from Chicken_gut_metagenome_c298474.

SEQ ID NO: 30 is an exemplary Cas13d sequence from Gut_metagenome_contig1516000227.

SEQ ID NO: 31 is an exemplary Cas13d sequence from Gut_metagenome_contig1838000319.

SEQ ID NO: 32 is an exemplary Cas13d sequence from Gut_metagenome_contig13123000268.

SEQ ID NO: 33 is an exemplary Cas13d sequence from Gut_metagenome_contig5294000434.

SEQ ID NO: 34 is an exemplary Cas13d sequence from Gut_metagenome_contig6415000192.

SEQ ID NO: 35 is an exemplary Cas13d sequence from Gut_metagenome_contig6144000300.

SEQ ID NO: 36 is an exemplary Cas13d sequence from Gut_metagenome_contig9118000041.

SEQ ID NO: 37 is an exemplary Cas13d sequence from Activated_sludge_metagenome_transcript_124486.

SEQ ID NO: 38 is an exemplary Cas13d sequence from Gut_metagenome_contig1322000437.

SEQ ID NO: 39 is an exemplary Cas13d sequence from Gut_metagenome_contig4582000531.

SEQ ID NO: 40 is an exemplary Cas13d sequence from Gut_metagenome_contig9190000283.

SEQ ID NO: 41 is an exemplary Cas13d sequence from Gut_metagenome_contig1709000510.

SEQ ID NO: 42 is an exemplary Cas13d sequence from M24_(LSQX01212483_Anaerobic_digester_metagenome) with a HEPN domain.

SEQ ID NO: 43 is an exemplary Cas13d sequence from Gut_metagenome_contig3833000494.

SEQ ID NO: 44 is an exemplary Cas13d sequence from Activated_sludge_metagenome_transcript_117355.

SEQ ID NO: 45 is an exemplary Cas13d sequence from Gut_metagenome_contig11061000330.

SEQ ID NO: 46 is an exemplary Cas13d sequence from Gut_metagenome_contig338000322 from sheep gut metagenome.

SEQ ID NO: 47 is an exemplary Cas13d sequence from human gut metagenome.

SEQ ID NO: 48 is an exemplary Cas13d sequence from Gut_metagenome_contig9530000097.

SEQ ID NO: 49 is an exemplary Cas13d sequence from Gut_metagenome_contig1750000258.

SEQ ID NO: 50 is an exemplary Cas13d sequence from Gut_metagenome_contig5377000274.

SEQ ID NO: 51 is an exemplary Cas13d sequence from gut_metagenome_P19E0k2120140920_c248000089.

SEQ ID NO: 52 is an exemplary Cas13d sequence from Gut_metagenome_contig11400000031.

SEQ ID NO: 53 is an exemplary Cas13d sequence from Gut_metagenome_contig7940000191.

SEQ ID NO: 54 is an exemplary Cas13d sequence from Gut_metagenome_contig6049000251.

SEQ ID NO: 55 is an exemplary Cas13d sequence from Gut_metagenome_contig1137000500.

SEQ ID NO: 56 is an exemplary Cas13d sequence from Gut_metagenome_contig9368000105.

SEQ ID NO: 57 is an exemplary Cas13d sequence from Gut_metagenome_contig546000275.

SEQ ID NO: 58 is an exemplary Cas13d sequence from Gut_metagenome_contig7216000573.

SEQ ID NO: 59 is an exemplary Cas13d sequence from Gut_metagenome_contig4806000409.

SEQ ID NO: 60 is an exemplary Cas13d sequence from Gut_metagenome_contig10762000480.

SEQ ID NO: 61 is an exemplary Cas13d sequence from Gut_metagenome_contig4114000374.

SEQ ID NO: 62 is an exemplary Cas13d sequence from Ruminococcus_flavefaciens_FD1.

SEQ ID NO: 63 is an exemplary Cas13d sequence from Gut_metagenome_contig7093000170.

SEQ ID NO: 64 is an exemplary Cas13d sequence from Gut_metagenome_contig11113000384.

SEQ ID NO: 65 is an exemplary Cas13d sequence from Gut_metagenome_contig6403000259.

SEQ ID NO: 66 is an exemplary Cas13d sequence from Gut_metagenome_contig6193000124.

SEQ ID NO: 67 is an exemplary Cas13d sequence from Gut_metagenome_contig721000619.

SEQ ID NO: 68 is an exemplary Cas13d sequence from Gut_metagenome_contig1666000270.

SEQ ID NO: 69 is an exemplary Cas13d sequence from Gut_metagenome_contig2002000411.

SEQ ID NO: 70 is an exemplary Cas13d sequence from Ruminococcus albus.

SEQ ID NO: 71 is an exemplary Cas13d sequence from Gut_metagenome_contig13552000311.

SEQ ID NO: 72 is an exemplary Cas13d sequence from Gut_metagenome_contig10037000527.

SEQ ID NO: 73 is an exemplary Cas13d sequence from Gut_metagenome_contig238000329.

SEQ ID NO: 74 is an exemplary Cas13d sequence from Gut_metagenome_contig2643000492.

SEQ ID NO: 75 is an exemplary Cas13d sequence from Gut_metagenome_contig874000057.

SEQ ID NO: 76 is an exemplary Cas13d sequence from Gut_metagenome_contig4781000489.

SEQ ID NO: 77 is an exemplary Cas13d sequence from Gut_metagenome_contig12144000352.

SEQ ID NO: 78 is an exemplary Cas13d sequence from Gut_metagenome_contig5590000448.

SEQ ID NO: 79 is an exemplary Cas13d sequence from Gut_metagenome_contig9269000031.

SEQ ID NO: 80 is an exemplary Cas13d sequence from Gut_metagenome_contig8537000520.

SEQ ID NO: 81 is an exemplary Cas13d sequence from Gut_metagenome_contig1845000130.

SEQ ID NO: 82 is an exemplary Cas13d sequence from gut_metagenome_P13E0k2120140920_c3000072.

SEQ ID NO: 83 is an exemplary Cas13d sequence from gut_metagenome_P1E0k2120140920_cI000078.

SEQ ID NO: 84 is an exemplary Cas13d sequence from Gut_metagenome_contig12990000099.

SEQ ID NO: 85 is an exemplary Cas13d sequence from Gut_metagenome_contig525000349.

SEQ ID NO: 86 is an exemplary Cas13d sequence from Gut_metagenome_contig7229000302.

SEQ ID NO: 87 is an exemplary Cas13d sequence from Gut_metagenome_contig3227000343.

SEQ ID NO: 88 is an exemplary Cas13d sequence from Gut_metagenome_contig7030000469.

SEQ ID NO: 89 is an exemplary Cas13d sequence from Gut_metagenome_contig5149000068.

SEQ ID NO: 90 is an exemplary Cas13d sequence from Gut_metagenome_contig400200045.

SEQ ID NO: 91 is an exemplary Cas13d sequence from Gut_metagenome_contig10420000446.

SEQ ID NO: 92 is an exemplary Cas13d sequence from new_flavefaciens_strain_XPD3002 (CasRx).

SEQ ID NO: 93 is an exemplary Cas13d sequence from M26_Gut_metagenome_contig698000307.

SEQ ID NO: 94 is an exemplary Cas13d sequence from M36_Uncultured_Eubacterium_sp_TS28_c40956.

SEQ ID NO: 95 is an exemplary Cas13d sequence from M12_gut_metagenome_P25C0k2120140920_c134000066.

SEQ ID NO: 96 is an exemplary Cas13d sequence from human gut metagenome.

SEQ ID NO: 97 is an exemplary Cas13d sequence from MlO_gut_metagenome P25C90k2120 1 40920_c2800004 1.

SEQ ID NO: 98 is an exemplary Cas13d sequence from 30 Ml I_gut_metagenome_P25C7k2120140920_c4078000105.

SEQ ID NO: 99 is an exemplary Cas13d sequence from gut_metagenome_P25C0k2120140920_c32000045.

SEQ ID NO: 100 is an exemplary Cas13d sequence from M13_gut_metagenome P23C7k2120140920_c3000067.

SEQ ID NO: 101 is an exemplary Cas13d sequence from M5_gut_metagenome_Pl8E90k2120140920.

SEQ ID NO: 102 is an exemplary Cas13d sequence from M21_gut_metagenome_Pl8Ek2120140920.

SEQ ID NO: 103 is an exemplary Cas13d sequence from M7_gut_metagenome P38C7k2120 1 40920_c484 1 000003.

SEQ ID NO: 104 is an exemplary Cas13d sequence from Ruminococcus_bicirculans.

SEQ ID NO: 105 is an exemplary Cas13d sequence.

SEQ ID NO: 106 is an exemplary Cas13d consensus sequence.

SEQ ID NO: 107 is an exemplary Cas13d sequence from M18_gut_metagenome P22EOk2120140920_c3395000078.

SEQ ID NO: 108 is an exemplary Cas13d sequence from M17_gut_metagenome_P22E90k2120140920_c114.

SEQ ID NO: 109 is an exemplary Cas13d sequence from Ruminococcus_sp_CAG57.

SEQ ID NO: 110 is an exemplary Cas13d sequence from gut_metagenome_Pl 1E90k2120140920_c43000123.

SEQ ID NO: 111 is an exemplary Cas13d sequence from M6_gut_metagenome_P13E90k2120 1 40920_c7000009.

SEQ ID NO: 112 is an exemplary Cas13d sequence from Ml9_gut_metagenome_Pl7E90k2120140920.

SEQ ID NO: 113 is an exemplary Cas13d sequence from gut_metagenome_P17E0k2120140920,_c87000043.

SEQ ID NO: 114 is an exemplary human codon optimized Eubacterium siraeum Cas13d nucleic acid sequence.

SEQ ID NO: 115 is an exemplary human codon optimized Eubacterium siraeum Cas13d nucleic acid sequence with a mutant HEPN domain.

SEQ ID NO: 116 is an exemplary human codon-optimized Eubacterium siraeum Cas13d nucleic acid sequence with N-terminalNLS.

SEQ ID NO: 117 is an exemplary human codon-optimized Eubacterium siraeum Cas13d nucleic acid sequence with N- and C-terminal NLS tags.

SEQ ID NO: 118 is an exemplary human codon-optimized uncultured Ruminococcus sp. Cas13d 30 nucleic acid sequence.

SEQ ID NO: 119 is an exemplary human codon-optimized uncultured Ruminococcus sp. Cas13d nucleic acid sequence with a mutant HEPN domain.

SEQ ID NO: 120 is an exemplary human codon-optimized uncultured Ruminococcus sp. Cas13d nucleic acid sequence with N-terminal NLS.

SEQ ID NO: 121 is an exemplary human codon-optimized uncultured Ruminococcus sp. Cas13d nucleic acid sequence with N- and C-terminal NLS tags.

SEQ ID NO: 122 is an exemplary human codon-optimized uncultured Ruminococcus flavefaciens FD1 Cas13d nucleic acid sequence.

SEQ ID NO: 123 is an exemplary human codon-optimized uncultured Ruminococcus flavefaciens FD1 Cas13d nucleic acid sequence with mutated HEPN domain.

SEQ ID NO: 124 is an exemplary Cas13d nucleic acid sequence from Ruminococcus bicirculans.

SEQ ID NO: 125 is an exemplary Cas13d nucleic acid sequence from Eubacterium siraeum.

SEQ ID NO: 126 is an exemplary Cas13d nucleic acid sequence from Ruminococcus flavefaciens FD1.

SEQ ID NO: 127 is an exemplary Cas13d nucleic acid sequence from Ruminococcus albus.

SEQ ID NO: 128 is an exemplary Cas13d nucleic acid sequence from Ruminococcus flavefaciens XPD.

SEQ ID NO: 129 is an exemplary consensus DR nucleic acid sequence for E. siraeum Cas13d.

SEQ ID NO: 130 is an exemplary consensus DR nucleic acid sequence for Rum. Sp. Cas13d.

SEQ ID NO: 131 is an exemplary consensus DR nucleic acid sequence for Rum. Flavefaciens strain XPD3002 Cas13d (CasRx).

SEQ ID NOS: 132-137 are exemplary consensus DR nucleic acid sequences.

SEQ ID NO: 138 is an exemplary 50% consensus sequence for seven full-length Cas13d orthologues.

SEQ ID NO: 139 is an exemplary Cas13d nucleic acid sequence from Gut metagenome PlEO.

SEQ ID NO: 140 is an exemplary Cas13d nucleic acid sequence from Anaerobic digester.

SEQ ID NO: 141 is an exemplary Cas13d nucleic acid sequence from Ruminococcus sp. CAG:57.

SEQ ID NO: 142 is an exemplary human codon-optimized uncultured Gut metagenome PlEO Cas13d nucleic acid sequence.

SEQ ID NO: 143 is an exemplary human codon-optimized Anaerobic Digester Cas13d nucleic acid sequence.

SEQ ID NO: 144 is an exemplary human codon-optimized Ruminococcus flavefaciens XPD Cas13d nucleic acid sequence.

SEQ ID NO: 145 is an exemplary human codon-optimized Ruminococcus albus Cas13d nucleic acid sequence.

SEQ ID NO: 146 is an exemplary processing of the Ruminococcus sp. CAG:57 CRISPR array.

SEQ ID NO: 147 is an exemplary Cas13d protein sequence from contig emb |OBVH01003037.1, human gut metagenome sequence (also found in WGS contigs emb |OBXZ01000094. 1| and emb |OBJFO1000033.1.

SEQ ID NO: 148 is an exemplary consensus DR nucleic acid sequence (goes with SEQ ID NO: 147).

SEQ ID NO: 149 is an exemplary Cas13d protein sequence from contig tpg |DBYI01000091.1| (Uncultivated Ruminococcus flavefaciens UBA1190 assembled from bovine gut metagenome).

SEQ ID NOS: 150-152 are exemplary consensus DR nucleic acid sequences (goes with SEQ ID NO: 149).

SEQ ID NO: 153 is an exemplary Cas13d protein sequence from contig tpg |DJXD01000002.1| (uncultivated Ruminococcus assembly, UBA7013, from sheep gutmetagenome).

SEQ ID NO: 154 is an exemplary consensus DR nucleic acid sequence (goes with SEQ ID NO: 153).

SEQ ID NO: 155 is an exemplary Cas13d protein sequence from contig OGZC01000639.1 (human gut metagenome assembly).

SEQ ID NOS: 156-177 are exemplary consensus DR nucleic acid sequences (goes with SEQ ID NO: 155).

SEQ ID NO: 158 is an exemplary Cas13d protein sequence from contig emb |OHBM01000764.1 (human gut metagenome assembly).

SEQ ID NO: 159 is an exemplary consensus DR nucleic acid sequence (goes with SEQ ID NO: 158).

SEQ ID NO: 160 is an exemplary Cas13d protein sequence from contig emb |0HCP01000044.1 (human gut metagenome assembly).

SEQ ID NO: 161 is an exemplary consensus DR nucleic acid sequence (goes with SEQ ID NO: 160).

SEQ ID NO: 162 is an exemplary Cas13d protein sequence from contig embl0GDF01008514.1 (human gut metagenome assembly).

SEQ ID NO: 163 is an exemplary consensus DR nucleic acid sequence (goes with SEQ ID NO: 162).

SEQ ID NO: 164 is an exemplary Cas13d protein sequence from contig emb |OGPN01002610.1 (human gut metagenome assembly).

SEQ ID NO: 165 is an exemplary consensus DR nucleic acid sequence (goes with SEQ ID NO: 164).

SEQ ID NO: 166 is an exemplary Cas13d protein sequence from contig NFIR01000008. 1 (Eubacterium sp. An3, from chicken gut metagenome).

SEQ ID NO: 167 is an exemplary consensus DR nucleic acid sequence (goes with SEQ ID NO: 166).

SEQ ID NO: 168 is an exemplary Cas13d protein sequence from contig NFLV01000009.1 (Eubacterium sp. An 11 from chicken gut metagenome).

SEQ ID NO: 169 is an exemplary consensus DR nucleic acid sequence (goes with SEQ ID NO: 168).

SEQ ID NOS: 171-174 are an exemplary Cas13d motifsequences.

SEQ ID NO: 175 is an exemplary Cas13d protein sequence from contig OJMM01002900 human gut metagenome sequence.

SEQ ID NO: 176 is an exemplary consensus DR nucleic acid sequence (goes with SEQ ID NO: 175).

SEQ ID NO: 177 is an exemplary Cas13d protein sequence from contig ODAI011611274.1 gut metagenome sequence.

SEQ ID NO: 178 is an exemplary consensus DR nucleic acid sequence (goes with SEQ ID NO: 177).

SEQ ID NO: 179 is an exemplary Cas13d protein sequence from contig OIZX01000427.1.

SEQ ID NO: 180 is an exemplary consensus DR nucleic acid sequence (goes with SEQ ID NO: 179).

SEQ ID NO: 181 is an exemplary Cas13d protein sequence from contig emb |OCVV012889144.1|.

SEQ ID NO: 182 is an exemplary consensus DR nucleic acid sequence (goes with SEQ ID NO: 181).

SEQ ID NO: 183 is an exemplary Cas13d protein sequence from contig OCTW011587266.1

SEQ ID NO: 184 is an exemplary consensus DR nucleic acid sequence (goes with SEQ ID NO: 183).

SEQ ID NO: 185 is an exemplary Cas13d protein sequence from contig emb |OGNFO 1009141.1.

SEQ ID NO: 186 is an exemplary consensus DR nucleic acid sequence (goes with SEQ ID NO: 185).

SEQ ID NO: 187 is an exemplary Cas13d protein sequence from contig emb |OIEN01002196.1.

SEQ ID NO: 188 is an exemplary consensus DR nucleic acid sequence (goes with SEQ ID NO: 187).

SEQ ID NO: 189 is an exemplary Cas13d protein sequence from contig e-k87_11092736.

SEQ ID NOS: 190-193 are exemplary consensus DR nucleic acid sequences (goes with SEQ ID NO: 189).

SEQ ID NO: 194 is an exemplary Cas13d sequence from Gut_metagenome_contig6893000291.

SEQ ID NOS: 195-197 are exemplary Cas13d motif sequences.

SEQ ID NO: 198 is an exemplary Cas13d protein sequence from Ga0224415_10007274.

SEQ ID NO: 199 is an exemplary consensus DR nucleic acid sequence (goes with SEQ ID NO: 198).

SEQ ID NO: 200 is an exemplary Cas13d protein sequence from EMG_10003641.

SEQ ID NO: 202 is an exemplary Cas13d protein sequence from Ga0129306_1000735.

SEQ ID NO: 201 is an exemplary consensus DR nucleic acid sequence (goes with SEQ ID NO: 200).

SEQ ID NO: 202 is an exemplary Cas13d protein sequence from Ga0129306_1000735.

SEQ ID NO: 203 is an exemplary consensus DR nucleic acid sequence (goes with SEQ ID NO: 203

SEQ ID NO: 204 is an exemplary Cas13d protein sequence from GaO129317_1 008067.

SEQ ID NO: 205 is an exemplary consensus DR nucleic acid sequence (goes with SEQ ID NO: 204).

SEQ ID NO: 206 is an exemplary Cas13d protein sequence from Ga0224415_10048792.

SEQ ID NO: 207 is an exemplary consensus DR nucleic acid sequence (goes with SEQ ID NO: 206).

SEQ ID NO: 208 is an exemplary Cas13d protein sequence from 160582958_gene49834.

SEQ ID NO: 209 is an exemplary consensus DR nucleic acid sequence (goes with SEQ ID NO: 208).

SEQ ID NO: 210 is an exemplary Cas13d protein sequence from 250twins_35838_GL0110300.

SEQ ID NO: 211 is an exemplary consensus DR nucleic acid sequence (goes with SEQ ID NO: 210).

SEQ ID NO: 212 is an exemplary Cas13d protein sequence from 250twins_36050_GLOI58985.

SEQ ID NO: 213 is an exemplary consensus DR nucleic acid sequence (goes with SEQ ID NO: 212).

SEQ ID NO: 214 is an exemplary Cas13d protein sequence from 31009_GL0034153.

SEQ ID NO: 215 is an exemplary consensus DR nucleic acid sequence (goes with SEQ ID NO: 214).

SEQ ID NO: 216 is an exemplary Cas13d protein sequence from 530373_GL0023589.

SEQ ID NO: 217 is an exemplary consensus DR nucleic acid sequence (goes with SEQ ID NO: 216).

SEQ ID NO: 218 is an exemplary Cas13d protein sequence from BMZ-1 1B_GL0037771.

SEQ ID NO: 219 is an exemplary consensus DR nucleic acid sequence (goes with SEQ ID NO: 218).

SEQ ID NO: 220 is an exemplary Cas13d protein sequence from BMZ-1 1B_GL0037915.

SEQ ID NO: 221 is an exemplary consensus DR nucleic acid sequence (goes with SEQ ID NO: 220).

SEQ ID NO: 222 is an exemplary Cas13d protein sequence from BMZ-1 1B_GL00696 1 7.

SEQ ID NO: 223 is an exemplary consensus DR nucleic acid sequence (goes with SEQ ID NO: 222).

SEQ ID NO: 224 is an exemplary Cas13d protein sequence from DLF014_GL0011914.

SEQ ID NO: 225 is an exemplary consensus DR nucleic acid sequence (goes with SEQ ID NO: 224).

SEQ ID NO: 226 is an exemplary Cas13d protein sequence from EYZ-362B_GL0088915.

SEQ ID NO: 227-228 are exemplary consensus DR nucleic acid sequences (goes with SEQ ID NO: 226).

SEQ ID NO: 229 is an exemplary Cas13d protein sequence from Ga0099364 10024192.

SEQ ID NO: 230 is an exemplary consensus DR nucleic acid sequence (goes with SEQ ID NO: 229).

SEQ ID NO: 231 is an exemplary Cas13d protein sequence from Ga0187910_10006931.

SEQ ID NO: 232 is an exemplary consensus DR nucleic acid sequence (goes with SEQ ID NO: 231).

SEQ ID NO: 233 is an exemplary Cas13d protein sequence from Ga0187910_10015336.

SEQ ID NO: 234 is an exemplary consensus DR nucleic acid sequence (goes with SEQ ID NO: 233).

SEQ ID NO: 235 is an exemplary Cas13d protein sequence from Ga0187910_10040531.

SEQ ID NO: 236 is an exemplary consensus DR nucleic acid sequence (goes with SEQ ID NO: 23).

SEQ ID NO: 237 is an exemplary Cas13d protein sequence from Ga0187911_10069260.

SEQ ID NO: 238 is an exemplary consensus DR nucleic acid sequence (goes with SEQ ID NO: 237).

SEQ ID NO: 239 is an exemplary Cas13d protein sequence from MH0288_GL0082219.

SEQ ID NO: 240 is an exemplary consensus DR nucleic acid sequence (goes with SEQ ID NO: 239).

SEQ ID NO: 241 is an exemplary Cas13d protein sequence from 02.UC29-0_GL0096317.

SEQ ID NO: 242 is an exemplary consensus DR nucleic acid sequence (goes with SEQ ID NO: 241).

SEQ ID NO: 243 is an exemplary Cas13d protein sequence from PIG-014_GL0226364.

SEQ ID NO: 244 is an exemplary consensus DR nucleic acid sequence (goes with SEQ ID NO: 243).

SEQ ID NO: 245 is an exemplary Cas13d protein sequence from PIG-018_GL0023397.

SEQ ID NO: 246 is an exemplary consensus DR nucleic acid sequence (goes with SEQ ID NO: 245).

SEQ ID NO: 247 is an exemplary Cas13d protein sequence from PIG-025_GL0099734.

SEQ ID NO: 248 is an exemplary consensus DR nucleic acid sequence (goes with SEQ ID NO: 247).

SEQ ID NO: 249 is an exemplary Cas13d protein sequence from PIG-028_GL0185479.

SEQ ID NO: 250 is an exemplary consensus DR nucleic acid sequence (goes with SEQ ID NO: 249).

SEQ ID NO: 251 is an exemplary Cas13d protein sequence from —Ga0224422_10645759.

SEQ ID NO: 252 is an exemplary consensus DR nucleic acid sequence (goes with SEQ ID NO: 251).

SEQ ID NO: 253 is an exemplary Cas13d protein sequence from ODAI chimera.

SEQ ID NO: 254 is an exemplary consensus DR nucleic acid sequence (goes with SEQ ID NO: 253).

SEQ ID NO: 255 is an HEPN motif.

SEQ ID NOs: 256 and 257 are exemplary Cas13d nuclear localization signal amino acid and nucleic acid sequences, respectively.

SEQ ID NOs: 258 and 260 are exemplary SV40 large T antigen nuclear localization signal amino acid and nucleic acid sequences, respectively.

SEQ ID NO: 259 is a dCas9 target sequence.

SEQ ID NO: 261 is an artificial Eubacterium siraeum nCasl array targeting ccdB.

SEQ ID NO: 262 is a full 36 nt direct repeat.

SEQ ID NOs: 263-266 are spacer sequences.

SEQ ID NO: 267 is an artificial uncultured Ruminoccus sp. nCasl array targeting ccdB.

SEQ ID NO: 268 is a full 36 nt direct repeat.

SEQ ID NOs: 269-272 are spacer sequences.

SEQ ID NO: 273 is a ccdB target RNA sequence.

SEQ ID NOs: 274-277 are spacer sequences.

SEQ ID NO: 278 is a mutated Cas13d sequence, NLS-Ga_0531(trunc)-NLS-HA. This mutant has a deletion of the non-conserved N-terminus.

SEQ ID NO: 279 is a mutated Cas13d sequence, NES-Ga_0531(trunc)-NES-HA. This mutant has a deletion of the non-conserved N-terminus.

SEQ ID NO: 280 is a full-length Cas13d sequence, NLS-RfxCas13d-NLS-HA.

SEQ ID NO: 281 is a mutated Cas13d sequence, NLS-RfxCas13d(del5)-NLS-HA. This mutant has a deletion of amino acids 558-587.

SEQ ID NO: 282 is a mutated Cas13d sequence, NLS-RfxCas13d(del5.12)-NLS-HA. This mutant has a deletion of amino acids 558-587 and 953-966.

SEQ ID NO: 283 is a mutated Cas13d sequence, NLS-RfxCas13d(del5.13)-NLS-HA. This mutant has a deletion of amino acids 376-392 and 558-587.

SEQ ID NO: 284 is a mutated Cas13d sequence, NLS-RfxCas13d(del5.12+5. 13)-NLS-HA. This mutant has a deletion of amino acids 376-392, 558-587, and 953-966.

SEQ ID NO: 285 is a mutated Cas13d sequence, NLS-RfxCas13d(dell3)-NLS-HA. This mutant has a deletion of amino acids 376-392.

SEQ ID NO: 286 is an effector sequence used to edit expression of ADAR2. Amino acids 1 to 969 are dRfxCas13, aa 970 to 991 are an NLS sequence, and amino acids 992 to 1378 are ADAR2DD.

SEQ ID NO: 287 is an exemplary HIV NES protein sequence.

SEQ ID NOS: 288-291 are exemplary Cas13d motif sequences.

SEQ ID NO: 292 is Cas13d ortholog sequence MH_4866.

SEQ ID NO: 293 is an exemplary Cas13d protein sequence from 037_-_emblOIZA01000315.11

SEQ ID NO: 294 is an exemplary Cas13d protein sequence from PIG-022 GL002635 1.

SEQ ID NO: 295 is an exemplary Cas13d protein sequence from PIG-046_GL0077813.

SEQ ID NO: 296 is an exemplary Cas13d protein sequence from pig chimera.

SEQ ID NO: 297 is an exemplary nuclease-inactive or dead Cas13d (dCas13d) protein sequence from Ruminococcus flavefaciens XPD3002 (CasRx)

SEQ ID NO: 298 is an exemplary Cas13d protein sequence.

SEQ ID NO: 299 is an exemplary Cas13d protein sequence from (contig tpg|DJXD01000002.1|; uncultivated Ruminococcus assembly, UBA7013, from sheep gut metagenome).

SEQ ID NO: 300 is an exemplary Cas13d direct repeat nucleotide sequence from Cas13d (contig tpg|DJXD01000002.1|; uncultivated Ruminococcus assembly, UBA7013, from sheep gut metagenome (goes with SEQ ID NO: 299).

SEQ ID NO: 301 is an exemplary Cas13d protein contig emb|OBLI01020244.

Yan et al. (2018) Mol Cell. 70(2):327-339 (doi: 10.1016/j.molcel.2018.02.2018) and Konermann et al. (2018) Cell 173(3):665-676 (doi: 10.1016/j.cell/2018.02.033) have described Cas13d proteins and both of which are incorporated by reference herein in their entireties. Also see WO Publication Nos. WO2018/183403 (CasM, which is Cas13d) and WO2019/006471 (Cas13d), which are incorporated herein by reference in their entirety.

SEQ ID NO: 586 is an exemplary cas13d with no catalytic activity, referred to as deactivatedCas13d or dCas13d.

SEQ ID NO: 587 is an exemplary cas13d with no catalytic activity, referred to as deactivatedCas13d or dCas13d.

SEQ ID NO: 588 is an exemplary cas13d with no catalytic activity, referred to as deactivatedCas13d or dCas13d.

SEQ ID NO: 589 is an exemplary cas13d with no catalytic activity, referred to as deactivatedCas13d or dCas13d.

SEQ ID NO: 303 is an exemplary CasM protein from Eubacterium siraeum.

SEQ ID NO: 304 is an exemplary CasM protein from Ruminococcus sp., isolate 2789STDY5834971.

SEQ ID NO: 305 is an exemplary CasM protein from Ruminococcus bicirculans.

SEQ ID NO: 306 is an exemplary CasM protein from Ruminococcus sp., isolate 2789STDY5608892.

SEQ ID NO: 307 is an exemplary CasM protein from Ruminococcus sp. CAG:57.

SEQ ID NO: 308 is an exemplary CasM protein from Ruminococcus flavefaciens FD-1.

SEQ ID NO: 309 is an exemplary CasM protein from Ruminococcus albus strain KH2T6.

SEQ ID NO: 310 is an exemplary CasM protein from Ruminococcus flavefaciens strain XPD3002.

SEQ ID NO: 311 is an exemplary CasM protein from Ruminococcus sp., isolate 2789STDY5834894.

SEQ ID NO: 312 is an exemplary RtcB homolog.

SEQ ID NO: 313 is an exemplary WYL from Eubacterium siraeum+C-terminal NLS.

SEQ ID NO: 314 is an exemplary WYL from Ruminococcus sp. isolate 2789STDY5834971+C-term NLS.

SEQ ID NO: 315 is an exemplary WYL from Ruminococcus bicirculans+C-term NLS.

SEQ ID NO: 316 is an exemplary WYL from Ruminococcus sp. isolate 2789STDY5608892+C-term NLS.

SEQ ID NO: 317 is an exemplary WYL from Ruminococcus sp. CAG:57+C-term NLS.

SEQ ID NO: 318 is an exemplary WYL from Ruminococcus flavefaciens FD-1+C-term NLS.

SEQ ID NO: 319 is an exemplary WYL from Ruminococcus albus strain KH2T6+C-term NLS.

SEQ ID NO: 320 is an exemplary WYL from Ruminococcus flavefaciens strain XPD3002+C-term NLS.

SEQ ID NO: 321 is an exemplary RtcB from Eubacterium siraeum+C-term NLS.

SEQ ID NO: 322 is an exemplary direct repeat sequence of Ruminococcus flavefaciens XPD3002 Cas13d (CasRx).

SEQ ID NO: 530 is an exemplary Cas13d nucleic acid sequence, seq198.

SEQ ID NO: 535 is an exemplary Cas13d nucleic acid sequence, seq179.

SEQ ID NO: 538 is an exemplary Cas13d nucleic acid sequence, seq42.

SEQ ID NO: 540 is an exemplary Cas13d nucleic acid sequence, seq212.

SEQ ID NO: 537 is an exemplary nucleic acid sequence encoding an exemplary DR nucleic acid sequence corresponding to SEQ ID NO: 538.

Exemplary wild type Cas13d proteins of the disclosure may comprise or consist of the amino acid sequence SEQ ID NO: 92 or SEQ ID NO: 298 (Cas13d protein also known as CasRx).

An exemplary direct repeat sequence of Ruminococcus flavefaciens XPD3002 Cas13d (CasRx) comprises the nucleic acid sequence:

(SEQ ID NO: 302)
AACCCCTACCAACTGGTCGGGGTTTGAAAC.

gRNA Target Sequences

The compositions of the disclosure bind and destroy a target sequence of an RNA molecule comprising a pathogenic repeat sequence. In one embodiment, the target RNA comprises a sequence motif corresponding to a spacer sequence of the guide RNA corresponding to the RNA-guided RNA-binding protein. In some embodiments, one or more spacer sequences are used to target one or more target sequences. In some embodiments, multiple spacers are used to target multiple target RNAs. Such target RNAs can be different target sites within the same RNA molecule or can be different target sites within different RNA molecules. Spacer sequences can also target non-coding RNA. In some embodiments, multiple promoters, e.g., Pol III promoters) can be used to drive multiple spacers in a gRNA for targeting multiple target RNAs. In one embodiment, the destruction of the target RNA(s) or target sequence motif(s) reduces expression of pathogenic CUG repeat RNA thereby treating DM1 and/or ameliorating one or more symptoms associated with DM1.

In some embodiments of the compositions and methods of the disclosure, the sequence motif of the target RNA is a signature of a disease or disorder.

A sequence motif of the disclosure may be isolated or derived from a sequence of foreign or exogenous sequence found in a genomic sequence, and therefore translated into an mRNA molecule of the disclosure or a sequence of foreign or exogenous sequence found in an RNA sequence of the disclosure.

A target sequence motif of the disclosure may comprise, consist of, be situated by, or be associated with a mutation in an endogenous sequence that causes a disease or disorder. The mutation may comprise or consist of a sequence substitution, inversion, deletion, insertion, transposition, or any combination thereof.

A target sequence motif of the disclosure may comprise or consist of a repeated sequence. In some embodiments, the repeated sequence may be associated with a microsatellite instability (MSI). MSI at one or more loci results from impaired DNA mismatch repair mechanisms of a cell of the disclosure. A hypervariable sequence of DNA may be transcribed into an mRNA of the disclosure comprising a target sequence comprising or consisting of the hypervariable sequence.

A target sequence motif of the disclosure may comprise or consist of a biomarker. The biomarker may indicate a risk of developing a disease or disorder. The biomarker may indicate a healthy gene (low or no determinable risk of developing a disease or disorder. The biomarker may indicate an edited gene. Exemplary biomarkers include, but are not limited to, single nucleotide polymorphisms (SNPs), sequence variations or mutations, epigenetic marks, splice acceptor sites, exogenous sequences, heterologous sequences, and any combination thereof.

A target sequence motif of the disclosure may comprise or consist of a secondary, tertiary or quaternary structure. The secondary, tertiary or quaternary structure may be endogenous or naturally occurring. The secondary, tertiary or quaternary structure may be induced or non-naturally occurring. The secondary, tertiary or quaternary structure may be encoded by an endogenous, exogenous, or heterologous sequence.

In some embodiments of the compositions and methods of the disclosure, a target sequence of an RNA molecule comprises or consists of between 2 and 100 nucleotides or nucleic acid bases, inclusive of the endpoints. In some embodiments, the target sequence of an RNA molecule comprises or consists of between 2 and 50 nucleotides or nucleic acid bases, inclusive of the endpoints. In some embodiments, the target sequence of an RNA molecule comprises or consists of between 2 and 20 nucleotides or nucleic acid bases, inclusive of the endpoints. In some embodiments, the target sequence of an RNA molecule comprises or consists of between 20-30 nucleotides or nucleic acid bases, inclusive of the endpoints. In some embodiments, the target sequence of an RNA molecule comprises or consists of about 26 nucleotides or nucleic acid bases, inclusive of the endpoints.

In some embodiments of the compositions and methods of the disclosure, a target sequence of an RNA molecule is continuous. In some embodiments, the target sequence of an RNA molecule is discontinuous. For example, the target sequence of an RNA molecule may comprise or consist of one or more nucleotides or nucleic acid bases that are not contiguous because one or more intermittent nucleotides are positioned in between the nucleotides of the target sequence.

In some embodiments of the compositions and methods of the disclosure, a target sequence of an RNA molecule is naturally occurring. In some embodiments, the target sequence of an RNA molecule is non-naturally occurring. Exemplary non-naturally occurring target sequences may comprise or consist of sequence variations or mutations, chimeric sequences, exogenous sequences, heterologous sequences, chimeric sequences, recombinant sequences, sequences comprising a modified or synthetic nucleotide or any combination thereof.

In some embodiments of the compositions and methods of the disclosure, a target sequence of an RNA molecule binds to a guide RNA of the disclosure. In some embodiments of the compositions and methods of the disclosure, one or more target sequences of an RNA molecule binds to one or more guide RNA spacer sequences of the disclosure.

In some embodiments of the compositions and methods of the disclosure, a target sequence of an RNA molecule binds to a first RNA binding protein of the disclosure.

In some embodiments of the compositions and methods of the disclosure, a target sequence of an RNA molecule binds to a second RNA binding protein of the disclosure.

Compositions of the disclosure comprise a gRNA comprising a spacer sequence that specifically binds to a target toxic CUG RNA repeat sequence. In some embodiments, the spacer which binds the target CUG RNA repeat sequence comprises or consists of about 20-30 nucleotides. In some embodiments, a gRNA comprises one or more spacer sequences.

Exemplary gRNA spacer sequences of the disclosure that specifically bind to a target CUG sequence of an RNA molecule are set forth in SEQ ID NOs 457-459.

### Endonucleases

In some embodiments, the compositions of the disclosure comprise a second RNA binding protein which comprises or consists of a nuclease or endonuclease domain. In some embodiments, the second RNA-binding protein is an effector protein. In some embodiments, the second RNA binding protein binds RNA in a manner in which it associates with RNA. In some embodiments, the second RNA binding protein associates with RNA in a manner in which it cleaves RNA. In some embodiments, the second RNA-binding protein is fused to a first RNA-binding protein which is a PUF, PUMBY, or PPR-based protein. In one embodiment, the second RNA-binding protein is fused to a first RNA-binding protein which is a deactivated Cas-based (dCas-based) protein.

In some embodiments of the compositions of the disclosure, the second RNA binding protein comprises or consists of an RNase.

In some embodiments, the second RNA binding protein comprises or consists of an RNase1. In some embodiments, the RNase1 protein comprises or consists of SEQ ID NO: 325.

In some embodiments, the second RNA binding protein comprises or consists of an RNase4. In some embodiments, the RNase4 protein comprises or consists of SEQ ID NO: 326.

In some embodiments, the second RNA binding protein comprises or consists of an RNase6. In some embodiments, the RNase6 protein comprises or consists of SEQ ID NO: 327.

In some embodiments, the second RNA binding protein comprises or consists of an RNase7. In some embodiments, the RNase7 protein comprises or consists of SEQ ID NO: 328.

In some embodiments, the second RNA binding protein comprises or consists of an RNase8. In some embodiments, the RNase8 protein comprises or consists of SEQ ID NO: 329.

In some embodiments, the second RNA binding protein comprises or consists of an RNase2. In some embodiments, the RNase2 protein comprises or consists of SEQ ID NO: 330.

In some embodiments, the second RNA binding protein comprises or consists of an RNase6PL. In some embodiments, the RNase6PL protein comprises or consists of SEQ ID NO: 331.

In some embodiments, the second RNA binding protein comprises or consists of an RNaseL. In some embodiments, the RNaseL protein comprises or consists of SEQ ID NO: 332.

In some embodiments, the second RNA binding protein comprises or consists of an RNaseT2. In some embodiments, the RNaseT2 protein comprises or consists of SEQ ID NO: 333.

In some embodiments, the second RNA binding protein comprises or consists of an RNase1 1. In some embodiments, the RNase1 1 protein comprises or consists of SEQ ID NO: 334.

In some embodiments, the second RNA binding protein comprises or consists of an RNaseT2-like. In some embodiments, the RNaseT2-like protein comprises or consists of SEQ ID NO: 335.

In some embodiments of the compositions of the disclosure, the second RNA binding protein comprises or consists of a mutated RNase.

In some embodiments, the second RNA binding protein comprises or consists of a mutated RNase1 (RNase1(K41R)) polypeptide. In some embodiments, the RNase1(K41R) polypeptide comprises or consists of SEQ ID NO: 336.

In some embodiments, the second RNA binding protein comprises or consists of a mutated RNase1 (RNase1(K41R, D121E)) polypeptide. In some embodiments, the RNase1 (RNase1(K41R, D121E)) polypeptide comprises or consists of SEQ ID NO: 337.

In some embodiments, the second RNA binding protein comprises or consists of a mutated RNase1 (RNase1(K41R, D121E, H119N)) polypeptide. In some embodiments, the RNase1 (RNase1(K41R, D121E, H119N)) polypeptide comprises or consists of SEQ ID NO: 338.

In some embodiments, the second RNA binding protein comprises or consists of a mutated RNase1. In some embodiments, the second RNA binding protein comprises or consists of a mutated RNase1 (RNase1(H119N)) polypeptide. In some embodiments, the RNase1 (RNase1(H119N)) polypeptide comprises or consists of SEQ ID NO: 339.

In some embodiments, the second RNA binding protein comprises or consists of a mutated RNase1 (RNase1(R39D, N67D, N88A, G89D, R91D, H119N)) polypeptide.

In some embodiments, the RNase1 (RNase1(R39D, N67D, N88A, G89D, R91D, H119N)) polypeptide comprises or consists of SEQ ID NO: 340.

In some embodiments, the second RNA binding protein comprises or consists of a mutated RNase1 (RNase1(R39D, N67D, N88A, G89D, R91D, H119N)) polypeptide. In some embodiments, the RNase1 (RNase1(R39D, N67D, N88A, G89D, R91D, Hi19N, K41R, D121E)) polypeptide comprises or consists of SEQ ID NO: 341. In some embodiments, the second RNA binding protein comprises or consists of a mutated RNase1 (RNase1(R39D, N67D, N88A, G89D, R91D, HI19N)) polypeptide. In some embodiments, the RNase1 (RNase1(R39D, N67D, N88A, G89D, R91D)) polypeptide comprises or consists of SEQ ID NO: 342.

In some embodiments, the second RNA binding protein comprises or consists of a mutated RNase1 (RNase1 (R39D, N67D, N88A, G89D, R91D, H119N, K41R, D121E)) polypeptide that comprises or consists of SEQ ID NO: 343.

In some embodiments of the compositions of the disclosure, the second RNA binding protein comprises or consists of a NOB1 polypeptide. In some embodiments, the NOB1 polypeptide comprises or consists of SEQ ID NO: 344.

In some embodiments of the compositions of the disclosure, the second RNA binding protein comprises or consists of an endonuclease. In some embodiments, the second RNA binding protein comprises or consists of an endonuclease V (ENDOV). In some embodiments, the ENDOV protein comprises or consists of SEQ ID NO: 345.

In some embodiments, the second RNA binding protein comprises or consists of an endonuclease G (ENDOG). In some embodiments, the ENDOG protein comprises or consists of SEQ ID NO: 346.

In some embodiments, the second RNA binding protein comprises or consists of an endonuclease D1 (ENDOD1). In some embodiments, the ENDOD1 protein comprises or consists of SEQ ID NO: 347.

In some embodiments, the second RNA binding protein comprises or consists of a Human flap endonuclease-1 (hFEN1). In some embodiments, the hFEN1 polypeptide comprises or consists of SEQ ID NO: 348.

In some embodiments, the second RNA binding protein comprises or consists of a DNA repair endonuclease XPF (ERCC4) polypeptide. In some embodiments, the ERCC4 polypeptide comprises or consists of SEQ ID NO: 349.

In some embodiments of the compositions of the disclosure, the second RNA binding protein comprises or consists of an Endonuclease III-like protein 1 (NTHL) polypeptide. In some embodiments, the NTHL polypeptide comprises or consists of SEQ ID NO: 340.

In some embodiments of the compositions of the disclosure, the second RNA binding protein comprises or consists of a human Schlafen 14 (hSLFN14) polypeptide. In some embodiments, the hSLFN14 polypeptide comprises or consists of SEQ ID NO: 351.

In some embodiments of the compositions of the disclosure, the second RNA binding protein comprises or consists of a human beta-lactamase-like protein 2 (hLACTB2) polypeptide. In some embodiments, the hLACTB2 polypeptide comprises or consists of SEQ ID NO: 352.

In some embodiments of the compositions of the disclosure, the second RNA binding protein comprises or consists of an apurinic/apyrimidinic (AP) endodeoxyribonuclease (APEX) polypeptide. In some embodiments, the second RNA binding protein comprises or consists of an apurinic/apyrimidinic (AP) endodeoxyribonuclease (APEX2) polypeptide. In some embodiments, the APEX2 polypeptide comprises or consists of SEQ ID NO: 353.

In some embodiments, the APEX2 polypeptide comprises or consists of SEQ ID NO: 354.

In some embodiments, the second RNA binding protein comprises or consists of an apurinic or apyrimidinic site lyase (APEX1) polypeptide. In some embodiments, the APEX1 polypeptide comprises or consists of SEQ ID NO: 355.

In some embodiments of the compositions of the disclosure, the second RNA binding protein comprises or consists of an angiogenin (ANG) polypeptide. In some embodiments, the ANG polypeptide comprises or consists of SEQ ID NO: 356.

In some embodiments of the compositions of the disclosure, the second RNA binding protein comprises or consists of a heat responsive protein 12 (HRSP12) polypeptide. In some embodiments, the HRSP12 polypeptide comprises or consists of SEQ ID NO: 357.

In some embodiments of the compositions of the disclosure, the second RNA binding protein comprises or consists of a Zinc Finger CCCH-Type Containing 12A (ZC3H12A) polypeptide. In some embodiments, the ZC3H12A polypeptide is an endonuclease domain of the Z3H12A polypeptide which comprises or consists of SEQ ID NO: 358, also referred to as E17 herein. In some embodiments, the ZC3H12A polypeptide comprises or consists of SEQ ID NO: 359.

In some embodiments of the compositions of the disclosure, the second RNA binding protein comprises or consists of a Reactive Intermediate Imine Deaminase A (RIDA) polypeptide. In some embodiments, the RIDA polypeptide comprises or consists of SEQ ID NO: 360.

In some embodiments of the compositions of the disclosure, the second RNA binding protein comprises or consists of a Phospholipase D Family Member 6 (PDL6) polypeptide. In some embodiments, the PDL6 polypeptide comprises or consists of SEQ ID NO: 361.

In some embodiments of the compositions of the disclosure, the second RNA binding protein comprises or consists of a mitochondrial ribonuclease P catalytic subunit (KIAA0391) polypeptide. In some embodiments, the KIAA0391 polypeptide comprises or consists of SEQ ID NO: 362.

In some embodiments of the compositions of the disclosure, the second RNA binding protein comprises or consists of an argonaute 2 (AGO2) polypeptide. In some embodiments of the compositions of the disclosure, the AGO2 polypeptide comprises or consists of SEQ ID NO: 363.

In some embodiments of the compositions of the disclosure, the second RNA binding protein comprises or consists of a mitochondrial nuclease EXOG (EXOG) polypeptide. In some embodiments, the EXOG polypeptide comprises or consists of SEQ ID NO: 364.

In some embodiments of the compositions of the disclosure, the second RNA binding protein comprises or consists of a Zinc Finger CCCH-Type Containing 12D (ZC3H12D) polypeptide. In some embodiments, the ZC3H12D polypeptide comprises or consists of SEQ ID NO: 365.

In some embodiments of the compositions of the disclosure, the second RNA binding protein comprises or consists of an endoplasmic reticulum to nucleus signaling 2 (ERN2) polypeptide. In some embodiments, the ERN2 polypeptide comprises or consists of SEQ ID NO: 366.

In some embodiments of the compositions of the disclosure, the second RNA binding protein comprises or consists of a pelota mRNA surveillance and ribosome rescue factor (PELO) polypeptide. In some embodiments, the PELO polypeptide comprises or consists of SEQ ID NO: 367.

In some embodiments of the compositions of the disclosure, the second RNA binding protein comprises or consists of a YBEY metallopeptidase (YBEY) polypeptide. In some embodiments, the YBEY polypeptide comprises or consists of SEQ ID NO: 368.

In some embodiments of the compositions of the disclosure, the second RNA binding protein comprises or consists of a cleavage and polyadenylation specific factor 4 like (CPSF4L) polypeptide. In some embodiments, the CPSF4L polypeptide comprises or consists of SEQ ID NO: 369.

In some embodiments of the compositions of the disclosure, the second RNA binding protein comprises or consists of an hCG_2002731 polypeptide. In some embodiments, the hCG_2002731 polypeptide comprises or consists of SEQ ID NO: 370.

In some embodiments, the hCG_2002731 polypeptide comprises or consists of SEQ ID NO: 371.

In some embodiments of the compositions of the disclosure, the second RNA binding protein comprises or consists of an Excision Repair Cross-Complementation Group 1 (ERCC1) polypeptide. In some embodiments, the ERCC1 polypeptide comprises or consists of SEQ ID NO: 372.

In some embodiments of the compositions of the disclosure, the second RNA binding protein comprises or consists of a ras-related C3 botulinum toxin substrate 1 isoform (RAC1) polypeptide. In some embodiments, the RAC1 polypeptide comprises or consists of SEQ ID NO: 373.

In some embodiments of the compositions of the disclosure, the second RNA binding protein comprises or consists of a Ribonuclease A A1 (RAA1) polypeptide. In some embodiments, the RAA1 polypeptide comprises or consists of SEQ ID NO: 374.

In some embodiments of the compositions of the disclosure, the second RNA binding protein comprises or consists of a Ras Related Protein (RAB1) polypeptide. In some embodiments, the RAB1 polypeptide comprises or consists of SEQ ID NO: 375.

In some embodiments of the compositions of the disclosure, the second RNA binding protein comprises or consists of a DNA Replication Helicase/Nuclease 2 (DNA2) polypeptide. In some embodiments, the DNA2 polypeptide comprises or consists of SEQ ID NO: 376.

In some embodiments of the compositions of the disclosure, the second RNA binding protein comprises or consists of a FLJ35220 polypeptide. In some embodiments, the FLJ35220 polypeptide comprises or consists of SEQ ID NO: 377.

In some embodiments of the compositions of the disclosure, the second RNA binding protein comprises or consists of a FLJ13173 polypeptide. In some embodiments, the FLJ13173 polypeptide comprises or consists of SEQ ID NO: 378.

In some embodiments of the compositions of the disclosure, the second RNA binding protein comprises or consists of Teneurin Transmembrane Protein (TENM) polypeptide. In some embodiments, the second RNA binding protein comprises or consists of Teneurin Transmembrane Protein 1 (TENM1) polypeptide. In some embodiments, the TENM1 polypeptide comprises or consists of SEQ ID NO: 379.

In some embodiments, the second RNA binding protein comprises or consists of Teneurin Transmembrane Protein 2 (TENM2) polypeptide. In some embodiments, the TENM2 polypeptide comprises or consists of SEQ ID NO: 380.

In some embodiments of the compositions of the disclosure, the second RNA binding protein comprises or consists of a Ribonuclease Kappa (RNaseK) polypeptide. In some embodiments, the RNaseK polypeptide comprises or consists of SEQ ID NO: 381.

In some embodiments of the compositions of the disclosure, the second RNA binding protein comprises or consists of a transcription activator-like effector nuclease (TALEN) polypeptide or a nuclease domain thereof In some embodiments, the TALEN polypeptide comprises or consists of SEQ ID NO: 382. In some embodiments, the TALEN polypeptide comprises or consists of SEQ ID NO: 383.

In some embodiments of the compositions of the disclosure, the second RNA binding protein comprises or consists a zinc finger nuclease polypeptide or a nuclease domain thereof In some embodiments, the second RNA binding protein comprises or consists of a ZNF638 polypeptide or a nuclease domain thereof In some embodiments, the ZNF638 polypeptide comprises or consists of SEQ ID NO: 384.

In some embodiments of the compositions of the disclosure, the second RNA binding protein comprises or consists of a PIN domain derived from the human SMG6 protein, also commonly known as telomerase-binding protein EST1A isoform 3, NCBI Reference Sequence: NP_001243756.1. In some embodiments, the PIN from hSMG6 is used herein in the form of a Cas fusion protein and as an internal control, for example, and without limitation. In some embodiments, the PIN polypeptide comprises or consists of SEQ ID NO: 598.

In some embodiments of the compositions of the disclosure, the composition further comprises (a) a sequence comprising a gRNA that specifically binds within an RNA molecule and (b) a sequence encoding a nuclease. In some embodiments, a nuclease comprises a sequence isolated or derived from a CRISPR/Cas protein. In some embodiments, a nuclease comprises a sequence isolated or derived from a TALEN or a nuclease domain thereof In some embodiments, a nuclease comprises a sequence isolated or derived from a zinc finger nuclease or a nuclease domain thereof.

### AAV Vectors

An “AAV vector” as used herein refers to a vector comprising, consisting essentially of, or consisting of one or more nucleic acid molecules and one or more AAV inverted terminal repeat sequences (ITRs). In some aspects, the nucleic acid molecule encodes for a CAG-repeat targeting protein and/or composition of the disclosure. Such AAV vectors can be replicated and packaged into infectious viral particles when present in a host cell that provides the functionality of rep and cap gene products; for example, by transfection of the host cell. In some aspects, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing AAV vectors may also contain elements for manufacturing purposes, e.g., antibiotic resistance genes, origin of replication sequences etc., but these are not encapsidated and thus do not form part of the AAV particle.

In some aspects, an AAV vector can comprise at least one nucleic acid molecule encoding a CUG-repeat targeting composition of the disclosure. In some aspects, an AAV vector can comprise at least one regulatory sequence. In some aspects, an AAV vector can comprise at least one AAV inverted terminal (ITR) sequence. In some aspects, an AAV vector can comprise a first ITR sequence and a second ITR sequence. In some aspects, an AAV vector can comprise at least one promoter sequence. In some aspects, an AAV vector can comprise at least one enhancer sequence. In some aspects, an AAV vector can comprise at least one polyA sequence. In some aspects, an AAV vector can comprise at least one linker sequence. In some aspects, an AAV vector of the disclosure can comprise at least on nuclear localization signals. In some aspects, an AAV vector of the disclosure can comprise a CUG-repeat targeting PUF or PUMBY protein, peptide, or fragment thereof In some aspects, an AAV vector of the disclosure can comprise a Cas protein, peptide, or fragment thereof. In some aspects, an AAV vector of the disclosure can comprise an endonuclease protein, peptide, or fragment thereof In some aspects, an AAV vector of the disclosure can comprise a guide RNA, in some cases a CUG-repeat targeting guide RNA. In some aspects, AAV vectors of the disclosure can comprise a fusion protein comprising one or more elements of the disclosure, including, but not limited to, a CUG-repeat targeting protein (such as a Cas, PUF, or PUMBY) and an endonuclease. Optionally, fusion proteins of the AAV vector can further comprise a linker amino acid sequence between the one or more elements of the disclosure.

In some aspects, a AAV vector can comprise a first AAV ITR sequence, a promoter sequence, a CUG-repeat targeting composition nucleic acid molecule, a regulatory sequence and a second AAV ITR sequence. In some aspects, an AAV vector can comprise, in the 5′ to 3′ direction, a first AAV ITR sequence, a promoter sequence, a transgene nucleic acid molecule, and a second AAV ITR sequence.

### CUG-Targeting Cas13d Vectors

In some embodiments of the compositions of the disclosure, CUG-targeting Cas13d compositions are packaged as AAV unitary vectors. In some embodiments, CUG-targeting Cas13d compositions packaged as AAV unitary vectors are set forth in SEQ ID NOs 518, 528, 534, 536, and 539.

In some embodiments, a CUG-targeting Cas13d composition comprises from 5′ to 3′: a human U6 promoter, a cas13d gRNA, wherein the gRNA comprises a direct repeat sequence and a CUG targeting spacer sequence, an EFS promoter, a kozak sequence, a SV-40 NLS sequence, a linker sequences, a sequence encoding Cas13d, a linker sequence, a SV40 NLS sequence, a linker sequence, an HA tag sequence, and a BGH poly a sequence. In some embodiments, a nucleic acid encoding a CUG-targeting Cas13d composition is set forth in SEQ ID NO: 518. In some embodiments, the CUG-targeting Cas13d composition is arranged as depicted in Table 3.

TABLE 3
CUG-targeting Cas13d composition for packaging in AAV unitary
vectors

| Plasmid Element | Amino Acid Sequences |
|---|---|
| Human U6 promoter | gagggcctatttcccatgattccttcatatttgcatatacgatacaaggctgttagagagataattggaatta |
| | atttgactgtaaacacaaagatattagtacaaaatacgtgacgtagaaagtaataatttcttgggtagtttgc |
| | agttttaaaattatgttttaaaatggactatcatatgcttaccgtaacttgaaagtatttcgatttcttggct |
| | ttatatatcttGTGGAAAGGACGAAACACC (SEQ ID NO: 519) |
| CasRx direct repeat | AACCCCTACCAACTGGTCGGGGTTTGAAAC (SEQ ID NO: 302) |
| (DR) | |
| Spacer (CTG guide 3) | CAGCAGCAGCAGCAGCAGCAGCAGCA (SEQ ID NO: 459) |
| EFS promoter | TAGGTCTTGAAAGGAGTGGGAATTGGCTCCGGTGCCCGTCAGTGGGCAGAGCGCA |
| | CATCGCCCACAGTCCCCGAGAAGTTGGGGGGAGGGGTCGGCAATTGATCCGGTGC |
| | CTAGAGAAGGTGGCGCGGGGTAAACTGGGAAAGTGATGTCGTGTACTGGCTCCGC |
| | CTTTTTCCCGAGGGTGGGGGAGAACCGTATATAAGTGCAGTAGTCGCCGTGAACGT |
| | TCTTTTTCGCAACGGGTTTGCCGCCAGAACACAGG (SEQ ID NO: 520) |
| Kozak Sequence | AGAACCATG (SEQ ID NO: 546) |
| SV-40 NLS | CCCAAGAAgAAGAGAAAGGTG (SEQ ID NO: 524) |
| Linker | GAGGCCAGC (SEQ ID NO: 521) |
| CasRx | ATCGAAAAAAAAAAGTCCTTCGCCAAGGGCATGGGCGTGAAGTCCACACTCGTGT |
| | CCGGCTCCAAAGTGTACATGACAACCTTCGCCGAAGGCAGCGACGCCAGGCTGGA |
| | AAAGATCGTGGAGGGCGACAGCATCAGGAGCGTGAATGAGGGCGAGGCCTTCAG |
| | CGCTGAAATGGCCGATAAAAACGCCGGCTATAAGATCGGCAACGCCAAATTCAGC |
| | CATCCTAAGGGCTACGCCGTGGTGGCTAACAACCCTCTGTATACAGGACCCGTCCA |
| | GCAGGATATGCTCGGCCTGAAGGAAACTCTGGAAAAGAGGTACTTCGGCGAGAGC |
| | GCTGATGGCAATGACAATATTTGTATCCAGGTGATCCATAACATCCTGGACATTGA |
| | AAAAATCCTCGCCGAATACATTACCAACGCCGCCTACGCCGTCAACAATATCTCCG |
| | GCCTGGATAAGGACATTATTGGATTCGGCAAGTTCTCCACAGTGTATACCTACGAC |
| | GAATTCAAAGACCCCGAGCACCATAGGGCCGCTTTCAACAATAACGATAAGCTCA |
| | TCAACGCCATCAAGGCCCAGTATGACGAGTTCGACAACTTCCTCGATAACCCCAGA |
| | CTCGGCTATTTCGGCCAGGCCTTTTTCAGCAAGGAGGGCAGAAATTACATCATCAA |
| | TTACGGCAACGAATGCTATGACATTCTGGCCCTCCTGAGCGGACTGAGGCACTGGG |
| | TGGTCCATAACAACGAAGAAGAGTCCAGGATCTCCAGGACCTGGCTCTACAACCT |
| | CGATAAGAACCTCGACAACGAATACATCTCCACCCTCAACTACCTCTACGACAGG |
| | ATCACCAATGAGCTGACCAACTCCTTCTCCAAGAACTCCGCCGCCAACGTGAACTA |
| | TATTGCCGAAACTCTGGGAATCAACCCTGCCGAATTCGCCGAACAATATTTCAGAT |
| | TCAGCATTATGAAAGAGCAGAAAAACCTCGGATTCAATATCACCAAGCTCAGGGA |
| | AGTGATGCTGGACAGGAAGGATATGTCCGAGATCAGGAAAAATCATAAGGTGTTC |
| | GACTCCATCAGGACCAAGGTCTACACCATGATGGACTTTGTGATTTATAGGTATTA |
| | CATCGAAGAGGATGCCAAGGTGGCTGCCGCCAATAAGTCCCTCCCCGATAATGAG |
| | AAGTCCCTGAGCGAGAAGGATATCTTTGTGATTAACCTGAGGGGCTCCTTCAACGA |
| | CGACCAGAAGGATGCCCTCTACTACGATGAAGCTAATAGAATTTGGAGAAAGCTC |
| | GAAAATATCATGCACAACATCAAGGAATTTAGGGGAAACAAGACAAGAGAGTAT |
| | AAGAAGAAGGACGCCCCTAGACTGCCCAGAATCCTGCCCGCTGGCCGTGATGTTT |
| | CCGCCTTCAGCAAACTCATGTATGCCCTGACCATGTTCCTGGATGGCAAGGAGATC |
| | AACGACCTCCTGACCACCCTGATTAATAAATTCGATAACATCCAGAGCTTCCTGAA |
| | GGTGATGCCTCTCATCGGAGTCAACGCTAAGTTCGTGGAGGAATACGCCTTTTTCA |
| | AAGACTCCGCCAAGATCGCCGATGAGCTGAGGCTGATCAAGTCCTTCGCTAGAAT |
| | GGGAGAACCTATTGCCGATGCCAGGAGGGCCATGTATATCGACGCCATCCGTATTT |
| | TAGGAACCAACCTGTCCTATGATGAGCTCAAGGCCCTCGCCGACACCTTTTCCCTG |
| | GACGAGAACGGAAACAAGCTCAAGAAAGGCAAGCACGGCATGAGAAATTTCATT |
| | ATTAATAACGTGATCAGCAATAAAAGGTTCCACTACCTGATCAGATACGGTGATCC |
| | TGCCCACCTCCATGAGATCGCCAAAAACGAGGCCGTGGTGAAGTTCGTGCTCGGC |
| | AGGATCGCTGACATCCAGAAAAAACAGGGCCAGAACGGCAAGAACCAGATCGAC |
| | AGGTACTACGAAACTTGTATCGGAAAGGATAAGGGCAAGAGCGTGAGCGAAAAG |
| | GTGGACGCTCTCACAAAGATCATCACCGGAATGAACTACGACCAATTCGACAAGA |
| | AAAGGAGCGTCATTGAGGACACCGGCAGGGAAAACGCCGAGAGGGAGAAGTTTA |
| | AAAAGATCATCAGCCTGTACCTCACCGTGATCTACCACATCCTCAAGAATATTGTC |
| | AATATCAACGCCAGGTACGTCATCGGATTCCATTGCGTCGAGCGTGATGCTCAACT |
| | GTACAAGGAGAAAGGCTACGACATCAATCTCAAGAAACTGGAAGAGAAGGGATT |
| | CAGCTCCGTCACCAAGCTCTGCGCTGGCATTGATGAAACTGCCCCCGATAAGAGA |
| | AAGGACGTGGAAAAGGAGATGGCTGAAAGAGCCAAGGAGAGCATTGACAGCCTC |
| | GAGAGCGCCAACCCCAAGCTGTATGCCAATTACATCAAATACAGCGACGAGAAGA |
| | AAGCCGAGGAGTTCACCAGGCAGATTAACAGGGAGAAGGCCAAAACCGCCCTGA |
| | ACGCCTACCTGAGGAACACCAAGTGGAATGTGATCATCAGGGAGGACCTCCTGAG |
| | AATTGACAACAAGACATGTACCCTGTTCAGAAACAAGGCCGTCCACCTGGAAGTG |
| | GCCAGGTATGTCCACGCCTATATCAACGACATTGCCGAGGTCAATTCCTACTTCCA |
| | ACTGTACCATTACATCATGCAGAGAATTATCATGAATGAGAGGTACGAGAAAAGC |
| | AGCGGAAAGGTGTCCGAGTACTTCGACGCTGTGAATGACGAGAAGAAGTACAACG |
| | ATAGGCTCCTGAAACTGCTGTGTGTGCCTTTCGGCTACTGTATCCCCAGGTTTAAG |
| | AACCTGAGCATCGAGGCCCTGTTCGATAGGAACGAGGCCGCCAAGTTCGACAAGG |
| | AGAAAAAGAAGGTGTCCGGCAATTCC (SEQ ID NO: 144) |
| Linker | GGATCCgga (SEQ ID NO: 523) |
| SV40 NLS | Cctaagaaaaagaggaaggtg (SEQ ID NO: 532) |
| Linker | Gcggccgct (SEQ ID NO: 525) |
| HA tag | TACCCATACGATGTTCCAGATTACGCT (SEQ ID NO: 526) |
| BGH polyA | TGTGCCTTCTAGTTGCCAGCCATCTGTTGTTTGCCCCTCCCCCGTGCCTTCCTTGAC |
| | CCTGGAAGGTGCCACTCCCACTGTCCTTTCCTAATAAAATGAGGAAATTGCATCGC |
| | ATTGTCTGAGTAGGTGTCATTCTATTCTGGGGGGTGGGGGGGGCAGGACAGCAA |
| | GGGGGAGGATTGGGAAGACAATAGCAGGCATGCTGGGGATGCGGTGGGCTCTATG |
| | GC (SEQ ID NO: 527) |

In some embodiments, an AAV vector comprising a CUG-targeting Cas13d composition comprises from 5′ to 3′: a human U6 promoter, a cas13d gRNA, wherein the gRNA comprises a direct repeat sequence and a CUG targeting spacer sequence, an EFS promoter, a kozak sequence, a sequence encoding Cas13d, a linker sequence, a SV40 NLS sequence, and a SV40 poly a sequence. In some embodiments, a nucleic acid encoding a CUG-targeting Cas13d composition is set forth in SEQ ID NO: 528. In some embodiments, the CUG-targeting Cas13d composition is arranged as depicted in Table 4.

TABLE 4
CUG-targeting Cas13d composition for packaging in AAV unitary
vectors

| Plasmid Element | Amino Acid Sequences |
|---|---|
| Human U6 promoter | GAGGGCCTATTTCCCATGATTCCTTCATATTTGCATATACGATACAAGGCTGTTAG |
| | AGAGATAATTAGAATTAATTTGACTGTAAACACAAAGATATTAGTACAAAATACG |
| | TGACGTAGAAAGTAATAATTTCTTGGGTAGTTTGCAGTTTTAAAATTATGTTTTAA |
| | AATGGACTATCATATGCTTACCGTAACTTGAAAGTATTTCGATTTCTTGGCTTTATA |
| | TATCTTGTGGAAAGGACGAAACACC (SEQ ID NO: 519) |
| Seq198 direct repeat | Caagtaaacccctaccaagtggtcggggtttgaaac (SEQ ID NO: 199) |
| (DR) | |
| Spacer (CTG guide 3) | CAGCAGCAGCAGCAGCAGCAGCAGCA (SEQ ID NO: 459) |
| EFS promoter | TAGGTCTTGAAAGGAGTGGGAATTGGCTCCGGTGCCCGTCAGTGGGCAGAGCGCA |
| | CATCGCCCACAGTCCCCGAGAAGTTGGGGGGAGGGGTCGGCAATTGATCCGGTGC |
| | CTAGAGAAGGTGGCGCGGGGTAAACTGGGAAAGTGATGTCGTGTACTGGCTCCGC |
| | CTTTTTCCCGAGGGTGGGGGAGAACCGTATATAAGTGCAGTAGTCGCCGTGAACGT |
| | TCTTTTTCGCAACGGGTTTGCCGCCAGAACACAGG (SEQ ID NO: 520) |
| Kozak Sequence | gccgccaccATG (SEQ ID NO: 529) |
| Cas13d Seq198 | ATCGAAAAGAAGAAGAGCTACGCTAAAGGAATGGGCCTGAAAAGCACCATCGTGT |
| | CCGGCTCTAAAGTGTACATGACAACCTTCGGCGATGGCAGCGAGGCCAGACTGGA |
| | GAAGGTGGTTGAGAACGATAGCATCAAGACCCTGCACGAGGGCGAGGCCTTCAGC |
| | GCTGAGATGACCGACAACAACGCCGGCTATAAGATCGGAAACGTGAAGTTCTCCC |
| | ACCCTAACGGCTACGACGTGGTCGCCAACAACCCTTTCTACACCGGCCCTGTGCAG |
| | CAGGACATGCTGGGCCTGAAAGAAATCCTGGAAAGACGGTACTTTGGATCTAGCA |
| | CAGACGGTAACAATACCATCTGCATCCAAATCATCCACAATATCCTGGATATCGAA |
| | AAAATTCTGGCAGAGTACATCACAAACGCAGTGTACGCCACCAACAACATCATCG |
| | ATCCTGATAACGACGTGATCGGCGGCAAGAAGTTCACCAGCATTAAAACCTTCGC |
| | CCAGTTCTCCGCCAGCGACAGCAGCAACGATTTCGAGCAGTTCCTGAAAAATCCCA |
| | GACTCGGCTACCTGGGCAAAGCCTTTTTCTACAAGGACGGCAAGCGGAACAACAG |
| | ACAGAAGGATCCTATCGAGTGTTACCACCTGCTGGCCCTGCTGTGCGGCCTGCGTA |
| | ATTGGGTTGTGCACAACAACGAGGAAAAGGACCTGATCAAGTACACCTGGTTGTA |
| | TAACCTGGACAAGTACCTGGATGCCGAGTACATCACCACCCTGAACTACATGTACA |
| | ACGACATCGGCGACGAGTTGACGGACTCTTTCTCCAAGAACAGCGCCGCCAATAT |
| | CAACTACATCGCCGAGACCCTGGGAATCGACCCCAAGACCTTCGCCGAGCAATAC |
| | TTCCGGTTCTCTATCATGAAGGAACAGAAAAACCTGGGATTCAACCTGACCAAGCT |
| | GAGAGAGGTGATGCTGGACCGGAAGGACATGAGCGAGATCAGAGAGAACCACAA |
| | CGACTTCGATTCTATCAGAGCCAAGGTGTACACAATGATGGACTTCGTGATCTATC |
| | GGTACTACATCGAAGAGGCCGCTAAGGTGAACGCCGCCAACAAGAGCCTGCCCGA |
| | CAACGAGAAGAGCCTGAGCGAGAAAGACATCTTCGTGATTTCACTCAGAGGCAGC |
| | TTCAACGAAGATCAGAAGGATCGGCTGTACTACGACGAGGCGCAAAGACTGTGGT |
| | CCAAGGTGGGCAAACTGATGCTGAAAATCAAGAAGTTCCGGGGCAAGGACACCAG |
| | AAAGTACAAGAATATGGGCACACCTAGAATCCGGAGGCTGATCCCTGAGGGCAGA |
| | GATATCAGCACCTTCTCCAAGCTGATGTACGCTCTGACTATGTTCCTGGACGGCAA |
| | GGAGATCAATGACCTGCTGACCACACTGATCAACAAATTCGACAACATCCAGAGC |
| | TTCTTAAAGGTGATGCCTCTGATCGGCGTGAACGCCAAATTTGCCGAAGAATATAG |
| | TTTCTTCAACAACTCTGAAAAAATCGCCGACGAACTGCGGCTGATCAAGAGCTTTG |
| | CTAGAATGGGAGAACCCGTGGCTGACGCCAGAAGAGCCATGTATATCGACGCAAT |
| | TCGCATCCTGGGCACCGATCTCTCCGACGACGAGCTGAAGGCCCTGGCTGATTCTT |
| | TTAGCCTGGACGAGAACGGCAATAAGCTGGGGAAGGGCAAGCACGGCATGAGAA |
| | ACTTCATCATTAACAACGTGATAACAAATAAGAGATTCCACTACCTGATCCGGTAC |
| | GGCAACCCAGTCCACCTGCATGAGATCGCCAAGAATGAAGCCGTGGTCAAGTTTG |
| | TGCTGGGAAGAATCGCCGATATCCAGAAGAAACAGGGCCAGAACGGCAAGAACC |
| | AGATCGATAGATACTACGAGACATGCATCGGCAAGGACTCTTCTAAAAGCGTGGC |
| | CGAGAAGGTGAACGCCCTGACCAAGATCATCACAGGCATGAACTACGACCAGTTC |
| | GACAGCAGACGGAACGTGATCGAAAACACCGGCGCCGGCAACGCCGAGAGAGAA |
| | AAGTACAAGAAGATCATCAGCCTGTACCTGACAGTGATCTACCACATCCTGAAGA |
| | ACATTGTTAATATCAACTCAAGATACGTGATCGGATTTCACTGCGTGGAGAGAGAT |
| | GCCCAGCTGTATAAGGAAAAGGGCTACGACATTAATCTGAAAAAGCTGAAAGACA |
| | AGGGATTCACAAGCGTGACCAAGCTGTGCGCCGGAATCGACGAGGAATGCAAGGA |
| | CGTCGAAAAGGAAATGACCGAGCGGGCCAAGGCCTCTTTCGCTGCCCTGGAAACC |
| | GCCAACCCCAAGCTGTACGCCACATACATCAACTACTCTGATGAAGAGAAGAATG |
| | CCGAACTGAGAAAGCAGATCAATAGAGAGAAGGCCAAAACCGCCCTGAACGCTC |
| | ATCTGCGCAACACCAAGTGGAACGTGATCATCCGGGAAGATCTTCTGAGAAGAGA |
| | TAACAAGGCTTGTAAAATCTTCAGAAATAAGGTCGCCCACCTGGAGGCCATCCGA |
| | TACGCTCACCTGTACATCAACGACATCGCTGAGGTGAATAGCTATTTTCAGTTTTA |
| | CCACTACATCATGCAGCGGAGGATCATGGCCGAACGGTACGACAAGAGCAGCGGC |
| | AAGGTTAGAGAATACTTCGACGCCGTGAACAATGAGAAAAAATACAACGATAGAC |
| | TGCTGAAGCTCCTCTGTGTGCCATTCGGCTACTGCATCCCTAGATTCAAGAATCTG |
| | AGCATCGAGGCCCTGTTCGACATGAACGAGGCCGTGAAGTTTGATAAGGAAAAGA |
| | AG (SEQ ID NO: 530) |
| Linker | GGATCC (SEQ ID NO: 531) |
| SV40 NLS | CCCAAAAAAAAAAGGAAGGTG (SEQ ID NO: 532) |
| SV-40 poly A | AACTTGTTTATTGCAGCTTATAATGGTTACAAATAAAGCAATAGCATCACAAATTT |
| | CACAAATAAAGCATTTTTTTCACTGCATTCTAGTTGTGGTTTGTCCAAACTCATCAA |
| | TGTATCTTA (SEQ ID NO: 533) |

In some embodiments, an AAV vector comprising a CUG-targeting Cas13d composition comprises from 5′ to 3′: a human U6 promoter, a cas13d gRNA, wherein the gRNA comprises a direct repeat sequence and a CUG targeting spacer sequence, an EFS promoter, a kozak sequence, a sequence encoding Cas13d, a linker sequence, a SV40 NLS sequence, and anSV40 poly a sequence. In some embodiments, a nucleic acid encoding a CUG-targeting Cas13d composition is set forth in SEQ ID NO: 534. In some embodiments, the CUG-targeting Cas13d composition is arranged as depicted in Table 5.

TABLE 5
CUG-targeting Cas13d composition for packaging in AAV unitary
vectors

| Plasmid Element | Amino Acid Sequences |
|---|---|
| Human U6 promoter | GAGGGCCTATTTCCCATGATTCCTTCATATTTGCATATACGATACAAGGCTGTTAG |
| | AGAGATAATTAGAATTAATTTGACTGTAAACACAAAGATATTAGTACAAAATACG |
| | TGACGTAGAAAGTAATAATTTCTTGGGTAGTTTGCAGTTTTAAAATTATGTTTTAA |
| | AATGGACTATCATATGCTTACCGTAACTTGAAAGTATTTCGATTTCTTGGCTTTATA |
| | TATCTTGTGGAAAGGACGAAACACC (SEQ ID NO: 519) |
| Seq179 direct repeat | Actatagccctgccggaaatgacagggttctacaac (SEQ ID NO: 180) |
| (DR) | |
| Spacer (CTG guide 3) | CAGCAGCAGCAGCAGCAGCAGCAGCA (SEQ ID NO: 459) |
| EFS promoter | TAGGTCTTGAAAGGAGTGGGAATTGGCTCCGGTGCCCGTCAGTGGGCAGAGCGCA |
| | CATCGCCCACAGTCCCCGAGAAGTTGGGGGGAGGGGTCGGCAATTGATCCGGTGC |
| | CTAGAGAAGGTGGCGCGGGGTAAACTGGGAAAGTGATGTCGTGTACTGGCTCCGC |
| | CTTTTTCCCGAGGGTGGGGGAGAACCGTATATAAGTGCAGTAGTCGCCGTGAACGT |
| | TCTTTTTCGCAACGGGTTTGCCGCCAGAACACAGG (SEQ ID NO: 520) |
| Kozak Sequence | gccgccaccATG (SEQ ID NO: 529) |
| Cas 13d Seq179 | GCCAAAAAGAAGAAAACCGCTCGCCAACTGAGAGAAGAAATGCAACAACAGCGG |
| | AAACAGGCCATTCAGAAGCAACAAGAACAGAGACAAGAGAAAGCCGCCGCCGCT |
| | CGCGAGACAGCCGCCCCCGAACAGCCTGCTGCCGCTCCTGTGCCAAAGCGGCAAA |
| | GAAAATCTCTGGCCAAAGCCGCCGGACTGAAGTCCAACTTCATCTTGGACCCACA |
| | GAGAAGAACAACAGTGATGACAGCTTTTGGCCAGGGCAGCACCGCCATCCTGGAG |
| | AAGCAGATCGTGGACAGAGCCATCAGCGACCTGCAGCCGGTTCAGCAGTTCCAAG |
| | TGGAACCTGCCAGTGCCGCCAAGTACAGGCTGAAGAATAGCCGGGTGAGATTCCC |
| | CAACGTGACAGCTGACGATCCTCTGTATAGACGGAAGGATGGCGGCTTCGTGCCT |
| | GGCATGGACGCCCTCAGAAGAAAGAACGTACTGGAACAGAGATTCTTCGGCAAGT |
| | CTTTCGCCGATAACATCCACATCCAGATGATCTACAGCATCCTGGACATCCACAAG |
| | ATCCTCGCTGCCGCGAGCGGCCACATCGTGCACCTGCTCAATATCGTGAATGGCTC |
| | AAAAGATAGAGACTTCATCGGCATGCTGGCCGCCCACGTGCTGTACAATGAGCTG |
| | AACGAGGAGGCCAAGCGGAGCATCGCCGACTTTTGCAAGAGTCCCAGACTGATCT |
| | ACTACTCTGCTGCTTTCTACGAGACATTGGACAACGGCAAGAGCGAGCGACGGTCT |
| | AACGAGGACATCTTCAACATCCTGGCCCTGATGACCTGTCTGAGAAATTTCAGCAG |
| | CCACCACAGCATCGCCATCAAGGTGAAGGACTACAGCGCCGCTGGCCTGTACAAC |
| | CTGCGGAGACTGGGACCTGACATGAAGAAAATGCTGGACACCTTCTACACCGAGG |
| | CCTTCATCCAGCTTAACCAGAGCTTCCAGGACCACAACACCACAAACCTGACATGT |
| | CTGTTCGATATCCTGAACATCTCTGATAGCGCCAGACAGAAGCAGCTGGCTGAGG |
| | AATTTTATAGATACGTGGTGTTCAAGGAACAAAAGAACTTGGGATTCTCCGTGCGG |
| | AAGCTGAGAGAGGAAATGCTGCTGCTGCCAGACGCTGCCGTGATCGCCGATAAGC |
| | GGTACGACACCTGCAGATCCAAGCTGTACAACCTGATGGACTTCCTGATCCTGAGA |
| | GTGTACAGAACCGGCAGAGCCGACAGATGCGACAAGCTGCCTGAGGCCCTGCGGG |
| | CCGCCCTGACCGACGAGGAAAAGGCCGTGGTGTACCACAAAGAAGCCCTGAGCCT |
| | GTGGAACGAGATGAGAACCCTGATCCTCGACGGCCTGCTGCCTCAGATGACACCT |
| | GAGAACCTGAGCAGACTGTCCGGTCAGAAAAGAAAGGGCGAACTGTCTCTGGATG |
| | ACGCCATGCTGAAAGAGTGCCTGTACGAGCCCGGACCTGTGCCCGAGGATGCTGC |
| | CCCTGAGGAAGCCAACGCCGAGTACTTCTGCCGGATGATCTACCTGGCCACCCTGT |
| | TTATGGATGGCAAGGAGATCAACACCCTGCTGACCACCCTGATTAGCAAATTCGA |
| | GAACATCGCCGCCTTCCTGCAGACCATGGAACAGCTGAACATCGAGGCCGAGCTG |
| | GGCCCTGAATACGCCATGTTTACCAGAAGCAGAGCCGTAGCCGAGCAGCTGAGAG |
| | TGATCAACAGCTTCGCCCTGATGAAGAAGCCTCAGGTGAATGCCAAGCAGCAGCT |
| | GTACAGAGCCGCTGTCACCCTGCTGGGAACAGAGGACCCTGACGGCGTGACCGAT |
| | GAGATGCTGTGCATCGACCCCGTGACCGGCAAGATGCTGCCTCCTAACCAGAGGC |
| | ATCATGGCGACACCGGCTTACGGAACTTCATCGCAAACAACGTGGTGGAAAGCCG |
| | GAGATTCCAGTACTTAATCCGGTACAGCGATCCTGCTCAGCTGCACCAGCTCGCCA |
| | GCAACAAGAAGCTGGTCAGATTCGTGCTGAGCAGCATCCCCGACACACAGATCAA |
| | CAGATACTATGAAACCTGTGGCCAGACCAGACTGGCCGGCAGAGCCGCCAAGGTG |
| | GAATTCCTGACAGACATGATTGCCGCCATCAGATTCGACCAGTTTCGGGATGTCAA |
| | TCAGAAAGAGCGCGGCGCCAATACTCAGAAAGAAAGATATAAGGCCATGCTTGGC |
| | CTGTACCAGACCGTGCTGTACCTGGCTGTTAAAAATCTGGTGAACATTAACGCCAG |
| | ATACGTGATGGCCTTCCACTGCGTGGAGCGGGATATGTTTCTGTATGACGGCGAGC |
| | TGACAGATCCCAAGGGCGAGAGCGTGTCTGCTTTCCTGGCTGTGAATGGAAAGAA |
| | GGGCGTGCAGCCTCAGTACCTGCTGCTGACCCAGCTGTTTATCCGGCGGGATTACC |
| | TTAAGCGGAGTGCATGCGAGCAGATCCAGCACAACATGGAAAACATCTCCGACCG |
| | GCTGCTGCGGGAATACCGGAACGCCGTCGCCCACCTGAATGTGATAGCCCATCTG |
| | GCTGACTACTCTGCCGACATGAGAGAAATCACCAGCTACTACGGCTTGTATCACTA |
| | CCTGATGCAGAGACACCTCTTCAAAAGACACGCCTGGCAGATCAGACAGCCTGAA |
| | AGGCCAACTGAGGAGGAACAGAAGCTCATCGAGCAGGAGCAGAAGCAGCTGGCC |
| | TGGGAGAAGGCCCTGTTTGACAAGACCCTGCAGTACCACAGCTACAACAAGGACC |
| | TGGTGAAGGCTCTTAACGCCCCCTTCGGATACAACCTGGCAAGATACAAGAACCT |
| | GTCTATCGAGCCTCTGTTCAGCAAAGAAGCCGCTCCTGCCGCCGAGATCAAGGCCA |
| | CACACGCC (SEQ ID NO: 535) |
| Linker | GGATCC (SEQ ID NO: 531) |
| SV40 NLS | CCCAAAAAAAAAAGGAAGGTG (SEQ ID NO: 532) |
| SV-40 poly A | AACTTGTTTATTGCAGCTTATAATGGTTACAAATAAAGCAATAGCATCACAAATTT |
| | CACAAATAAAGCATTTTTTTCACTGCATTCTAGTTGTGGTTTGTCCAAACTCATCAA |
| | TGTATCTTA (SEQ ID NO: 533) |

In some embodiments, an AAV vector comprising a CUG-targeting Cas13d composition comprises from 5′ to 3′: a human U6 promoter, a cas13d gRNA, wherein the gRNA comprises a direct repeat sequence and a CUG targeting spacer sequence, an EFS promoter, a kozak sequence, a sequence encoding Cas13d, a linker sequence, a SV40 NLS sequence, and anSV40 poly a sequence. In some embodiments, a nucleic acid encoding a CUG-targeting Cas13d composition is set forth in SEQ ID NO: 536. In some embodiments, the CUG-targeting Cas13d composition is arranged as depicted in Table 6.

TABLE 6
CUG-targeting Cas13d composition for packaging in AAV unitary
vectors

| Plasmid Element | Amino Acid Sequences |
|---|---|
| Human U6 promoter | GAGGGCCTATTTCCCATGATTCCTTCATATTTGCATATACGATACAAGGCTGTTAG |
| | AGAGATAATTAGAATTAATTTGACTGTAAACACAAAGATATTAGTACAAAATACG |
| | TGACGTAGAAAGTAATAATTTCTTGGGTAGTTTGCAGTTTTAAAATTATGTTTTAA |
| | AATGGACTATCATATGCTTACCGTAACTTGAAAGTATTTCGATTTCTTGGCTTTATA |
| | TATCTTGTGGAAAGGACGAAACACC (SEQ ID NO: 519) |
| Seq42 direct repeat | GACCAACACCTCTGCAAAACTGCAGGGGTCTAAAAC (SEQ ID NO: 537) |
| (DR) | |
| Spacer (CTG guide 3) | CAGCAGCAGCAGCAGCAGCAGCAGCA (SEQ ID NO: 459) |
| EFS promoter | TAGGTCTTGAAAGGAGTGGGAATTGGCTCCGGTGCCCGTCAGTGGGCAGAGCGCA |
| | CATCGCCCACAGTCCCCGAGAAGTTGGGGGGAGGGGTCGGCAATTGATCCGGTGC |
| | CTAGAGAAGGTGGCGCGGGGTAAACTGGGAAAGTGATGTCGTGTACTGGCTCCGC |
| | CTTTTTCCCGAGGGTGGGGGAGAACCGTATATAAGTGCAGTAGTCGCCGTGAACGT |
| | TCTTTTTCGCAACGGGTTTGCCGCCAGAACACAGG (SEQ ID NO: 520) |
| Kozak Sequence | gccgccaccATG (SEQ ID NO: 529) |
| Cas13d Seq42 | AACAATAAGAGAAAGACAAAGGCCAAGGCCGCTGGACTGAAGAGCGTCTTTTTTG |
| | ATCAGAAGCAAGCCGTGCTGACCACATTCGCCAAGGGCAACAACTCCCAGATCGA |
| | GAAGAAAGTGGTCAACAGCGAGGTCAAAGATCTGAGACAGCCTCCCGCCTTTGAT |
| | CTGGAACTGAAGGAGAAGACCTTCTATATCTCCGGCAAGAACAACATTAACACAT |
| | CTAGGGAGAACCCTCTGGCTAGCGCTTCTCTGCCTCTCTCCAAGAGGCAAAGGATT |
| | AGAGCCGAGAGGATCAAGAGAGCTAGAGAAGAAAATAGACCCTACCATAATGTC |
| | AAGAGGGTGGGAGAGGACGATCTGAGAGCCAAGGCTGACCTCGAGAAACACTAC |
| | TTCGGCAAGGAGTACAGCGATAATCTGAAAATTCAGATTATTTATAATATCCTCGA |
| | CATCAACAAAATCATCAGCCCCTATATCAATGACATCGTCTACTCCATGAACAATC |
| | TGGCTAGAAACGACGAGTATATCGATGGAAAAATCGACGTGATCGGCTCCCTCTC |
| | CTCCACCACAGACTACTCCTCCTTCATGAGCCCCAACAAGGATCTGGAAAAGGAA |
| | AAAAAGTTTTCCTTCCATAGAGAAAACTACAAAAAATTCGTCGAGGCCAGCAAGC |
| | CCTACATGAGGTACTATGGAAAGGTGTTTATTAGAGACGTGAAGAAAAGCAAGCT |
| | CTCCACCGGAAAGGGCGAGAAGATTGAGGTGATGTATAGATCCGACGAGGAAATT |
| | TTCACCATTTTTCAAATTCTGAGCTATGTGAGACAATCCATCATGCACAACGACAT |
| | CGGAAACAAGAGCAGCATTCTGGCCATCGAAAAGTACCCCGCCAGATTCGTCGGC |
| | TTTCTGAGCGACCTCCTCAAAACCAAGACAAACGATGTCAATAGAATGTTCATTGA |
| | CAATAACAGCCAAACAAACTTCTGGGTGCTCTTCAGCATCTTCGGACTGCAAGATC |
| | ACACCAGCGGAGCCGACAAGATCTGTAGAAATTTCTACGACTTCGTGATCAAGGC |
| | CGACAGCAAAAACCTCGGATTCTCCCTCAAGAAGATCAGAGAGCTGATGCTCGAT |
| | CTGCCTAACGCCAACATGCTGAGAGATCACCAATTCGATACCGTGAGGAGCAAGT |
| | TTTATACCCTCCTCGACTTCATTATCTATCAACACTATCTCGAGGAGAAGTCCAGA |
| | ATCGACAACATGGTGGAGAAGCTGAGGATGACCCTCAAGGAAGAGGAAAAGGAA |
| | GTGCTCTACGCTGCCGAGGCCAAGATTGTGTGGAATGCCATCGGAGCCAAGGTCA |
| | TCAACAAGCTCGTGCCCATGATGAATGGCGATGCTCTGAAGGAGATCAAGAGAAA |
| | AAATAGAGATAGAAAGCTCCCTCAGAGCGTGATCGCCACAGTGCAAGTGAATTCC |
| | GACGCCAATGTGTTCTCCGGACTGATCTACTTTCTGACACTGTTTCTCGACGGCAA |
| | GGAGATCAACGAGATGGTGAGCAACCTCATCACCAAGTTCGAGAACATTGACTCT |
| | CTGCTGCATGTCGATAGAGAAATCTACAAGTCCGACGAGAAGGATCTGGATCTCG |
| | AGATCGAGAAGCTGGCCCTCTTTTTCAAGGGCGTGGTGAGGCCTAATGCCAAGAC |
| | AGATACCGGCGCCGGAGAGATCTCCAAGAGCTTCTCCATCTTCCAGAGCGCCGAA |
| | AGGATTATCGAGGAACTGAAGTTCATTAAGAACGTCACAAGAATGGATAACGAGA |
| | TCTTCCCTAGCGAGGGCGTGTTCCTCGATGCCGCTAACGTGCTCGGCGTCAGAGGC |
| | GATGACTTTGACTTTAGCAATGAGTTTGTCGGAGACGATCTGCACAGCGACGCTAA |
| | TAAGAAGATTATTAACAAGATCAATGGCACCAAGGAGGACAGAAATCTGAGGAAC |
| | TTTATTATTAATAACGTCGTGAAGAGCAGAAGGTTTCAGTATATCGCTAGACACAT |
| | GAATACACACTACGTCAAGCAGCTCGCCAATAACGAGACACTGAATAGATTCGTG |
| | CTGAACAAGATGGGAGACGCCAAGATCATCAATAGGTACTACGAGTCCATCTCCG |
| | GCAATACCCCCAATATTGAGGTCAGAAGCCAAATCGACTACCTCGTCAAGAGACT |
| | GAGGAGCTTCAGCTTCGAAGACCTCAACGACGTCAAGCAAAAGGTGAGACCCGGC |
| | ACCAATGAGAGCATCGAGAAGGAGAAGAAAAAGGCCCTCGTCGGACTGTGCCTCA |
| | CAATTCAGTACCTCGTGTATAAAAATCTGGTGAATATCAACGCTAGGTACACCACC |
| | GCTTTCTACTGTCTGGAGAGGGACTCCAAACTGAAAGGCTTTGGCGTGGACGTGTG |
| | GAGAGATTTCGAATCCTACACCGCTCTGACCAATCACTTTATCAAAGAAGGCTATC |
| | TGCCCGTGAGAAAGGCTGAAATTCTGAGGGCCAATCTGAAGCATCTGGACTGTGA |
| | AGACGGCTTCAAATATTACAGAAACCAAGTGACCCACCTCAACGCCATTAGAGTC |
| | GCCTATAAATATATCAACGAGATTAAATCCGTGCACAGCTACTTCGCCCTCTACCA |
| | CTACATCATGCAGAGACATCTGTACGACAGCCTCCAAGCCAAAGCTAAGGACTCC |
| | TCCGGCTTCGTGATCGACGCTCTGAAGAAATCCTTCGAGCACAAGATCTACAGCAA |
| | AGATCTGCTCCACGTGCTGCACTCCCCCTTCGGCTATAATACCGCTAGATATAAAA |
| | ATCTGAGCATCGAGGCCCTCTTCGACAAGAACGAATCCAGACCCGAGGTGAATCC |
| | CCTCTCCACCAATGAT (SEQ ID NO: 538) |
| Linker | GGATCC (SEQ ID NO: 531) |
| SV40 NLS | CCCAAAAAAAAAAGGAAGGTG (SEQ ID NO: 532) |
| SV-40 poly A | AACTTGTTTATTGCAGCTTATAATGGTTACAAATAAAGCAATAGCATCACAAATTT |
| | CACAAATAAAGCATTTTTTTCACTGCATTCTAGTTGTGGTTTGTCCAAACTCATCAA |
| | TGTATCTTA (SEQ ID NO: 533) |

In some embodiments, an AAV vector comprising a CUG-targeting Cas13d composition comprises from 5′ to 3′: a human U6 promoter, a cas13d gRNA, wherein the gRNA comprises a direct repeat sequence and a CUG targeting spacer sequence, an EFS promoter, a kozak sequence, a sequence encoding Cas13d, a linker sequence, a SV40 NLS sequence, and anSV40 poly a sequence. In some embodiments, a nucleic acid encoding a CUG-targeting Cas13d composition is set forth in SEQ ID NO: 539. In some embodiments, the CUG-targeting Cas13d composition is arranged as depicted in Table 7.

TABLE 7
CUG-targeting Cas13d compositions for packaging in AAV unitary
vectors

| Plasmid Element | Amino Acid Sequences |
|---|---|
| Human U6 promoter | GAGGGCCTATTTCCCATGATTCCTTCATATTTGCATATACGATACAAGGCTGTTAG |
| | AGAGATAATTAGAATTAATTTGACTGTAAACACAAAGATATTAGTACAAAATACG |
| | TGACGTAGAAAGTAATAATTTCTTGGGTAGTTTGCAGTTTTAAAATTATGTTTTAA |
| | AATGGACTATCATATGCTTACCGTAACTTGAAAGTATTTCGATTTCTTGGCTTTATA |
| | TATCTTGTGGAAAGGACGAAACACC (SEQ ID NO: 519) |
| Seq212 direct repeat | gtacaatagccctgcagtaaggcagggttctaAGAC (SEQ ID NO: 213) |
| Spacer (CTG guide 3) | CAGCAGCAGCAGCAGCAGCAGCAGCA (SEQ ID NO: 459) |
| EFS promoter | TAGGTCTTGAAAGGAGTGGGAATTGGCTCCGGTGCCCGTCAGTGGGCAGAGCGCA |
| | CATCGCCCACAGTCCCCGAGAAGTTGGGGGGAGGGGTCGGCAATTGATCCGGTGC |
| | CTAGAGAAGGTGGCGCGGGGTAAACTGGGAAAGTGATGTCGTGTACTGGCTCCGC |
| | CTTTTTCCCGAGGGTGGGGGAGAACCGTATATAAGTGCAGTAGTCGCCGTGAACGT |
| | TCTTTTTCGCAACGGGTTTGCCGCCAGAACACAGG (SEQ ID NO: 520) |
| Kozak Sequence | gccgccaccATG (SEQ ID NO: 529) |
| Cas13d Seq212 | AAGAAGAAGCACCAGAGCGCCGCCGAGAAGAGGCAAGTGAAGAAGCTCAAGAAT |
| | CAAGAGAAGGCCCAGAAGTACGCTAGCGAGCCTTCCCCCCTCCAGAGCGATACAG |
| | CTGGCGTGGAATGCTCCCAGAAAAAGACAGTCGTCAGCCACATTGCCAGCTCCAA |
| | GACACTGGCCAAGGCTATGGGACTCAAATCCACACTGGTCATGGGCGACAAGCTG |
| | GTCATCACCAGCTTTGCTGCTAGCAAGGCTGTCGGAGGCGCTGGCTACAAAAGCG |
| | CTAACATTGAAAAAATCACAGATCTGCAAGGAAGGGTCATTGAGGAGCACGAAAG |
| | GATGTTTAGCGCCGATGTCGGAGAGAAAAATATCGAACTGAGCAAGAATGACTGC |
| | CACACCAACGTCAACAACCCCGTGGTGACCAACATCGGAAAGGATTACATCGGAC |
| | TGAAATCTAGGCTGGAGCAAGAGTTTTTCGGCAAGACATTCGAGAATGACAATCT |
| | GCATGTGCAGCTGGCCTACAATATCCTCGACATCAAGAAAATTCTGGGAACCTATG |
| | TGAACAATATCATTTATATCTTCTACAATCTGAATAGGGCTGGCACCGGCAGAGAT |
| | GAGAGGATGTATGACGACCTCATCGGCACACTGTACGCTTACAAACCCATGGAGG |
| | CTCAACAGACCTATCTGCTCAAAGGCGACAAGGATATGAGGAGGTTTGAGGAGGT |
| | GAAACAGCTGCTGCAAAACACCTCCGCTTACTATGTGTATTACGGCACACTGTTCG |
| | AGAAGGTGAAGGCTAAGAGCAAGAAGGAACAGAGGGCTAAGGAGGCCGAAATCG |
| | ACGCTTGTACCGCCCATAACTACGATGTGCTGAGACTGCTGTCCCTCATGAGGCAG |
| | CTGTGCATGCACTCCGTCGCTGGAACAGCCTTTAAGCTGGCTGAGTCCGCTCTGTT |
| | CAACATTGAGGATGTGCTCAGCGCCGATCTGAAGGAAATCCTCGATGAAGCCTTCT |
| | CCGGCGCCGTGAACAAGCTCAATGACGGATTCGTGCAGCACTCCGGCAACAATCT |
| | GTACGTGCTCCAGCAGCTGTACCCTAATGAGACCATCGAGAGAATCGCCGAGAAG |
| | TACTACAGACTCACCGTGAGGAAGGAGGATCTGAACATGGGAGTCAACATTAAAA |
| | AGCTGAGGGAGCTGATCGTGGGCCAATACTTTCCCGAGGTCCTCGACAAAGAATA |
| | CGACCTCTCCAAGAATGGAGACAGCGTGGTGACATACAGAAGCAAGATTTATACC |
| | GTGATGAATTACATTCTGCTGTATTACCTCGAGGACCACGACTCCAGCAGAGAAAG |
| | CATGGTCGAAGCTCTGAGACAAAACAGAGAGGGCGATGAAGGCAAGGAGGAGAT |
| | CTATAGACAGTTTGCCAAGAAGGTGTGGAACGGCGTGTCCGGACTGTTTGGCGTGT |
| | GTCTGAACCTCTTCAAGACCGAAAAGAGAAACAAGTTTAGGAGCAAAGTCGCCCT |
| | CCCCGATGTGTCCGGCGCTGCCTATATGCTCTCCTCCGAGAACATCGACTACTTTGT |
| | CAAGATGCTCTTCTTTGTGTGTAAGTTTCTGGATGGCAAAGAAATCAACGAGCTGC |
| | TGTGCGCTCTGATCAACAAATTTGATAATATTGCCGATATTCTGGATGCTGCCGCT |
| | CAATGTGGCTCCTCCGTCTGGTTCGTGGACAGCTATAGGTTCTTCGAGAGATCTAG |
| | GAGGATTAGCGCCCAGATTAGAATCGTGAAGAACATCGCTTCCAAGGATTTTAAG |
| | AAATCCAAGAAGGATTCCGATGAGAGCTACCCCGAGCAGCTGTATCTGGATGCTC |
| | TGGCTCTGCTCGGAGACGTCATCTCCAAGTACAAGCAGAATAGAGATGGCAGCGT |
| | CGTCATCGATGACCAAGGCAATGCCGTGCTGACAGAGCAATACAAGAGGTTTAGA |
| | TATGAATTTTTCGAGGAGATCAAGAGGGACGAAAGCGGCGGCATCAAGTACAAGA |
| | AGTCCGGAAAACCCGAGTACAACCATCAGAGAAGGAATTTTATTCTGAATAATGT |
| | GCTGAAAAGCAAATGGTTTTTCTATGTGGTGAAGTACAATAGGCCCAGCAGCTGC |
| | AGAGAACTGATGAAGAATAAGGAAATTCTGAGGTTCGTGCTGAGAGACATCCCCG |
| | ACTCCCAAGTGAGAAGATACTTTAAGGCCGTCCAAGGAGAGGAAGCTTACGCTAG |
| | CGCCGAAGCTATGAGGACAAGACTGGTCGACGCTCTGTCCCAATTTAGCGTCACA |
| | GCTTGTCTGGATGAAGTGGGCGGCATGACAGACAAGGAATTCGCCTCCCAGAGGG |
| | CCGTCGATAGCAAAGAAAAACTGAGAGCCATCATCAGACTGTATCTGACAGTCGC |
| | CTATCTGATTACCAAGAGCATGGTGAAGGTGAATACAAGGTTTAGCATTGCCTTTA |
| | GCGTGCTGGAGAGGGACTACTATCTGCTCATTGACGGCAAGAAGAAATCCAGCGA |
| | CTACACCGGAGAGGATATGCTGGCTCTGACCAGAAAATTTGTGGGCGAAGATGCT |
| | GGACTGTATAGAGAGTGGAAAGAGAAGAACGCTGAAGCCAAGGACAAATATTTTG |
| | ACAAGGCCGAAAGGAAGAAGGTGCTGAGACAGAACGATAAGATGATCAGAAAGA |
| | TGCACTTCACACCCCACTCCCTCAATTACGTCCAAAAGAATCTCGAAAGCGTCCAG |
| | AGCAACGGACTGGCCGCCGTCATCAAGGAATATAGAAATGCCGTCGCTCACCTCA |
| | ATATCATCAATAGACTGGACGAGTACATTGGCTCCGCTAGGGCTGATAGCTACTAC |
| | TCTCTGTACTGTTACTGCCTCCAAATGTATCTGAGCAAGAACTTCAGCGTGGGCTA |
| | CCTCATCAACGTGCAAAAGCAGCTGGAGGAGCACCACACCTACATGAAGGATCTC |
| | ATGTGGCTGCTCAACATCCCCTTCGCTTACAACCTCGCCAGATACAAAAATCTGTC |
| | CAACGAAAAACTCTTTTACGACGAGGAAGCCGCCGCCGAAAAGGCTGACAAGGCT |
| | GAGAACGAGAGAGGCGAA (SEQ ID NO: 540) |
| Linker | GGAAGC (SEQ ID NO: 531) |
| SV40 NLS | CCCAAGAAGAAAAGGAAGGTC (SEQ ID NO: 532) |
| SV-40 poly A | AACTTGTTTATTGCAGCTTATAATGGTTACAAATAAAGCAATAGCATCACAAATTT |
| | CACAAATAAAGCATTTTTTTCACTGCATTCTAGTTGTGGTTTGTCCAAACTCATCAA |
| | TGTATCTTA (SEQ ID NO: 533) |

In some embodiments, nucleic acid sequences encoding CUG-targeting Cas13d proteins of the disclosure are codon optimized nucleic acid sequences. In some embodiments, the codon optimized sequence encoding a CUG-targeting Cas13d protein exhibits at least 5%, at least 10%, at least 20%, at least 30%, at least 50%, at least 75%, at least 100%, at least 200%, at least 300%, at least 500%, or at least 1000% increased translation in a human subject relative to a wild-type or non-codon optimized nucleic acid sequence.

In some aspects, a codon optimized nucleic acid sequence encoding a CUG-targeting Cas13d protein such as those put forth in SEQ ID NOs: 518, 528, 534, 536, and 539 exhibits increased stability. In some aspects, a codon optimized nucleic acid sequence encoding a CUG-targeting Cas13d protein exhibits increased stability through increased resistance to hydrolysis. In some embodiments, the codon optimized sequence encoding a CUG-targeting Cas13d protein exhibits at least 5%, at least 10%, at least 20%, at least 30%, at least 50%, at least 75%, at least 100%, at least 200%, at least 300%, at least 500%, or at least 1000% increased stability relative to a wild-type or non-codon optimized nucleic acid sequence. In some embodiments, the codon optimized sequence encoding a CUG-targeting Cas13d protein exhibits at least 5%, at least 10%, at least 20%, at least 30%, at least 50%, at least 75%, at least 100%, at least 200%, at least 300%, at least 500%, or at least 1000% increased resistance to hydrolysis in a human subject relative to a wild-type or non-codon optimized nucleic acid sequence.

In some aspects, a codon optimized nucleic acid sequence encoding a CUG-targeting Cas13d protein such as those put forth in SEQ ID NOs: 518, 528, 534, 536, and 539, can comprise no donor splice sites. In some aspects, a codon optimized nucleic acid sequence encoding a CUG-targeting Cas13d protein can comprise no more than about one, or about two, or about three, or about four, or about five, or about six, or about seven, or about eight, or about nine, or about ten donor splice sites. In some aspects, a codon optimized nucleic acid sequence encoding a CUG-targeting Cas13d protein comprises at least one, or at least two, or at least three, or at least four, or at least five, or at least six, or at least seven, or at least eight, or at least nine, or at least ten fewer donor splice sites as compared to a non-codon optimized nucleic acid sequence encoding the CUG-targeting Cas13d protein.

Without wishing to be bound by theory, the removal of donor splice sites in the codon optimized nucleic acid sequence can unexpectedly and unpredictably increase expression of the CUG-targeting Cas13d protein in vivo, as cryptic splicing is prevented. Moreover, cryptic splicing may vary between different subjects, meaning that the expression level of the CUG-targeting Cas13d protein comprising donor splice sites may unpredictably vary between different subjects. Such unpredictability is unacceptable in the context of human therapy. Accordingly, the codon optimized nucleic acid sequences put forth in SEQ ID NOs: 518, 528, 534, 536, and 539, which lacks donor splice sites, unexpectedly and surprisingly allows for increased expression of the CUG-targeting Cas13d protein in human subjects and regularizes expression of the CUG-targeting Cas13d protein across different human subjects.

In some aspects, a codon optimized nucleic acid sequence encoding a CUG-targeting Cas13d protein, such as those put forth in SEQ ID NOs: 518, 528, 534, 536, and 539, can have a GC content that differs from the GC content of the non-codon optimized nucleic acid sequence encoding the CUG-targeting Cas13d protein. In some aspects, the GC content of a codon optimized nucleic acid sequence encoding a CUG-targeting Cas13d protein is more evenly distributed across the entire nucleic acid sequence, as compared to the non-codon optimized nucleic acid sequence encoding the CUG-targeting Cas13d protein.

Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects, a codon optimized nucleic acid sequence encoding a CUG-targeting Cas13d protein, such as those put forth in SEQ ID NOs: 518, 528, 534, 536, and 539, can have fewer repressive microRNA target binding sites as compared to the non-codon optimized nucleic acid sequence encoding the CUG-targeting Cas13d protein. In some aspects, a codon optimized nucleic acid sequence encoding a CUG-targeting Cas13d protein can have at least one, or at least two, or at least three, or at least four, or at least five, or at least six, or at least seven, or at least eight, or at least nine, or at least ten, or at least ten fewer repressive microRNA target binding sites as compared to the non-codon optimized nucleic acid sequence the CUG-targeting Cas13d protein.

Without wishing to be bound by theory, by having fewer repressive microRNA target binding sites, the codon optimized nucleic acid sequence encoding a CUG-targeting Cas13d protein unexpectedly exhibits increased expression in a human subject.

### Fusion Proteins

In some embodiments of the compositions and methods of the disclosure, the composition comprises a sequence encoding a target RNA-binding fusion protein comprising (a) a sequence encoding a first RNA-binding polypeptide or portion thereof; and optionally (b) a sequence encoding a second RNA-binding polypeptide, wherein the first RNA-binding polypeptide binds a target RNA, and wherein the second RNA-binding polypeptide comprises RNA-nuclease activity.

In some embodiments, a target RNA-binding fusion protein is an RNA-guided target RNA-binding fusion protein. RNA-guided target RNA-binding fusion proteins comprise at least one RNA-binding polypeptide which corresponds to a gRNA which guides the RNA-binding polypeptide to target RNA. RNA-guided target RNA-binding fusion proteins include without limitation, RNA-binding polypeptides which are CRISPR/Cas-based RNA-binding polypeptides or portions thereof.

Signal Sequences

In some embodiments, a target RNA-binding fusion protein of the disclosure comprises a signal sequence. In some embodiments, a target RNA-binding fusion protein comprises one or more signal sequences. In some embodiments, the signal sequence is a nuclear localization sequence (NLS), a nuclear export signal (NES), or a combination thereof In some embodiments, the signal sequence comprises one or more nuclear localization sequences (NLSs). In some embodiments, one or more NLS sequence comprises a sequence listed in Table 8. In some embodiments, the NLS signal sequence is a SV40 NLS signal sequence. In some embodiments, the SV40 NLS signal sequence is PKKKRKV (SEQ ID NO: 437).

TABLE 8
Nuclear Localization Sequences of the disclosure

| Name | Amino acid Sequence | SEQ ID NO: |
|---|---|---|
| SV40-NLS | PKKKRKV | 437 |
| human H2B-NLS | GKKRKRSRK | 438 |
| yeast H2B-NLS | GKKRSKV | 439 |
| human p53-NLS | KRALPNNTSSSPQPKKKP | 440 |
| human-cmyc-NLS | PAAKRVKLD | 441 |
| human pRB-NLS | KRSAEGSNPPKPLKKLR | 442 |
| human Nucleoplasmin-NLS | KRPAATKKAGQAKKKKLDK | 443 |
| Human pRB-NLS (extended version) | DRVLKRSAEGSNPPKPLKKLR | 543 |

In some embodiments, the signal sequence comprises one or more NES sequences. In some embodiments, the one or more NES sequence comprises a sequence listed in Table 9.

TABLE 9
Nuclear Export Sequences of the disclosure

| | | SEQ ID |
|---|---|---|
| Name | Amino acid Sequence | NO: |
| HIV REV NES | LPPLERLTLD | 544 |
| Human PKI NES | LALKLAGLDI | 545 |

In some embodiments, a target RNA-binding fusion protein of the disclosure comprises a tag sequence. In some embodiments, the tag sequence is a FLAG tag. In some embodiments, the FLAG tag sequence is DYKDDDDK (SEQ ID NO: 436).

### Linker Sequences

In some embodiments, a target RNA-binding fusion protein comprises a linker sequence. In some embodiments, the linker sequence may comprise or consist of 1, 2, 3, 4, 5, 6, 7, 8, 9, 10, 15, 20, 25, 30, 35, 40, 45, 50 or any number of amino acids in between. In some embodiments, the linker sequence comprises a linker sequence listed in Table 10.

Table 10
Linker Sequences of the disclosure

| Linker Sequence (amino acid) | SEQ ID NO: |
|---|---|
| GGS | 410 |
| VDTANGS | 411 |
| VDTGNGS | 412 |
| SGSETPGTSESATPES | 413 |
| GGGGSGGGGS | 414 |
| GGGGSGGGGSGGGGS | 415 |
| GGGGSGGGGSGGGGSGG | 416 |
| GGS | |
| EAAAKEAAAK | 417 |
| EAAAKEAAAKEAAAK | 418 |
| EAAAKEAAAKEAAAKEAA | 419 |
| AK | |
| APAPAPAP | 420 |
| APAPAPAPAPAP | 421 |
| APAPAPAPAPAPAPAPAP | 422 |
| GGGGSEAAAK | 423 |
| EAAAKGGGGS | 424 |
| GGGGSGGGGSEAAAKEAA | 425 |
| AK | |
| EAAAKEAAAKGGGGSGGG | 426 |
| GS | |
| RQTSPDPCPQLPLVPR | 427 |
| VDTGNWF | 428 |
| VDTANGSVDTGNGS | 429 |
| ARNVEERLCL | 430 |
| AIELNPSNA | 431 |
| ICGSRNL | 432 |
| VLATDMSKH | 434 |
| FLRELPEP | 435 |
| LIPKDQYYC | 436 |
| AEAAAKEAAAKA | 620 |
| AEAAAKEAAAKEAAAKA | 621 |
| AEAAAKEAAAKEAAAKEAAAKA | 622 |
| YVEFEGEQGVDEGGVSGGGS | 623 |
| GSRNLDFQALEETTEYDGGY | 624 |
| ASSTSPVEISEWLDQKLTKSDRPEL | 625 |
| VNQCRRQSEDSTFYLG | 626 |
| AVSPLLLTTTNSSEGLSMGNY | 627 |
| LDEAYPGKKLLPDDPYEKACQ | 628 |
| SAAAATPAVRTVPQYKYAAGVRN | 629 |
| PQQHLNAQPQVTMQQPAVHVQ | |
| GQEPL | |
| GGGGSEAAAKGGGGS | 630 |
| GGGGSEAAAKGGGGSEAAAKGG | 631 |
| GGS | |
| GGGGSSGSETGGTSESATGESGG | 632 |
| GGS | |
| SGSETPGTSESATPES | 633 |

### Promoter Sequences

In aspects, CUG targeting compositions of the disclosure comprise a promoter sequence. In some embodiments, any promoter disclosed herein can be substituted for any of the other promoters recited in the RNA-targeting constructs disclosed herein. In some aspects, CUG targeting compositions comprise a truncated CAG (tCAG) promoter (SEQ ID NO: 385). In some aspects, CUG targeting compositions comprise a short EF1-alpha (EFS) promoter as set forth in SEQ ID NO: 520. In some aspects, CUG targeting compositions comprise a human U6 promoter as set forth in SEQ ID NO: 519. In some aspects, CUG targeting compositions comprise an EFS-UBB promoter set forth in SEQ ID NO: 609. In some aspects, CUG targeting compositions comprise a muscle specific promoter. In some aspects, the CUG-targeting compositions comprise a muscle specific promoter which is a desmin promoter set forth in SEQ ID NO: 568 (full-length), SEQ ID NO: 608 (full-length) or SEQ ID NO: 569 (truncated). In some aspects, CAG targeting compositions comprise a synapsin promoter set forth in SEQ ID NO:619. In some embodiments, promoter sequences of the disclosure comprise a human EF1-alpha core promoter (SEQ ID NO: 642). In some embodiments, promoter sequences of the disclosure comprise a modified UBB intron (SEQ ID NO: 643). In some embodiments, promoter sequences of the disclosure comprise a modified CMV enhancer sequence (SEQ ID NO: 644). In some embodiments, promoter sequences of the disclosure comprise an eCMV-EFS-UBB promoter sequence (SEQ ID NO: 645).

. In some embodiments, expression control by a promoter is constitutive or ubiquitous. Non-limiting exemplary promoters include a Pol III promoter such as, e.g., U6 and H1 promoters and/or a Pol II promoter e.g., SV40, CMV (optionally including the CMV enhancer), RSV (Rous Sarcoma Virus LTR promoter (optionally including RSV enhancer), CBA (hybrid CMV enhancer/chicken ß-actin), CAG (hybrid CMV enhancer fused to chicken ß-actin), truncated CAG, Cbh (hybrid CBA), EF-1a (human elongation factor alpha-1) or EFS (short intron-less EF-1 alpha), PGK (phosphoglycerol kinase), CEF (chicken embryo fibroblasts), UBC (ubiquitin C), GUSB (lysosomal enzyme beta-glucuronidase), UCOE (ubiquitous chromatin opening element), hAAT (alpha-1 antitrypsin), TBG (thyroxine binding globulin), Desmin (full-length or truncated), MCK (muscle creatine kinase), C5-12 (synthetic muscle promoter), CK8e (creatin kinase 8), NSE (neuron-specific enolase), Synapsin, Synapsin-1 (SYN-1), opsin, PDGF (platelet-derived growth factor), PDGF-A, MecP2 (methyl CpG-binding protein 2), CaMKII (Calcium/Calmodulin-dependent protein kinase II), mGluR2 (metabotropic glutamate receptor 2), NFL (neurofilament light), NFH (neurofilament heavy), nβ2, PPE (rat preproenkephalin), ENK (preproenkephalin), Preproenkephalin-neurofilament chimeric promoter, EAAT2 (glutamate transporter), GFAP (glial fibrillary acidic protein), MBP (myelin basic protein), human rhodopsin kinase promoter (hGRK1), ß-actin promoter, dihydrofolate reductase promoter, MHCK7 (hybrid promoter of enhancer/promoter regions of muscle creatine kinase and alpha myosin heavy-chain genes) and combinations thereof. An “enhancer” is a region of DNA that can be bound by activating proteins to increase the likelihood or frequency of transcription. Non-limiting exemplary enhancers and posttranscriptional regulatory elements include the CMV enhancer, MCK enhancer, R-U5′ segment in LTR of HTLV-1, SV40 enhancer, the intron sequence between exons 2 and 3 of rabbit ß-globin, and WPRE. In some embodiments an intron is used to enhance promoter activity such as a UBB intron. In some embodiments, the UBB intron is used with an EFS promoter. In some embodiments, enhancer sequences can be added in the 5′ or 3′ UTR. In some embodiments, a 5′ enhancer can be Hsp70 as set forth in SEQ ID NO: 652:

TAACGGCTAGCCTGAGGAGCTGCTGCGACAGTCCACTACCTTTTTCGAG
AGTGACTCCCGTTGTCCCAAGGCTTCCCAGAGCGAACCTGTGCGGCTGC
AGGCACCGGCGCGTCGAGTTTCCGGCGTCCGGAAGGACCGAGCTCTTCT
CGCGGATCCAGTGTTCCGTTTCCAGCCCCCAATCTCAGAGCGGAGCCGA
CAGAGAGCAGGGAACCGGC.

### Non-Guided RNA-Binding Fusion Proteins

In some embodiments, a target RNA-binding fusion protein is not an RNA-guided target RNA-binding fusion protein and as such comprises at least one RNA-binding polypeptide which is capable of binding a target RNA without a corresponding gRNA sequence. Such non-guided RNA-binding polypeptides include, without limitation, at least one RNA-binding protein or RNA-binding portion thereof which is a PUF (Pumilio and FBF homology family) protein. This type RNA-binding polypeptide can be used instead of a gRNA-guided RNA binding protein such as CRISPR/Cas. The unique RNA recognition mode of PUF proteins (named for Drosophila Pumilio and C. elegans fem-3 binding factor) that are involved in mediating mRNA stability and translation are well known in the art. The PUF domain of human Pumilio1, also known in the art, binds tightly to cognate RNA sequences and its specificity can be modified. It contains eight PUF modules that recognize eight consecutive RNA bases with each module recognizing a single base. Since two amino acid side chains in each module recognize the Watson-Crick edge of the corresponding base and determine the specificity of that module, a PUF protein can be designed to specifically bind most 8 to 16-nt RNA. Wang et al., Nat Methods. 2009; 6(11): 825-830. See also WO2012/068627 which is incorporated by reference herein in its entirety.

The modular nature of the PUF-RNA interaction has been used to rationally engineer the binding specificity of PUF domains (Cheong, C. G. & Hall, T. M. (2006) PNAS 103: 13635-13639; Wang, X. et al (2002) Cell 110: 501-512). However, only the successful design of PUF domains with modules that recognize adenine, guanine or uracil have been reported prior to the teachings of WO2012/06827 supra. While the wild-type PumHD does not bind cytosine (C), molecular engineering has shown that some of the Pum units can be mutated to bind C with good yield and specificity. See e.g., Dong, S. et al. Specific and modular binding code for cytosine recognition in Pumilio/FBF (PUF) RNA-binding domains, The Journal of biological chemistry 286, 26732-26742 (2011). Accordingly, PumHD is a modified version of the WT Pumilio protein that exhibits programmable binding to arbitrary 8-base sequences of RNA. Each of the eight units of PumHD can bind to all four RNA bases, and the RNA bases flanking the target sequence do not affect binding. See also the following for art-recognized RNA-binding rules of PUF design: Filipovska A, Razif M F, Nygdrd K K, & Rackham O. A universal code for RNA recognition by PUF proteins. Nature chemical biology, 7(7), 425-427 (2011); Filipovska A, & Rackham O. Modular recognition of nucleic acids by PUF, TALE and PPR proteins. Molecular Bio Systems, 8(3), 699-708 (2012); Abil Z, Denard CA, & Zhao H. Modular assembly of designer PUF proteins for specific post-transcriptional regulation of endogenous RNA. Journal of biological engineering, 8(1), 7 (2014); Zhao Y, Mao M, Zhang W, Wang J, Li H, Yang Y, Wang Z, & Wu J. Expanding RNA binding specificity and affinity of engineered PUF domains. Nucleic Acids Research, 46(9), 4771-4782 (2018); Shinoda K, Tsuji S, Futaki S, & Irnanishi M. Nested PUF Proteins: Extending Target RNA Elements for Gene Regulation. ChemBioChem, 19(2), 171176 (2018); Koh Y Y, Wang Y, Qiu C, Opperman L, Gross L, Tanaka Hall™, & Wickens M. Stacking Interactions in PUF-RNA Complexes. RNA, 17(4), 718-727 (2011).

As such, it is well known in the art that human PUM1 (1186 amino acids) contains an RNA-binding domain (RBD) in the C-terminus of the protein (also known as Pumilio homology domain PUM-HD amino acid 828-amino acid 1175) and that PUFs are based on the RBD of human PUM1. There are 8 structural modules of 36 amino acids (except module 7 which has 43 amino acids) for RNA binding and flanking N- and C-terminal regions important for protein structure and stability. Within each module, amino acids 12, 13, and 16 are important for RNA binding with 12 and 16 responsible for RNA base recognition. Amino acid 13 stacks with RNA bases and can be modified to tune specificity and affinity. Alternatively, the PUF design may maintain amino acid 13 as human PUM1's native residue. In some embodiments of the PUF(CUG) or PUMBY(CUG) compositions disclosed herein, amino acid 13 (for stacking) will be engineered with an H and in other embodiments, will be engineered with a Y. In some embodiments, stacking residues may be modified to improve binding and specificity. Recognition occurs in reverse orientation as N- to C-terminal PUF recognizes 3′ to 5′ RNA. Accordingly, PUF engineering of 8 modules (8PUF), as known in the art, mimics a human protein. An exemplary 8-mer RNA recognition (8PUF) would be designed as follows: R1′-R1-R2-R3-R4-R5-R6-R7-R8-R8′. In one embodiment, an 8PUF is used as the RBD. In another embodiment, a variation of the 8PUF design is used to create a 14-mer RNA recognition (14PUF) RBD, 15-mer RNA recognition (15PUF) RBD, or a 16-mer RNA recognition (16PUF) RBD. In another embodiment, the PUF can be engineered to comprise a 4-mer, 5-mer, 6-mer, 7-mer, 8-mer, 9-mer, 10-mer, 11-mer, 12-mer, 13-mer, 14-mer, 15-mer, 16-mer, 24-mer, 30-mer, 36-mer, or any number of modules between. Shinoda et al., 2018; Criscuolo et al., 2020. Repeats 1-8 of wild type human PUM1 are provided herewith at SEQ ID NOS: 462-469, respectively. The nucleic acid sequence encoding the PUF domain from human PUM1 is SEQ ID NO: 470 and the amino acid sequence of the PUF domain from human PUM1 amino acids 828-1176 is SEQ ID NO: 471. See also U.S. Pat. No. 9,580,714 which is incorporated herein in its entirety.

In some embodiments of the non-guided RNA-binding fusion proteins of the disclosure, the fusion protein comprises at least one RNA-binding protein or RNA-binding portion thereof which is a PUMBY (Pumilio-based assembly) protein. RNA-binding protein PumHD, which has been widely used in native and modified form for targeting RNA, has been engineered into a protein architecture designed to yield a set of four canonical protein modules, each of which targets one RNA base. These modules (i.e., Pumby, for Pumilio-based assembly) are concatenated in chains of varying composition and length, to bind desired target RNAs. In essence, PUMBY is a more simple and modular form of PumHD, in which a single protein unit of PumHD is concatenated into arrays of arbitrary size and binding sequence specificity. The specificity of such Pumby-RNA interactions is high, with undetectable binding of a Pumby chain to RNA sequences that bear three or more mismatches from the target sequence. Katarzyna et al., PNAS, 2016; 113(19): E2579-E2588. See also US 2016/0238593 which is incorporated by reference herein in its entirety.

In some embodiments of the compositions of the disclosure, the first RNA binding protein comprises a Pumilio and FBF (PUF) protein. In some embodiments, the first RNA binding protein comprises a Pumilio-based assembly (PUMBY) protein. In some embodiments, the PUF or PUMBY RNA-binding proteins are fused with a nuclease domain such as E17 (SEQ ID NO: 358). In another embodiment, the single vector comprises a dCas13d RNA-binding system fused with a nuclease domain from ZC3H12A, such as E17 (SEQ ID NO: 359).

In some embodiments of the compositions of the disclosure, at least one of the RNA-binding proteins or RNA-binding portions thereof is a PPR protein. PPR proteins (proteins with pentatricopeptide repeat (PPR) motifs derived from plants) are nuclear-encoded and exclusively controlled at the RNA level organelles (chloroplasts and mitochondria), cutting, translation, splicing, RNA editing, genes specifically acting on RNA stability. PPR proteins are typically a motif of 35 amino acids and have a structure in which a PPR motif is about 10 contiguous amino acids. The combination of PPR motifs can be used for sequence-selective binding to RNA. PPR proteins are often comprised of PPR motifs of about 10 repeat domains. PPR domains or RNA-binding domains may be configured to be catalytically inactive. WO 2013/058404 incorporated herein by reference in its entirety.

In some embodiments, the fusion protein disclosed herein comprises a linker between the at least two RNA-binding polypeptides. In some embodiments, the linker is a peptide linker. In one embodiment, the linker is VDTANGS (SEQ ID NO: 411). In some embodiments, the peptide linker comprises one or more repeats of the tri-peptide GGS. In other embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the at least one RNA-binding protein does not require multimerization for RNA-binding activity. In some embodiments, the at least one RNA-binding protein is not a monomer of a multimer complex. In some embodiments, a multimer protein complex does not comprise the RNA binding protein. In some embodiments, the at least one of RNA-binding protein selectively binds to a target sequence within the RNA molecule. In some embodiments, the at least one RNA-binding protein does not comprise an affinity for a second sequence within the RNA molecule. In some embodiments, the at least one RNA-binding protein does not comprise a high affinity for or selectively bind a second sequence within the RNA molecule. In some embodiments, the at least one RNA-binding protein comprises between 2 and 1300 amino acids, inclusive of the endpoints.

In some embodiments, the at least one RNA-binding protein of the fusion proteins disclosed herein further comprises a sequence encoding a nuclear localization signal (NLS). In some embodiments, a nuclear localization signal (NLS) is positioned at the N-terminus of the RNA binding protein. In some embodiments, the at least one RNA-binding protein comprises an NLS at a C-terminus of the protein. In some embodiments, the at least one RNA-binding protein further comprises a first sequence encoding a first NLS and a second sequence encoding a second NLS. In some embodiments, the first NLS or the second NLS is positioned at the N-terminus of the RNA-binding protein. In some embodiments, the at least one RNA-binding protein comprises the first NLS or the second NLS at a C-terminus of the protein. In some embodiments, the at least one RNA-binding protein further comprises an NES (nuclear export signal) or other peptide tag or secretory signal. In one embodiment, the tag is a FLAG tag.

In some embodiments, a fusion protein disclosed herein comprises the at least one RNA-binding protein as a first RNA-binding protein together with a second RNA-binding protein comprising or consisting of a nuclease domain.

In some embodiments, the second RNA-binding polypeptide is operably configured to the first RNA-binding polypeptide at the C-terminus of the first RNA-binding polypeptide. In some embodiments, the second RNA-binding polypeptide is operably configured to the first RNA-binding polypeptide at the N-terminus of the first RNA-binding polypeptide. In one embodiment, an exemplary fusion protein is a PUF or PUMBY-based first RNA-binding protein fused to a second RNA-binding protein which is a zinc-finger endonuclease known as ZC3H12A or truncation of it is shown in SEQ ID NO: 358 (also termed E17).

An exemplary 14-mer RNA recognition (14PUMBY) targeting

(SEQ ID NO: 454)
UGCUGCUGCUGCUG

comprises the amino acid sequence:

(SEQ ID NO: 547)
GRSRLLEDFRNNRYPNLQLREIAGHTEQLVQDQYGSYVIEHVLEHGRPE
DKSKIVAEIRGHTEQLVQDQYGNYVIQHVLEHGRPEDKSKIVAEIRGHT
EQLVQDQYGSYVIRHVLEHGRPEDKSKIVAEIRGHTEQLVQDQYGSYVI
EHVLEHGRPEDKSKIVAEIRGHTEQLVQDQYGNYVIQHVLEHGRPEDKS
KIVAEIRGHTEQLVQDQYGSYVIRHVLEHGRPEDKSKIVAEIRGHTEQL
VQDQYGSYVIEHVLEHGRPEDKSKIVAEIRGHTEQLVQDQYGNYVIQHV
LEHGRPEDKSKIVAEIRGHTEQLVQDQYGSYVIRHVLEHGRPEDKSKIV
AEIRGHTEQLVQDQYGSYVIEHVLEHGRPEDKSKIVAEIRGHTEQLVQD
QYGNYVIQHVLEHGRPEDKSKIVAEIRGHTEQLVQDQYGSYVIRHVLEH
GRPEDKSKIVAEIRGHTEQLVQDQYGSYVIEHVLEHGRPEDKSKIVAEI
RGHTEQLVQDQYGNYVIQHVLEHGRPEDKSKIVAEIRGHIATLRKYTYG
KHILAKLEKYYMKNGVDLG.

In some aspects, SEQ ID NO: 547 comprises an architecture proceeding from the N-terminus to the C-terminus according to: R1′-R1-R2-R3-R4-R5-R6-R7-R8-R8′. In some aspects, SEQ ID NO: 547 is comprised of the sequences detailed in Table 11.

TABLE 11
8PUF protein according to SEQ ID NO: 547

| PUF | RNA | | SEQ ID |
|---|---|---|---|
| Module | Recognition | Amino Acid Sequence | NO |
| PUF R1′ | — | GRSRLLEDFRNNRYPNLQLREIAG | 495 |
| PUF R1 | G | HIMEFSQDQHGSRFIELKLERATPAERQLVFNEILQ | 498 |
| PUF R2 | U | AAYQLMVDVFGNYVIQKFFEFGSLEQKLALAERIRG | 490 |
| PUF R3 | C | HVLSLALQMYGSYVIRKALEFIPSDQQNEMVRELDG | 508 |
| PUF R4 | G | HVLKCVKDQNGSYVVEKCIECVQPQSLQFIIDAFKG | 504 |
| PUF R5 | U | QVFALSTHPYGNRVIQRILEHCLPDQTLPILEELHQ | 512 |
| PUF R6 | C | HTEQLVQDQYGSYVIRHVLEHGRPEDKSKIVAEIRG | 502 |
| PUF R7 | G | NVLVLSQHKFASNVVEKCVTHASRTERAVLIDEVCTMN | 510 |
| | | DGPHS | |
| PUF R8 | U | ALYTMMKDQYANYVVQKMIDVAEPGQRKIVMHKIRP | 493 |
| PUF R8′ | — | HIATLRKYTYGKHILAKLEKYYMKNGVDLG | 496 |

An exemplary 14-mer RNA recognition (14PUMBY) targeting

(SEQ ID NO: 454)
UGCUGCUGCUGCUG

comprises the amino acid sequence:

(SEQ ID NO: 547)
GRSRLLEDFRNNRYPNLQLREIAGHTEQLVQDQYGSYVIEHVLEHGRPE
DKSKIVAEIRGHTEQLVQDQYGNYVIQHVLEHGRPEDKSKIVAEIRGHT
EQLVQDQYGSYVIRHVLEHGRPEDKSKIVAEIRGHTEQLVQDQYGSYVI
EHVLEHGRPEDKSKIVAEIRGHTEQLVQDQYGNYVIQHVLEHGRPEDKS
KIVAEIRGHTEQLVQDQYGSYVIRHVLEHGRPEDKSKIVAEIRGHTEQL
VQDQYGSYVIEHVLEHGRPEDKSKIVAEIRGHTEQLVQDQYGNYVIQHV
LEHGRPEDKSKIVAEIRGHTEQLVQDQYGSYVIRHVLEHGRPEDKSKIV
AEIRGHTEQLVQDQYGSYVIEHVLEHGRPEDKSKIVAEIRGHTEQLVQD
QYGNYVIQHVLEHGRPEDKSKIVAEIRGHTEQLVQDQYGSYVIRHVLEH
GRPEDKSKIVAEIRGHTEQLVQDQYGSYVIEHVLEHGRPEDKSKIVAEI
RGHTEQLVQDQYGNYVIQHVLEHGRPEDKSKIVAEIRGHIATLRKYTYG
KHILAKLEKYYMKNGVDLG.

In some aspects, SEQ ID NO: 547 comprises an architecture proceeding from the N-terminus to the C-terminus according to: R1′-R6-R6-R6-R6-R6-R6-R6-R6-R6-R6-R6-R6-R6-R6-R8′. In some aspects, SEQ ID NO: 547 is comprised of the sequences detailed in Table 12.

TABLE 12
14Pumby protein according to SEQ ID NO: 547

| PUF | RNA | | SEQ ID |
|---|---|---|---|
| Module | Recognition | Amino Acid Sequence | NO: |
| PUF R1′ | — | GRSRLLEDFRNNRYPNLQLREIAG | 495 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| PUF R8′ | — | HIATLRKYTYGKHILAKLEKYYMKNGVDLG | 496 |

An exemplary 14-mer RNA recognition (14PUMBY) targeting

(SEQ ID NO: 473)
CUGCUGCUGCUGCU

comprises the amino acid sequence:

(SEQ ID NO: 548)
GRSRLLEDFRNNRYPNLQLREIAGHTEQLVQDQYGNYVIQHVLEHGRPE
DKSKIVAEIRGHTEQLVQDQYGSYVIRHVLEHGRPEDKSKIVAEIRGHT
EQLVQDQYGSYVIEHVLEHGRPEDKSKIVAEIRGHTEQLVQDQYGNYVI
QHVLEHGRPEDKSKIVAEIRGHTEQLVQDQYGSYVIRHVLEHGRPEDKS
KIVAEIRGHTEQLVQDQYGSYVIEHVLEHGRPEDKSKIVAEIRGHTEQL
VQDQYGNYVIQHVLEHGRPEDKSKIVAEIRGHTEQLVQDQYGSYVIRHV
LEHGRPEDKSKIVAEIRGHTEQLVQDQYGSYVIEHVLEHGRPEDKSKIV
AEIRGHTEQLVQDQYGNYVIQHVLEHGRPEDKSKIVAEIRGHTEQLVQD
QYGSYVIRHVLEHGRPEDKSKIVAEIRGHTEQLVQDQYGSYVIEHVLEH
GRPEDKSKIVAEIRGHTEQLVQDQYGNYVIQHVLEHGRPEDKSKIVAEI
RGHTEQLVQDQYGSYVIRHVLEHGRPEDKSKIVAEIRGHIATLRKYTYG
KHILAKLEKYYMKNGVDLG.

In some aspects, SEQ ID NO: 548 comprises an architecture proceeding from the N-terminus to the C-terminus according to: R1′-R6-R6-R6-R6-R6-R6-R6-R6-R6-R6-R6-R6-R6-R6-R8′. In some aspects, SEQ ID NO: 548 is comprised of the sequences detailed in Table 13.

TABLE 13
14Pumby protein according to SEQ ID NO: 548

| PUF | RNA | | SEQ ID |
|---|---|---|---|
| Module | Recognition | Amino Acid Sequence | NO: |
| PUF R1′ | | GRSRLLEDFRNNRYPNLQLREIAG | 495 |
| R6 | U | HTEQLVQDQYGNYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| R6 | C | HTEQLVQDQYGSYVIRHVLEHGRPEDKSKIVAEIRG | 502 |
| PUF R8′ | | HIATLRKYTYGKHILAKLEKYYMKNGVDLG | 496 |

An exemplary 14-mer RNA recognition (14PUMBY) targeting

(SEQ ID NO: 477)
GCUGCUGCUGCUGC

comprises the amino acid sequence:

(SEQ ID NO: 558)
GRSRLLEDFRNNRYPNLQLREIAGHTEQLVQDQYGSYVIRHVLEHGRPE
DKSKIVAEIRGHTEQLVQDQYGSYVIEHVLEHGRPEDKSKIVAEIRGHT
EQLVQDQYGNYVIQHVLEHGRPEDKSKIVAEIRGHTEQLVQDQYGSYVI
RHVLEHGRPEDKSKIVAEIRGHTEQLVQDQYGSYVIEHVLEHGRPEDKS
KIVAEIRGHTEQLVQDQYGNYVIQHVLEHGRPEDKSKIVAEIRGHTEQL
VQDQYGSYVIRHVLEHGRPEDKSKIVAEIRGHTEQLVQDQYGSYVIEHV
LEHGRPEDKSKIVAEIRGHTEQLVQDQYGNYVIQHVLEHGRPEDKSKIV
AEIRGHTEQLVQDQYGSYVIRHVLEHGRPEDKSKIVAEIRGHTEQLVQD
QYGSYVIEHVLEHGRPEDKSKIVAEIRGHTEQLVQDQYGNYVIQHVLEH
GRPEDKSKIVAEIRGHTEQLVQDQYGSYVIRHVLEHGRPEDKSKIVAEI
RGHTEQLVQDQYGSYVIEHVLEHGRPEDKSKIVAEIRGHIATLRKYTYG
KHILAKLEKYYMKNGVDLG.

In some aspects, SEQ ID NO: 558 comprises an architecture proceeding from the N-terminus to the C-terminus according to: R1′-R6-R6-R6-R6-R6-R6-R6-R6-R6-R6-R6-R6-R6-R6-R8′. In some aspects, SEQ ID NO: 558 is comprised of the sequences detailed in Table 14.

TABLE 14
14Pumby protein according to SEQ ID NO: 558

| PUF | RNA | | SEQ ID |
|---|---|---|---|
| Module | Recognition | Amino Acid Sequence | NO: |
| PUF R1′ | — | GRSRLLEDFRNNRYPNLQLREIAG | 495 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| PUF R8′ | | HIATLRKYTYGKHILAKLEKYYMKNGVDLG | 496 |

An exemplary 8-mer RNA recognition (8PUF) targeting

(SEQ ID NO: 453)
UGCUGCUG

comprises the amino acid sequence:

(SEQ ID NO: 444)
GRSRLLEDFRNNRYPNLQLREIAGHIMEFSQDQHGSRFIEL
KLERATPAERQLVFNEILQAAYQLMVDVFGNYVIQKFFEF
GSLEQKLALAERIRGHVLSLALOMYGSYVIRKALEFIPSD
QQNEMVRELDGHVLKCVKDQNGSYVVEKCIECVQPQSLQF
IIDAFKGQVFALSTHPYGNRVIQRILEHCLPDQTLPILEE
LHQHTEQLVQDQYGSYVIRHVLEHGRPEDKSKIVAEIRGN
VLVLSQHKFASNVVEKCVTHASRTERAVLIDEVCTMNDGP
HSALYTMMKDQYANYVVQKMIDVAEPGQRKIVMHKIRPHI
ATLRKYTYGKHILAKLEKYYMKNGVDLG.

In some aspects, SEQ ID NO: 444 comprises an architecture proceeding from the N-terminus to the C-terminus according to: R1′-R1-R2-R3-R4-R5-R6-R7-R8-R8′. In some aspects, SEQ ID NO: 444 is comprised of the sequences detailed in Table 15.

TABLE 15
8PUF protein according to SEQ ID NO: 444

| | | | SEQ |
|---|---|---|---|
| PUF | RNA | Amino Acid | ID |
| Module | Recognition | Sequence | NO: |
| PUF R1′ | — | GRSRLLEDFR | 495 |
| | | NNRYPNLQLR | |
| | | EIAG | |
| PUF R1 | G | HIMEFSQDQH | 498 |
| | | GSRFIELKLE | |
| | | RATPAERQLV | |
| | | FNEILQ | |
| PUF R2 | U | AAYQLMVDVF | 490 |
| | | GNYVIQKFFE | |
| | | FGSLEQKLAL | |
| | | AERIRG | |
| PUF R3 | C | HVLSLALQMY | 507 |
| | | GSYVIRKALE | |
| | | FIPSDQQNEM | |
| | | VRELDG | |
| PUF R4 | G | HVLKCVKDQN | 503 |
| | | GSYVVEKCIE | |
| | | CVQPQSLQFI | |
| | | IDAFKG | |
| PUF R5 | U | QVFALSTHPY | 511 |
| | | GNRVIQRIL | |
| | | EHCLPDQTLP | |
| | | ILEELHQ | |
| PUF R6 | C | HTEQLVQDQY | 501 |
| | | GSYVIRHVLE | |
| | | HGRPEDKSKI | |
| | | VAEIRG | |
| PUF R7 | G | NVLVLSQHKF | 509 |
| | | ASNVVEKCVT | |
| | | HASRTERAVL | |
| | | IDEVCTMNDG | |
| | | PHS | |
| PUF R8 | U | ALYTMMKDQY | 493 |
| | | ANYVVQKMID | |
| | | VAEPGQRKIV | |
| | | MHKIRP | |
| PUF R8′ | — | HIATLRKYTY | 496 |
| | | GKHILAKLEK | |
| | | YYMKNGVDLG | |

In some embodiments, nucleic acid sequences encoding PUF proteins of the disclosure are codon optimized nucleic acid sequences. In some embodiments, the codon optimized sequence encoding a PUF protein exhibits at least 5%, at least 10%, at least 20%, at least 30%, at least 50%, at least 75%, at least 100%, at least 200%, at least 300%, at least 500%, or at least 1000% increased expression in a human subject relative to a wild-type or non-codon optimized nucleic acid sequence. In some embodiments, an 8PUF protein of the disclosure is encoded by a nucleic acid sequences comprising SEQ ID NO: 452. In some embodiments, a nucleotide sequence encoding a fusion protein comprising a CUG targeting 8PUF and an E17 nuclease comprises SEQ ID NO: 460. In some embodiments, a nucleotide sequence encoding a CUG-targeting fusion protein comprises, from 5′ to 3′: a flag tag, SV-40 nuclear localization sequence, an 8PUF, and an E17 nuclease is set forth in SEQ ID NO: 515. In some embodiments, a nucleotide sequence encoding a CUG-targeting fusion protein comprises, from 5′ to 3′: a SV-40 nuclear localization sequence, an 8PUF, and an E17 nuclease is set forth in SEQ ID NO: 517. In some embodiments, a nucleotide sequence encoding a CUG-targeting fusion protein comprises, from 5′ to 3′: an 8PUF and an E17 nuclease is set forth in SEQ ID NO: 516.

In some embodiments, nucleic acid sequences encoding PUF proteins of the disclosure are codon optimized nucleic acid sequences. In some embodiments, the codon optimized sequence encoding a PUF protein exhibits at least 5%, at least 10%, at least 20%, at least 30%, at least 50%, at least 75%, at least 100%, at least 200%, at least 300%, at least 500%, or at least 1000% increased translation in a human subject relative to a wild-type or non-codon optimized nucleic acid sequence.

In some aspects, a codon optimized nucleic acid sequence encoding a PUF protein such as those put forth in SEQ ID NOs: 452 and 515-517exhibits increased stability. In some aspects, a codon optimized nucleic acid sequence encoding a PUF protein exhibits increased stability through increased resistance to hydrolysis. In some embodiments, the codon optimized sequence encoding a PUF protein exhibits at least 5%, at least 10%, at least 20%, at least 30%, at least 50%, at least 75%, at least 100%, at least 200%, at least 300%, at least 500%, or at least 1000% increased stability relative to a wild-type or non-codon optimized nucleic acid sequence. In some embodiments, the codon optimized sequence encoding a PUF protein exhibits at least 5%, at least 10%, at least 20%, at least 30%, at least 50%, at least 75%, at least 100%, at least 200%, at least 300%, at least 500%, or at least 1000% increased resistance to hydrolysis in a human subject relative to a wild-type or non-codon optimized nucleic acid sequence.

In some aspects, a codon optimized nucleic acid sequence encoding a PUF protein such as those put forth in SEQ ID NOs: 452 and 515-517, can comprise no donor splice sites. In some aspects, a codon optimized nucleic acid sequence encoding a PUF protein can comprise no more than about one, or about two, or about three, or about four, or about five, or about six, or about seven, or about eight, or about nine, or about ten donor splice sites. In some aspects, a codon optimized nucleic acid sequence encoding a PUF protein comprises at least one, or at least two, or at least three, or at least four, or at least five, or at least six, or at least seven, or at least eight, or at least nine, or at least ten fewer donor splice sites as compared to a non-codon optimized nucleic acid sequence encoding the PUF protein.

Without wishing to be bound by theory, the removal of donor splice sites in the codon optimized nucleic acid sequence can unexpectedly and unpredictably increase expression of the PUF protein in vivo, as cryptic splicing is prevented. Moreover, cryptic splicing may vary between different subjects, meaning that the expression level of the PUF protein comprising donor splice sites may unpredictably vary between different subjects. Such unpredictability is unacceptable in the context of human therapy. Accordingly, the codon optimized nucleic acid sequences put forth in SEQ ID NOs: 452 and 515-517, which lacks donor splice sites, unexpectedly and surprisingly allows for increased expression of the PUF protein in human subjects and regularizes expression of the PUF protein across different human subjects.

In some aspects, a codon optimized nucleic acid sequence encoding a PUF protein, such as those put forth in SEQ ID NOs: 452 and 515-517, can have a GC content that differs from the GC content of the non-codon optimized nucleic acid sequence encoding the PUF protein. In some aspects, the GC content of a codon optimized nucleic acid sequence encoding a PUF protein is more evenly distributed across the entire nucleic acid sequence, as compared to the non-codon optimized nucleic acid sequence encoding the PUF protein.

Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects, a codon optimized nucleic acid sequence encoding a PUF protein, such as those put forth in SEQ ID NOs: 452 and 515-517, can have fewer repressive microRNA target binding sites as compared to the non-codon optimized nucleic acid sequence encoding the PUF protein. In some aspects, a codon optimized nucleic acid sequence encoding a PUF protein can have at least one, or at least two, or at least three, or at least four, or at least five, or at least six, or at least seven, or at least eight, or at least nine, or at least ten, or at least ten fewer repressive microRNA target binding sites as compared to the non-codon optimized nucleic acid sequence the PUF protein.

Without wishing to be bound by theory, by having fewer repressive microRNA target binding sites, the codon optimized nucleic acid sequence encoding a PUF protein unexpectedly exhibits increased expression in a human subject.

In some embodiments, an 8PUF protein can be encoded by a nucleic acid sequence comprising:

(SEQ ID NO: 452)
GGACGAAGCCGACTCTTGGAAGACTTCAGAAACAATCGGT
ATCCGAACCTTCAGCTGAGAGAAATTGCTGGTCACATCAT
GGAATTTTCTCAAGATCAACATGGAAGCCGGTTTATTGAA
CTTAAACTCGAACGAGCCACCCCGGCCGAAAGGCAATTGG
TGTTCAATGAAATTCTTCAGGCCGCATACCAACTCATGGT
TGATGTTTTTGGGAACTATGTTATTCAAAAGTTTTTTGAG
TTCGGGTCACTGGAGCAAAAGTTGGCATTGGCAGAGCGAA
TCCGGGGCCATGTTCTGAGCCTCGCTCTCCAAATGTACGG
TAGTTATGTCATTCGCAAAGCACTCGAGTTCATACCATCA
GATCAACAGAATGAGATGGTGCGGGAGCTGGATGGGCATG
TTTTGAAATGCGTGAAAGACCAAAACGGTAGCTACGTAGT
TGAGAAATGCATCGAATGCGTCCAACCACAGTCTCTCCAA
TTTATTATAGATGCATTTAAGGGTCAGGTTTTCGCGCTTT
CTACGCACCCGTATGGGAACCGAGTGATTCAGAGAATCTT
GGAGCACTGCCTGCCGGATCAGACACTCCCTATCTTGGAG
GAATTGCACCAGCATACCGAACAATTGGTGCAAGATCAAT
ACGGTTCATATGTTATTCGGCACGTTCTTGAGCATGGAAG
GCCAGAGGACAAGTCAAAGATCGTCGCTGAGATTAGAGGT
AACGTATTGGTGCTCTCACAACACAAATTTGCATCTAATG
TGGTGGAGAAATGTGTTACTCATGCTTCTAGAACGGAAAG
GGCAGTTCTCATAGACGAAGTTTGCACAATGAATGATGGT
CCTCATAGCGCACTTTATACCATGATGAAGGACCAGTATG
CAAACTATGTCGTCCAGAAAATGATCGATGTGGCGGAGCC
CGGTCAACGGAAAATCGTGATGCACAAAATCCGACCTCAC
ATTGCTACACTCAGAAAATACACGTATGGAAAACATATTC
TGGCTAAGCTGGAGAAATATTACATGAAGAATGGAGTGGA
TCTGGGG.

An exemplary 14-mer RNA recognition (14PUF) targeting

(SEQ ID NO: 454)
UGCUGCUGCUGCUG

comprises the amino acid sequence:

(SEQ ID NO: 445)
GRSRLLEDFRNNRYPNLQLREIAGHIMEFSQDQHGSRFIE
LKLERATPAERQLVFNEILQAAYQLMVDVFGNYVIQKFFE
FGSLEQKLALAERIRGHVLSLALQMYGSYVIRKALEFIPS
DQQNEMVRELDGHVLKCVKDQNGSYVVEKCIECVQPQSLQ
FIIDAFKGQVFALSTHPYGNRVIQRILEHCLPDQTLPILE
ELHQHIMEFSQDQHGSRFIRLKLERATPAERQLVFNEILQ
AAYQLMVDVFGSYVIEKFFEFGSLEQKLALAERIRGHVLS
LALQMYGNRVIQKALEFIPSDQQNEMVRELDGHVLKCVKD
QNGSYVVRKCIECVQPQSLQFIIDAFKGQVFALSTHPYGS
RVIERILEHCLPDQTLPILEELHQHTEQLVQDQYGNYVIQ
HVLEHGRPEDKSKIVAEIRGHTEQLVQDQYGSYVIRHVL
EHGRPEDKSKIVAEIRGNVLVLSQHKFASNVVEKCVTHAS
RTERAVLIDEVCTMNDGPHSALYTMMKDQYANYVVQKMID
VAEPGQRKIVMHKIRPHIATLRKYTYGKHILAKLEKYYMK
NGVDLG

In some aspects, SEQ ID NO: 445 comprises an architecture proceeding from the N-terminus to the C-terminus according to: R1′-R1-R2-R3-R4-R5-R1-R2-R3-R4-R5-R6-R6-R7-R8-R8′. In some aspects, SEQ ID NO: 445 is comprised of the sequences detailed in Table 16.

TABLE 16
14PUF protein according to SEQ ID NO: 445

| | | | SEQ |
|---|---|---|---|
| PUF | RNA | Amino Acid | ID |
| Module | Recognition | Sequence | NO |
| PUF R1′ | — | GRSRLLEDFRNNRYP | 495 |
| | | NLQLREIAG | |
| PUF R1 | G | HIMEFSQDQHGSRFI | 498 |
| | | ELKLERATPAERQLV | |
| | | FNEILQ | |
| PUF R2 | U | AAYQLMVDVFGNYVI | 490 |
| | | QKFFEFGSLEQKLAL | |
| | | AERIRG | |
| PUF R3 | C | HVLSLALQMYGSYVI | 507 |
| | | RKALEFIPSDQQNEM | |
| | | VRELDG | |
| PUF R4 | G | HVLKCVKDQNGSYVV | 503 |
| | | EKCIECVQPQSLQFI | |
| | | IDAFKG | |
| PUF R5 | U | QVFALSTHPYGNRV | 511 |
| | | IQRILEHCLPDQTLP | |
| | | ILEELHQ | |
| PUF R1 | C | HIMEFSQDQHGSRFI | 498 |
| | | RLKLERATPAERQLV | |
| | | FNEILQ | |
| PUF R2 | G | AAYQLMVDVFGSYVI | 491 |
| | | EKFFEFGSLEQKLAL | |
| | | AERIRG | |
| PUF R3 | U | HVLSLALQMYGNRVI | 505 |
| | | QKALEFIPSDQQNEM | |
| | | VRELDG | |
| PUF R4 | C | HVLKCVKDQNGSYVV | 504 |
| | | RKCIECVQPQSLQFI | |
| | | IDAFKG | |
| PUF R5 | G | QVFALSTHPYGSRV | 512 |
| | | IERILEHCLPDQTLP | |
| | | ILEELHQ | |
| PUF R6 | U | HTEQLVQDQYGNYVI | 499 |
| | | QHVLEHGRPEDKSKI | |
| | | VAEIRG | |
| PUF R6 | C | HTEQLVQDQYGSYVI | 501 |
| | | RHVLEHGRPEDKSKI | |
| | | VAEIRG | |
| PUF R7 | G | NVLVLSQHKFASNVV | 509 |
| | | EKCVTHASRTERAVL | |
| | | IDEVCTMNDGPHS | |
| PUF R8 | U | ALYTMMKDQYANYVV | 493 |
| | | QKMIDVAEPGQRKIV | |
| | | MHKIRP | |
| PUF R8′ | — | HIATLRKYTYGKHIL | 496 |
| | | AKLEKYYMKNGVDLG | |

An exemplary 14-mer RNA recognition (14PUF) targeting

(SEQ ID NO: 454)
UGCUGCUGCUGCUG

comprises the amino acid sequence:

(SEQ ID NO: 446)
GRSRLLEDFRNNRYPNLQLREIAGHIMEFSQDQHGSRFIEL
KLERATPAERQLVFNEILQAAYQLMVDVFGNYVIQKFFEF
GSLEQKLALAERIRGHVLSLALQMYGSYVIRKALEFIPSD
QQNEMVRELDGHVLKCVKDQNGSYVVEKCIECVQPQSLQF
IIDAFKGQVFALSTHPYGNRVIQRILEHCLPDQTLPILEE
LHQHTEQLVQDQYGSYVIRHVLEHGRPEDKSKIVAEIRGH
IMEFSQDQHGSRFIELKLERATPAERQLVFNEILQAAYQL
MVDVFGNYVIQKFFEFGSLEQKLALAERIRGHVLSLALQM
YGSYVIRKALEFIPSDQQNEMVRELDGHVLKCVKDQNGSY
VVEKCIECVQPQSLQFIIDAFKGQVFALSTHPYGNRVIQR
ILEHCLPDQTLPILEELHQHTEQLVQDQYGSYVIRHVLEH
GRPEDKSKIVAEIRGNVLVLSQHKFASNVVEKCVTHASRT
ERAVLIDEVCTMNDGPHSALYTMMKDQYANYVVQKMIDVA
EPGQRKIVMHKIRPHIATLRKYTYGKHILAKLEKYYMKNG
VDLG.

In some aspects, SEQ ID NO: 446 comprises an architecture proceeding from the N-terminus to the C-terminus according to: R1′-R1-R2-R3-R4-R5-R6-R1-R2-R3-R4-R5-R6-R7-R8-R8′. In some aspects, SEQ ID NO: 446 is comprised of the sequences detailed in Table 17.

TABLE 17
14PUF protein according to SEQ ID NO: 446

| | | | SEQ |
|---|---|---|---|
| PUF | RNA | Amino Acid | ID |
| Module | Recognition | Sequence | NO |
| PUF R1′ | — | GRSRLLEDFRNNRYP | 495 |
| | | NLQLREIAG | |
| PUF R1 | G | HIMEFSQDQHGSRFI | 498 |
| | | ELKLERATPAERQLV | |
| | | FNEILQ | |
| PUF R2 | U | AAYQLMVDVFGNYVI | 490 |
| | | QKFFEFGSLEQKLAL | |
| | | AERIRG | |
| PUF R3 | C | HVLSLALQMYGSYVI | 507 |
| | | RKALEFIPSDQQNEM | |
| | | VRELDG | |
| PUF R4 | G | HVLKCVKDQNGSYVV | 503 |
| | | EKCIECVQPQSLQFI | |
| | | IDAFKG | |
| PUF R5 | U | QVFALSTHPYGNRVI | 511 |
| | | QRILEHCLPDQTLPI | |
| | | LEELHQ | |
| PUF R6 | C | HTEQLVQDQYGSYVI | 501 |
| | | RHVLEHGRPEDKSKI | |
| | | VAEIRG | |
| PUF R1 | G | HIMEFSQDQHGSRFI | 498 |
| | | ELKLERATPAERQLV | |
| | | FNEILQ | |
| PUF R2 | U | AAYQLMVDVFGNYVI | 490 |
| | | QKFFEFGSLEQKLAL | |
| | | AERIRG | |
| PUF R3 | C | HVLSLALQMYGSYVI | 507 |
| | | RKALEFIPSDQQNEM | |
| | | VRELDG | |
| PUF R4 | G | HVLKCVKDQNGSYVV | 503 |
| | | EKCIECVQPQSLQFI | |
| | | IDAFKG | |
| PUF R5 | U | QVFALSTHPYGNRVI | 511 |
| | | QRILEHCLPDQTLPI | |
| | | LEELHQ | |
| PUF R6 | C | HTEQLVQDQYGSYVI | 501 |
| | | RHVLEHGRPEDKSKI | |
| | | VAEIRG | |
| PUF R7 | G | NVLVLSQHKFASNVV | 509 |
| | | EKCVTHASRTERAVL | |
| | | IDEVCTMNDGPHS | |
| PUF R8 | U | ALYTMMKDQYANYVV | 493 |
| | | QKMIDVAEPGQRKIV | |
| | | MHKIRP | |
| PUF R8′ | — | HIATLRKYTYGKHIL | 496 |
| | | AKLEKYYMKNGVDLG | |

An exemplary 15-mer RNA recognition (15PUF) targeting

(SEQ ID NO: 455)
UGCUGCUGCUGCUGC

comprises the amino acid sequence:

(SEQ ID NO: 447)
GRSRLLEDFRNNRYPNLQLREIAGHIMEFSQDQHGSRFIR
LKLERATPAERQLVFNEILQAAYQLMVDVFGSYVIEKFFE
FGSLEQKLALAERIRGHVLSLALQMYGNRVIQKALEFIPS
DQQNEMVRELDGHVLKCVKDQNGSYVVRKCIECVQPQSLQ
FIIDAFKGQVFALSTHPYGSRVIERILEHCLPDQTLPILE
ELHQHIMEFSQDQHGNRFIQLKLERATPAERQLVFNEILQ
AAYQLMVDVFGSYVIRKFFEFGSLEQKLALAERIRGHVLS
LALQMYGSRVIEKALEFIPSDQQNEMVRELDGHVLKCVKD
QNGNHVVQKCIECVQPQSLQFIIDAFKGQVFALSTHPYGS
RVIRRILEHCLPDQTLPILEELHQHTEQLVQDQYGSYVIE
HVLEHGRPEDKSKIVAEIRGNVLVLSQHKFANNVVQKCVT
HASRTERAVLIDEVCTMNDGPHSHTEQLVQDQYGSYVIRH
VLEHGRPEDKSKIVAEIRGNVLVLSQHKFASNVVEKCVTH
ASRTERAVLIDEVCTMNDGPHSALYTMMKDQYANYVVQK
MIDVAEPGQRKIVMHKIRPHIATLRKYTYGKHILAKLEKY
YMKNGVDLG.

In some aspects, SEQ ID NO: 447 comprises an architecture proceeding from the N-terminus to the C-terminus according to: R1′-R1-R2-R3-R4-R5-R1-R2-R3-R4-R5-R6-R7-R6-R7-R8-R8′. In some aspects, SEQ ID NO: 447 is comprised of the sequences detailed in Table 18.

TABLE 18
15PUF protein according to SEQ ID NO: 447

| | | | SEQ |
|---|---|---|---|
| PUF | RNA | Amino Acid | ID |
| Module | Recognition | Sequence | NO |
| PUF R1′ | — | GRSRLLEDFRNNRYP | 495 |
| | | NLQLREIAG | |
| PUF R1 | C | HIMEFSQDQHGSRFI | 498 |
| | | RLKLERATPAERQLV | |
| | | FNEILQ | |
| PUF R2 | G | AAYQLMVDVFGSYVI | 491 |
| | | EKFFEFGSLEQKLAL | |
| | | AERIRG | |
| PUF R3 | U | HVLSLALQMYGNRVI | 505 |
| | | QKALEFIPSDQQNEM | |
| | | VRELDG | |
| PUF R4 | C | HVLKCVKDQNGSYVV | 504 |
| | | RKCIECVQPQSLQFI | |
| | | IDAFKG | |
| PUF R5 | G | QVFALSTHPYGSRVI | 512 |
| | | ERILEHCLPDQTLPI | |
| | | LEELHQ | |
| PUF R1 | U | HIMEFSQDQHGNRFI | 497 |
| | | QLKLERATPAERQLV | |
| | | FNEILQ | |
| PUF R2 | C | AAYQLMVDVFGSYVI | 492 |
| | | RKFFEFGSLEQKLAL | |
| | | AERIRG | |
| PUF R3 | G | HVLSLALQMYGSRVI | 506 |
| | | EKALEFIPSDQQNEM | |
| | | VRELDG | |
| PUF R4 | U | HVLKCVKDQNGNHVV | 502 |
| | | QKCIECVQPQSLQFI | |
| | | IDAFKG | |
| PUF R5 | C | QVFALSTHPYGSRVI | 513 |
| | | RRILEHCLPDQTLPI | |
| | | LEELHQ | |
| PUF R6 | G | HTEQLVQDQYGSYVI | 500 |
| | | EHVLEHGRPEDKSKI | |
| | | VAEIRG | |
| PUF R7 | U | NVLVLSQHKFANNVV | 508 |
| | | QKCVTHASRTERAVL | |
| | | IDEVCTMNDGPHS | |
| PUF R6 | C | HTEQLVQDQYGSYVI | 501 |
| | | RHVLEHGRPEDKSKI | |
| | | VAEIRG | |
| PUF R7 | G | NVLVLSQHKFASNVV | 509 |
| | | EKCVTHASRTERAVL | |
| | | IDEVCTMNDGPHS | |
| PUF R8 | U | ALYTMMKDQYANYVV | 493 |
| | | QKMIDVAEPGQRKIV | |
| | | MHKIRP | |
| PUF R8′ | — | HIATLRKYTYGKHIL | 496 |
| | | AKLEKYYMKNGVDLG | |

An exemplary 15-mer RNA recognition (15PUF) targeting

(SEQ ID NO: 455)
UGCUGCUGCUGCUGC

comprises tile amino acid sequence:

(SEQ ID NO: 448)
GRSRLLEDFRNNRYPNLQLREIAGHIMEFSQDQHGSRFIRL
KLERATPAERQLVFNEILQAAYQLMVDVFGSYVIEKFFEF
GSLEQKLALAERIRGHVLSLALQMYGNRVIQKALEFIPSD
QQNEMVRELDGHVLKCVKDQNGSYVVRKCIECVQPQSLQF
IIDAFKGQVFALSTHPYGSRVIERILEHCLPDQTLPILEE
LHQHTEQLVQDQYGNYVIQHVLEHGRPEDKSKIVAEIRGH
IMEFSQDQHGSRFIRLKLERATPAERQLVFNEILQAAYQL
MVDVFGSYVIEKFFEFGSLEQKLALAERIRGHVLSLALQM
YGNRVIQKALEFIPSDQQNEMVRELDGHVLKCVKDQNGSY
VVRKCIECVQPQSLQFIIDAFKGQVFALSTHPYGSRVIER
ILEHCLPDQTLPILEELHQHTEQLVQDQYGNYVIQHVLEH
GRPEDKSKIVAEIRGNVLVLSQHKFASYVVRKCVTHASRT
ERAVLIDEVCTMNDGPHSNVLVLSQHKFASNVVEKCVTHA
SRTERAVLIDEVCTMNDGPHSALYTMMKDQYANYVVQKMI
DVAEPGQRKIVMHKIRPHIATLRKYTYGKHILAKLEKYYM
KNGVDLG.

In some aspects, SEQ ID NO: 448 comprises an architecture proceeding from the N-terminus to the C-terminus according to: R1′-R1-R2-R3-R4-R5-R6-R1-R2-R3-R4-R5-R6-R7-R7-R8-R8′. In some aspects, SEQ ID NO: 448 is comprised of the sequences detailed in Table 19.

TABLE 19
15PUF protein according to SEQ ID NO: 448

| | | | SEQ |
|---|---|---|---|
| PUF | RNA | Amino Acid | ID |
| Module | Recognition | Sequence | NO |
| PUF R1′ | — | GRSRLLEDFRNNRYP | 495 |
| | | NLQLREIAG | |
| PUF R1 | C | HIMEFSQDQHGSRFI | 498 |
| | | RLKLERATPAERQLV | |
| | | FNEILQ | |
| PUF R2 | G | AAYQLMVDVFGSYVI | 491 |
| | | EKFFEFGSLEQKLAL | |
| | | AERIRG | |
| PUF R3 | U | HVLSLALQMYGNRVI | 505 |
| | | QKALEFIPSDQQNEM | |
| | | VRELDG | |
| PUF R4 | C | HVLKCVKDQNGSYVV | 504 |
| | | RKCIECVQPQSLQFI | |
| | | IDAFKG | |
| PUF R5 | G | QVFALSTHPYGSRV | 512 |
| | | IERILEHCLPDQTLP | |
| | | ILEELHQ | |
| PUF R6 | U | HTEQLVQDQYGNYVI | 499 |
| | | QHVLEHGRPEDKSKI | |
| | | VAEIRG | |
| PUF R1 | C | HIMEFSQDQHGSRFI | 498 |
| | | RLKLERATPAERQLV | |
| | | FNEILQ | |
| PUF R2 | G | AAYQLMVDVFGSYVI | 491 |
| | | EKFFEFGSLEQKLAL | |
| | | AERIRG | |
| PUF R3 | U | HVLSLALQMYGNRVI | 505 |
| | | QKALEFIPSDQQNE | |
| | | MVRELDG | |
| PUF R4 | C | HVLKCVKDQNGSYVV | 504 |
| | | RKCIECVQPQSLQFI | |
| | | IDAFKG | |
| PUF R5 | G | QVFALSTHPYGSRVI | 512 |
| | | ERILEHCLPDQTLP | |
| | | ILEELHQ | |
| PUF R6 | U | HTEQLVQDQYGNYVI | 499 |
| | | QHVLEHGRPEDKSKI | |
| | | VAEIRG | |
| PUF R7 | C | NVLVLSQHKFASYVV | 510 |
| | | RKCVTHASRTERAVL | |
| | | IDEVCTMNDGPHS | |
| PUF R7 | G | NVLVLSQHKFASNVV | 509 |
| | | EKCVTHASRTERAVL | |
| | | IDEVCTMNDGPHS | |
| PUF R8 | U | ALYTMMKDQYANYVV | 493 |
| | | QKMIDVAEPGQRKIV | |
| | | MHKIRP | |
| PUF R8′ | — | HIATLRKYTYGKHIL | 496 |
| | | AKLEKYYMKNGVDLG | |

An exemplary 15-mer RNA recognition (15PUF) targeting

(SEQ ID NO: 455)
UGCUGCUGCUGCUGC

comprises the amino acid sequence:

(SEQ ID NO: 461)
GRSRLLEDFRNNRYPNLQLREIAGHIMEFSQDQHGSRFIRLKLERATPA
ERQLVFNEILQAAYQLMVDVFGSYVIEKFFEFGSLEQKLALAERIRGHV
LSLALQMYGNRVIQKALEFIPSDQQNEMVRELDGHVLKCVKDQNGSYVV
RKCIECVQPQSLQFIIDAFKGQVFALSTHPYGSRVIERILEHCLPDQTL
PILEELHQHTEQLVQDQYGNYVIQHVLEHGRPEDKSKIVAEIRGNVLVL
SQHKFASYVVRKCVTHASRTERAVLIDEVCTMNDGPHSHIMEFSQDQHG
SRFIELKLERATPAERQLVFNEILQAAYQLMVDVFGNYVIQKFFEFGSL
EQKLALAERIRGHVLSLALQMYGSYVIRKALEFIPSDQQNEMVRELDGH
VLKCVKDQNGSYVVEKCIECVQPQSLQFIIDAFKGQVFALSTHPYGNRV
IQRILEHCLPDQTLPILEELHQHTEQLVQDQYGSYVIRHVLEHGRPEDK
SKIVAEIRGNVLVLSQHKFASNVVEKCVTHASRTERAVLIDEVCTMNDG
PHSALYTMMKDQYANYVVQKMIDVAEPGQRKIVMHKIRPHIATLRKYTY
GKHILAKLEKYYMKNGVDLG.

In some aspects, SEQ ID NO: 461 comprises an architecture proceeding from the N-terminus to the C-terminus according to: R1′-R1-R2-R3-R4-R5-R6-R7-R1-R2-R3-R4-R5-R6-R7-R8-R8′. In some aspects, SEQ ID NO: 461 is comprised of the sequences detailed in Table 20.

TABLE 20
15PUF protein according to SEQ ID NO: 461

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| PUF R1 | C | HIMEFSQDQHGSRFIRLKLERATPAERQLVFNEILQ | 498 |
| PUF R2 | G | AAYQLMVDVFGSYVIEKFFEFGSLEQKLALAERIRG | 491 |
| PUF R3 | U | HVLSLALQMYGNRVIQKALEFIPSDQQNEMVRELDG | 505 |
| PUF R4 | C | HVLKCVKDQNGSYVVRKCIECVQPQSLQFIIDAFKG | 504 |
| PUF R5 | G | QVFALSTHPYGSRVIERILEHCLPDQTLPILEELHQ | 512 |
| PUF R6 | U | HTEQLVQDQYGNYVIQHVLEHGRPEDKSKIVAEIRG | 499 |
| PUF R7 | C | NVLVLSQHKFASYVVRKCVTHASRTERAVLIDEVCTMND | 510 |
| | | GPHS | |
| PUF R1 | G | HIMEFSQDQHGSRFIELKLERATPAERQLVFNEILQ | 498 |
| PUF R2 | U | AAYQLMVDVFGNYVIQKFFEFGSLEQKLALAERIRG | 490 |
| PUF R3 | C | HVLSLALQMYGSYVIRKALEFIPSDQQNEMVRELDG | 507 |
| PUF R4 | G | HVLKCVKDQNGSYVVEKCIECVQPQSLQFIIDAFKG | 503 |
| PUF R5 | U | QVFALSTHPYGNRVIQRILEHCLPDQTLPILEELHQ | 511 |
| PUF R6 | C | HTEQLVQDQYGSYVIRHVLEHGRPEDKSKIVAEIRG | 501 |
| PUF R7 | G | NVLVLSQHKFASNVVEKCVTHASRTERAVLIDEVCTMND | 509 |
| | | GPHS | |
| PUF R8 | U | ALYTMMKDQYANYVVQKMIDVAEPGQRKIVMHKIRP | 493 |
| PUF R8′ | | HIATLRKYTYGKHILAKLEKYYMKNGVDLG | 496 |

An exemplary 16-mer RNA recognition (16PUF) targeting

(SEQ ID NO: 456)
UGCUGCUGCUGCUGCU

comprises the amino acid sequence:

(SEQ ID NO: 449)
GRSRLLEDFRNNRYPNLQLREIAGHIMEFSQDQHGNRFIQLKLERATPA
ERQLVFNEILQAAYQLMVDVFGSYVIRKFFEFGSLEQKLALAERIRGHV
LSLALQMYGSRVIEKALEFIPSDQQNEMVRELDGHVLKCVKDQNGNHVV
QKCIECVQPQSLQFIIDAFKGQVFALSTHPYGSRVIRRILEHCLPDQTL
PILEELHQHIMEFSQDQHGSRFIELKLERATPAERQLVFNEILQAAYQL
MVDVFGNYVIQKFFEFGSLEQKLALAERIRGHVLSLALQMYGSYVIRKA
LEFIPSDQQNEMVRELDGHVLKCVKDQNGSYVVEKCIECVQPQSLQFII
DAFKGQVFALSTHPYGNRVIQRILEHCLPDQTLPILEELHQHTEQLVQD
QYGSYVIRHVLEHGRPEDKSKIVAEIRGNVLVLSQHKFASNVVEKCVTH
ASRTERAVLIDEVCTMNDGPHSALYTMMKDQYANYVVQKMIDVAEPGQR
KIVMHKIRPHTEQLVQDQYGSYVIRHVLEHGRPEDKSKIVAEIRGNVLV
LSQHKFASNVVEKCVTHASRTERAVLIDEVCTMNDGPHSALYTMMKDQY
ANYVVQKMIDVAEPGQRKIVMHKIRPHIATLRKYTYGKHILAKLEKYYM
KNGVDLG.

In some aspects, SEQ ID NO: 449 comprises an architecture proceeding from the N-terminus to the C-terminus according to: R1′-R1-R2-R3-R4-R5-R1-R2-R3-R4-R5-R6-R7-R8-R6-R7-R8-R8′. In some aspects, SEQ ID NO: 449 is comprised of the sequences detailed in Table 21.

TABLE 21
16PUF protein according to SEQ ID NO: 449

| PUF | RNA | | SEQ |
|---|---|---|---|
| Module | Recognition | Amino Acid Sequence | ID NO |
| PUF R1′ | | GRSRLLEDFRNNRYPNLQLREIAG | 495 |
| PUF R1 | U | HIMEFSQDQHGNRFIQLKLERATPAERQLVFNEILQ | 497 |
| PUF R2 | C | AAYQLMVDVFGSYVIRKFFEFGSLEQKLALAERIRG | 492 |
| PUF R3 | G | HVLSLALQMYGSRVIEKALEFIPSDQQNEMVRELDG | 506 |
| PUF R4 | U | HVLKCVKDQNGNHVVQKCIECVQPQSLQFIIDAFKG | 502 |
| PUF R5 | C | QVFALSTHPYGSRVIRRILEHCLPDQTLPILEELHQ | 513 |
| PUF R1 | G | HIMEFSQDQHGSRFIELKLERATPAERQLVFNEILQ | 498 |
| PUF R2 | U | AAYQLMVDVFGNYVIQKFFEFGSLEQKLALAERIRG | 490 |
| PUF R3 | C | HVLSLALQMYGSYVIRKALEFIPSDQQNEMVRELDG | 507 |
| PUF R4 | G | HVLKCVKDQNGSYVVEKCIECVQPQSLQFIIDAFKG | 503 |
| PUF R5 | U | QVFALSTHPYGNRVIQRILEHCLPDQTLPILEELHQ | 511 |
| PUF R6 | C | HTEQLVQDQYGSYVIRHVLEHGRPEDKSKIVAEIRG | 501 |
| PUF R7 | G | NVLVLSQHKFASNVVEKCVTHASRTERAVLIDEVCTMN | 509 |
| | | DGPHS | |
| PUF R8 | U | ALYTMMKDQYANYVVQKMIDVAEPGQRKIVMHKIRP | 493 |
| PUF R6 | C | HTEQLVQDQYGSYVIRHVLEHGRPEDKSKIVAEIRG | 501 |
| PUF R7 | G | NVLVLSQHKFASNVVEKCVTHASRTERAVLIDEVCTMN | 509 |
| | | DGPHS | |
| PUF R8 | U | ALYTMMKDQYANYVVQKMIDVAEPGQRKIVMHKIRP | 493 |
| PUF R8′ | | HIATLRKYTYGKHILAKLEKYYMKNGVDLG | 496 |

An exemplary 16-mer RNA recognition (16PUF)

(SEQ ID NO: 456)
UGCUGCUGCUGCUGCU

comprises the amino acid sequence:

(SEQ ID NO: 450)
GRSRLLEDFRNNRYPNLQLREIAGHIMEFSQDQHGNRFIQLKLERATPA
ERQLVFNEILQAAYQLMVDVFGSYVIRKFFEFGSLEQKLALAERIRGHV
LSLALQMYGSRVIEKALEFIPSDQQNEMVRELDGHVLKCVKDQNGNHVV
QKCIECVQPQSLQFIIDAFKGQVFALSTHPYGSRVIRRILEHCLPDQTL
PILEELHQHTEQLVQDQYGSYVIEHVLEHGRPEDKSKIVAEIRGHIMEF
SQDQHGNRFIQLKLERATPAERQLVFNEILQAAYQLMVDVFGSYVIRKF
FEFGSLEQKLALAERIRGHVLSLALQMYGSRVIEKALEFIPSDQQNEMV
RELDGHVLKCVKDQNGNHVVQKCIECVQPQSLQFIIDAFKGQVFALSTH
PYGSRVIRRILEHCLPDQTLPILEELHQHTEQLVQDQYGSYVIEHVLEH
GRPEDKSKIVAEIRGNVLVLSQHKFANNVVQKCVTHASRTERAVLIDEV
CTMNDGPHSALYTMMKDQYASYVVRKMIDVAEPGQRKIVMHKIRPNVLV
LSQHKFASNVVEKCVTHASRTERAVLIDEVCTMNDGPHSALYTMMKDQY
ANYVVQKMIDVAEPGQRKIVMHKIRPHIATLRKYTYGKHILAKLEKYYM
KNGVDLG.

In some aspects, SEQ ID NO: 450 comprises an architecture proceeding from the N-terminus to the C-terminus according to: R1′-R1-R2-R3-R4-R5-R6-R1-R2-R3-R4-R5-R6-R7-R8-R7-R8-R8′. In some aspects, SEQ ID NO: 450 is comprised of the sequences detailed in Table 22.

TABLE 22
16PUF protein according to SEQ ID NO: 450

| PUF | RNA | | SEQ |
|---|---|---|---|
| Module | Recognition | Amino Acid Sequence | ID NO |
| PUF R1′ | | GRSRLLEDFRNNRYPNLQLREIAG | 495 |
| PUF R1 | U | HIMEFSQDQHGNRFIQLKLERATPAERQLVFNEILQ | 497 |
| PUF R2 | C | AAYQLMVDVFGSYVIRKFFEFGSLEQKLALAERIRG | 492 |
| PUF R3 | G | HVLSLALQMYGSRVIEKALEFIPSDQQNEMVRELDG | 506 |
| PUF R4 | U | HVLKCVKDQNGNHVVQKCIECVQPQSLQFIIDAFKG | 502 |
| PUF R5 | C | QVFALSTHPYGSRVIRRILEHCLPDQTLPILEELHQ | 513 |
| PUF R6 | G | HTEQLVQDQYGSYVIEHVLEHGRPEDKSKIVAEIRG | 500 |
| PUF R1 | U | HIMEFSQDQHGNRFIQLKLERATPAERQLVFNEILQ | 497 |
| PUF R2 | C | AAYQLMVDVFGSYVIRKFFEFGSLEQKLALAERIRG | 492 |
| PUF R3 | G | HVLSLALQMYGSRVIEKALEFIPSDQQNEMVRELDG | 506 |
| PUF R4 | U | HVLKCVKDQNGNHVVQKCIECVQPQSLQFIIDAFKG | 502 |
| PUF R5 | C | QVFALSTHPYGSRVIRRILEHCLPDQTLPILEELHQ | 513 |
| PUF R6 | G | HTEQLVQDQYGSYVIEHVLEHGRPEDKSKIVAEIRG | 500 |
| PUF R7 | U | NVLVLSQHKFANNVVQKCVTHASRTERAVLIDEVCTM | 508 |
| | | NDGPHS | |
| PUF R8 | C | ALYTMMKDQYASYVVRKMIDVAEPGQRKIVMHKIRP | 494 |
| PUF R7 | G | NVLVLSQHKFASNVVEKCVTHASRTERAVLIDEVCTMN | 509 |
| | | DGPHS | |
| PUF R8 | U | ALYTMMKDQYANYVVQKMIDVAEPGQRKIVMHKIRP | 493 |
| PUF R8′ | | HIATLRKYTYGKHILAKLEKYYMKNGVDLG | 496 |

An exemplary 16-mer RNA recognition (16PUF) targeting

(SEQ ID NO: 456)
UGCUGCUGCUGCUGCU

comprises the amino acid sequence:

(SEQ ID NO: 451)
GRSRLLEDFRNNRYPNLQLREIAGHIMEFSQDQHGNRFIQLKLERATPA
ERQLVFNEILQAAYQLMVDVFGSYVIRKFFEFGSLEQKLALAERIRGHV
LSLALQMYGSRVIEKALEFIPSDQQNEMVRELDGHVLKCVKDQNGNHVV
QKCIECVQPQSLQFIIDAFKGQVFALSTHPYGSRVIRRILEHCLPDQTL
PILEELHQHTEQLVQDQYGSYVIEHVLEHGRPEDKSKIVAEIRGNVLVL
SQHKFANNVVQKCVTHASRTERAVLIDEVCTMNDGPHSALYTMMKDQYA
SYVVRKMIDVAEPGQRKIVMHKIRPHIMEFSQDQHGSRFIELKLERATP
AERQLVFNEILQAAYQLMVDVFGNYVIQKFFEFGSLEQKLALAERIRGH
VLSLALQMYGSYVIRKALEFIPSDQQNEMVRELDGHVLKCVKDQNGSYV
VEKCIECVQPQSLQFIIDAFKGQVFALSTHPYGNRVIQRILEHCLPDQT
LPILEELHQHTEQLVQDQYGSYVIRHVLEHGRPEDKSKIVAEIRGNVLV
LSQHKFASNVVEKCVTHASRTERAVLIDEVCTMNDGPHSALYTMMKDQY
ANYVVQKMIDVAEPGQRKIVMHKIRPHIATLRKYTYGKHILAKLEKYYM
KNGVDLG.

In some aspects, SEQ ID NO: 451 comprises an architecture proceeding from the N-terminus to the C-terminus according to: R1′-R1-R2-R3-R4-R5-R6-R7-R8-R1-R2-R3-R4-R5-R6-R7-R8-R8′. In some aspects, SEQ ID NO: 451 is comprised of the sequences detailed in Table 23.

TABLE 23
16PUF protein according to SEQ ID NO: 451

| PUF | RNA | | SEQ |
|---|---|---|---|
| Module | Recognition | Amino Acid Sequence | ID NO |
| PUF R1′ | | GRSRLLEDFRNNRYPNLQLREIAG | 495 |
| PUF R1 | U | HIMEFSQDQHGNRFIQLKLERATPAERQLVFNEILQ | 497 |
| PUF R2 | C | AAYQLMVDVFGSYVIRKFFEFGSLEQKLALAERIRG | 492 |
| PUF R3 | G | HVLSLALQMYGSRVIEKALEFIPSDQQNEMVRELDG | 506 |
| PUF R4 | U | HVLKCVKDQNGNHVVQKCIECVQPQSLQFIIDAFKG | 502 |
| PUF R5 | C | QVFALSTHPYGSRVIRRILEHCLPDQTLPILEELHQ | 513 |
| PUF R6 | G | HTEQLVQDQYGSYVIEHVLEHGRPEDKSKIVAEIRG | 500 |
| PUF R7 | U | NVLVLSQHKFANNVVQKCVTHASRTERAVLIDEVCTM | 508 |
| | | NDGPHS | |
| PUF R8 | C | ALYTMMKDQYASYVVRKMIDVAEPGQRKIVMHKIRP | 494 |
| PUF R1 | G | HIMEFSQDQHGSRFIELKLERATPAERQLVFNEILQ | 498 |
| PUF R2 | U | AAYQLMVDVFGNYVIQKFFEFGSLEQKLALAERIRG | 490 |
| PUF R3 | C | HVLSLALQMYGSYVIRKALEFIPSDQQNEMVRELDG | 507 |
| PUF R4 | G | HVLKCVKDQNGSYVVEKCIECVQPQSLQFIIDAFKG | 503 |
| PUF R5 | U | QVFALSTHPYGNRVIQRILEHCLPDQTLPILEELHQ | 511 |
| PUF R6 | C | HTEQLVQDQYGSYVIRHVLEHGRPEDKSKIVAEIRG | 501 |
| PUF R7 | G | NVLVLSQHKFASNVVEKCVTHASRTERAVLIDEVCTMN | 509 |
| | | DGPHS | |
| PUF R8 | U | ALYTMMKDQYANYVVQKMIDVAEPGQRKIVMHKIRP | 493 |
| PUF R8′ | — | HIATLRKYTYGKHILAKLEKYYMKNGVDLG | 496 |

An exemplary 8-mer RNA recognition (8PUF) targeting

(SEQ ID NO: 472)
CUGCUGCU

comprises the amino acid sequence:

(SEQ ID NO: 480)
GRSRLLEDFRNNRYPNLQLREIAGHIMEFSQDQHGNRFIQLKLERATPA
ERQLVFNEILQAAYQLMVDVFGSYVIRKFFEFGSLEQKLALAERIRGHV
LSLALQMYGSRVIEKALEFIPSDQQNEMVRELDGHVLKCVKDQNGNHVV
QKCIECVQPQSLQFIIDAFKGQVFALSTHPYGSRVIRRILEHCLPDQTL
PILEELHQHTEQLVQDQYGSYVIEHVLEHGRPEDKSKIVAEIRGNVLVL
SQHKFANNVVQKCVTHASRTERAVLIDEVCTMNDGPHSALYTMMKDQYA
SYVVRKMIDVAEPGQRKIVMHKIRPHIATLRKYTYGKHILAKLEKYYMK
NGVDLG.

In some aspects SEQ ID NO: 480 comprises an architecture proceeding from the N-terminus to the C-terminus according to: R1′-R1-R2-R3-R4-R5-R6-R7-R8-R8′. In some aspects, SEQ ID NO: 480 is comprised of the sequences detailed in Table 24.

TABLE 24
8PUF protein according to SEQ ID NO: 480

| PUF | RNA | | SEQ |
|---|---|---|---|
| Module | Recognition | Amino Acid Sequence | ID NO |
| PUF R1′ | | GRSRLLEDFRNNRYPNLQLREIAG | 495 |
| PUF R1 | U | HIMEFSQDQHGNRFIQLKLERATPAERQLVFNEILQ | 497 |
| PUF R2 | C | AAYQLMVDVFGSYVIRKFFEFGSLEQKLALAERIRG | 492 |
| PUF R3 | G | HVLSLALQMYGSRVIEKALEFIPSDQQNEMVRELDG | 507 |
| PUF R4 | U | HVLKCVKDQNGNHVVQKCIECVQPQSLQFIIDAFKG | 503 |
| PUF R5 | C | QVFALSTHPYGSRVIRRILEHCLPDQTLPILEELHQ | 514 |
| PUF R6 | G | HTEQLVQDQYGSYVIEHVLEHGRPEDKSKIVAEIRG | 501 |
| PUF R7 | U | NVLVLSQHKFANNVVQKCVTHASRTERAVLIDEVCTMND | 509 |
| | | GPHS | |
| PUF R8 | C | ALYTMMKDQYASYVVRKMIDVAEPGQRKIVMHKIRP | 494 |
| PUF R8′ | | HIATLRKYTYGKHILAKLEKYYMKNGVDLG | 496 |

An exemplary 14-mer RNA recognition (14PUF) targeting

(SEQ ID NO: 473)
CUGCUGCUGCUGCU

comprises the amino acid sequence:

(SEQ ID NO: 481)
GRSRLLEDFRNNRYPNLQLREIAGHIMEFSQDQHGNRFIQLKLERATPA
ERQLVFNEILQAAYQLMVDVFGSYVIRKFFEFGSLEQKLALAERIRGHV
LSLALQMYGSRVIEKALEFIPSDQQNEMVRELDGHVLKCVKDQNGNHVV
QKCIECVQPQSLQFIIDAFKGQVFALSTHPYGSRVIRRILEHCLPDQTL
PILEELHQHIMEFSQDQHGSRFIELKLERATPAERQLVFNEILQAAYQL
MVDVFGNYVIQKFFEFGSLEQKLALAERIRGHVLSLALQMYGSYVIRKA
LEFIPSDQQNEMVRELDGHVLKCVKDQNGSYVVEKCIECVQPQSLQFII
DAFKGQVFALSTHPYGNRVIQRILEHCLPDQTLPILEELHQHTEQLVQD
QYGSYVIRHVLEHGRPEDKSKIVAEIRGHTEQLVQDQYGSYVIEHVLEH
GRPEDKSKIVAEIRGNVLVLSQHKFANNVVQKCVTHASRTERAVLIDEV
CTMNDGPHSALYTMMKDQYASYVVRKMIDVAEPGQRKIVMHKIRPHIAT
LRKYTYGKHILAKLEKYYMKNGVDLG.

In some aspects, SEQ ID NO: 481 comprises an architecture proceeding from the N-terminus to the C-terminus according to: R1′-R-2R-45-1R-3R-5R-6 R-2R-4R-1R-3R5R-6R7-R8-R8′. In some aspects, SEQ ID NO: 481 is comprised of the sequences detailed in Table 25.

TABLE 25
14PUF protein according to SEQ ID NO: 481

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| PUF R1 | U | HIMEFSQDQHGNRFIQLKLERATPAERQLVFNEILQ | 497 |
| PUF R2 | C | AAYQLMVDVFGSYVIRKFFEFGSLEQKLALAERIRG | 492 |
| PUF R3 | G | HVLSLALQMYGSRVIEKALEFIPSDQQNEMVRELDG | 507 |
| PUF R4 | U | HVLKCVKDQNGNHVVQKCIECVQPQSLQFIIDAFKG | 503 |
| PUF R5 | C | QVFALSTHPYGSRVIRRILEHCLPDQTLPILEELHQ | 514 |
| PUF R1 | G | HIMEFSQDQHGSRFIELKLERATPAERQLVFNEILQ | 501 |
| PUF R2 | U | AAYQLMVDVFGNYVIQKFFEFGSLEQKLALAERIRG | 509 |
| PUF R3 | C | HVLSLALQMYGSYVIRKALEFIPSDQQNEMVRELDG | 508 |
| PUF R4 | G | HVLKCVKDQNGSYVVEKCIECVQPQSLQFIIDAFKG | 504 |
| PUF R5 | U | QVFALSTHPYGNRVIQRILEHCLPDQTLPILEELHQ | 512 |
| PUF R6 | C | HTEQLVQDQYGSYVIRHVLEHGRPEDKSKIVAEIRG | 502 |
| PUF R6 | G | HTEQLVQDQYGSYVIEHVLEHGRPEDKSKIVAEIRG | 501 |
| PUF R7 | U | NVLVLSQHKFANNVVQKCVTHASRTERAVLIDEVCTMND | 509 |
| | | GPHS | |
| PUF R8 | C | ALYTMMKDQYASYVVRKMIDVAEPGQRKIVMHKIRP | 494 |
| PUF R8′ | | HIATLRKYTYGKHILAKLEKYYMKNGVDLG | 496 |

An exemplary 14-mer RNA recognition (14PUF) targeting

(SEQ ID NO: 473)
CUGCUGCUGCUGCU

comprises the amino acid sequence:

(SEQ ID NO: 482)
GRSRLLEDFRNNRYPNLQLREIAGHIMEFSQDQHGNRFIQLKLERATPA
ERQLVFNEILQAAYQLMVDVFGSYVIRKFFEFGSLEQKLALAERIRGHV
LSLALQMYGSRVIEKALEFIPSDQQNEMVRELDGHVLKCVKDQNGNHVV
QKCIECVQPQSLQFIIDAFKGQVFALSTHPYGSRVIRRILEHCLPDQTL
PILEELHQHTEQLVQDQYGSYVIEHVLEHGRPEDKSKIVAEIRGHIMEF
SQDQHGNRFIQLKLERATPAERQLVFNEILQAAYQLMVDVFGSYVIRKF
FEFGSLEQKLALAERIRGHVLSLALQMYGSRVIEKALEFIPSDQQNEMV
RELDGHVLKCVKDQNGNHVVQKCIECVQPQSLQFIIDAFKGQVFALSTH
PYGSRVIRRILEHCLPDQTLPILEELHQHTEQLVQDQYGSYVIEHVLEH
GRPEDKSKIVAEIRGNVLVLSQHKFANNVVQKCVTHASRTERAVLIDEV
CTMNDGPHSALYTMMKDQYASYVVRKMIDVAEPGQRKIVMHKIRPHIAT
LRKYTYGKHILAKLEKYYMKNGVDLG.

In some aspects, SEQ ID NO: 482 comprises an architecture proceeding from the N-terminus to the C-terminus according to: R1′-R1-R2-R3-R4-R5-R6-R1-R2-R3-R4-R5-R6-R7-R8-R8′. In some aspects, SEQ ID NO: 482 is comprised of the sequences detailed in Table 26.

TABLE 26
14PUF protein according to SEQ ID NO: 482

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| PUF R1 | U | HIMEFSQDQHGNRFIQLKLERATPAERQLVFNEILQ | 497 |
| PUF R2 | C | AAYQLMVDVFGSYVIRKFFEFGSLEQKLALAERIRG | 492 |
| PUF R3 | G | HVLSLALQMYGSRVIEKALEFIPSDQQNEMVRELDG | 507 |
| PUF R4 | U | HVLKCVKDQNGNHVVQKCIECVQPQSLQFIIDAFKG | 503 |
| PUF R5 | C | QVFALSTHPYGSRVIRRILEHCLPDQTLPILEELHQ | 514 |
| PUF R6 | G | HTEQLVQDQYGSYVIEHVLEHGRPEDKSKIVAEIRG | 501 |
| PUF RI | U | HIMEFSQDQHGNRFIQLKLERATPAERQLVFNEILQ | 497 |
| PUF R2 | C | AAYQLMVDVFGSYVIRKFFEFGSLEQKLALAERIRG | 492 |
| PUF R3 | G | HVLSLALQMYGSRVIEKALEFIPSDQQNEMVRELDG | 507 |
| PUF R4 | U | HVLKCVKDQNGNHVVQKCIECVQPQSLQFIIDAFKG | 503 |
| PUF R5 | C | QVFALSTHPYGSRVIRRILEHCLPDQTLPILEELHQ | 514 |
| PUF R6 | G | HTEQLVQDQYGSYVIEHVLEHGRPEDKSKIVAEIRG | 501 |
| PUF R7 | U | NVLVLSQHKFANNVVQKCVTHASRTERAVLIDEVCTMND | 509 |
| | | GPHS | |
| PUF R8 | C | ALYTMMKDQYASYVVRKMIDVAEPGQRKIVMHKIRP | 494 |
| PUF R8′ | — | HIATLRKYTYGKHILAKLEKYYMKNGVDLG | 496 |

An exemplary 15-mer RNA recognition (15PUF) targeting

(SEQ ID NO: 474)
CUGCUGCUGCUGCUG

comprises the amino acid sequence:

(SEQ ID NO: 483)
GRSRLLEDFRNNRYPNLQLREIAGHIMEFSQDQHGSRFIELKLERATPA
ERQLVFNEILQAAYQLMVDVFGNYVIQKFFEFGSLEQKLALAERIRGHV
LSLALQMYGSYVIRKALEFIPSDQQNEMVRELDGHVLKCVKDQNGSYVV
EKCIECVQPQSLQFIIDAFKGQVFALSTHPYGNRVIQRILEHCLPDQTL
PILEELHQHIMEFSQDQHGSRFIRLKLERATPAERQLVFNEILQAAYQL
MVDVFGSYVIEKFFEFGSLEQKLALAERIRGHVLSLALQMYGNRVIQKA
LEFIPSDQQNEMVRELDGHVLKCVKDQNGSYVVRKCIECVQPQSLQFII
DAFKGQVFALSTHPYGSRVIERILEHCLPDQTLPILEELHQHTEQLVQD
QYGNYVIQHVLEHGRPEDKSKIVAEIRGNVLVLSQHKFASYVVRKCVTH
ASRTERAVLIDEVCTMNDGPHSHTEQLVQDQYGSYVIEHVLEHGRPEDK
SKIVAEIRGNVLVLSQHKFANNVVQKCVTHASRTERAVLIDEVCTMNDG
PHSALYTMMKDQYASYVVRKMIDVAEPGQRKIVMHKIRPHIATLRKYTY
GKHILAKLEKYYMKNGVDLG

In some aspects, SEQ ID NO: 483 comprises an architecture proceeding from the N-terminus to the C-terminus according to: R1′-R1-R2-R3-R4-R5-R1-R2-R3-R4-R5-R6-R7-R6-R7-R8-R8′. In some aspects, SEQ ID NO: 483 is comprised of the sequences detailed in Table 27.

TABLE 27
15PUF protein according to SEQ ID NO: 483

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | G | HIMEFSQDQHGSRFIELKLERATPAERQLVFNEILQ | 498 |
| R2 | U | AAYQLMVDVFGNYVIQKFFEFGSLEQKLALAERIRG | 490 |
| R3 | C | HVLSLALQMYGSYVIRKALEFIPSDQQNEMVRELDG | 508 |
| R4 | G | HVLKCVKDQNGSYVVEKCIECVQPQSLQFIIDAFKG | 504 |
| R5 | U | QVFALSTHPYGNRVIQRILEHCLPDQTLPILEELHQ | 512 |
| R1 | C | HIMEFSQDQHGSRFIRLKLERATPAERQLVFNEILQ | 499 |
| R2 | G | AAYQLMVDVFGSYVIEKFFEFGSLEQKLALAERIRG | 491 |
| R3 | U | HVLSLALQMYGNRVIQKALEFIPSDQQNEMVRELDG | 506 |
| R4 | C | HVLKCVKDQNGSYVVRKCIECVQPQSLQFIIDAFKG | 505 |
| R5 | G | QVFALSTHPYGSRVIERILEHCLPDQTLPILEELHQ | 513 |
| R6 | U | HTEQLVQDQYGNYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R6 | G | HTEQLVQDQYGSYVIEHVLEHGRPEDKSKIVAEIRG | 501 |
| R7 | U | NVLVLSQHKFANNVVQKCVTHASRTERAVLIDEVCTMND | 509 |
| | | GPHS | |
| R8 | C | ALYTMMKDQYASYVVRKMIDVAEPGQRKIVMHKIRP | 494 |
| PUF R8′ | — | HIATLRKYTYGKHILAKLEKYYMKNGVDLG | 496 |

An exemplary 15-mer RNA recognition (15PUF) targeting

(SEQ ID NO: 474)
CUGCUGCUGCUGCUG

comprises the amino acid sequence:

(SEQ ID NO: 484)
GRSRLLEDFRNNRYPNLQLREIAGHIMEFSQDQHGSRFIELKLERATPA
ERQLVFNEILQAAYQLMVDVFGNYVIQKFFEFGSLEQKLALAERIRGHV
LSLALQMYGSYVIRKALEFIPSDQQNEMVRELDGHVLKCVKDQNGSYVV
EKCIECVQPQSLQFIIDAFKGQVFALSTHPYGNRVIQRILEHCLPDQTL
PILEELHQHTEQLVQDQYGSYVIRHVLEHGRPEDKSKIVAEIRGHIMEF
SQDQHGSRFIELKLERATPAERQLVFNEILQAAYQLMVDVFGNYVIQKF
FEFGSLEQKLALAERIRGHVLSLALQMYGSYVIRKALEFIPSDQQNEMV
RELDGHVLKCVKDQNGSYVVEKCIECVQPQSLQFIIDAFKGQVFALSTH
PYGNRVIQRILEHCLPDQTLPILEELHQHTEQLVQDQYGSYVIRHVLEH
GRPEDKSKIVAEIRGNVLVLSQHKFASNVVEKCVTHASRTERAVLIDEV
CTMNDGPHSNVLVLSQHKFANNVVQKCVTHASRTERAVLIDEVCTMNDG
PHSALYTMMKDQYASYVVRKMIDVAEPGQRKIVMHKIRPHIATLRKYTY
GKHILAKLEKYYMKNGVDLG

In some aspects, SEQ ID NO: 484 comprises an architecture proceeding from the N-terminus to the C-terminus according to: R1′-R1-R2-R3-R4-R5-R6-R1-R2-R3-R4-R5-R6-R7-R7-R8-R8′. In some aspects, SEQ ID NO: 484 is comprised of the sequences detailed in Table 28.

TABLE 28
15PUF protein according to SEQ ID NO: 484

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | G | HIMEFSQDQHGSRFIELKLERATPAERQLVFNEILQ | 498 |
| R2 | U | AAYQLMVDVFGNYVIQKFFEFGSLEQKLALAERIRG | 490 |
| R3 | C | HVLSLALQMYGSYVIRKALEFIPSDQQNEMVRELDG | 508 |
| R4 | G | HVLKCVKDQNGSYVVEKCIECVQPQSLQFIIDAFKG | 504 |
| R5 | U | QVFALSTHPYGNRVIQRILEHCLPDQTLPILEELHQ | 512 |
| R6 | C | HTEQLVQDQYGSYVIRHVLEHGRPEDKSKIVAEIRG | 502 |
| R1 | G | HIMEFSQDQHGSRFIELKLERATPAERQLVFNEILQ | 498 |
| R2 | U | AAYQLMVDVFGNYVIQKFFEFGSLEQKLALAERIRG | 490 |
| R3 | C | HVLSLALQMYGSYVIRKALEFIPSDQQNEMVRELDG | 508 |
| R4 | G | HVLKCVKDQNGSYVVEKCIECVQPQSLQFIIDAFKG | 504 |
| R5 | U | QVFALSTHPYGNRVIQRILEHCLPDQTLPILEELHQ | 512 |
| R6 | C | HTEQLVQDQYGSYVIRHVLEHGRPEDKSKIVAEIRG | 502 |
| R7 | G | NVLVLSQHKFASNVVEKCVTHASRTERAVLIDEVCTMNDG | 510 |
| | | PHS | |
| R7 | U | NVLVLSQHKFANNVVQKCVTHASRTERAVLIDEVCTMND | 509 |
| | | GPHS | |
| R8 | C | ALYTMMKDQYASYVVRKMIDVAEPGQRKIVMHKIRP | 494 |
| PUF R8′ | — | HIATLRKYTYGKHILAKLEKYYMKNGVDLG | 496 |

An exemplary 15-mer RNA recognition (15PUF) targeting

(SEQ ID NO: 474)
CUGCUGCUGCUGCUG

comprises the amino acid sequence:

(SEQ ID NO: 485)
GRSRLLEDFRNNRYPNLQLREIAGHIMEFSQDQHGSRFIELKLERATPA
ERQLVFNEILQAAYQLMVDVFGNYVIQKFFEFGSLEQKLALAERIRGHV
LSLALQMYGSYVIRKALEFIPSDQQNEMVRELDGHVLKCVKDQNGSYVV
EKCIECVQPQSLQFIIDAFKGQVFALSTHPYGNRVIQRILEHCLPDQTL
PILEELHQHTEQLVQDQYGSYVIRHVLEHGRPEDKSKIVAEIRGNVLVL
SQHKFASNVVEKCVTHASRTERAVLIDEVCTMNDGPHSHIMEFSQDQHG
NRFIQLKLERATPAERQLVFNEILQAAYQLMVDVFGSYVIRKFFEFGSL
EQKLALAERIRGHVLSLALQMYGSRVIEKALEFIPSDQQNEMVRELDGH
VLKCVKDQNGNHVVQKCIECVQPQSLQFIIDAFKGQVFALSTHPYGSRV
IRRILEHCLPDQTLPILEELHQHTEQLVQDQYGSYVIEHVLEHGRPEDK
SKIVAEIRGNVLVLSQHKFANNVVQKCVTHASRTERAVLIDEVCTMNDG
PHSALYTMMKDQYASYVVRKMIDVAEPGQRKIVMHKIRPHIATLRKYTY
GKHILAKLEKYYMKNGVDLG

In some aspects, SEQ ID NO: 485 comprises an architecture proceeding from the N-terminus to the C-terminus according to: R1′-R1-R2-R3-R4-R5-R6-R7-R1-R2-R3-R4-R5-R6-R7-R8-R8′. In some aspects, SEQ ID NO: 485 is comprised of the sequences detailed in Table 29.

TABLE 29
15PUF protein according to SEQ ID NO: 485

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | G | HIMEFSQDQHGSRFIELKLERATPAERQLVFNEILQ | 498 |
| R2 | U | AAYQLMVDVFGNYVIQKFFEFGSLEQKLALAERIRG | 490 |
| R3 | C | HVLSLALQMYGSYVIRKALEFIPSDQQNEMVRELDG | 508 |
| R4 | G | HVLKCVKDQNGSYVVEKCIECVQPQSLQFIIDAFKG | 504 |
| R5 | U | QVFALSTHPYGNRVIQRILEHCLPDQTLPILEELHQ | 512 |
| R6 | C | HTEQLVQDQYGSYVIRHVLEHGRPEDKSKIVAEIRG | 502 |
| R7 | G | NVLVLSQHKFASNVVEKCVTHASRTERAVLIDEVCTMNDG | 510 |
| | | PHS | |
| R1 | U | HIMEFSQDQHGNRFIQLKLERATPAERQLVFNEILQ | 497 |
| R2 | C | AAYQLMVDVFGSYVIRKFFEFGSLEQKLALAERIRG | 492 |
| R3 | G | HVLSLALQMYGSRVIEKALEFIPSDQQNEMVRELDG | 507 |
| R4 | U | HVLKCVKDQNGNHVVQKCIECVQPQSLQFIIDAFKG | 503 |
| R5 | C | QVFALSTHPYGSRVIRRILEHCLPDQTLPILEELHQ | 514 |
| R6 | G | HTEQLVQDQYGSYVIEHVLEHGRPEDKSKIVAEIRG | 501 |
| R7 | U | NVLVLSQHKFANNVVQKCVTHASRTERAVLIDEVCTMND | 509 |
| | | GPHS | |
| R8 | C | ALYTMMKDQYASYVVRKMIDVAEPGQRKIVMHKIRP | 494 |
| PUF R8′ | — | HIATLRKYTYGKHILAKLEKYYMKNGVDLG | 496 |

An exemplary 16-mer RNA recognition (16PUF) targeting

(SEQ ID NO: 475)
CUGCUGCUGCUGCUGC

comprises the amino acid sequence:

(SEQ ID NO: 486)
GRSRLLEDFRNNRYPNLQLREIAGHIMEFSQDQHGSRFIRLKLERATPA
ERQLVFNEILQAAYQLMVDVFGSYVIEKFFEFGSLEQKLALAERIRGHV
LSLALQMYGNRVIQKALEFIPSDQQNEMVRELDGHVLKCVKDQNGSYVV
RKCIECVQPQSLQFIIDAFKGQVFALSTHPYGSRVIERILEHCLPDQTL
PILEELHQHIMEFSQDQHGNRFIQLKLERATPAERQLVFNEILQAAYQL
MVDVFGSYVIRKFFEFGSLEQKLALAERIRGHVLSLALQMYGSRVIEKA
LEFIPSDQQNEMVRELDGHVLKCVKDQNGNHVVQKCIECVQPQSLQFII
DAFKGQVFALSTHPYGSRVIRRILEHCLPDQTLPILEELHQHTEQLVQD
QYGSYVIEHVLEHGRPEDKSKIVAEIRGNVLVLSQHKFANNVVQKCVTH
ASRTERAVLIDEVCTMNDGPHSALYTMMKDQYASYVVRKMIDVAEPGQR
KIVMHKIRPHTEQLVQDQYGSYVIEHVLEHGRPEDKSKIVAEIRGNVLV
LSQHKFANNVVQKCVTHASRTERAVLIDEVCTMNDGPHSALYTMMKDQY
ASYVVRKMIDVAEPGQRKIVMHKIRPHIATLRKYTYGKHILAKLEKYYM
KNGVDLG.

In some aspects, SEQ ID NO: 486 comprises an architecture proceeding from the N-terminus to the C-terminus according to: R1′-R1-R2-R3-R4-R5-R1-R2-R3-R4-R5-R6-R7-R8-R6-R7-R8-R8′. In some aspects, SEQ ID NO: 486 is comprised of the sequences detailed in Table 30.

TABLE 30
16PUF protein according to SEQ ID NO: 486

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | C | HIMEFSQDQHGSRFIRLKLERATPAERQLVFNEILQ | 499 |
| R2 | G | AAYQLMVDVFGSYVIEKFFEFGSLEQKLALAERIRG | 491 |
| R3 | U | HVLSLALQMYGNRVIQKALEFIPSDQQNEMVRELDG | 506 |
| R4 | C | HVLKCVKDQNGSYVVRKCIECVQPQSLQFIIDAFKG | 505 |
| R5 | G | QVFALSTHPYGSRVIERILEHCLPDQTLPILEELHQ | 513 |
| R1 | U | HIMEFSQDQHGNRFIQLKLERATPAERQLVFNEILQ | 497 |
| R2 | C | AAYQLMVDVFGSYVIRKFFEFGSLEQKLALAERIRG | 492 |
| R3 | G | HVLSLALQMYGSRVIEKALEFIPSDQQNEMVRELDG | 507 |
| R4 | U | HVLKCVKDQNGNHVVQKCIECVQPQSLQFIIDAFKG | 503 |
| R5 | C | QVFALSTHPYGSRVIRRILEHCLPDQTLPILEELHQ | 514 |
| R6 | G | HTEQLVQDQYGSYVIEHVLEHGRPEDKSKIVAEIRG | 501 |
| R7 | U | NVLVLSQHKFANNVVQKCVTHASRTERAVLIDEVCTMND | 509 |
| | | GPHS | |
| R8 | C | ALYTMMKDQYASYVVRKMIDVAEPGQRKIVMHKIRP | 494 |
| R6 | G | HTEQLVQDQYGSYVIEHVLEHGRPEDKSKIVAEIRG | 501 |
| R7 | U | NVLVLSQHKFANNVVQKCVTHASRTERAVLIDEVCTMND | 509 |
| | | GPHS | |
| R8 | C | ALYTMMKDQYASYVVRKMIDVAEPGQRKIVMHKIRP | 494 |
| PUF R8′ | - | HIATLRKYTYGKHILAKLEKYYMKNGVDLG | 496 |

An exemplary 16-mer RNA recognition 16PUF targeting

(SEQ ID NO: 475)
CUGCUGCUGCUGCUGC

comprises the amino acid sequence:

(SEQ ID NO: 487)
GRSRLLEDFRNNRYPNLQLREIAGHIMEFSQDQHGSRFIRLKLERATPA
ERQLVFNEILQAAYQLMVDVFGSYVIEKFFEFGSLEQKLALAERIRGHV
LSLALQMYGNRVIQKALEFIPSDQQNEMVRELDGHVLKCVKDQNGSYVV
RKCIECVQPQSLQFIIDAFKGQVFALSTHPYGSRVIERILEHCLPDQTL
PILEELHQHTEQLVQDQYGNYVIQHVLEHGRPEDKSKIVAEIRGHIMEF
SQDQHGSRFIRLKLERATPAERQLVFNEILQAAYQLMVDVFGSYVIEKF
FEFGSLEQKLALAERIRGHVLSLALQMYGNRVIQKALEFIPSDQQNEMV
RELDGHVLKCVKDQNGSYVVRKCIECVQPQSLQFIIDAFKGQVFALSTH
PYGSRVIERILEHCLPDQTLPILEELHQHTEQLVQDQYGNYVIQHVLEH
GRPEDKSKIVAEIRGNVLVLSQHKFASYVVRKCVTHASRTERAVLIDEV
CTMNDGPHSALYTMMKDQYASYVVEKMIDVAEPGQRKIVMHKIRPNVLV
LSQHKFANNVVQKCVTHASRTERAVLIDEVCTMNDGPHSALYTMMKDQY
ASYVVRKMIDVAEPGQRKIVMHKIRPHIATLRKYTYGKHILAKLEKYYM
KNGVDLG

In some aspects, SEQ ID NO: 487 comprises an architecture proceeding from the N-terminus to the C-terminus according to: R1′-R1-R2-R3-R4-R5-R6-R1-R2-R3-R4-R5-R6-R7-R8-R7-R8-R8′. In some aspects, SEQ ID NO: 487 is comprised of the sequences detailed in Table 31.

TABLE 31
16PUF protein according to SEQ ID NO: 487

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | C | HIMEFSQDQHGSRFIRLKLERATPAERQLVFNEILQ | 498 |
| R2 | G | AAYQLMVDVFGSYVIEKFFEFGSLEQKLALAERIRG | 490 |
| R3 | U | HVLSLALQMYGNRVIQKALEFIPSDQQNEMVRELDG | 508 |
| R4 | C | HVLKCVKDQNGSYVVRKCIECVQPQSLQFIIDAFKG | 504 |
| R5 | G | QVFALSTHPYGSRVIERILEHCLPDQTLPILEELHQ | 512 |
| R6 | U | HTEQLVQDQYGNYVIQHVLEHGRPEDKSKIVAEIRG | 500 |
| R1 | C | HIMEFSQDQHGSRFIRLKLERATPAERQLVFNEILQ | 499 |
| R2 | G | AAYQLMVDVFGSYVIEKFFEFGSLEQKLALAERIRG | 491 |
| R3 | U | HVLSLALQMYGNRVIQKALEFIPSDQQNEMVRELDG | 506 |
| R4 | C | HVLKCVKDQNGSYVVRKCIECVQPQSLQFIIDAFKG | 505 |
| R5 | G | QVFALSTHPYGSRVIERILEHCLPDQTLPILEELHQ | 513 |
| R6 | U | HTEQLVQDQYGNYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| R7 | U | NVLVLSQHKFANNVVQKCVTHASRTERAVLIDEVCTMND | 509 |
| | | GPHS | |
| R8 | C | ALYTMMKDQYASYVVRKMIDVAEPGQRKIVMHKIRP | 494 |
| PUF R8′ | — | HIATLRKYTYGKHILAKLEKYYMKNGVDLG | 496 |

An exemplary 16-mer RNA recognition (16PUF) targeting

(SEQ ID NO: 475)
CUGCUGCUGCUGCUGC

comprises ne amino acid sequence:

(SEQ ID NO: 488)
GRSRLLEDFRNNRYPNLQLREIAGHIMEFSQDQHGSRFIRLKLERATPA
ERQLVFNEILQAAYQLMVDVFGSYVIEKFFEFGSLEQKLALAERIRGHV
LSLALQMYGNRVIQKALEFIPSDQQNEMVRELDGHVLKCVKDQNGSYVV
RKCIECVQPQSLQFIIDAFKGQVFALSTHPYGSRVIERILEHCLPDQTL
PILEELHQHTEQLVQDQYGNYVIQHVLEHGRPEDKSKIVAEIRGNVLVL
SQHKFASYVVRKCVTHASRTERAVLIDEVCTMNDGPHSALYTMMKDQYA
SYVVEKMIDVAEPGQRKIVMHKIRPHIMEFSQDQHGNRFIQLKLERATP
AERQLVFNEILQAAYQLMVDVFGSYVIRKFFEFGSLEQKLALAERIRGH
VLSLALQMYGSRVIEKALEFIPSDQQNEMVRELDGHVLKCVKDQNGNHV
VQKCIECVQPQSLQFIIDAFKGQVFALSTHPYGSRVIRRILEHCLPDQT
LPILEELHQHTEQLVQDQYGSYVIEHVLEHGRPEDKSKIVAEIRGNVLV
LSQHKFANNVVQKCVTHASRTERAVLIDEVCTMNDGPHSALYTMMKDQY
ASYVVRKMIDVAEPGQRKIVMHKIRPHIATLRKYTYGKHILAKLEKYYM
KNGVDLG

In some aspects, SEQ ID NO: 488 comprises an architecture proceeding from the N-terminus to the C-terminus according to: R1′-R1-R2-R3-R4-R5-R6-R7-R8-R1-R2-R3-R4-R5-R6-R7-R8-R8′. In some aspects, SEQ ID NO: 488 is comprised of the sequences detailed in Table 32.

TABLE 32
16PUF protein according to SEQ ID NO: 488

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | C | HIMEFSQDQHGSRFIRLKLERATPAERQLVFNEILQ | 498 |
| R2 | G | AAYQLMVDVFGSYVIEKFFEFGSLEQKLALAERIRG | 490 |
| R3 | U | HVLSLALQMYGNRVIQKALEFIPSDQQNEMVRELDG | 508 |
| R4 | C | HVLKCVKDQNGSYVVRKCIECVQPQSLQFIIDAFKG | 504 |
| R5 | G | QVFALSTHPYGSRVIERILEHCLPDQTLPILEELHQ | 512 |
| R6 | U | HTEQLVQDQYGNYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| R1 | U | HIMEFSQDQHGNRFIQLKLERATPAERQLVFNEILQ | 497 |
| R2 | C | AAYQLMVDVFGSYVIRKFFEFGSLEQKLALAERIRG | 492 |
| R3 | G | HVLSLALQMYGSRVIEKALEFIPSDQQNEMVRELDG | 507 |
| R4 | U | HVLKCVKDQNGNHVVQKCIECVQPQSLQFIIDAFKG | 503 |
| R5 | C | QVFALSTHPYGSRVIRRILEHCLPDQTLPILEELHQ | 514 |
| R6 | G | HTEQLVQDQYGSYVIEHVLEHGRPEDKSKIVAEIRG | 501 |
| R7 | U | NVLVLSQHKFANNVVQKCVTHASRTERAVLIDEVCTMND | 509 |
| | | GPHS | |
| R8 | C | ALYTMMKDQYASYVVRKMIDVAEPGQRKIVMHKIRP | 494 |
| PUF R8′ | — | HIATLRKYTYGKHILAKLEKYYMKNGVDLG | 496 |

An exemplary 8-mer RNA recognition (8PUF) targeting

(SEQ ID NO: 476)
GCUGCUGC

comprises the amino acid sequence:

(SEQ ID NO: 549)
GRSRLLEDFRNNRYPNLQLREIAGHIMEFSQDQHGSRFIRLKLERATPA
ERQLVFNEILQAAYQLMVDVFGSYVIEKFFEFGSLEQKLALAERIRGHV
LSLALQMYGNRVIQKALEFIPSDQQNEMVRELDGHVLKCVKDQNGSYVV
RKCIECVQPQSLQFIIDAFKGQVFALSTHPYGSRVIERILEHCLPDQTL
PILEELHQHTEQLVQDQYGNYVIQHVLEHGRPEDKSKIVAEIRGNVLVL
SQHKFASYVVRKCVTHASRTERAVLIDEVCTMNDGPHSALYTMMKDQYA
SYVVEKMIDVAEPGQRKIVMHKIRPHIATLRKYTYGKHILAKLEKYYMK
NGVDLG.

In some aspects, SEQ ID NO: 549 comprises an architecture proceeding from the N-terminus to the C-terminus according to: R1′-R1-R2-R3-R4-R5-R6-R1-R2-R3-R4-R5-R6-R7-R8-R8′. In some aspects, SEQ ID NO: 549 is comprised of the sequences detailed in Table 33.

TABLE 33
8PUF protein according to SEQ ID NO: 549

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | C | HIMEFSQDQHGSRFIRLKLERATPAERQLVFNEILQ | 499 |
| R2 | G | AAYQLMVDVFGSYVIEKFFEFGSLEQKLALAERIRG | 491 |
| R3 | U | HVLSLALQMYGNRVIQKALEFIPSDQQNEMVRELDG | 506 |
| R4 | C | HVLKCVKDQNGSYVVRKCIECVQPQSLQFIIDAFKG | 505 |
| R5 | G | QVFALSTHPYGSRVIERILEHCLPDQTLPILEELHQ | 513 |
| R6 | U | HTEQLVQDQYGNYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 14-mer RNA recognition (14PUF) targeting

(SEQ ID NO: 477)
GCUGCUGCUGCUGC

comprises the amino acid sequence:

(SEQ ID NO: 550)
GRSRLLEDFRNNRYPNLQLREIAGHIMEFSQDQHGSRFIRLKLERATPA
ERQLVFNEILQAAYQLMVDVFGSYVIEKFFEFGSLEQKLALAERIRGHV
LSLALQMYGNRVIQKALEFIPSDQQNEMVRELDGHVLKCVKDQNGSYVV
RKCIECVQPQSLQFIIDAFKGQVFALSTHPYGSRVIERILEHCLPDQTL
PILEELHQHIMEFSQDQHGNRFIQLKLERATPAERQLVFNEILQAAYQL
MVDVFGSYVIRKFFEFGSLEQKLALAERIRGHVLSLALQMYGSRVIEKA
LEFIPSDQQNEMVRELDGHVLKCVKDQNGNHVVQKCIECVQPQSLQFII
DAFKGQVFALSTHPYGSRVIRRILEHCLPDQTLPILEELHQHTEQLVQD
QYGSYVIEHVLEHGRPEDKSKIVAEIRGHTEQLVQDQYGNYVIQHVLEH
GRPEDKSKIVAEIRGNVLVLSQHKFASYVVRKCVTHASRTERAVLIDEV
CTMNDGPHSALYTMMKDQYASYVVEKMIDVAEPGQRKIVMHKIRPHIAT
LRKYTYGKHILAKLEKYYMKNGVDLG.

In some aspects, SEQ ID NO: 550 comprises an architecture proceeding from the N-terminus to the C-terminus according to: R1′-R-R2-R3-R4-R5-R1-R2-R3-R4-R5-R6-R6-R7-R8-R8′. In some aspects, SEQ ID NO: 550 is comprised of the sequences detailed in Table 34.

TABLE 34
14PUF protein according to SEQ ID NO: 550

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | C | HIMEFSQDQHGSRFIRLKLERATPAERQLVFNEILQ | 499 |
| R2 | G | AAYQLMVDVFGSYVIEKFFEFGSLEQKLALAERIRG | 491 |
| R3 | U | HVLSLALQMYGNRVIQKALEFIPSDQQNEMVRELDG | 506 |
| R4 | C | HVLKCVKDQNGSYVVRKCIECVQPQSLQFIIDAFKG | 505 |
| R5 | G | QVFALSTHPYGSRVIERILEHCLPDQTLPILEELHQ | 513 |
| R1 | U | HIMEFSQDQHGNRFIQLKLERATPAERQLVFNEILQ | 497 |
| R2 | C | AAYQLMVDVFGSYVIRKFFEFGSLEQKLALAERIRG | 492 |
| R3 | G | HVLSLALQMYGSRVIEKALEFIPSDQQNEMVRELDG | 507 |
| R4 | U | HVLKCVKDQNGNHVVQKCIECVQPQSLQFIIDAFKG | 503 |
| R5 | C | QVFALSTHPYGSRVIRRILEHCLPDQTLPILEELHQ | 514 |
| R6 | G | HTEQLVQDQYGSYVIEHVLEHGRPEDKSKIVAEIRG | 501 |
| R6 | U | HTEQLVQDQYGNYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 14-mer RNA recognition (14PUF) targeting

(SEQ ID NO: 477)
GCUGCUGCUGCUGC

comprises the amino acid sequence:

(SEQ ID NO: 551)
GRSRLLEDFRNNRYPNLQLREIAGHIMEFSQDQHGSRFIRLKLERATPA
ERQLVFNEILQAAYQLMVDVFGSYVIEKFFEFGSLEQKLALAERIRGHV
LSLALQMYGNRVIQKALEFIPSDQQNEMVRELDGHVLKCVKDQNGSYVV
RKCIECVQPQSLQFIIDAFKGQVFALSTHPYGSRVIERILEHCLPDQTL
PILEELHQHTEQLVQDQYGNYVIQHVLEHGRPEDKSKIVAEIRGHIMEF
SQDQHGSRFIRLKLERATPAERQLVFNEILQAAYQLMVDVFGSYVIEKF
FEFGSLEQKLALAERIRGHVLSLALQMYGNRVIQKALEFIPSDQQNEMV
RELDGHVLKCVKDQNGSYVVRKCIECVQPQSLQFIIDAFKGQVFALSTH
PYGSRVIERILEHCLPDQTLPILEELHQHTEQLVQDQYGNYVIQHVLEH
GRPEDKSKIVAEIRGNVLVLSQHKFASYVVRKCVTHASRTERAVLIDEV
CTMNDGPHSALYTMMKDQYASYVVEKMIDVAEPGQRKIVMHKIRPHIAT
LRKYTYGKHILAKLEKYYMKNGVDLG.

In some aspects, SEQ ID NO: 551 comprises an architecture proceeding from the N-terminus to the C-terminus according to: R1′-R1-R2-R3-R4-R5-R6-R1-R2-R3-R4-R5-R6-R7-R8-R8′. In some aspects, SEQ ID NO: 551 is comprised of the sequences detailed in Table 35.

TABLE 35
14PUF protein according to SEQ ID NO: 551

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | C | HIMEFSQDQHGSRFIRLKLERATPAERQLVFNEILQ | 499 |
| R2 | G | AAYQLMVDVFGSYVIEKFFEFGSLEQKLALAERIRG | 491 |
| R3 | U | HVLSLALQMYGNRVIQKALEFIPSDQQNEMVRELDG | 506 |
| R4 | C | HVLKCVKDQNGSYVVRKCIECVQPQSLQFIIDAFKG | 505 |
| R5 | G | QVFALSTHPYGSRVIERILEHCLPDQTLPILEELHQ | 513 |
| R6 | U | HTEQLVQDQYGNYVIQHVLEHGRPEDKSKIVAEIRG | 500 |
| R1 | C | HIMEFSQDQHGSRFIRLKLERATPAERQLVFNEILQ | 499 |
| R2 | G | AAYQLMVDVFGSYVIEKFFEFGSLEQKLALAERIRG | 491 |
| R3 | U | HVLSLALQMYGNVIQKALEFIPSDQQNEMVRELDG | 506 |
| R4 | C | HVLKCVKDQNGSYVVRKCIECVQPQSLQFIIDAFKG | 505 |
| R5 | G | QVFALSTHPYGSRVIERILEHCLPDQTLPILEELHQ | 513 |
| R6 | U | HTEQLVQDQYGNYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 15-mer RNA recognition (15PUF) targeting

(SEQ ID NO: 478)
GCUGCUGCUGCUGCU

comprises the amino acid sequence:

(SEQ ID NO: 552)
GRSRLLEDFRNNRYPNLQLREIAGHIMEFSQDQHGNRFIQLKLERATPA
ERQLVFNEILQAAYQLMVDVFGSYVIRKFFEFGSLEQKLALAERIRGHV
LSLALQMYGSRVIEKALEFIPSDQQNEMVRELDGHVLKCVKDQNGNHVV
QKCIECVQPQSLQFIIDAFKGQVFALSTHPYGSRVIRRILEHCLPDQTL
PILEELHQHIMEFSQDQHGSRFIELKLERATPAERQLVFNEILQAAYQL
MVDVFGNYVIQKFFEFGSLEQKLALAERIRGHVLSLALQMYGSYVIRKA
LEFIPSDQQNEMVRELDGHVLKCVKDQNGSYVVEKCIECVQPQSLQFII
DAFKGQVFALSTHPYGNRVIQRILEHCLPDQTLPILEELHQHTEQLVQD
QYGSYVIRHVLEHGRPEDKSKIVAEIRGNVLVLSQHKFASNVVEKCVTH
ASRTERAVLIDEVCTMNDGPHSHTEQLVQDQYGNYVIQHVLEHGRPEDK
SKIVAEIRGNVLVLSQHKFASYVVRKCVTHASRTERAVLIDEVCTMNDG
PHSALYTMMKDQYASYVVEKMIDVAEPGQRKIVMHKIRPHIATLRKYTY
GKHILAKLEKYYMKNGVDLG.

In some aspects, SEQ ID NO: 552 comprises an architecture proceeding from the N-terminus to the C-terminus according to: R1′-R1-R2-R3-R4-R5-R1-R2-R3-R4-R5-R6-R7-R6-R7-R8-R8′. In some aspects, SEQ ID NO: 552 is comprised of the sequences detailed in Table 36.

TABLE 36
15PUF protein according to SEQ ID NO: 552

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | U | HIMEFSQDQHGNRFIQLKLERATPAERQLVFNEILQ | 499 |
| R2 | C | AAYQLMVDVFGSYVIRKFFEFGSLEQKLALAERIRG | 491 |
| R3 | G | HVLSLALQMYGSRVIEKALEFIPSDQQNEMVRELDG | 506 |
| R4 | U | HVLKCVKDQNGNHVVQKCIECVQPQSLQFIIDAFKG | 505 |
| R5 | C | QVFALSTHPYGSRVIRRILEHCLPDQTLPILEELHQ | 513 |
| R1 | G | HIMEFSQDQHGSRFIELKLERATPAERQLVFNEILQ | 498 |
| R2 | U | AAYQLMVDVFGNYVIQKFFEFGSLEQKLALAERIRG | 490 |
| R3 | C | HVLSLALQMYGSYVIRKALEFIPSDQQNEMVRELDG | 508 |
| R4 | G | HVLKCVKDQNGSYVVEKCIECVQPQSLQFIIDAFKG | 54 |
| R5 | U | QVFALSTHPYGNRVIQRILEHCLPDQTLPILEELHQ | 512 |
| R6 | C | HTEQLVQDQYGSYVIRHVLEHGRPEDKSKIVAEIRG | 502 |
| R7 | G | NVLVLSQHKFASNVVEKCVTHASRTERAVLIDEVCTMNDG | 510 |
| | | PHS | |
| R6 | U | HTEQLVQDQYGNYVIQHVLEHGRPEDKSKIVAEIRG | 50 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 15-mer RNA recognition (15PUF) targeting

(SEQ ID NO: 478)
GCUGCUGCUGCUGCU

comprises the amino acid sequence:

(SEQ ID NO: 553)
GRSRLLEDFRNNRYPNLQLREIAGHIMEFSQDQHGNRFIQLKLERATPA
ERQLVFNEILQAAYQLMVDVFGSYVIRKFFEFGSLEQKLALAERIRGHV
LSLALQMYGSRVIEKALEFIPSDQQNEMVRELDGHVLKCVKDQNGNHVV
QKCIECVQPQSLQFIIDAFKGQVFALSTHPYGSRVIRRILEHCLPDQTL
PILEELHQHTEQLVQDQYGSYVIEHVLEHGRPEDKSKIVAEIRGHIMEF
SQDQHGNRFIQLKLERATPAERQLVFNEILQAAYQLMVDVFGSYVIRKF
FEFGSLEQKLALAERIRGHVLSLALQMYGSRVIEKALEFIPSDQQNEMV
RELDGHVLKCVKDQNGNHVVQKCIECVQPQSLQFIIDAFKGQVFALSTH
PYGSRVIRRILEHCLPDQTLPILEELHQHTEQLVQDQYGSYVIEHVLEH
GRPEDKSKIVAEIRGNVLVLSQHKFANNVVQKCVTHASRTERAVLIDEV
CTMNDGPHSNVLVLSQHKFASYVVRKCVTHASRTERAVLIDEVCTMNDG
PHSALYTMMKDQYASYVVEKMIDVAEPGQRKIVMHKIRPHIATLRKYTY
GKHILAKLEKYYMKNGVDLG.

In some aspects, SEQ ID NO: 553 comprises an architecture proceeding from the N-terminus to the C-terminus according to: R1′-R1-R2-R3-R4-R5-R6-R1-R2-R3-R4-R5-R6-R7-R7-R8-R8′. In some aspects, SEQ ID NO: 553 is comprised of the sequences detailed in Table 37.

TABLE 37
15PUF protein according to SEQ ID NO: 553

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | U | HIMEFSQDQHGNRFIQLKLERATPAERQLVFNEILQ | 499 |
| R2 | C | AAYQLMVDVFGSYVIRKFFEFGSLEQKLALAERIRG | 491 |
| R3 | G | HVLSLALQMYGSRVIEKALEFIPSDQQNEMVRELDG | 506 |
| R4 | U | HVLKCVKDQNGNHVVQKCIECVQPQSLQFIIDAFKG | 505 |
| R5 | C | QVFALSTHPYGSRVIRRILEHCLPDQTLPILEELHQ | 513 |
| R6 | G | HTEQLVQDQYGSYVIEHVLEHGRPEDKSKIVAEIRG | 500 |
| R1 | U | HIMEFSQDQHGNRFIQLKLERATPAERQLVFNEILQ | 497 |
| R2 | C | AAYQLMVDVFGSYVIRKFFEFGSLEQKLALAERIRG | 492 |
| R3 | G | HVLSLALQMYGSRVIEKALEFIPSDQQNEMVRELDG | 507 |
| R4 | U | HVLKCVKDQNGNHVVQKCIECVQPQSLQFIIDAFKG | 503 |
| R5 | C | QVFALSTHPYGSRVIRRILEHCLPDQTLPILEELHQ | 514 |
| R6 | G | HTEQLVQDQYGSYVIEHVLEHGRPEDKSKIVAEIRG | 501 |
| R7 | U | NVLVLSQHKFANNVVQKCVTHASRTERAVLIDEVCTMND | 509 |
| | | GPHS | |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 15-mer RNA recognition (15PUF) targeting

(SEQ ID NO: 478)
GCUGCUGCUGCUGCU

comprises the amino acid sequence:

(SEQ ID NO: 554)
GRSRLLEDFRNNRYPNLQLREIAGHIMEFSQDQHGNRFIQLKLERATPA
ERQLVFNEILQAAYQLMVDVFGSYVIRKFFEFGSLEQKLALAERIRGHV
LSLALQMYGSRVIEKALEFIPSDQQNEMVRELDGHVLKCVKDQNGNHVV
QKCIECVQPQSLQFIIDAFKGQVFALSTHPYGSRVIRRILEHCLPDQTL
PILEELHQHTEQLVQDQYGSYVIEHVLEHGRPEDKSKIVAEIRGNVLVL
SQHKFANNVVQKCVTHASRTERAVLIDEVCTMNDGPHSHIMEFSQDQHG
SRFIRLKLERATPAERQLVFNEILQAAYQLMVDVFGSYVIEKFFEFGSL
EQKLALAERIRGHVLSLALQMYGNRVIQKALEFIPSDQQNEMVRELDGH
VLKCVKDQNGSYVVRKCIECVQPQSLQFIIDAFKGQVFALSTHPYGSRV
IERILEHCLPDQTLPILEELHQHTEQLVQDQYGNYVIQHVLEHGRPEDK
SKIVAEIRGNVLVLSQHKFASYVVRKCVTHASRTERAVLIDEVCTMNDG
PHSALYTMMKDQYASYVVEKMIDVAEPGQRKIVMHKIRPHIATLRKYTY
GKHILAKLEKYYMKNGVDLG.

In some aspects, SEQ ID NO: 554 comprises an architecture proceeding from the N-terminus to the C-terminus according to: R1′-R1-R2-R3-R4-R5-R6-R7-R1-R2-R3-R4-R5-R6-R7-R8-R8′. In some aspects, SEQ ID NO: 554 is comprised of the sequences detailed in Table 38.

TABLE 38
15PUF protein according to SEQ ID NO: 554

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | U | HIMEFSQDQHGNRFIQLKLERATPAERQLVFNEILQ | 499 |
| R2 | C | AAYQLMVDVFGSYVIRKFFEFGSLEQKLALAERIRG | 491 |
| R3 | G | HVLSLALQMYGSRVIEKALEFIPSDQQNEMVRELDG | 506 |
| R4 | U | HVLKCVKDQNGNHVVQKCIECVQPQSLQFIIDAFKG | 505 |
| R5 | C | QVFALSTHPYGSRVIRRILEHCLPDQTLPILEELHQ | 513 |
| R6 | G | HTEQLVQDQYGSYVIEHVLEHGRPEDKSKIVAEIRG | 500 |
| R7 | U | NVLVLSQHKFANNVVQKCVTHASRTERAVLIDEVCTMND | 509 |
| | | GPHS | |
| R1 | C | HIMEFSQDQHGSRFIRLKLERATPAERQLVFNEILQ | 499 |
| R2 | G | AAYQLMVDVFGSYVIEKFFEFGSLEQKLALAERIRG | 491 |
| R3 | U | HVLSLALQMYGNRVIQKALEFIPSDQQNEMVRELDG | 506 |
| R4 | C | HVLKCVKDQNGSYVVRKCIECVQPQSLQFIIDAFKG | 505 |
| R5 | G | QVFALSTHPYGSRVIERILEHCLPDQTLPILEELHQ | 513 |
| R6 | U | HTEQLVQDQYGNYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 16-mer RNA recognition (16PUF) targeting

(SEQ ID NO: 479)
GCUGCUGCUGCUGCUG

comprises the amino acid sequence:

(SEQ ID NO: 555)
GRSRLLEDFRNNRYPNLQLREIAGHIMEFSQDQHGSRFIELKLERATPA
ERQLVFNEILQAAYQLMVDVFGNYVIQKFFEFGSLEQKLALAERIRGHV
LSLALOMYGSYVIRKALEFIPSDQQNEMVRELDGHVLKCVKDQNGSYVV
EKCIECVQPQSLQFIIDAFKGQVFALSTHPYGNRVIQRILEHCLPDQTL
PILEELHQHIMEFSQDQHGSRFIRLKLERATPAERQLVFNEILQAAYQL
MVDVFGSYVIEKFFEFGSLEQKLALAERIRGHVLSLALQMYGNRVIQKA
LEFIPSDQQNEMVRELDGHVLKCVKDQNGSYVVRKCIECVQPQSLQFII
DAFKGQVFALSTHPYGSRVIERILEHCLPDQTLPILEELHQHTEQLVQD
QYGNYVIQHVLEHGRPEDKSKIVAEIRGNVLVLSQHKFASYVVRKCVTH
ASRTERAVLIDEVCTMNDGPHSALYTMMKDQYASYVVEKMIDVAEPGQR
KIVMHKIRPHTEQLVQDQYGNYVIQHVLEHGRPEDKSKIVAEIRGNVLV
LSQHKFASYVVRKCVTHASRTERAVLIDEVCTMNDGPHSALYTMMKDQY
ASYVVEKMIDVAEPGQRKIVMHKIRPHIATLRKYTYGKHILAKLEKYYM
KNGVDLG.

In some aspects, SEQ ID NO: 555 comprises an architecture proceeding from the N-terminus to the C-terminus according to: R1′-R1-R2-R3-R4-R5-R1-R2-R3-R4-R5-R6-R7-R8-R6-R7-R8-R8′. In some aspects, SEQ ID NO: 555 is comprised of the sequences detailed in Table 39.

TABLE 39
16PUF protein according to SEQ ID NO: 555

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | G | HIMEFSQDQHGSRFIELKLERATPAERQLVFNEILQ | 499 |
| R2 | U | AAYQLMVDVFGNYVIQKFFEFGSLEQKLALAERIRG | 491 |
| R3 | C | HVLSLALQMYGSYVIRKALEFIPSDQQNEMVRELDG | 506 |
| R4 | G | HVLKCVKDQNGSYVVEKCIECVQPQSLQFIIDAFKG | 505 |
| R5 | U | QVFALSTHPYGNRVIQRILEHCLPDQTLPILEELHQ | 513 |
| R1 | C | HIMEFSQDQHGSRFIRLKLERATPAERQLVFNEILQ | 499 |
| R2 | G | AAYQLMVDVFGSYVIEKFFEFGSLEQKLALAERIRG | 491 |
| R3 | U | HVLSLALQMYGNRVIQKALEFIPSDQQNEMVRELDG | 506 |
| R4 | C | HVLKCVKDQNGSYVVRKCIECVQPQSLQFIIDAFKG | 505 |
| R5 | G | QVFALSTHPYGSRVIERILEHCLPDQTLPILEELHQ | 513 |
| R6 | U | HTEQLVQDQYGNYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| R6 | U | HTEQLVQDQYGNYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 16-mer RNA recognition (16PUF) targeting

(SEQ ID NO: 479)
GCUGCUGCUGCUGCUG

comprises the amino acid sequence:

(SEQ ID NO: 556)
GRSRLLEDFRNNRYPNLQLREIAGHIMEFSQDQHGSRFIELKLERATPA
ERQLVFNEILQAAYQLMVDVFGNYVIQKFFEFGSLEQKLALAERIRGHV
LSLALQMYGSYVIRKALEFIPSDQQNEMVRELDGHVLKCVKDQNGSYVV
EKCIECVQPQSLQFIIDAFKGQVFALSTHPYGNRVIQRILEHCLPDQTL
PILEELHQHTEQLVQDQYGSYVIRHVLEHGRPEDKSKIVAEIRGHIMEF
SQDQHGSRFIELKLERATPAERQLVFNEILQAAYQLMVDVFGNYVIQKF
FEFGSLEQKLALAERIRGHVLSLALQMYGSYVIRKALEFIPSDQQNEMV
RELDGHVLKCVKDQNGSYVVEKCIECVQPQSLQFIIDAFKGQVFALSTH
PYGNRVIQRILEHCLPDQTLPILEELHQHTEQLVQDQYGSYVIRHVLEH
GRPEDKSKIVAEIRGNVLVLSQHKFASNVVEKCVTHASRTERAVLIDEV
CTMNDGPHSALYTMMKDQYANYVVQKMIDVAEPGQRKIVMHKIRPNVLV
LSQHKFASYVVRKCVTHASRTERAVLIDEVCTMNDGPHSALYTMMKDQY
ASYVVEKMIDVAEPGQRKIVMHKIRPHIATLRKYTYGKHILAKLEKYYM
KNGVDLG

In some aspects, SEQ ID NO: 556 comprises an architecture proceeding from the N-terminus to the C-terminus according to: R1′-R1-R2-R3-R4-R5-R6-R1-R2-R3-R4-R5-R6-R7-R8-R7-R8-R8′. In some aspects, SEQ ID NO: 556 is comprised of the sequences detailed in Table 40.

TABLE 40
16PUF protein according to SEQ ID NO: 556

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | G | HIMEFSQDQHGSRFIELKLERATPAERQLVFNEILQ | 499 |
| R2 | U | AAYQLMVDVFGNYVIQKFFEFGSLEQKLALAERIRG | 491 |
| R3 | C | HVLSLALQMYGSYVIRKALEFIPSDQQNEMVRELDG | 506 |
| R4 | G | HVLKCVKDQNGSYVVEKCIECVQPQSLQFIIDAFKG | 505 |
| R5 | U | QVFALSTHPYGNRVIQRILEHCLPDQTLPILEELHQ | 513 |
| R6 | C | HTEQLVQDQYGSYVIRHVLEHGRPEDKSKIVAEIRG | 500 |
| R1 | G | HIMEFSQDQHGSRFIELKLERATPAERQLVFNEILQ | 498 |
| R2 | U | AAYQLMVDVFGNYVIQKFFEFGSLEQKLALAERIRG | 490 |
| R3 | C | HVLSLALQMYGSYVIRKALEFIPSDQQNEMVRELDG | 508 |
| R4 | G | HVLKCVKDQNGSYVVEKCIECVQPQSLQFIIDAFKG | 504 |
| R5 | U | QVFALSTHPYGNRVIQRILEHCLPDQTLPILEELHQ | 512 |
| R6 | C | HTEQLVQDQYGSYVIRHVLEHGRPEDKSKIVAEIRG | 52 |
| R7 | G | NVLVLSQHKFASNVVEKCVTHASRTERAVLIDEVCTMNDG | 510 |
| | | PHS | |
| R8 | U | ALYTMMKDQYANYVVQKMIDVAEPGQRKIVMHKIRP | 493 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 16-mer RNA recognition (16PUF) targeting

(SEQ ID NO: 479)
GCUGCUGCUGCUGCUG

comprises the amino acid sequence:

(SEQ ID NO: 557)
GRSRLLEDFRNNRYPNLQLREIAGHIMEFSQDQHGSRFIELKLERATPA
ERQLVFNEILQAAYQLMVDVFGNYVIQKFFEFGSLEQKLALAERIRGHV
LSLALQMYGSYVIRKALEFIPSDQQNEMVRELDGHVLKCVKDQNGSYVV
EKCIECVQPQSLQFIIDAFKGQVFALSTHPYGNRVIQRILEHCLPDQTL
PILEELHQHTEQLVQDQYGSYVIRHVLEHGRPEDKSKIVAEIRGNVLVL
SQHKFASNVVEKCVTHASRTERAVLIDEVCTMNDGPHSALYTMMKDQYA
NYVVQKMIDVAEPGQRKIVMHKIRPHIMEFSQDQHGSRFIRLKLERATP
AERQLVFNEILQAAYQLMVDVFGSYVIEKFFEFGSLEQKLALAERIRGH
VLSLALQMYGNRVIQKALEFIPSDQQNEMVRELDGHVLKCVKDQNGSYV
VRKCIECVQPQSLQFIIDAFKGQVFALSTHPYGSRVIERILEHCLPDQT
LPILEELHQHTEQLVQDQYGNYVIQHVLEHGRPEDKSKIVAEIRGNVLV
LSQHKFASYVVRKCVTHASRTERAVLIDEVCTMNDGPHSALYTMMKDQY
ASYVVEKMIDVAEPGQRKIVMHKIRPHIATLRKYTYGKHILAKLEKYYM
KNGVDLG.

In some aspects, SEQ ID NO: 557 comprises an architecture proceeding from the N-terminus to the C-terminus according to: R1′-R1-R2-R3-R4-R5-R6-R7-R8-R1-R2-R3-R4-R5-R6-R7-R8-R8′. In some aspects, SEQ ID NO: 557 is comprised of the sequences detailed in Table 41.

TABLE 41
16PUF protein according to SEQ ID NO: 557

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | G | HIMEFSQDQHGSRFIELKLERATPAERQLVFNEILQ | 499 |
| R2 | U | AAYQLMVDVFGNYVIQKFFEFGSLEQKLALAERIRG | 491 |
| R3 | C | HVLSLALQMYGSYVIRKALEFIPSDQQNEMVRELDG | 506 |
| R4 | G | HVLKCVKDQNGSYVVEKCIECVQPQSLQFIIDAFKG | 505 |
| R5 | U | QVFALSTHPYGNRVIQRILEHCLPDQTLPILEELHQ | 513 |
| R6 | C | HTEQLVQDQYGSYVIRHVLEHGRPEDKSKIVAEIRG | 500 |
| R7 | G | NVLVLSQHKFASNVVEKCVTHASRTERAVLIDEVCTMNDG | 510 |
| | | PHS | |
| R8 | U | ALYTMMKDQYANYVVQKMIDVAEPGQRKIVMHKIRP | 493 |
| R1 | C | HIMEFSQDQHGSRFIRLKLERATPAERQLVFNEILQ | 499 |
| R2 | G | AAYQLMVDVFGSYVIEKFFEFGSLEQKLALAERIRG | 491 |
| R3 | U | HVLSLALQMYGNRVIQKALEFIPSDQQNEMVRELDG | 506 |
| R4 | C | HVLKCVKDQNGSYVVRKCIECVQPQSLQFIIDAFKG | 505 |
| R5 | G | QVFALSTHPYGSRVIERILEHCLPDQTLPILEELHQ | 513 |
| R6 | U | HTEQLVQDQYGNYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

In some aspects, fusion proteins of the disclosure comprise a PUF according to SEQ ID NOs: 444-451, 461, 480-488, 547-558, 570, or 638-649. In some aspects, fusion proteins of the disclosure comprise a PUF according to SEQ ID NO: 444. In some aspects, fusion proteins of the disclosure comprise, from N-terminus to C-terminus: a human NLS sequence, a PUF according to SEQ ID NO: 444; a linker sequence; and an endonuclease. In some aspects, an exemplary 8PUF targeting CUG fusion protein of the disclosure is arranged from N-terminus to C-terminus according to elements listed in any one of Tables 42-50. In some embodiments, a CUG-targeting fusion protein comprising an 8PUF protein of the disclosure comprises SEQ ID NO: 559. In some embodiments, a CUG-targeting fusion protein comprising an 14PUF protein of the disclosure comprises SEQ ID NO: 560. In some embodiments, a CUG-targeting fusion protein comprising an 14PUF protein of the disclosure comprises SEQ ID NO: 561. In some embodiments, a CUG-targeting fusion protein comprising an 15PUF protein of the disclosure comprises SEQ ID NO: 562. In some embodiments, a CUG-targeting fusion protein comprising an 15PUF protein of the disclosure comprises SEQ ID NO: 563. In some embodiments, a CUG-targeting fusion protein comprising an 15PUF protein of the disclosure comprises SEQ ID NO: 567. In some embodiments, a CUG-targeting fusion protein comprising an 16PUF protein of the disclosure comprises SEQ ID NO: 565. In some embodiments, a CUG-targeting fusion protein comprising an 16PUF protein of the disclosure comprises SEQ ID NO: 566. In some embodiments, a CUG-targeting fusion protein comprising an 16PUF protein of the disclosure comprises SEQ ID NO: 567.

TABLE 42
Exemplary 8PUF targeting CUG Fusion Protein

| Plasmid | RNA | |
|---|---|---|
| Element | Recognition | Amino Acid Sequence |
| SV40 NLS | — | PKKKRKV (SEQ ID NO: 437) |
| 8PUF | UGCUGCUG | GRSRLLEDFRNNRYPNLQLREIAGHIMEFSQDQHGSRFIE |
| | | LKLERATPAERQLVFNEILQAAYQLMVDVFGNYVIQKFF |
| | | EFGSLEQKLALAERIRGHVLSLALQMYGSYVIRKALEFIP |
| | | SDQQNEMVRELDGHVLKCVKDQNGSYVVEKCIECVQP |
| | | QSLQFIIDAFKGQVFALSTHPYGNRVIQRILEHCLPDQTLP |
| | | ILEELHQHTEQLVQDQYGSYVIRHVLEHGRPEDKSKIVA |
| | | EIRGNVLVLSQHKFASNVVEKCVTHASRTERAVLIDEVC |
| | | TMNDGPHSALYTMMKDQYANYVVQKMIDVAEPGQRKI |
| | | VMHKIRPHIATLRKYTYGKHILAKLEKYYMKNGVDLG |
| | | (SEQ ID NO: 444) |
| Linker | — | VDTANGS (SEQ ID NO: 411) |
| E17 | — | GGGTPKAPNLEPPLPEEEKEGSDLRPVVIDGSNVAMSHG |
| endonuclease | | NKEVFSCRGILLAVNWFLERGHTDITVFVPSWRKEQPRP |
| | | DVPITDQHILRELEKKKILVFTPSRRVGGKRVVCYDDRFI |
| | | VKLAYESDGIVVSNDTYRDLQGERQEWKRFIEERLLMYS |
| | | FVNDKFMPPDDPLGRHGPSLDNFLRKKPLTLE (SEQ ID |
| | | NO: 358) |

TABLE 43
Exemplary 14PUF targeting CUG Fusion Protein

| Plasmid | RNA | |
|---|---|---|
| Element | Recognition | Amino Acid Sequence |
| SV40 NLS | — | PKKKRKV (SEQ ID NO: 437) |
| 14PUF | UGCUGCUGCU | GRSRLLEDFRNNRYPNLQLREIAGHIMEFSQDQHGSRFI |
| | GCUG | ELKLERATPAERQLVFNEILQAAYQLMVDVFGNYVIQK |
| | | FFEFGSLEQKLALAERIRGHVLSLALQMYGSYVIRKALE |
| | | FIPSDQQNEMVRELDGHVLKCVKDQNGSYVVEKCIECV |
| | | QPQSLQFIIDAFKGQVFALSTHPYGNRVIQRILEHCLPDQ |
| | | TLPILEELHQHIMEFSQDQHGSRFIRLKLERATPAERQLV |
| | | FNEILQAAYQLMVDVFGSYVIEKFFEFGSLEQKLALAER |
| | | IRGHVLSLALQMYGNRVIQKALEFIPSDQQNEMVRELD |
| | | GHVLKCVKDQNGSYVVRKCIECVQPQSLQFIIDAFKGQ |
| | | VFALSTHPYGSRVIERILEHCLPDQTLPILEELHQHTEQL |
| | | VQDQYGNYVIQHVLEHGRPEDKSKIVAEIRGHTEQLVQ |
| | | DQYGSYVIRHVLEHGRPEDKSKIVAEIRGNVLVLSQHK |
| | | FASNVVEKCVTHASRTERAVLIDEVCTMNDGPHSALYT |
| | | MMKDQYANYVVQKMIDVAEPGQRKIVMHKIRPHIATL |
| | | RKYTYGKHILAKLEKYYMKNGVDLG |
| | | (SEQ ID NO: 445) |
| Linker | — | VDTANGS (SEQ ID NO: 411) |
| E17 | — | GGGTPKAPNLEPPLPEEEKEGSDLRPVVIDGSNVAMSHG |
| endonuclease | | NKEVFSCRGILLAVNWFLERGHTDITVFVPSWRKEQPRP |
| | | DVPITDQHILRELEKKKILVFTPSRRVGGKRVVCYDDRFI |
| | | VKLAYESDGIVVSNDTYRDLQGERQEWKRFIEERLLMYS |
| | | FVNDKFMPPDDPLGRHGPSLDNFLRKKPLTLE (SEQ ID |
| | | NO: 358) |

TABLE 44
Exemplary 14PUF targeting CUG Fusion Protein

| Plasmid | RNA | |
|---|---|---|
| Element | Recognition | Amino Acid Sequence |
| SV40 NLS | — | PKKKRKV (SEQ ID NO: 437) |
| 14PUF | UGCUGCUGCU | GRSRLLEDFRNNRYPNLQLREIAGHIMEFSQDQHGSRFIE |
| | GCUG | LKLERATPAERQLVFNEILQAAYQLMVDVFGNYVIQKFF |
| | | EFGSLEQKLALAERIRGHVLSLALQMYGSYVIRKALEFIP |
| | | SDQQNEMVRELDGHVLKCVKDQNGSYVVEKCIECVQPQ |
| | | SLQFIIDAFKGQVFALSTHPYGNRVIQRILEHCLPDQTLPIL |
| | | EELHQHTEQLVQDQYGSYVIRHVLEHGRPEDKSKIVAEI |
| | | RGHIMEFSQDQHGSRFIELKLERATPAERQLVFNEILQAA |
| | | YQLMVDVFGNYVIQKFFEFGSLEQKLALAERIRGHVLSL |
| | | ALQMYGSYVIRKALEFIPSDQQNEMVRELDGHVLKCVK |
| | | DQNGSYVVEKCIECVQPQSLQFIIDAFKGQVFALSTHPYG |
| | | NRVIQRILEHCLPDQTLPILEELHQHTEQLVQDQYGSYVI |
| | | RHVLEHGRPEDKSKIVAEIRGNVLVLSQHKFASNVVEKC |
| | | VTHASRTERAVLIDEVCTMNDGPHSALYTMMKDQYAN |
| | | YVVQKMIDVAEPGQRKIVMHKIRPHIATLRKYTYGKHIL |
| | | AKLEKYYMKNGVDLG |
| | | (SEQ ID NO: 446) |
| Linker | — | VDTANGS (SEQ ID NO: 411) |
| E17 | — | GGGTPKAPNLEPPLPEEEKEGSDLRPVVIDGSNVAMSHG |
| endonuclease | | NKEVFSCRGILLAVNWFLERGHTDITVFVPSWRKEQPRP |
| | | DVPITDQHILRELEKKKILVFTPSRRVGGKRVVCYDDRFI |
| | | VKLAYESDGIVVSNDTYRDLQGERQEWKRFIEERLLMYS |
| | | FVNDKFMPPDDPLGRHGPSLDNFLRKKPLTLE (SEQ ID |
| | | NO: 358) |

TABLE 45
Exemplary 15PUF targeting CUG Fusion Protein

| Plasmid | RNA | |
|---|---|---|
| Element | Recognition | Amino Acid Sequence |
| SV40 NLS | — | PKKKRKV (SEQ ID NO: 437) |
| 15PUF | UGCUGCUGCU | GRSRLLEDFRNNRYPNLQLREIAGHIMEFSQDQHGSRFIR |
| | GCUGC | LKLERATPAERQLVFNEILQAAYQLMVDVFGSYVIEKFFE |
| | | FGSLEQKLALAERIRGHVLSLALQMYGNRVIQKALEFIPS |
| | | DQQNEMVRELDGHVLKCVKDQNGSYVVRKCIECVQPQS |
| | | LQFIIDAFKGQVFALSTHPYGSRVIERILEHCLPDQTLPILE |
| | | ELHQHIMEFSQDQHGNRFIQLKLERATPAERQLVFNEILQ |
| | | AAYQLMVDVFGSYVIRKFFEFGSLEQKLALAERIRGHVL |
| | | SLALQMYGSRVIEKALEFIPSDQQNEMVRELDGHVLKCV |
| | | KDQNGNHVVQKCIECVQPQSLQFIIDAFKGQVFALSTHP |
| | | YGSRVIRRILEHCLPDQTLPILEELHQHTEQLVQDQYGSY |
| | | VIEHVLEHGRPEDKSKIVAEIRGNVLVLSQHKFANNVVQ |
| | | KCVTHASRTERAVLIDEVCTMNDGPHSHTEQLVQDQYG |
| | | SYVIRHVLEHGRPEDKSKIVAEIRGNVLVLSQHKFASNVV |
| | | EKCVTHASRTERAVLIDEVCTMNDGPHSALYTMMKDQY |
| | | ANYVVQKMIDVAEPGQRKIVMHKIRPHIATLRKYTYGK |
| | | HILAKLEKYYMKNGVDLG |
| | | (SEQ ID NO: 447) |
| Linker | — | VDTANGS (SEQ ID NO: 411) |
| E17 | — | GGGTPKAPNLEPPLPEEEKEGSDLRPVVIDGSNVAMSHG |
| endonuclease | | NKEVFSCRGILLAVNWFLERGHTDITVFVPSWRKEQPRP |
| | | DVPITDQHILRELEKKKILVFTPSRRVGGKRVVCYDDRFI |
| | | VKLAYESDGIVVSNDTYRDLQGERQEWKRFIEERLLMYS |
| | | FVNDKFMPPDDPLGRHGPSLDNFLRKKPLTLE (SEQ ID |
| | | NO: 358) |

TABLE 46
Exemplary 15PUF targeting CUG Fusion Protein

| Plasmid | RNA | |
|---|---|---|
| Element | Recognition | Amino Acid Sequence |
| SV40 NLS | — | PKKKRKV (SEQ ID NO: 437) |
| 15PUF | UGCUGCUGCU | GRSRLLEDFRNNRYPNLQLREIAGHIMEFSQDQHGSRFIR |
| | GCUGC | LKLERATPAERQLVFNEILQAAYQLMVDVFGSYVIEKFF |
| | | EFGSLEQKLALAERIRGHVLSLALQMYGNRVIQKALEFIP |
| | | SDQQNEMVRELDGHVLKCVKDQNGSYVVRKCIECVQP |
| | | QSLQFIIDAFKGQVFALSTHPYGSRVIERILEHCLPDQTLP |
| | | ILEELHQHTEQLVQDQYGNYVIQHVLEHGRPEDKSKIVA |
| | | EIRGHIMEFSQDQHGSRFIRLKLERATPAERQLVFNEILQ |
| | | AAYQLMVDVFGSYVIEKFFEFGSLEQKLALAERIRGHVL |
| | | SLALQMYGNRVIQKALEFIPSDQQNEMVRELDGHVLKC |
| | | VKDQNGSYVVRKCIECVQPQSLQFIIDAFKGQVFALSTH |
| | | PYGSRVIERILEHCLPDQTLPILEELHQHTEQLVQDQYGN |
| | | YVIQHVLEHGRPEDKSKIVAEIRGNVLVLSQHKFASYVV |
| | | RKCVTHASRTERAVLIDEVCTMNDGPHSNVLVLSQHKF |
| | | ASNVVEKCVTHASRTERAVLIDEVCTMNDGPHSALYTM |
| | | MKDQYANYVVQKMIDVAEPGQRKIVMHKIRPHIATLRK |
| | | YTYGKHILAKLEKYYMKNGVDLG |
| | | (SEQ ID NO: 448) |
| Linker | — | VDTANGS (SEQ ID NO: 411) |
| E17 | — | GGGTPKAPNLEPPLPEEEKEGSDLRPVVIDGSNVAMSHG |
| endonuclease | | NKEVFSCRGILLAVNWFLERGHTDITVFVPSWRKEQPRP |
| | | DVPITDQHILRELEKKKILVFTPSRRVGGKRVVCYDDRFI |
| | | VKLAYESDGIVVSNDTYRDLQGERQEWKRFIEERLLMYS |
| | | FVNDKFMPPDDPLGRHGPSLDNFLRKKPLTLE (SEQ ID |
| | | NO: 358) |

TABLE 47
Exemplary 15PUF targeting CUG Fusion Protein

| Plasmid | RNA | |
|---|---|---|
| Element | Recognition | Amino Acid Sequence |
| SV40 NLS | — | PKKKRKV (SEQ ID NO: 437) |
| 15PUF | UGCUGCUGCU | GRSRLLEDFRNNRYPNLQLREIAGHIMEFSQDQHGSRFIR |
| | GCUGC | LKLERATPAERQLVFNEILQAAYQLMVDVFGSYVIEKFF |
| | | EFGSLEQKLALAERIRGHVLSLALQMYGNRVIQKALEFIP |
| | | SDQQNEMVRELDGHVLKCVKDQNGSYVVRKCIECVQP |
| | | QSLQFIIDAFKGQVFALSTHPYGSRVIERILEHCLPDQTLPI |
| | | LEELHQHTEQLVQDQYGNYVIQHVLEHGRPEDKSKIVAE |
| | | IRGNVLVLSQHKFASYVVRKCVTHASRTERAVLIDEVCT |
| | | MNDGPHSHIMEFSQDQHGSRFIELKLERATPAERQLVFN |
| | | EILQAAYQLMVDVFGNYVIQKFFEFGSLEQKLALAERIR |
| | | GHVLSLALQMYGSYVIRKALEFIPSDQQNEMVRELDGH |
| | | VLKCVKDQNGSYVVEKCIECVQPQSLQFIIDAFKGQVFA |
| | | LSTHPYGNRVIQRILEHCLPDQTLPILEELHQHTEQLVQD |
| | | QYGSYVIRHVLEHGRPEDKSKIVAEIRGNVLVLSQHKFA |
| | | SNVVEKCVTHASRTERAVLIDEVCTMNDGPHSALYTMM |
| | | KDQYANYVVQKMIDVAEPGQRKIVMHKIRPHIATLRKY |
| | | TYGKHILAKLEKYYMKNGVDLG |
| | | (SEQ ID NO: 461) |
| Linker | — | VDTANGS (SEQ ID NO: 411) |
| E17 | — | GGGTPKAPNLEPPLPEEEKEGSDLRPVVIDGSNVAMSHG |
| endonuclease | | NKEVFSCRGILLAVNWFLERGHTDITVFVPSWRKEQPRP |
| | | DVPITDQHILRELEKKKILVFTPSRRVGGKRVVCYDDRFI |
| | | VKLAYESDGIVVSNDTYRDLQGERQEWKRFIEERLLMYS |
| | | FVNDKFMPPDDPLGRHGPSLDNFLRKKPLTLE (SEQ ID |
| | | NO: 358) |

TABLE 48
Exemplary 16PUF targeting CUG Fusion Protein

| Plasmid | RNA | |
|---|---|---|
| Element | Recognition | PUF Amino Acid Sequence |
| SV40 NLS | — | PKKKRKV (SEQ ID NO: 437) |
| 16PUF | UGCUGCUGCU | GRSRLLEDFRNNRYPNLQLREIAGHIMEFSQDQHGNRFIQ |
| | GCUGCU (SEQ | LKLERATPAERQLVFNEILQAAYQLMVDVFGSYVIRKFFE |
| | ID NO: 456) | FGSLEQKLALAERIRGHVLSLALQMYGSRVIEKALEFIPSD |
| | | QQNEMVRELDGHVLKCVKDQNGNHVVQKCIECVQPQSL |
| | | QFIIDAFKGQVFALSTHPYGSRVIRRILEHCLPDQTLPILEE |
| | | LHQHIMEFSQDQHGSRFIELKLERATPAERQLVFNEILQA |
| | | AYQLMVDVFGNYVIQKFFEFGSLEQKLALAERIRGHVLS |
| | | LALQMYGSYVIRKALEFIPSDQQNEMVRELDGHVLKCVK |
| | | DQNGSYVVEKCIECVQPQSLQFIIDAFKGQVFALSTHPYG |
| | | NRVIQRILEHCLPDQTLPILEELHQHTEQLVQDQYGSYVIR |
| | | HVLEHGRPEDKSKIVAEIRGNVLVLSQHKFASNVVEKCV |
| | | THASRTERAVLIDEVCTMNDGPHSALYTMMKDQYANYV |
| | | VQKMIDVAEPGQRKIVMHKIRPHTEQLVQDQYGSYVIRH |
| | | VLEHGRPEDKSKIVAEIRGNVLVLSQHKFASNVVEKCVT |
| | | HASRTERAVLIDEVCTMNDGPHSALYTMMKDQYANYVV |
| | | QKMIDVAEPGQRKIVMHKIRPHIATLRKYTYGKHILAKLE |
| | | KYYMKNGVDLG |
| | | (SEQ ID NO: 449) |
| Linker | — | VDTANGS (SEQ ID NO: 411) |
| E17 | — | GGGTPKAPNLEPPLPEEEKEGSDLRPVVIDGSNVAMSHG |
| endonuclease | | NKEVFSCRGILLAVNWFLERGHTDITVFVPSWRKEQPRP |
| | | DVPITDQHILRELEKKKILVFTPSRRVGGKRVVCYDDRFI |
| | | VKLAYESDGIVVSNDTYRDLQGERQEWKRFIEERLLMYS |
| | | FVNDKFMPPDDPLGRHGPSLDNFLRKKPLTLE (SEQ ID |
| | | NO: 358) |

TABLE 49
Exemplary 16PUF targeting CUG Fusion Protein

| Plasmid | RNA | |
|---|---|---|
| Element | Recognition | Amino Acid Sequence |
| SV40 NLS | — | PKKKRKV (SEQ ID NO: 437) |
| 16PUF | UGCUGCUGCU | GRSRLLEDFRNNRYPNLQLREIAGHIMEFSQDQHGNRFI |
| | GCUGCU (SEQ | QLKLERATPAERQLVFNEILQAAYQLMVDVFGSYVIRKF |
| | ID NO: 456) | FEFGSLEQKLALAERIRGHVLSLALQMYGSRVIEKALEFI |
| | | PSDQQNEMVRELDGHVLKCVKDQNGNHVVQKCIECVQ |
| | | PQSLQFIIDAFKGQVFALSTHPYGSRVIRRILEHCLPDQTL |
| | | PILEELHQHTEQLVQDQYGSYVIEHVLEHGRPEDKSKIV |
| | | AEIRGHIMEFSQDQHGNRFIQLKLERATPAERQLVFNEIL |
| | | QAAYQLMVDVFGSYVIRKFFEFGSLEQKLALAERIRGHV |
| | | LSLALQMYGSRVIEKALEFIPSDQQNEMVRELDGHVLKC |
| | | VKDQNGNHVVQKCIECVQPQSLQFIIDAFKGQVFALSTH |
| | | PYGSRVIRRILEHCLPDQTLPILEELHQHTEQLVQDQYGS |
| | | YVIEHVLEHGRPEDKSKIVAEIRGNVLVLSQHKFANNVV |
| | | QKCVTHASRTERAVLIDEVCTMNDGPHSALYTMMKDQ |
| | | YASYVVRKMIDVAEPGQRKIVMHKIRPNVLVLSQHKFA |
| | | SNVVEKCVTHASRTERAVLIDEVCTMNDGPHSALYTMM |
| | | KDQYANYVVQKMIDVAEPGQRKIVMHKIRPHIATLRKY |
| | | TYGKHILAKLEKYYMKNGVDLG |
| | | (SEQ ID NO: 450) |
| Linker | — | VDTANGS (SEQ ID NO: 411) |
| E17 | — | GGGTPKAPNLEPPLPEEEKEGSDLRPVVIDGSNVAMSHG |
| endonuclease | | NKEVFSCRGILLAVNWFLERGHTDITVFVPSWRKEQPRP |
| | | DVPITDQHILRELEKKKILVFTPSRRVGGKRVVCYDDRFI |
| | | VKLAYESDGIVVSNDTYRDLQGERQEWKRFIEERLLMYS |
| | | FVNDKFMPPDDPLGRHGPSLDNFLRKKPLTLE (SEQ ID |
| | | NO: 358) |

| Plasmid | RNA | |
|---|---|---|
| Element | Recognition | Amino Acid Sequence |
| SV40 NLS | — | PKKKRKV (SEQ ID NO: 437) |
| 16PUF | UGCUGCUGCU | GRSRLLEDFRNNRYPNLQLREIAGHIMEFSQDQHGNRFIQ |
| | GCUGCU (SEQ | LKLERATPAERQLVFNEILQAAYQLMVDVFGSYVIRKFF |
| | ID NO: 456) | EFGSLEQKLALAERIRGHVLSLALQMYGSRVIEKALEFIP |
| | | SDQQNEMVRELDGHVLKCVKDQNGNHVVQKCIECVQP |
| | | QSLQFIIDAFKGQVFALSTHPYGSRVIRRILEHCLPDQTLPI |
| | | LEELHQHTEQLVQDQYGSYVIEHVLEHGRPEDKSKIVAEI |
| | | RGNVLVLSQHKFANNVVQKCVTHASRTERAVLIDEVCT |
| | | MNDGPHSALYTMMKDQYASYVVRKMIDVAEPGQRKIV |
| | | MHKIRPHIMEFSQDQHGSRFIELKLERATPAERQLVFNEI |
| | | LQAAYQLMVDVFGNYVIQKFFEFGSLEQKLALAERIRGH |
| | | VLSLALQMYGSYVIRKALEFIPSDQQNEMVRELDGHVLK |
| | | CVKDQNGSYVVEKCIECVQPQSLQFIIDAFKGQVFALSTH |
| | | PYGNRVIQRILEHCLPDQTLPILEELHQHTEQLVQDQYGS |
| | | YVIRHVLEHGRPEDKSKIVAEIRGNVLVLSQHKFASNVV |
| | | EKCVTHASRTERAVLIDEVCTMNDGPHSALYTMMKDQY |
| | | ANYVVQKMIDVAEPGQRKIVMHKIRPHIATLRKYTYGK |
| | | HILAKLEKYYMKNGVDLG |
| | | (SEQ ID NO: 451) |
| Linker | — | VDTANGS (SEQ ID NO: 411) |
| E17 | — | GGGTPKAPNLEPPLPEEEKEGSDLRPVVIDGSNVAMSHG |
| endonuclease | | NKEVFSCRGILLAVNWFLERGHTDITVFVPSWRKEQPRP |
| | | DVPITDQHILRELEKKKILVFTPSRRVGGKRVVCYDDRFI |
| | | VKLAYESDGIVVSNDTYRDLQGERQEWKRFIEERLLMYS |
| | | FVNDKFMPPDDPLGRHGPSLDNFLRKKPLTLE (SEQ ID |
| | | NO: 358) |

Additional PUF(CUG) RNA-targeting compositions are as follows:

16PUFN5 targeting CUGf3 DM1 w/or w/o Endonuclease

| | Protein | | Target | |
|---|---|---|---|---|
| Construct | Type | Elements | Sequence | Amino Acid Sequence |
| n/a | 16PUFN5 | N- | UGCUGC | GRSRLLEDFRNNRYPNLQLREIAGHIME |
| | | terminal | UGCUGC | FSQDQHGNYFIQLKLERATPAERQLVFN |
| | | Rb NLS, | UGCU | EILQAAYQLMVDVFGSYVIRKFFEFGSL |
| | | Linker | (SEQ ID | EQKLALAERIRGHVLSLALQMYGSNVIE |
| | | between | NO: 456) | KALEFIPSDQQNEMVRELDGHVLKCVK |
| | | NLS and | | DQNGNHVVQKCIECVQPQSLQFIIDAFK |
| | | 16PUF, C- | | GQVFALSTHPYGSYVIRRILEHCLPDQTL |
| | | terminal | | PILEELHQHIMEFSQDQHGSNFIELKLER |
| | | E17 with | | ATPAERQLVFNEILQAAYQLMVDVFGN |
| | | linker | | YVIQKFFEFGSLEQKLALAERIRGHVLSL |
| | | between | | ALQMYGSYVIRKALEFIPSDQQNEMVRE |
| | | 16PUF | | LDGHVLKCVKDQNGSNVVEKCIECVQP |
| | | and E17 | | QSLQFIIDAFKGQVFALSTHPYGNYVIQR |
| | | | | ILEHCLPDQTLPILEELHQHTEQLVQDQY |
| | | | | GSYVIRHVLEHGRPEDKSKIVAEIRGNVL |
| | | | | VLSQHKFASNVVEKCVTHASRTERAVLI |
| | | | | DEVCTMNDGPHSALYTMMKDQYANYV |
| | | | | VQKMIDVAEPGQRKIVMHKIRPHTEQLV |
| | | | | QDQYGSYVIRHVLEHGRPEDKSKIVAEI |
| | | | | RGNVLVLSQHKFASNVVEKCVTHASRT |
| | | | | ERAVLIDEVCTMNDGPHSALYTMMKDQ |
| | | | | YANYVVQKMIDVAEPGQRKIVMHKIRP |
| | | | | HIATLRKYTYGKHILAKLEKYYMKNGV |
| | | | | DLG (SEQ ID NO: 638) |

TABLE T
16PUF targeting CUG Fusion Protein (Blocking or *cleaving)

| Plasmid | |
|---|---|
| Element | Amino Acid Sequences |
| Rb-NLS | DRVLKRSAEGSNPPKPLKKLR (SEQ ID NO: 543) |
| Linker | GGS |
| 16PUFN5 | GRSRLLEDFRNNRYPNLQLREIAGHIMEFSQDQHGNYFIQLKLERATPAERQL |
| | VFNEILQAAYQLMVDVFGSYVIRKFFEFGSLEQKLALAERIRGHVLSLALQM |
| | YGSNVIEKALEFIPSDQQNEMVRELDGHVLKCVKDQNGNHVVQKCIECVQP |
| | QSLQFIIDAFKGQVFALSTHPYGSYVIRRILEHCLPDQTLPILEELHQHIMEFSQ |
| | DQHGSNFIELKLERATPAERQLVFNEILQAAYQLMVDVFGNYVIQKFFEFGSL |
| | EQKLALAERIRGHVLSLALQMYGSYVIRKALEFIPSDQQNEMVRELDGHVLK |
| | CVKDQNGSNVVEKCIECVQPQSLQFIIDAFKGQVFALSTHPYGNYVIQRILEH |
| | CLPDQTLPILEELHQHTEQLVQDQYGSYVIRHVLEHGRPEDKSKIVAEIRGNV |
| | LVLSQHKFASNVVEKCVTHASRTERAVLIDEVCTMNDGPHSALYTMMKDQY |
| | ANYVVQKMIDVAEPGQRKIVMHKIRPHTEQLVQDQYGSYVIRHVLEHGRPE |
| | DKSKIVAEIRGNVLVLSQHKFASNVVEKCVTHASRTERAVLIDEVCTMNDGP |
| | HSALYTMMKDQYANYVVQKMIDVAEPGQRKIVMHKIRPHIATLRKYTYGK |
| | HILAKLEKYYMKNGVDLG (SEQ ID NO: 638) |

(*with endonuclease e.g., E17 and linker, e.g., VDTANGS between 16 PUF and E17 for cleaving)

16PUFN6 Targeting CUGf3 (DM1) w/or w/o Endonuclease

| | Protein | | Target | |
|---|---|---|---|---|
| Construct | Type | Elements | Sequence | PUF Amino Acid Sequence |
| n/a | 16PUFN6 | N- | UGCUGC | GRSRLLEDFRNNRYPNLQLREIAGHIME |
| | | terminal | UGCUGC | FSQDQHGNYFIQLKLERATPAERQLVFN |
| | | Rb NLS, | UGCU | EILQAAYQLMVDVFGSYVIRKFFEFGSL |
| | | Linker | | EQKLALAERIRGHVLSLALQMYGSNVIE |
| | | between | | KALEFIPSDQQNEMVRELDGHVLKCVK |
| | | NLS and | | DQNGNHVVQKCIECVQPQSLQFIIDAFK |
| | | 16PUF, C- | | GQVFALSTHPYGSYVIRRILEHCLPDQTL |
| | | terminal | | PILEELHQHTEQLVQDQYGSNVIEHVLE |
| | | E17 with | | HGRPEDKSKIVAEIRGHIMEFSQDQHGN |
| | | linker | | YFIQLKLERATPAERQLVFNEILQAAYQL |
| | | between | | MVDVFGSYVIRKFFEFGSLEQKLALAERI |
| | | 16PUF | | RGHVLSLALQMYGSNVIEKALEFIPSDQ |
| | | and E17 | | QNEMVRELDGHVLKCVKDQNGNHVVQ |
| | | | | KCIECVQPQSLQFIIDAFKGQVFALSTHP |
| | | | | YGSYVIRRILEHCLPDQTLPILEELHQHT |
| | | | | EQLVQDQYGSNVIEHVLEHGRPEDKSKI |
| | | | | VAEIRGNVLVLSQHKFANYVVQKCVTH |
| | | | | ASRTERAVLIDEVCTMNDGPHSALYTM |
| | | | | MKDQYASYVVRKMIDVAEPGQRKIVM |
| | | | | HKIRPNVLVLSQHKFASNVVEKCVTHAS |
| | | | | RTERAVLIDEVCTMNDGPHSALYTMMK |
| | | | | DQYANYVVQKMIDVAEPGQRKIVMHKI |
| | | | | RPHIATLRKYTYGKHILAKLEKYYMKN |
| | | | | GVDLG (SEQ ID NO: 650) |

### Amino Acid Sequences of Transgene Elements in Order N-Terminal to C-Terminal (Blocking or *Cleaving)

| Plasmid | |
|---|---|
| Element | Amino Acid Sequences |
| Rb-NLS | DRVLKRSAEGSNPPKPLKKLR (SEQ ID NO: 543) |
| Linker | GGS |
| 16PUFN6 | GRSRLLEDFRNNRYPNLQLREIAGHIMEFSQDQHGNYFIQLKLERATPAERQL |
| | VFNEILQAAYQLMVDVFGSYVIRKFFEFGSLEQKLALAERIRGHVLSLALQM |
| | YGSNVIEKALEFIPSDQQNEMVRELDGHVLKCVKDQNGNHVVQKCIECVQP |
| | QSLQFIIDAFKGQVFALSTHPYGSYVIRRILEHCLPDQTLPILEELHQHTEQLVQ |
| | DQYGSNVIEHVLEHGRPEDKSKIVAEIRGHIMEFSQDQHGNYFIQLKLERATP |
| | AERQLVFNEILQAAYQLMVDVFGSYVIRKFFEFGSLEQKLALAERIRGHVLSL |
| | ALQMYGSNVIEKALEFIPSDQQNEMVRELDGHVLKCVKDQNGNHVVQKCIE |
| | CVQPQSLQFIIDAFKGQVFALSTHPYGSYVIRRILEHCLPDQTLPILEELHQHTE |
| | QLVQDQYGSNVIEHVLEHGRPEDKSKIVAEIRGNVLVLSQHKFANYVVQKCV |
| | THASRTERAVLIDEVCTMNDGPHSALYTMMKDQYASYVVRKMIDVAEPGQ |
| | RKIVMHKIRPNVLVLSQHKFASNVVEKCVTHASRTERAVLIDEVCTMNDGPH |
| | SALYTMMKDQYANYVVQKMIDVAEPGQRKIVMHKIRPHIATLRKYTYGKHI |
| | LAKLEKYYMKNGVDLG (SEQ ID NO: 650) |

(*with endonuclease e.g., E17 and linker, e.g., VDTANGS between 16 PUF and E17 for cleaving)

16PUFN0 Targeting CUGf3 DM1 w/or w/o Endonuclease

| | Protein | | Target | |
|---|---|---|---|---|
| Construct | Type | Elements | Sequence | PUF Amino Acid Sequence |
| n/a | 16PUFN0 | N- | UGCUGC | GRSRLLEDFRNNRYPNLQLREIAGHIME |
| | | terminal | UGCUGC | FSQDQHGNYFIQLKLERATPAERQLVFN |
| | | Rb NLS, | UGCU | EILQAAYQLMVDVFGSYVIRKFFEFGSL |
| | | Linker | (SEQ ID | EQKLALAERIRGHVLSLALQMYGSNVIE |
| | | between | NO: 456) | KALEFIPSDQQNEMVRELDGHVLKCVK |
| | | NLS and | | DQNGNHVVQKCIECVQPQSLQFIIDAFK |
| | | 16PUF, C- | | GQVFALSTHPYGSYVIRRILEHCLPDQTL |
| | | terminal | | PILEELHQHTEQLVQDQYGSNVIEHVLE |
| | | E17 with | | HGRPEDKSKIVAEIRGNVLVLSQHKFAN |
| | | linker | | YVVQKCVTHASRTERAVLIDEVCTMND |
| | | between | | GPHSALYTMMKDQYASYVVRKMIDVA |
| | | 16PUF | | EPGQRKIVMHKIRPHIMEFSQDQHGSNFI |
| | | and E17 | | ELKLERATPAERQLVFNEILQAAYQLMV |
| | | | | DVFGNYVIQKFFEFGSLEQKLALAERIRG |
| | | | | HVLSLALQMYGSYVIRKALEFIPSDQQN |
| | | | | EMVRELDGHVLKCVKDQNGSNVVEKCI |
| | | | | ECVQPQSLQFIIDAFKGQVFALSTHPYGN |
| | | | | YVIQRILEHCLPDQTLPILEELHQHTEQL |
| | | | | VQDQYGSYVIRHVLEHGRPEDKSKIVAE |
| | | | | IRGNVLVLSQHKFASNVVEKCVTHASRT |
| | | | | ERAVLIDEVCTMNDGPHSALYTMMKDQ |
| | | | | YANYVVQKMIDVAEPGQRKIVMHKIRP |
| | | | | HIATLRKYTYGKHILAKLEKYYMKNGV |
| | | | | DLG (SEQ ID NO: 639) |

### Amino Acid Sequences of Transgene Elements in Order N-Terminal to C-Terminal (Blocking or *Cleaving)

| Plasmid | |
|---|---|
| Element | Amino Acid Sequences |
| Rb-NLS | DRVLKRSAEGSNPPKPLKKLR (SEQ ID NO: 543) |
| Linker | GGS |
| 16PUFN0 | GRSRLLEDFRNNRYPNLQLREIAGHIMEFSQDQHGNYFIQLKLERATPAERQL |
| | VFNEILQAAYQLMVDVFGSYVIRKFFEFGSLEQKLALAERIRGHVLSLALQM |
| | YGSNVIEKALEFIPSDQQNEMVRELDGHVLKCVKDQNGNHVVQKCIECVQP |
| | QSLQFIIDAFKGQVFALSTHPYGSYVIRRILEHCLPDQTLPILEELHQHTEQLVQ |
| | DQYGSNVIEHVLEHGRPEDKSKIVAEIRGNVLVLSQHKFANYVVQKCVTHAS |
| | RTERAVLIDEVCTMNDGPHSALYTMMKDQYASYVVRKMIDVAEPGQRKIV |
| | MHKIRPHIMEFSQDQHGSNFIELKLERATPAERQLVFNEILQAAYQLMVDVFG |
| | NYVIQKFFEFGSLEQKLALAERIRGHVLSLALQMYGSYVIRKALEFIPSDQQN |
| | EMVRELDGHVLKCVKDQNGSNVVEKCIECVQPQSLQFIIDAFKGQVFALSTH |
| | PYGNYVIQRILEHCLPDQTLPILEELHQHTEQLVQDQYGSYVIRHVLEHGRPE |
| | DKSKIVAEIRGNVLVLSQHKFASNVVEKCVTHASRTERAVLIDEVCTMNDGP |
| | HSALYTMMKDQYANYVVQKMIDVAEPGQRKIVMHKIRPHIATLRKYTYGK |
| | HILAKLEKYYMKNGVDLG (SEQ ID NO: 639) |
| *Linker | VDTANGS (SEQ ID NO: 411) |
| *Endonuclease | GGGTPKAPNLEPPLPEEEKEGSDLRPVVIDGSNVAMSHGNKEVFSCRGILLAV |
| | NWFLERGHTDITVFVPSWRKEQPRPDVPITDQHILRELEKKKILVFTPSRRVGG |
| | KRVVCYDDRFIVKLAYESDGIVVSNDTYRDLQGERQEWKRFIEERLLMYSFV |
| | NDKFMPPDDPLGRHGPSLDNFLRKKPLTLE (SEQ ID NO: 358) |

8PUF Targeting CUGf3 (DM1) w/Stacking Mutations to C w/or w/o Endonuclease

| | Protein | | Target | |
|---|---|---|---|---|
| Construct | Type | Elements | Sequence | PUF Amino Acid Sequence |
| A02139 | 8PUF | N- | UGCUGCUG | GRSRLLEDFRNNRYPNLQLREIAG |
| | | terminal | (SEQ ID NO: | HIMEFSQDQHGSRFIELKLERATP |
| | | Rb NLS, | 453) | AERQLVFNEILQAAYQLMVDVFG |
| | | Linker | | NYVIQKFFEFGSLEQKLALAERIR |
| | | between | | GHVLSLALQMYGSRVIRKALEFIP |
| | | NLS and | | SDQQNEMVRELDGHVLKCVKDQ |
| | | 8PUF, C- | | NGSHVVEKCIECVQPQSLQFIIDAF |
| | | terminal | | KGQVFALSTHPYGNRVIQRILEHC |
| | | E17 with | | LPDQTLPILEELHQHTEQLVQDQY |
| | | linker | | GSYVIRHVLEHGRPEDKSKIVAEI |
| | | between | | RGNVLVLSQHKFASNVVEKCVTH |
| | | 8PUF and | | ASRTERAVLIDEVCTMNDGPHSA |
| | | E17 | | LYTMMKDQYANYVVQKMIDVAE |
| | | | | PGQRKIVMHKIRPHIATLRKYTYG |
| | | | | KHILAKLEKYYMKNGVDLG (SEQ |
| | | | | ID NO: 640) |

### Amino Acid Sequences of Transgene Elements in Order N-Terminal to C-Terminal (Blocking or * Cleaving)

| Plasmid | |
|---|---|
| Element | Amino Acid Sequences |
| Rb-NLS | DRVLKRSAEGSNPPKPLKKLR (SEQ ID NO: 543) |
| Linker | GGS |
| 8PUF | GRSRLLEDFRNNRYPNLQLREIAGHIMEFSQDQHGSRFIELKLERATPAERQL |
| | VFNEILQAAYQLMVDVFGNYVIQKFFEFGSLEQKLALAERIRGHVLSLALQM |
| | YGSRVIRKALEFIPSDQQNEMVRELDGHVLKCVKDQNGSHVVEKCIECVQPQ |
| | SLQFIIDAFKGQVFALSTHPYGNRVIQRILEHCLPDQTLPILEELHQHTEQLVQ |
| | DQYGSYVIRHVLEHGRPEDKSKIVAEIRGNVLVLSQHKFASNVVEKCVTHAS |
| | RTERAVLIDEVCTMNDGPHSALYTMMKDQYANYVVQKMIDVAEPGQRKIV |
| | MHKIRPHIATLRKYTYGKHILAKLEKYYMKNGVDLG (SEQ ID NO: 640) |
| *Linker | VDTANGS (SEQ ID NO: 411) |
| *Endonuclease | GGGTPKAPNLEPPLPEEEKEGSDLRPVVIDGSNVAMSHGNKEVFSCRGILLAV |
| | NWFLERGHTDITVFVPSWRKEQPRPDVPITDQHILRELEKKKILVFTPSRRVGG |
| | KRVVCYDDRFIVKLAYESDGIVVSNDTYRDLQGERQEWKRFIEERLLMYSFV |
| | NDKFMPPDDPLGRHGPSLDNFLRKKPLTLE (SEQ ID NO: 358) |

### 8PUF Targeting CUGf3 (DM1) w Stacking Mutations to C w/ or w/o Endonuclease

| | Protein | | Target | |
|---|---|---|---|---|
| Construct | Type | Elements | Sequence | PUF Amino Acid Sequence |
| A02140 | 8PUF | N- | UGCUGCUG | GRSRLLEDFRNNRYPNLQLREIAG |
| | | terminal | (SEQ ID NO: | HIMEFSQDQHGSRFIELKLERATP |
| | | Rb NLS, | 453) | AERQLVFNEILQAAYQLMVDVFG |
| | | Linker | | NYVIQKFFEFGSLEQKLALAERIR |
| | | between | | GHVLSLALQMYGSRVIRKALEFIP |
| | | NLS and | | SDQQNEMVRELDGHVLKCVKDQ |
| | | 8PUF, C- | | NGSYVVEKCIECVQPQSLQFIIDAF |
| | | terminal | | KGQVFALSTHPYGNRVIQRILEHC |
| | | E17 with | | LPDQTLPILEELHQHTEQLVQDQY |
| | | linker | | GSYVIRHVLEHGRPEDKSKIVAEI |
| | | between | | RGNVLVLSQHKFASNVVEKCVTH |
| | | 8PUF and | | ASRTERAVLIDEVCTMNDGPHSA |
| | | E17 | | LYTMMKDQYANYVVQKMIDVAE |
| | | | | PGQRKIVMHKIRPHIATLRKYTYG |
| | | | | KHILAKLEKYYMKNGVDLG (SEQ |
| | | | | ID NO: 641) |

### Amino Acid Sequences of Transgene Elements in Order N-Terminal to C-Terminal (Blocking or *Cleaving)

| Plasmid | |
|---|---|
| Element | Amino Acid Sequences |
| Rb-NLS | DRVLKRSAEGSNPPKPLKKLR (SEQ ID NO: 543) |
| Linker | GGS |
| 8PUF | GRSRLLEDFRNNRYPNLQLREIAGHIMEFSQDQHGSRFIELKLERATPAERQL |
| | VFNEILQAAYQLMVDVFGNYVIQKFFEFGSLEQKLALAERIRGHVLSLALQM |
| | YGSRVIRKALEFIPSDQQNEMVRELDGHVLKCVKDQNGSYVVEKCIECVQPQ |
| | SLQFIIDAFKGQVFALSTHPYGNRVIQRILEHCLPDQTLPILEELHQHTEQLVQ |
| | DQYGSYVIRHVLEHGRPEDKSKIVAEIRGNVLVLSQHKFASNVVEKCVTHAS |
| | RTERAVLIDEVCTMNDGPHSALYTMMKDQYANYVVQKMIDVAEPGQRKIV |
| | MHKIRPHIATLRKYTYGKHILAKLEKYYMKNGVDLG (SEQ ID NO: 641) |
| *Linker | VDTANGS (SEQ ID NO: 411) |
| *Endonuclease | GGGTPKAPNLEPPLPEEEKEGSDLRPVVIDGSNVAMSHGNKEVFSCRGILLAV |
| | NWFLERGHTDITVFVPSWRKEQPRPDVPITDQHILRELEKKKILVFTPSRRVGG |
| | KRVVCYDDRFIVKLAYESDGIVVSNDTYRDLQGERQEWKRFIEERLLMYSFV |
| | NDKFMPPDDPLGRHGPSLDNFLRKKPLTLE (SEQ ID NO: 358) |

### 8PUF Targeting CUGf3 (DM1 w/ Stacking Mutations to C w/ or w/o Endonuclease

| | Protein | | Target | |
|---|---|---|---|---|
| Construct | Type | Elements | Sequence | PUF Amino Acid Sequence |
| A02141 | 8PUF | N- | UGCUGCUG | GRSRLLEDFRNNRYPNLQLREIAG |
| | | terminal | (SEQ ID NO: | HIMEFSQDQHGSRFIELKLERATP |
| | | Rb NLS, | | AERQLVFNEILQAAYQLMVDVFG |
| | | 453) | | NYVIQKFFEFGSLEQKLALAERIR |
| | | Linker | | GHVLSLALQMYGSRVIRKALEFIP |
| | | between | | SDQQNEMVRELDGHVLKCVKDQ |
| | | NLS and | | NGSNVVEKCIECVQPQSLQFIIDAF |
| | | 8PUF, C- | | KGQVFALSTHPYGNRVIQRILEHC |
| | | terminal | | LPDQTLPILEELHQHTEQLVQDQY |
| | | E17 with | | GSYVIRHVLEHGRPEDKSKIVAEI |
| | | linker | | RGNVLVLSQHKFASNVVEKCVTH |
| | | between | | ASRTERAVLIDEVCTMNDGPHSA |
| | | 8PUF and | | LYTMMKDQYANYVVQKMIDVAE |
| | | E17 | | PGQRKIVMHKIRPHIATLRKYTYG |
| | | | | KHILAKLEKYYMKNGVDLG (SEQ |
| | | | | ID NO: 642) |

### Amino Acid Sequences of Transgene Elements in Order N-Terminal to C-Terminal (Blocking or *Cleaving)

| Plasmid | |
|---|---|
| Element | Amino Acid Sequences |
| Rb-NLS | DRVLKRSAEGSNPPKPLKKLR (SEQ ID NO: 543) |
| Linker | GGS |
| 8PUF | GRSRLLEDFRNNRYPNLQLREIAGHIMEFSQDQHGSRFIELKLERATPAERQL |
| | VFNEILQAAYQLMVDVFGNYVIQKFFEFGSLEQKLALAERIRGHVLSLALQM |
| | YGSRVIRKALEFIPSDQQNEMVRELDGHVLKCVKDQNGSNVVEKCIECVQPQ |
| | SLQFIIDAFKGQVFALSTHPYGNRVIQRILEHCLPDQTLPILEELHQHTEQLVQ |
| | DQYGSYVIRHVLEHGRPEDKSKIVAEIRGNVLVLSQHKFASNVVEKCVTHAS |
| | RTERAVLIDEVCTMNDGPHSALYTMMKDQYANYVVQKMIDVAEPGQRKIV |
| | MHKIRPHIATLRKYTYGKHILAKLEKYYMKNGVDLG (SEQ ID NO: 642) |
| *Linker | VDTANGS (SEQ ID NO: 411) |
| *Endonuclease | GGGTPKAPNLEPPLPEEEKEGSDLRPVVIDGSNVAMSHGNKEVFSCRGILLAV |
| | NWFLERGHTDITVFVPSWRKEQPRPDVPITDQHILRELEKKKILVFTPSRRVGG |
| | KRVVCYDDRFIVKLAYESDGIVVSNDTYRDLQGERQEWKRFIEERLLMYSFV |
| | NDKFMPPDDPLGRHGPSLDNFLRKKPLTLE (SEQ ID NO: 358) |

8PUF Targeting CUGf3 (DM1) w/Stacking Mutations to C w/or w/o Endonuclease

| | Protein | | Target | |
|---|---|---|---|---|
| Construct | Type | Elements | Sequence | PUF Amino Acid Sequence |
| A02142 | 8PUF | N- | UGCUGCUG | GRSRLLEDFRNNRYPNLQLREIAG |
| | | terminal | (SEQ ID NO: | HIMEFSQDQHGSRFIELKLERATP |
| | | Rb NLS, | 453) | AERQLVFNEILQAAYQLMVDVFG |
| | | Linker | | NYVIQKFFEFGSLEQKLALAERIR |
| | | between | | GHVLSLALQMYGSFVIRKALEFIP |
| | | NLS and | | SDQQNEMVRELDGHVLKCVKDQ |
| | | 8PUF, C- | | NGSFVVEKCIECVQPQSLQFIIDAF |
| | | terminal | | KGQVFALSTHPYGNRVIQRILEHC |
| | | E17 with | | LPDQTLPILEELHQHTEQLVQDQY |
| | | linker | | GSYVIRHVLEHGRPEDKSKIVAEI |
| | | between | | RGNVLVLSQHKFASNVVEKCVTH |
| | | 8PUF and | | ASRTERAVLIDEVCTMNDGPHSA |
| | | E17 | | LYTMMKDQYANYVVQKMIDVAE |
| | | | | PGQRKIVMHKIRPHIATLRKYTYG |
| | | | | KHILAKLEKYYMKNGVDLG (SEQ |
| | | | | ID NO: 643) |

### Amino Acid Sequences of Transgene Elements in Order N-Terminal to C-Terminal (Blocking or *Cleaving)

| Plasmid | |
|---|---|
| Element | Amino Acid Sequences |
| Rb-NLS | DRVLKRSAEGSNPPKPLKKLR (SEQ ID NO: 543) |
| Linker | GGS |
| 8PUF | GRSRLLEDFRNNRYPNLQLREIAGHIMEFSQDQHGSRFIELKLERATPAERQL |
| | VFNEILQAAYQLMVDVFGNYVIQKFFEFGSLEQKLALAERIRGHVLSLALQM |
| | YGSFVIRKALEFIPSDQQNEMVRELDGHVLKCVKDQNGSFVVEKCIECVQPQ |
| | SLQFIIDAFKGQVFALSTHPYGNRVIQRILEHCLPDQTLPILEELHQHTEQLVQ |
| | DQYGSYVIRHVLEHGRPEDKSKIVAEIRGNVLVLSQHKFASNVVEKCVTHAS |
| | RTERAVLIDEVCTMNDGPHSALYTMMKDQYANYVVQKMIDVAEPGQRKIV |
| | MHKIRPHIATLRKYTYGKHILAKLEKYYMKNGVDLG (SEQ ID NO: 643) |
| *Linker | VDTANGS (SEQ ID NO: 411) |
| *Endonuclease | GGGTPKAPNLEPPLPEEEKEGSDLRPVVIDGSNVAMSHGNKEVFSCRGILLAV |
| | NWFLERGHTDITVFVPSWRKEQPRPDVPITDQHILRELEKKKILVFTPSRRVGG |
| | KRVVCYDDRFIVKLAYESDGIVVSNDTYRDLQGERQEWKRFIEERLLMYSFV |
| | NDKFMPPDDPLGRHGPSLDNFLRKKPLTLE (SEQ ID NO: 358) |

8PUF Targeting CUGf3 (DM1) w/Stacking Mutations to C w/or w/o Endonuclease

| | Protein | | Target | |
|---|---|---|---|---|
| Construct | Type | Elements | Sequence | PUF Amino Acid Sequence |
| A02143 | 8PUF | N- | UGCUGCUG | GRSRLLEDFRNNRYPNLQLREIAG |
| | | terminal | (SEQ ID NO: | HIMEFSQDQHGSRFIELKLERATP |
| | | Rb NLS, | 453) | AERQLVFNEILQAAYQLMVDVFG |
| | | Linker | | NYVIQKFFEFGSLEQKLALAERIR |
| | | between | | GHVLSLALQMYGSFVIRKALEFIP |
| | | NLS and | | SDQQNEMVRELDGHVLKCVKDQ |
| | | 8PUF, C- | | NGSYVVEKCIECVQPQSLQFIIDAF |
| | | terminal | | KGQVFALSTHPYGNRVIQRILEHC |
| | | E17 with | | LPDQTLPILEELHQHTEQLVQDQY |
| | | linker | | GSYVIRHVLEHGRPEDKSKIVAEI |
| | | between | | RGNVLVLSQHKFASNVVEKCVTH |
| | | 8PUF and | | ASRTERAVLIDEVCTMNDGPHSA |
| | | E17 | | LYTMMKDQYANYVVQKMIDVAE |
| | | | | PGQRKIVMHKIRPHIATLRKYTYG |
| | | | | KHILAKLEKYYMKNGVDLG (SEQ |
| | | | | ID NO: 644) |

### Amino Acid Sequences of Transgene Elements in Order N-Terminal to C-Terminal (Blocking or *Cleaving)

| Plasmid | |
|---|---|
| Element | Amino Acid Sequences |
| Rb-NLS | DRVLKRSAEGSNPPKPLKKLR |
| | (SEQ ID NO: 543) |
| Linker | GGS |
| 8PUF | GRSRLLEDFRNNRYPNLQLR |
| | EIAGHIMEFSQDQHGSRFIE |
| | LKLERATPAERQLVFNEILQ |
| | AAYQLMVDVFGNYVIQKFFE |
| | FGSLEQKLALAERIRGHVLS |
| | LALOMYGSFVIRKALEFIPS |
| | DQQNEMVRELDGHVLKCVKD |
| | QNGSYVVEKCIECVQPQSLQ |
| | FIIDAFKGQVFALSTHPYGN |
| | RVIQRILEHCLPDQTLPILE |
| | ELHQHTEQLVQDQYGSYVIR |
| | HVLEHGRPEDKSKIVAEIRG |
| | NVLVLSQHKFASNVVEKCVT |
| | HASRTERAVLIDEVCTMNDG |
| | PHSALYTMMKDQYANYVVQK |
| | MIDVAEPGQRKIVMHKIRPH |
| | IATLRKYTYGKHILAKLEKY |
| | YMKNGVDLG |
| | (SEQ ID NO: 644) |
| *Linker | VDTANGS |
| | (SEQ ID NO: 411) |
| *Endonuclease | GGGTPKAPNLEPPLPEEEKE |
| | GSDLRPVVIDGSNVAMSHGN |
| | KEVFSCRGILLAVNWFLERG |
| | HTDITVFVPSWRKEQPRPDV |
| | PITDQHILRELEKKKILVFT |
| | PSRRVGGKRVVCYDDRFIVK |
| | LAYESDGIVVSNDTYRDLQG |
| | ERQEWKRFIEERLLMYSFVN |
| | DKFMPPDDPLGRHGPSLDNF |
| | LRKKPLTLE |
| | (SEQ ID NO: 358) |

8PUF Targeting CUGf3 (DM1) w/Stacking Mutations to C w/or w/o Endonuclease

| | Pro- | | Target | |
|---|---|---|---|---|
| Con- | tein | Ele- | Se- | PUF Amino Acid |
| struct | Type | ments | quence | Sequence |
| A02144 | 8PUF | N- | UGCUGCUG | GRSRLLEDFRNNRYP |
| | | terminal | (SEQ ID | NLQLREIAGHIMEFS |
| | | Rb NLS, | NO: 453) | QDQHGSRFIELKLER |
| | | Linker | | ATPAERQLVFNEILQ |
| | | between | | AAYQLMVDVFGNYVI |
| | | NLS and | | QKFFEFGSLEQKLAL |
| | | 8PUF, C- | | AERIRGHVLSLALQM |
| | | terminal | | YGSRVIRKALEFIPS |
| | | E17 with | | DQQNEMVRELDGHVL |
| | | linker | | KCVKDQNGSHVVEKC |
| | | between | | IECVQPQSLQFIIDA |
| | | 8PUF and | | FKGQVFALSTHPYGN |
| | | E17 | | RVIQRILEHCLPDQT |
| | | | | LPILEELHQHTEQLV |
| | | | | QDQYGSFVIRHVLEH |
| | | | | GRPEDKSKIVAEIRG |
| | | | | NVLVLSQHKFASNVV |
| | | | | EKCVTHASRTERAVL |
| | | | | IDEVCTMNDGPHSAL |
| | | | | YTMMKDQYANYVVQK |
| | | | | MIDVAEPGQRKIVMH |
| | | | | KIRPHIATLRKYTYG |
| | | | | KHILAKLEKYYMKNG |
| | | | | VDLG (SEQ |
| | | | | ID NO: 645) |

### Amino Acid Sequences of Transgene Elements in Order N-Terminal to C-Terminal (Blocking or *Cleaving)

| Plasmid | |
|---|---|
| Element | Amino Acid Sequences |
| Rb-NLS | (SEQ ID NO: 543) |
| Linker | GGS |
| 8PUF | GRSRLLEDFRNNRYPNLQLREIAGH |
| | IMEFSQDQHGSRFIELKLERATPAE |
| | RQLVFNEILQAAYQLMVDVFGNYVI |
| | QKFFEFGSLEQKLALAERIRGHVLS |
| | LALQMYGSRVIRKALEFIPSDQQN |
| | EMVRELDGHVLKCVKDQNGSHVVEK |
| | CIECVQPQSLQFIIDAFKGQVFALS |
| | THPYGNRVIQRILEHCLPDQTLPIL |
| | EELHQHTEQLVQDQYGSFVIRHVLE |
| | HGRPEDKSKIVAEIRGNVLVLSQHK |
| | FASNVVEKCVTHASRTERAVLIDEV |
| | CTMNDGPHSALYTMMKDQYANYVVQ |
| | KMIDVAEPGQRKIVMHKIRPHIATL |
| | RKYTYGKHILAKLEKYYMKNGVDLG |
| | (SEQ ID NO: 645) |
| *Linker | VDTANGS |
| *Endonuclease | GGGTPKAPNLEPPLPEEEKEGSDLR |
| | PVVIDGSNVAMSHGNKEVFSCRGIL |
| | LAVNWFLERGHTDITVFVPSWRKEQ |
| | PRPDVPITDQHILRELEKKKILVFT |
| | PSRRVGGKRVVCYDDRFIVKLAYES |
| | DGIVVSNDTYRDLQGERQEWKRFIE |
| | ERLLMYSFVNDKFMPPDDPLGRHGP |
| | SLDNFLRKKPLTLE |
| | (SEQ ID NO: 358) |

8PUF Targeting CUGf3 DM1 w/Stacking Mutations to C w/or w/o Endonuclease

| | Pro- | | Target | |
|---|---|---|---|---|
| Con- | tein | Ele- | Se- | PUF Amino Acid |
| struct | Type | ments | quence | Sequence |
| A02145 | 8PUF | N- | UGCUGCUG | GRSRLLEDFRNNRYP |
| | | terminal | (SEQ ID | NLQLREIAGHIMEFS |
| | | Rb NLS, | NO: 453) | QDQHGSRFIELKLER |
| | | Linker | | ATPAERQLVFNEILQ |
| | | between | | AAYQLMVDVFGNYVI |
| | | NLS and | | QKFFEFGSLEQKLAL |
| | | 8PUF, C- | | AERIRGHVLSLALQM |
| | | terminal | | YGSRVIRKALEFIPS |
| | | E17 with | | DQQNEMVRELDGHVL |
| | | linker | | KCVKDQNGSYVVEKC |
| | | between | | IECVQPQSLQFIIDA |
| | | 8PUF | | FKGQVFALSTHPYGN |
| | | and | | RVIQRILEHCLPDQT |
| | | E17 | | LPILEELHQHTEQL |
| | | | | VQDQYGSFVIRHVLE |
| | | | | HGRPEDKSKIVAEIR |
| | | | | GNVLVLSQHKFASNV |
| | | | | VEKCVTHASRTERAV |
| | | | | LIDEVCTMNDGPHSA |
| | | | | LYTMMKDQYANYVVQ |
| | | | | KMIDVAEPGQRKIVM |
| | | | | HKIRPHIATLRKYTY |
| | | | | GKHILAKLEKYYMKN |
| | | | | GVDLG |
| | | | | (SEQ ID |
| | | | | NO: 646) |

### Amino Acid Sequences of Transgene Elements in Order N-Terminal to C-Terminal (Blocking or *Cleaving)

| Plasmid | |
|---|---|
| Element | Amino Acid Sequences |
| Rb-NLS | DRVLKRSAEGSNPPKPLKKL |
| | R |
| | (SEQ ID NO: 543) |
| Linker | GGS |
| 8PUF | GRSRLLEDFRNNRYPNLQLR |
| | EIAGHIMEFSQDQHGSRFIE |
| | LKLERATPAERQLVFNEILQ |
| | AAYQLMVDVFGNYVIQKFFE |
| | FGSLEQKLALAERIRGHVLS |
| | LALQMYGSRVIRKALEFIPS |
| | DQQNEMVRELDGHVLKCVKD |
| | QNGSYVVEKCIECVQPQSLQ |
| | FIIDAFKGQVFALSTHPYGN |
| | RVIQRILEHCLPDQTLPILE |
| | ELHQHTEQLVQDQYGSFVIR |
| | HVLEHGRPEDKSKIVAEIRG |
| | NVLVLSQHKFASNVVEKCVT |
| | HASRTERAVLIDEVCTMNDG |
| | PHSALYTMMKDQYANYVVQK |
| | MIDVAEPGQRKIVMHKIRPH |
| | IATLRKYTYGKHILAKLEKY |
| | YMKNGVDLG |
| | (SEQ ID NO: 646) |
| *Linker | VDTANGS (SEQ ID NO: |
| | 411) |
| *Endonuclease | GGGTPKAPNLEPPLPEEEKE |
| | GSDLRPVVIDGSNVAMSHGN |
| | KEVFSCRGILLAVNWFLERG |
| | HTDITVFVPSWRKEQPRPDV |
| | PITDQHILRELEKKKILVFT |
| | PSRRVGGKRVVCYDDRFIVK |
| | LAYESDGIVVSNDTYRDLQG |
| | ERQEWKRFIEERLLMYSFVN |
| | DKFMPPDDPLGRHGPSLDNF |
| | LRKKPLTLE |
| | (SEQ ID NO: 358) |

### 8PUF Targeting CUGf3 (DM1) w/ Stacking Mutations to C w/ or w/o Endonuclease

| | Pro- | | Target | |
|---|---|---|---|---|
| Con- | tein | Ele- | Se- | PUF Amino Acid |
| struct | Type | ments | quence | Sequence |
| A02146 | 8PUF | N- | UGCUGCUG | GRSRLLEDFRNNRYP |
| | | terminal | (SEQ ID | NLQLREIAGHIMEFS |
| | | Rb NLS, | NO: 453) | QDQHGSRFIELKLER |
| | | Linker | | ATPAERQLVFNEILQ |
| | | between | | AAYQLMVDVFGNYVI |
| | | NLS and | | QKFFEFGSLEQKLAL |
| | | 8PUF, | | AERIRGHVLSLALQM |
| | | C- | | YGSRVIRKALEFIPS |
| | | terminal | | DQQNEMVRELDGHVL |
| | | E17 with | | KCVKDQNGSNVVEKC |
| | | linker | | IECVQPQSLQFIIDA |
| | | between | | FKGQVFALSTHPYGN |
| | | 8PUF and | | RVIQRILEHCLPDQT |
| | | E17 | | LPILEELHQHTEQL |
| | | | | VQDQYGSFVIRHVLE |
| | | | | HGRPEDKSKIVAEIR |
| | | | | GNVLVLSQHKFASNV |
| | | | | VEKCVTHASRTERAV |
| | | | | LIDEVCTMNDGPHSA |
| | | | | LYTMMKDQYANYVVQ |
| | | | | KMIDVAEPGQRKIVM |
| | | | | HKIRPHIATLRKYTY |
| | | | | GKHILAKLEKYYMKN |
| | | | | GVDLG |
| | | | | (SEQ ID |
| | | | | NO: 647) |

### Amino Acid Sequences of Transgene Elements in Order N-Terminal to C-Terminal (Blocking or * Cleaving)

| Plasmid | Amino Acid |
|---|---|
| Element | Sequences |
| Rb-NLS | DRVLKRSAEGSNPPK |
| | PLKKLR |
| | (SEQ ID NO: 543) |
| Linker | GGS |
| 8PUF | GRSRLLEDFRNNRYP |
| | NLQLREIAGHIMEFS |
| | QDQHGSRFIELKLER |
| | ATPAERQLVFNEILQ |
| | AAYQLMVDVFGNYVI |
| | QKFFEFGSLEQKLAL |
| | AERIRGHVLSLALOM |
| | YGSRVIRKALEFIPS |
| | DQQNEMVRELDGHVL |
| | KCVKDQNGSNVVEKC |
| | IECVQPQSLQFIIDA |
| | FKGQVFALSTHPYGN |
| | RVIQRILEHCLPDQT |
| | LPILEELHQHTEQLV |
| | QDQYGSFVIRHVLEH |
| | GRPEDKSKIVAEIRG |
| | NVLVLSQHKFASNVV |
| | EKCVTHASRTERAVL |
| | IDEVCTMNDGPHSAL |
| | YTMMKDQYANYVVQK |
| | MIDVAEPGQRKIVMH |
| | KIRPHIATLRKYTYG |
| | KHILAKLEKYYMKNG |
| | VDLG |
| | (SEQ ID NO: 647) |
| *Linker | VDTANGS (SEQ ID |
| | NO: 411) |
| *Endonuclease | GGGTPKAPNLEPPLP |
| | EEEKEGSDLRPVVID |
| | GSNVAMSHGNKEVFS |
| | CRGILLAVNWFLERG |
| | HTDITVFVPSWRKEQ |
| | PRPDVPITDQHILRE |
| | LEKKKILVFTPSRRV |
| | GGKRVVCYDDRFIVK |
| | LAYESDGIVVSNDTY |
| | RDLQGERQEWKRFIE |
| | ERLLMYSFVNDKFMP |
| | PDDPLGRHGPSLDNF |
| | LRKKPLTLE |
| | (SEQ ID NO: 358) |

### 8PUF Targeting CUGf3 (DM1) w/ Stacking Mutations to C w/ or w/o Endonuclease

| | Pro- | | Target | |
|---|---|---|---|---|
| Con- | tein | Ele- | Se- | PUF Amino Acid |
| struct | Type | ments | quence | Sequence |
| A02147 | 8PUF | N- | UGCUGCUG | GRSRLLEDFRNNRYP |
| | | terminal | (SEQ ID | NLQLREIAGHIMEFS |
| | | Rb NLS, | NO: 453) | QDQHGSRFIELKLER |
| | | Linker | | ATPAERQLVFNEILQ |
| | | between | | AAYQLMVDVFGNYVI |
| | | NLS and | | QKFFEFGSLEQKLAL |
| | | 8PUF, C- | | AERIRGHVLSLALQM |
| | | terminal | | YGSFVIRKALEFIPS |
| | | E17 with | | DQQNEMVRELDGHVL |
| | | linker | | KCVKDQNGSFVVEKC |
| | | between | | IECVQPQSLQFIIDA |
| | | 8PUF and | | FKGQVFALSTHPYGN |
| | | E17 | | RVIQRILEHCLPDQT |
| | | | | LPILEELHQHTEQLV |
| | | | | QDQYGSFVIRHVLEH |
| | | | | GRPEDKSKIVAEIRG |
| | | | | NVLVLSQHKFASNVV |
| | | | | EKCVTHASRTERAVL |
| | | | | IDEVCTMNDGPHSAL |
| | | | | YTMMKDQYANYVVQK |
| | | | | MIDVAEPGQRKIVMH |
| | | | | KIRPHIATLRKYTYG |
| | | | | KHILAKLEKYYMKNG |
| | | | | VDLG |
| | | | | (SEQ ID NO: 648) |

### Amino Acid Sequences of Transgene Elements in Order N-Terminal to C-Terminal (Blocking or *Cleaving)

| Plasmid | Amino Acid |
|---|---|
| Element | Sequences |
| Rb-NLS | DRVLKRSAEGSNPPK |
| | PLKKLR |
| | (SEQ ID NO: 543) |
| Linker | GGS |
| 8PUF | GRSRLLEDFRNNRYP |
| | NLQLREIAGHIMEFS |
| | QDQHGSRFIELKLER |
| | ATPAERQLVFNEILQ |
| | AAYQLMVDVFGNYVI |
| | QKFFEFGSLEQKLAL |
| | AERIRGHVLSLALOM |
| | YGSFVIRKALEFIPS |
| | DQQNEMVRELDGHVL |
| | KCVKDQNGSFVVEKC |
| | IECVQPQSLQFIIDA |
| | FKGQVFALSTHPYGN |
| | RVIQRILEHCLPDQT |
| | LPILEELHQHTEQLV |
| | QDQYGSFVIRHVLEH |
| | GRPEDKSKIVAEIRG |
| | NVLVLSQHKFASNVV |
| | EKCVTHASRTERAVL |
| | IDEVCTMNDGPHSAL |
| | YTMMKDQYANYVVQK |
| | MIDVAEPGQRKIVMH |
| | KIRPHIATLRKYTYG |
| | KHILAKLEKYYMKNG |
| | VDLG |
| | (SEQ ID NO: 648) |
| *Linker | VDTANGS |
| | (SEQ ID NO: 411) |
| *Endonuclease | GGGTPKAPNLEPPLP |
| | EEEKEGSDLRPVVID |
| | GSNVAMSHGNKEVFS |
| | CRGILLAVNWFLERG |
| | HTDITVFVPSWRKEQ |
| | PRPDVPITDQHILRE |
| | LEKKKILVFTPSRRV |
| | GGKRVVCYDDRFIVK |
| | LAYESDGIVVSNDTY |
| | RDLQGERQEWKRFIE |
| | ERLLMYSFVNDKFMP |
| | PDDPLGRHGPSLDNF |
| | LRKKPLTLE |
| | (SEQ ID NO: 358) |

8PUF Targeting CUGf3 (DM1) w/Stacking Mutations to C w/or w/o Endonuclease

| | Pro- | | Target | |
|---|---|---|---|---|
| Con- | tein | Ele- | Se- | PUF Amino Acid |
| struct | Type | ments | quence | Sequence |
| A02148 | 8PUF | N- | UGCUGCUG | GRSRLLEDFRNNRYP |
| | | terminal | (SEQ ID | NLQLREIAGHIMEFS |
| | | Rb NLS, | NO: 453) | QDQHGSRFIELKLER |
| | | Linker | | ATPAERQLVFNEILQ |
| | | between | | AAYQLMVDVFGNYVI |
| | | NLS and | | QKFFEFGSLEQKLAL |
| | | 8PUF, | | AERIRGHVLSLALQM |
| | | C-terminal | | YGSFVIRKALEFIPS |
| | | E17 with | | DQQNEMVRELDGHVL |
| | | linker | | KCVKDQNGSYVVEKC |
| | | between | | IECVQPQSLQFIIDA |
| | | 8PUF and | | FKGQVFALSTHPYGN |
| | | E17 | | RVIQRILEHCLPDQT |
| | | | | LPILEELHQHTEQL |
| | | | | VQDQYGSFVIRHVLE |
| | | | | HGRPEDKSKIVAEIR |
| | | | | GNVLVLSQHKFASNV |
| | | | | VEKCVTHASRTERAV |
| | | | | LIDEVCTMNDGPHSA |
| | | | | LYTMMKDQYANYVVQ |
| | | | | KMIDVAEPGQRKIVM |
| | | | | HKIRPHIATLRKYTY |
| | | | | GKHILAKLEKYYMKN |
| | | | | GVDLG |
| | | | | (SEQ ID |
| | | | | NO: 649) |

### Amino Acid Sequences of Transgene Elements in Order N-Terminal to C-Terminal

| Plasmid | Amino Acid |
|---|---|
| Element | Sequences |
| Rb-NLS | DRVLKRSAEGSNPPK |
| | PLKKLR |
| | (SEQ ID NO: 543) |
| Linker | GGS |
| 8PUF | GRSRLLEDFRNNRYP |
| | NLQLREIAGHIMEFS |
| | QDQHGSRFIELKLER |
| | ATPAERQLVFNEILQ |
| | AAYQLMVDVFGNYVI |
| | QKFFEFGSLEQKLAL |
| | AERIRGHVLSLALOM |
| | YGSFVIRKALEFIPS |
| | DQQNEMVRELDGHVL |
| | KCVKDQNGSYVVEKC |
| | IECVQPQSLQFIIDA |
| | FKGQVFALSTHPYGN |
| | RVIQRILEHCLPDQT |
| | LPILEELHQHTEQLV |
| | QDQYGSFVIRHVLEH |
| | GRPEDKSKIVAEIRG |
| | NVLVLSQHKFASNVV |
| | EKCVTHASRTERAVL |
| | IDEVCTMNDGPHSAL |
| | YTMMKDQYANYVVQK |
| | MIDVAEPGQRKIVMH |
| | KIRPHIATLRKYTYG |
| | KHILAKLEKYYMKNG |
| | VDLG |
| | (SEQ ID NO: 649) |
| *Linker | VDTANGS |
| | (SEQ ID NO: 411) |
| *Endonuclease | GGGTPKAPNLEPPLP |
| | EEEKEGSDLRPVVID |
| | GSNVAMSHGNKEVFS |
| | CRGILLAVNWFLERG |
| | HTDITVFVPSWRKEQ |
| | PRPDVPITDQHILRE |
| | LEKKKILVFTPSRRV |
| | GGKRVVCYDDRFIVK |
| | LAYESDGIVVSNDTY |
| | RDLQGERQEWKRFIE |
| | ERLLMYSFVNDKFMP |
| | PDDPLGRHGPSLDNF |
| | LRKKPLTLE |
| | (SEQ ID NO: 358) |

### 8PUF Targeting CUGf3 (DM1) w/ Stacking Mutations to C w/ or w/o Endonuclease

| | Pro- | | Target | |
|---|---|---|---|---|
| Con- | tein | Ele- | Se- | PUF Amino Acid |
| struct | Type | ments | quence | Sequence |
| A01926 | 8PUF | N- | UGCUGCUG | GRSRLLEDFRNNRYP |
| | | terminal | | NLQLREIAGHIMEFS |
| | | Rb NLS, | | QDQHGSNFIELKLER |
| | | Linker | | ATPAERQLVFNEILQ |
| | | between | | AAYQLMVDVFGNYVI |
| | | NLS and | | QKFFEFGSLEQKLAL |
| | | 8PUF, C- | | AERIRGHVLSLALQM |
| | | terminal | | YGSYVIRKALEFIPS |
| | | E17 with | | DQQNEMVRELDGHVL |
| | | linker | | KCVKDQNGSNVVEKC |
| | | between | | IECVQPQSLQFIIDA |
| | | 8PUF and | | FKGQVFALSTHPYGN |
| | | E17 | | YVIQRILEHCLPDQT |
| | | | | LPILEELHQHTEQLV |
| | | | | QDQYGSYVIRHVLEH |
| | | | | GRPEDKSKIVAEIRG |
| | | | | NVLVLSQHKFASNVV |
| | | | | EKCVTHASRTERAVL |
| | | | | IDEVCTMNDGPHSAL |
| | | | | YTMMKDQYANYVVQK |
| | | | | MIDVAEPGQRKIVMH |
| | | | | KIRPHIATLRKYTYG |
| | | | | KHILAKLEKYYMKNG |
| | | | | VDLG |
| | | | | (SEQ ID |
| | | | | NO: 651) |

### Amino Acid Sequences of Transgene Elements in Order N-Terminal to C-Terminal (Blocking or *Cleaving)

| Plasmid | Amino Acid |
|---|---|
| Element | Sequences |
| Rb-NLS | DRVLKRSAEGSNPPK |
| | PLKKLR |
| | (SEQ ID NO: 543) |
| Linker | GGS |
| 8PUF | GRSRLLEDFRNNRYP |
| | NLQLREIAGHIMEFS |
| | QDQHGSNFIELKLER |
| | ATPAERQLVFNEILQ |
| | AAYQLMVDVFGNYVI |
| | QKFFEFGSLEQKLAL |
| | AERIRGHVLSLALOM |
| | YGSYVIRKALEFIPS |
| | DQQNEMVRELDGHVL |
| | KCVKDQNGSNVVEKC |
| | IECVQPQSLQFIIDA |
| | FKGQVFALSTHPYGN |
| | YVIQRILEHCLPDQT |
| | LPILEELHQHTEQLV |
| | QDQYGSYVIRHVLEH |
| | GRPEDKSKIVAEIRG |
| | NVLVLSQHKFASNVV |
| | EKCVTHASRTERAVL |
| | IDEVCTMNDGPHSAL |
| | YTMMKDQYANYVVQK |
| | MIDVAEPGQRKIVMH |
| | KIRPHIATLRKYTYG |
| | KHILAKLEKYYMKNG |
| | VDLG |
| | (SEQ ID NO: 651) |
| *Linker | VDTANGS (SEQ ID |
| | NO: 411) |
| *Endonuclease | GGGTPKAPNLEPPLP |
| | EEEKEGSDLRPVVID |
| | GSNVAMSHGNKEVFS |
| | CRGILLAVNWFLERG |
| | HTDITVFVPSWRKEQ |
| | PRPDVPITDQHILRE |
| | LEKKKILVFTPSRRV |
| | GGKRVVCYDDRFIVK |
| | LAYESDGIVVSNDTY |
| | RDLQGERQEWKRFIE |
| | ERLLMYSFVNDKFMP |
| | PDDPLGRHGPSLDNF |
| | LRKKPLTLE |
| | (SEQ ID NO: 358) |

### Vectors

In some embodiments of the compositions and methods of the disclosure, a vector comprises a guide RNA of the disclosure. In some embodiments, the vector comprises at least one guide RNA of the disclosure. In some embodiments, the vector comprises one or more guide RNA(s) of the disclosure. In some embodiments, the vector comprises two or more guide RNAs of the disclosure. In one embodiment, the vector comprises three guide RNAs. In one embodiment, the vector comprises four guide RNAs. In some embodiments, the vector further comprises a guided or non-guided RNA-binding protein of the disclosure. In some embodiments, the vector further comprises an RNA-binding fusion protein of the disclosure. In some embodiments, the fusion protein comprises a first RNA binding protein and a second RNA binding protein. In some embodiments, the RNA-guided RNA-binding systems comprising an RNA-binding protein and a gRNA are in a single vector. In a particular embodiment, the single vector comprises the RNA-guided RNA-binding systems which are Cas13d RNA-guided RNA-binding systems or catalytic deactivated Cas13d (dCas13d) RNA-guided RNA-binding systems. In one embodiment, the single vector comprises the Cas13d RNA-guided RNA-binding systems which are CasRx or dCasRx RNA-guided RNA-binding systems. In another embodiment, the single vector comprises a non-guided RNA-binding system comprising a PUF or PUMBY-based protein fused with a nuclease domain from ZC3H12A, such as E17 (SEQ ID NO: 358). In another embodiment, the single vector comprises a dCas13d RNA-binding system fused with a nuclease domain from ZC3H12A, such as E17 (SEQ ID NO: 358).

In some embodiments of the compositions and methods of the disclosure, a first vector comprises a guide RNA of the disclosure and a second vector comprises an RNA-binding protein or RNA-binding fusion protein of the disclosure. In some embodiments, the first vector comprises at least one guide RNA of the disclosure. In some embodiments, the first vector comprises one or more guide RNA(s) of the disclosure. In some embodiments, the first vector comprises two or more guide RNA(s) of the disclosure. In some embodiments, the fusion protein comprises a first RNA binding protein and a second RNA binding protein. In some embodiments, the first vector and the second vector are identical vectors or vector serotypes. In some embodiments, the first vector and the second vector are not identical vectors or vector serotypes. In some embodiments of the compositions and methods of the disclosure, the RNA-binding systems capable of targeting toxic CUG RNA repeats are in a single vector.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector. Vectors are capable of autonomous replication in a host cell into which they are introduced such as e.g., bacterial vectors having a bacterial origin of replication and episomal mammalian vectors and other vectors such as, e.g., non-episomal mammalian vectors, are integrated into the genome of a host cell upon introduction into the host cell, and thereby are replicated along with the host genome.

In some embodiments, vectors such as e.g., expression vectors, are capable of directing the expression of genes to which they are operatively-linked. Common expression vectors are often in the form of plasmids. In some embodiments, recombinant expression vectors comprise a nucleic acid provided herein such as e.g., a guide RNA which can be expressed from a DNA sequence, and a nucleic acid encoding a Cas13d protein, in a form suitable for expression of a protein in a host cell. Recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence such as e.g., in an in vitro transcription/translation system or in a host cell when the vector is introduced into the host cell. Certain embodiments of a vector depend on factors such as the choice of the host cell to be transformed, and the level of expression desired. A vector can be introduced into host cells to thereby produce transcripts, proteins, or peptides, including fusion proteins or peptides, encoded by nucleic acids as described herein such as, e.g., CRISPR transcripts, proteins, enzymes, mutant forms thereof, fusion proteins thereof, etc.

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (560 to 65° C. for several hours), making cold preservation of AAV less critical. AAV may even be lyophilized. Finally, AAV-infected cells are not resistant to superinfection.

AAV (AAV or AAV vector) genomes of the invention comprise, consist essentially of, or consist of a nucleic acid molecule encoding a CUG-repeat targeting composition (such as a PUF, PUMBY, or RNA-guided protein) and one or more AAV ITRs flanking the nucleic acid molecule. Production of pseudotyped AAV is disclosed in, for example, WO2001083692. Other types of AAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAVrh.74, AAV1, AAV2, AAV3, AAV4, AAV5, AAV6, AAV7, AAV8, AAV9, AAV10 (AAVrh10), AAV11 or AAV12. In one embodiment, the AAV vector comprises a modified capsid. In one embodiment the AAV vector is an AAV2-Tyr mutant vector. In one embodiment the AAV vector comprises a capsid with a non-tyrosine amino acid at a position that corresponds to a surface-exposed tyrosine residue in position Tyr252, Tyr272, Tyr275, Tyr281, Tyr508, Tyr612, Tyr704, Tyr720, Tyr730 or Tyr673 of wild-type AAV2. See also WO 2008/124724 incorporated herein in its entirety. In some embodiments, the AAV vector comprises an engineered capsid. AAV vectors comprising engineered capsids include without limitation, AAV2.7m8, AAV9.7m8, AAV2 2tYF, and AAV8 Y733F). In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. In some embodiments, expression control by a promoter is constitutive or ubiquitous. Non-limiting exemplary promoters include a Pol III promoter such as, e.g., U6 and H1 promoters and/or a Pol II promoter e.g., SV40, CMV (optionally including the CMV enhancer), RSV (Rous Sarcoma Virus LTR promoter (optionally including RSV enhancer), CBA (hybrid CMV enhancer/chicken ß-actin), CAG (hybrid CMV enhancer fused to chicken ß-actin), truncated CAG, Cbh (hybrid CBA), EF-1a (human elongation factor alpha-1) or EFS (short intron-less EF-1 alpha), PGK (phosphoglycerol kinase), CEF (chicken embryo fibroblasts), UBC (ubiquitin C), GUSB (lysosomal enzyme beta-glucuronidase), UCOE (ubiquitous chromatin opening element), hAAT (alpha-1 antitrypsin), TBG (thyroxine binding globulin), Desmin (full-length or truncated), MCK (muscle creatine kinase), C5-12 (synthetic muscle promoter), CK8e (creatin kinase 8), NSE (neuron-specific enolase), Synapsin, Synapsin-1 (SYN-1), opsin, PDGF (platelet-derived growth factor), PDGF-A, MecP2 (methyl CpG-binding protein 2), CaMKII (Calcium/Calmodulin-dependent protein kinase II), mGluR2 (metabotropic glutamate receptor 2), NFL (neurofilament light), NFH (neurofilament heavy), nβ2, PPE (rat preproenkephalin), ENK (preproenkephalin), Preproenkephalin-neurofilament chimeric promoter, EAAT2 (glutamate transporter), GFAP (glial fibrillary acidic protein), MBP (myelin basic protein), human rhodopsin kinase promoter (hGRK1), ß-actin promoter, dihydrofolate reductase promoter, MHCK7 (hybrid promoter of enhancer/promoter regions of muscle creatine kinase and alpha myosin heavy-chain genes) and combinations thereof. An “enhancer” is a region of DNA that can be bound by activating proteins to increase the likelihood or frequency of transcription. Non-limiting exemplary enhancers and posttranscriptional regulatory elements include the CMV enhancer, MCK enhancer, R-U5′ segment in LTR of HTLV-1, SV40 enhancer, the intron sequence between exons 2 and 3 of rabbit ß-globin, and Woodchuck Hepatitis Virus (WHP) Posttranscriptional Regulatory Element (WPRE). In some embodiments an intron is used to enhance promoter activity such as a UBB intron. In some embodiments, the UBB intron is used with an EFS promoter.

In some embodiments of the compositions and methods of the disclosure, an expression vector, viral vector or non-viral vector provided herein, includes without limitation, vector elements such as an IRES or 2A peptide sites for configuration of “multicistronic” or “polycistronic” or “bicistronic” or tricistronic” constructs, i.e., having double or triple or multiple coding areas or exons, and as such will have the capability to express from mRNA two or more proteins from a single construct. Multicistronic vectors simultaneously express two or more separate proteins from the same mRNA. The two strategies most widely used for constructing multicistronic configurations are through the use of an IRES or a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or other sequences encoding the self-cleaving peptides.

In one embodiment, exemplary vector configurations are shown in FIGS. 4A-4C. Exemplary vector configurations comprise a promoter or regulatory sequence (promoter/enhancer combination) driving the expression of the nucleic acid encoding the CUG-targeting PUF-endonuclease fusion. In another embodiment, a vector configuration comprises a promoter driving expression of the RNA-guided Cas RNase RNA-binding protein, or dCas protein fusion in operable linkage with a second promoter driving expressing of a cognate gRNA. In another embodiment, the vector configuration comprises a linker and one or more tags.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV2-Tyr mutant vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector, an AAV-Tyr mutant vector, and any combinations or equivalents thereof In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIV_{SM}) vector, a modified sooty mangabey simian immunodeficiency virus (SIV_{SM}) vector, a African green monkey simian immunodeficiency virus (SIV_{AGM}) vector, a modified African green monkey simian immunodeficiency virus (SIV_{AGM}) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

### Nucleic Acids

Provided herein are the nucleic acid sequences encoding RNA-binding CUG repeat-targeting systems disclosed herein for use in gene transfer and expression techniques described herein. It should be understood, although not always explicitly stated that the sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

The nucleic acid sequences (e.g., polynucleotide sequences) disclosed herein may be codon-optimized which is a technique well known in the art. In some embodiments disclosed herein, exemplary Cas sequences, such as e.g., a nucleic acid sequence encoding SEQ ID NO: 92 (Cas13d known as CasRx) or the nucleic acid sequence encoding SEQ ID NO: 298 (Cas13d known as CasRx), are codon optimized for expression in human cells. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences coding for, e.g., a Cas protein, can be generated. In some embodiments, such a sequence is optimized for expression in a host or target cell, such as a host cell used to express the Cas protein or a cell in which the disclosed methods are practiced (such as in a mammalian cell, e.g., a human cell). Codon preferences and codon usage tables for a particular species can be used to engineer isolated nucleic acid molecules encoding a Cas protein (such as one encoding a protein having at least 80%, at least 85%, at least 90%, at least 92%, at least 95%, at least 96%, at least 97%, at least 98%, at least 99%, or 100% sequence identity to its corresponding wild-type protein) that takes advantage of the codon usage preferences of that particular species. For example, the Cas proteins disclosed herein can be designed to have codons that are preferentially used by a particular organism of interest. In one example, a Cas nucleic acid sequence is optimized for expression in human cells, such as one having at least 70%, at least 80%, at least 85%, at least 90%, at least 92%, at least 95%, at least 98%, or at least 99% sequence identity to its corresponding wild-type or originating nucleic acid sequence. In some embodiments, an isolated nucleic acid molecule encoding at least one Cas protein (which can be part of a vector) includes at least one Cas protein coding sequence that is codon optimized for expression in a eukaryotic cell, or at least one Cas protein coding sequence codon optimized for expression in a human cell. In one embodiment, such a codon optimized Cas coding sequence has at least 80%, at least 85%, at least 90%, at least 92%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes a Cas protein having at least 85%, at least 90%, at least 92%, at least 95%, at least 96%, at least 97%, at least 98%, at least 99%, or 100% sequence identity to its corresponding wild-type or originating protein. In another embodiment, a variety of clones containing functionally equivalent nucleic acids may be routinely generated, such as nucleic acids which differ in sequence but which encode the same Cas protein sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W. H. 5 Freeman and Co., NY).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10× SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Cells

In some embodiments of the compositions and methods of the disclosure, a cell of the disclosure is a prokaryotic cell.

In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell.

In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, a somatic cell of the disclosure is a muscle cell. In some embodiments, a muscle cell of the disclosure is a myoblast or a myocyte. In some embodiments, a muscle cell of the disclosure is a cardiac muscle cell, skeletal muscle cell or smooth muscle cell. In some embodiments, a muscle cell of the disclosure is a striated cell. In one embodiment, a cell or cells of a patient treated with compositions disclosed herein include, without limitation, skeletal muscle (developing and mature muscle fibers and satellite cells), neuromuscular junction, cardiomyocytes, smooth muscle cells, peripheral nervous system (neurons), peripheral motor neurons, and/or sensory neurons.

In some embodiments of the compositions and methods of the disclosure, a somatic cell of the disclosure is a fibroblast or an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### Methods of Use

The disclosure provides a method of modifying level of expression of an RNA molecule of the disclosure or a protein encoded by the RNA molecule comprising contacting the composition of the disclosure and the RNA molecule under conditions suitable for binding of one or more of the guide RNA or the RNA-binding protein or RNA-binding fusion protein (or a portion thereof) to the RNA molecule.

The disclosure provides a method of modifying an activity of a protein encoded by an RNA molecule comprising contacting the composition of the disclosure and the RNA molecule under conditions suitable for binding of one or more of the guide RNA or the RNA-binding protein or the fusion protein (or a portion thereof) to the RNA molecule.

The disclosure provides a method of modifying level of expression of an RNA molecule of the disclosure or a protein encoded by the RNA molecule comprising contacting the composition of the disclosure and a cell comprising the RNA molecule under conditions suitable for binding of one or more of the guide RNA or the RNA-binding protein or fusion protein (or a portion thereof) to the RNA molecule. In some embodiments, the cell is in vivo, in vitro, ex vivo or in situ. In some embodiments, the composition of the disclosure comprises a vector comprising a guide RNA of the disclosure and an RNA-binding protein or fusion protein of the disclosure. In some embodiments, the vector is an AAV.

The disclosure provides a method of modifying an activity of a protein encoded by an RNA molecule comprising contacting the composition of the disclosure and a cell comprising the RNA molecule under conditions suitable for binding of one or more of the guide RNA or the RNA-binding protein or fusion protein (or a portion thereof) to the RNA molecule.

The disclosure provides a method of modifying the level of expression of an RNA molecule of the disclosure or a protein encoded by the RNA molecule comprising contacting the composition of the disclosure and the RNA molecule under conditions suitable for RNA nuclease activity wherein the RNA-binding protein or fusion protein induces a break in the RNA molecule.

The disclosure provides a method of modifying an activity of a protein encoded by an RNA molecule comprising contacting the composition of the disclosure and the RNA molecule under conditions suitable for RNA nuclease activity wherein the RNA-binding protein or fusion protein induces a break in the RNA molecule.

The disclosure provides a method of modifying a level of expression of an RNA molecule of the disclosure or a protein encoded by the RNA molecule comprising contacting the composition of the disclosure and a cell comprising the RNA molecule under conditions suitable for RNA nuclease activity wherein the RNA-binding protein or fusion protein induces a break in the RNA molecule. In some embodiments, the cell is in vivo, in vitro, ex vivo or in situ. In some embodiments, the composition comprises a vector comprising composition comprising a guide RNA of the disclosure and an RNA-binding fusion protein of the disclosure. In some embodiments, the vector is an AAV.

The disclosure provides a method of modifying an activity of a protein encoded by an RNA molecule comprising contacting the composition and a cell comprising the RNA molecule under conditions suitable for RNA nuclease activity wherein the RNA-binding protein or fusion protein induces a break in the RNA molecule. In some embodiments, the cell is in vivo, in vitro, ex vivo or in situ. In some embodiments, the composition comprises a vector comprising composition comprising a guide RNA or a single guide RNA of the disclosure and a nucleic acid sequence encoding an RNA-binding protein or fusion protein of the disclosure. In some embodiments, the vector is an AAV.

The disclosure provides a method of treating a disease or disorder comprising administering to a subject a therapeutically effective amount of a composition of the disclosure. In one embodiment, the disclosure provides a method of treating DM1.

The disclosure provides a method of treating a DM1 in a patient in need of such treatment comprising administering to the patient a therapeutically effective amount of a composition of the disclosure, wherein the composition comprises a vector comprising a guide RNA of the disclosure and a nucleic acid sequence encoding an RNA-binding protein or an RNA-binding protein fusion protein of the disclosure, wherein the composition modifies, reduces, destroys, knocks down or ablates a level of expression of a toxic CUG repeat RNA (compared to the level of expression of a toxic CUG repeat RNA treated with a non-targeting (NT) control or compared to no treatment). In one embodiment, the level of reduction of the target toxic CUG repeat RNA or toxic repeats encoded by the target RNA is compared to the level of reduction of the target RNA or toxic repeats encoded by the target RNA when treated with an RCas9 system. In another embodiment, the level of reduction is 1-fold or greater. In another embodiment, the level of reduction is 2-fold, 3-fold, 4-fold, 5-fold, 6-fold, 7-fold, 8-fold, 9-fold or 10-fold. In another embodiment, the level of reduction is 10-fold or greater. In another embodiment, the level of reduction is between 10-fold and 20-fold. In another embodiment, the level of reduction is 11-fold, 12-fold, 13-fold, 14-fold, 15-fold, 16-fold, 17-fold, 18-fold, 19-fold, or 20-fold. In another embodiment, the gene therapy compositions disclosed herein when administered to a DM1 patient lead to 20%-100% destruction (or elimination) of the toxic CUG repeat RNA. In one embodiment, the % elimination of the toxic CUG repeat RNA is any of 20-99%, 25%-99%, 50%-99%, 80%-99%, 90%-99%, 95%-99%. In one embodiment, the % elimination is 90%, 91%, 92%, 93%, 94%, 95%, 96%, 97%, 98%, or 99%. In another embodiment, % elimination is complete elimination or 100% elimination of the toxic CUG repeat RNA.

In some embodiments of the compositions and methods of the disclosure, a disease or disorder of the patient to be treated includes, without limitation, a disease or disorder related to CTG microsatellite repeat expansion expression. In some embodiments, the disease or disorder is related to CTG microsatellite repeat expansion in the 3′ untranslated region of the DMPK gene. In some embodiments of the compositions and methods of the disclosure, a disease or disorder of the disclosure is myotonic dystrophy type 1 (DM1).

In some embodiments of the methods of the disclosure, a subject of the disclosure has been diagnosed with DM1. In some embodiments, the subject of the disclosure presents at least one sign or symptom of DM1. At least one DM1 sign or DM1 symptom includes, without limitation, myotonia, muscle atrophy, centralized myonuclei, muscle strength recovery, GI distress, cardiac conduction defects, swallowing difficulty, respiratory capacity. In one embodiment, at least one sign or symptom of DM1 is ameliorated by treatment with the compositions disclosed herein. In some embodiments, the subject has a biomarker predictive of a risk of developing DM1. In some embodiments, the biomarker is a genetic mutation.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure, a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure, a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure, a subject of the disclosure is a mammal. In some embodiments, a subject of the disclosure is a non-human mammal.

In some embodiments of the methods of the disclosure, a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure, a therapeutically effective amount eliminates the disease or disorder.

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the disclosure, a composition of the disclosure is administered to the subject intramuscularly. In some embodiments, the composition of the disclosure is administered to the subject by an intravenous route. In some embodiments, the composition of the disclosure is administered to the subject by an injection or an infusion. In some embodiments, the composition is administered systemically. In some embodiments of the methods of the disclosure, a composition of the disclosure is administered to the subject locally.

In some embodiments, the compositions disclosed herein are formulated as pharmaceutical compositions. Briefly, pharmaceutical compositions for use as disclosed herein may comprise a protein(s) or a polynucleotide encoding the protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for routes of administration, such as e.g., oral, enteral, topical, transdermal, intranasal, and/or inhalation; and for routes of administration via injection or infusion such as, e.g., intravenous, intramuscular, subpial, intrathecal, intrastriatal, subcutaneous, intradermal, intraperitoneal, intratumoral, intravenous, intraocular, and/or parenteral administration. In certain embodiments, the compositions of the present disclosure are formulated for intravenous administration.

### EXAMPLE EMBODIMENTS

Embodiment 1. A method of treating myotonic dystrophy type 1 (DM1) in a mammal comprising administering a composition to a toxic target CUG microsatellite repeat expansion (MRE) molecule in tissues of the mammal, wherein the composition comprises a nucleic acid sequence encoding a non-naturally occurring or engineered clustered regularly interspaced short palindromic repeats (CRISPR)-associated (Cas) system comprising: (a) at least one RNA-guided RNase Cas protein (or dCas protein); and (b) at least one cognate CRISPR-Cas system guide RNA (gRNA) capable of forming a complex with one of the at least one Cas proteins, wherein the gRNA comprises (i) a DR sequence and (ii) a spacer sequence, wherein the spacer sequence hybridizes with the target CUG MRE molecule, and whereby the complex formed by the composition directly targets and destroys or blocks the target CUG MRE molecule thereby treating DM1 in the mammal.

Embodiment 2: The method of any preceding embodiment, wherein the spacer sequence comprises a spacer sequence selected from the group consisting of: agcagcagcagcagcagcagcagcag (SEQ ID NO: 457), gcagcagcagcagcagcagcagcagc (SEQ ID NO: 458), and cagcagcagcagcagcagcagcagca (SEQ ID NO: 459).

Embodiment 3: The method of any preceding embodiment, wherein the composition is administered to the tissue of the mammal by intravenous administration.

Embodiment 4: The method of any preceding embodiment, wherein the RNA-guided RNase Cas protein is selected from the group consisting of Cas13a, Cas13b, Cas13c, Cas13d, and an RNA-binding portion thereof.

Embodiment 5: The method of any preceding embodiment, wherein the RNA-guided RNase Cas protein is Cas13d or an RNA-binding portion thereof.

Embodiment 6: The method of any preceding embodiment, wherein the RNA-guided RNase Cas protein which is deactivated (dCas).

Embodiment 7: The method of any preceding embodiment, wherein Cas13d is encoded by a nucleic acid sequence set forth in any one of SEQ ID NOs 522, 530, 535, 538, or 540.

Embodiment 8: The method of any preceding embodiment, wherein the dCas protein is linked to an endonuclease.

Embodiment 9: The method of any preceding embodiment, wherein the endonuclease is ZC3H12A zinc-finger endonuclease.

Embodiment 10: The method of any preceding embodiment, wherein the ZC3H12A zinc finger nuclease comprises the amino acid sequence set forth in SEQ ID NO: 358 or SEQ ID NO: 359.

Embodiment 11: A composition comprising a nucleic acid sequence encoding a non-naturally occurring or engineered clustered regularly interspaced short palindromic repeats (CRISPR)-associated (Cas) system comprising: (a) at least one RNA-guided RNase Cas protein; and b) at least one cognate CRISPR-Cas system guide RNA (gRNA) capable of forming a complex with one of the at least one Cas proteins, wherein the gRNA comprises (i) a DR sequence and (ii) a spacer sequence, wherein the spacer sequence hybridizes with the target CUG MRE molecule, and wherein the spacer sequence comprises a spacer sequence selected from the group consisting of: agcagcagcagcagcagcagcagcag (SEQ ID NO: 457), gcagcagcagcagcagcagcagcagc (SEQ ID NO: 458), and cagcagcagcagcagcagcagcagca (SEQ ID NO: 459), or a portion thereof.

Embodiment 12: An AAV vector comprising the composition of any preceding embodiment.

Embodiment 13: The AAV vector of any preceding embodiment, which is an AAV9 vector.

Embodiment 14: A method of treating myotonic dystrophy type 1 (DM1) in a mammal comprising administering a composition to a toxic target CUG microsatellite repeat expansion (MRE) molecule in tissues of the mammal, wherein the composition comprises a nucleic acid sequence encoding a non-guided RNA-binding fusion protein comprising a) a PUF or PUMBY RNA-binding sequence capable of binding a toxic target CUG RNA repeat sequence, wherein the toxic target CUG repeat sequence comprises UGCUGCUG (SEQ ID NO: 453), and b) an endonuclease capable of cleaving the toxic target CUG RNA repeat sequence, whereby the level of expression of the toxic target RNA is reduced.

Embodiment 15: The embodiment of any preceding embodiment, wherein administration of the composition is via intravenous administration.

Embodiment 16: A method of eliminating toxic CUG microsatellite repeat expansion (MRE) RNA in tissues of a mammal with DM1, comprising contacting a the CUG MRE RNA sequence with a composition under conditions suitable for binding of the composition to the CUG MRE RNA, wherein the composition comprises a nucleic acid sequence encoding a fusion protein comprising a) a PUF or PUMBY RNA-binding sequence capable of binding a toxic target sequence comprising UGCUGCUG (SEQ ID NO: 453) and b) an endonuclease capable of cleaving the toxic target RNA sequence, whereby the toxic target RNA is eliminated. In one embodiment, cleavage of the toxic target RNA thereby reduces the level of expression of the toxic target RNA. In another embodiment, reduction of the level of expression of the toxic target RNA thereby eliminates the toxic target RNA.

Embodiment 17: A composition comprising a nucleic acid sequence encoding a non-guided RNA-binding fusion protein comprising a) a PUF or PUMBY protein capable of binding capable of binding a toxic target sequence comprising UGCUGCUG (SEQ ID NO: 453) and b) an endonuclease capable of cleaving the toxic target RNA sequence.

Embodiment 18: An AAV vector comprises the composition of any preceding embodiment.

Embodiment 19: The AAV vector of any preceding embodiment wherein the vector is AAV9.

Embodiment 20: The method or composition of any preceding embodiment, wherein the endonuclease is selected from the group consisting of: RNase1, RNase4, RNase6, RNase7, RNase8, RNase2, RNase6PL, RNaseL, RNaseT2, RNase11, RNaseT2-like, NOB1, ENDOV, ENDOG, ENDOD1, hFEN1, hSLFN14, hLACTB2, APEX2, ANG, HRSP12, ZC3H12A, RIDA, PDL6, NTHL, KIAA0391, APEX1, AGO2, EXOG, ZC3H12D, ERN2, PELO, YBEY, CPSF4L, hCG_2002731, ERCC1, RAC1, RAA1, RAB1, DNA2, FLJ35220, FLJ13173, ERCC4, RNase1(K41R), RNase1(K41R, D121E), RNase1(K41R, D121E, H119N), RNase1(H119N), RNase1(R39D, N67D, N88A, G89D, R91D, H119N), RNase1(R39D, N67D, N88A, G89D, R91D, H119N, K41R, D121E), RNase1(R39D, N67D, N88A, G89D, R91D), TENM1, TENM2, RNaseK, TALEN, ZNF638, and hSMG6 (PIN). Embodiment XX: The method or composition of any preceding embodiment, wherein the endonuclease is

In some embodiments of the methods and compositions disclosed herein, the RNA-binding polypeptide is an RNA-guided RNase Cas protein. In some embodiments, the Cas protein is Cas13a, Cas13b, Cas13c, or Cas13d. In some embodiments, the Cas protein is Cas13d.

In some embodiments, the RNA-binding polypeptide is a non-guided RNA-binding polypeptide. In some embodiments, the non-guided RNA-binding polypeptide is a PUF protein, or a PUMBY protein. In some embodiments, the PUF or PUMBY protein is a human PUF or PUMBY protein. In some embodiments, the non-guided RNA-binding polypeptide is a PUF or PUMBY fusion protein. In one embodiment, a PUF or PUMBY-based first RNA-binding protein is fused to a second RNA-binding protein. In some embodiments the second RNA-binding protein is a nuclease domain of a zinc-finger endonuclease known as ZC3H12A of SEQ ID NO: 358 (also termed herein E17).

In some embodiments of the methods and compositions disclosed herein, the nucleic acid sequence comprises at least one promoter. In some embodiments, the at least one promoter is a constitutive promoter or a tissue-specific promoter. In one embodiment, the promoter is a CAG or truncated CAG (tCAG) promoter. In another embodiment, the promoter is an EFS promoter. In one embodiment, the tissue-specific promoter is a muscle-specific promoter. In another embodiment, the muscle-specific promoter is a MHCK7 promoter. In another embodiment, the muscle-specific promoter is a desmin promoter. In one embodiment, the desmin promoter is a full-length desmin promoter. In another embodiment, the desmin promoter is a truncated desmin promoter.

In some embodiments of the methods and compositions disclosed herein, the RNA-guided RNase Cas protein or the non-guided RNA-binding polypeptide is a first RNA-binding polypeptide which is fused with a second RNA-binding polypeptide. In one embodiment, the second RNA-binding polypeptide is capable of binding RNA in a manner in which it associates with RNA. In some embodiments, the second RNA-binding polypeptide is capable of associating with RNA in a manner in which it cleaves RNA. In some embodiments, the second RNA-binding polypeptide is selected from the group consisting of: RNase1, RNase4, RNase6, RNase7, RNase8, RNase2, RNase6PL, RNaseL, RNaseT2, RNase11, RNaseT2-like, NOB1, ENDOV, ENDOG, ENDOD1, hFEN1, hSLFN14, hLACTB2, APEX2, ANG, HRSP12, ZC3H12A, RIDA, PDL6, NTHL, KIAA0391, APEX1, AGO2, EXOG, ZC3H12D, ERN2, PELO, YBEY, CPSF4L, hCG_2002731, ERCC1, RAC1, RAA1, RAB1, DNA2, FLJ35220, FLJ13173, ERCC4, RNase1(K41R), RNase1(K41R, D121E), RNase1(K41R, D121E, H119N), RNase1(H119N), RNase1(R39D, N67D, N88A, G89D, R91D, H119N), RNase1(R39D, N67D, N88A, G89D, R91D, H119N, K41R, D121E), RNase1(R39D, N67D, N88A, G89D, R91D), TENM1, TENM2, RNaseK, TALEN, ZNF638, and hSMG6 (PIN). In one embodiment, the second RNA-binding polypeptide is a nuclease domain from ZC3H12A (E17). In some embodiments, the first RNA-binding protein is an RNA-guided RNase Cas protein which is deactivated.

In some embodiments disclosed herein is a vector comprising the CUG-targeting DM1 compositions disclosed herein. In some embodiments, the vector is selected from the group consisting of: adeno-associated virus, retrovirus, lentivirus, adenovirus, nanoparticle, micelle, liposome, lipoplex, polymersome, polyplex, and dendrimer. In one embodiment, an AAV vector comprises the CUG-targeting DM1 compositions disclosed herein. In one embodiment, the AAV vector is AAV9. In another embodiment, the AAV vector is AAVrh.74. In some embodiments disclosed herein is a cell comprising the vector or vectors disclosed herein.

In some embodiments of the compositions of the disclosure, the sequence comprising the gRNA further comprises a sequence encoding a promoter capable of expressing the gRNA in a eukaryotic cell.

In some embodiments of the compositions of the disclosure, the eukaryotic cell is an animal cell. In some embodiments, the animal cell is a mammalian cell. In some embodiments, the animal cell is a human cell. In some embodiments, the human cell is a muscle cell.

In some embodiments of the compositions of the disclosure, the promoter is a constitutively active promoter. In some embodiments, the promoter sequence is isolated or derived from a promoter capable of driving expression of an RNA polymerase. In some embodiments, the promoter sequence is a Pol III promoter. In some embodiments, the promoter sequence is isolated or derived from a human U6 promoter. In some embodiments, the promoter is a sequence isolated or derived from a promoter capable of driving expression of a transfer RNA (tRNA). In some embodiments, the promoter is isolated or derived from an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine tRNA promoter, a methionine tRNA promoter, a phenylalanine tRNA promoter, a proline tRNA promoter, a serine tRNA promoter, a threonine tRNA promoter, a tryptophan tRNA promoter, a tyrosine tRNA promoter, or a valine tRNA promoter. In some embodiments, the promoter is isolated or derived from a valine tRNA promoter.

In some embodiments of the compositions of the disclosure, the DR sequence of the gRNA is a Cas13d DR sequence.

In some embodiments of the compositions of the disclosure, the first RNA binding protein comprises a CRISPR-Cas protein which is not a Cas9 CRISPR-Cas protein. In some embodiments, the CRISPR-Cas protein comprises a native RNA nuclease activity. In some embodiments, the native RNA nuclease activity is reduced or inhibited. In some embodiments, the native RNA nuclease activity is increased or induced. In some embodiments, the CRISPR-Cas protein and/or the cognate gRNA comprises a mutation. In some embodiments, a nuclease domain of the CRISPR-Cas protein comprises the mutation. In some embodiments, the DR sequence of the gRNA comprises the mutation. In some embodiments, the mutation occurs in a nucleic acid encoding the CRISPR-Cas protein or the nucleic acid encoding the gRNA. In some embodiments, the mutation occurs in an amino acid encoding the CRISPR-Cas protein. In some embodiments, the mutation comprises a substitution, an insertion, a deletion, a frameshift, an inversion, or a transposition. In some embodiments, the mutation comprises a deletion of a nuclease domain, a binding site within the nuclease domain, an active site within the nuclease domain, or at least one essential amino acid residue within the nuclease domain. In some embodiments, the mutation is a point mutation in the DR sequence of the gRNA.

In some embodiments, the CRISPR-Cas protein is a Type VI CRISPR-Cas protein. In some embodiments, the RNA binding protein comprises a Cas13 polypeptide or an RNA-binding portion thereof In some embodiments, the RNA binding protein comprises a Cas13d polypeptide or an RNA-binding portion thereof In some embodiments, the CRISPR-Cas protein comprises a native RNA nuclease activity. In some embodiments, the native RNA nuclease activity is reduced or inhibited. In some embodiments, the native RNA nuclease activity is increased or induced. In some embodiments, the CRISPR-Cas protein and/or its cognate gRNA comprises a mutation. In some embodiments, a nuclease domain of the CRISPR-Cas protein comprises the mutation. In some embodiments, the mutation occurs in a nucleic acid encoding the CRISPR-Cas protein or in the nuclei acid encoding the gRNA. In some embodiments, the mutation occurs in an amino acid sequence of the CRISPR-Cas protein. In some embodiments, the mutation comprises a substitution, an insertion, a deletion, a frameshift, an inversion, or a transposition. In some embodiments, the mutation comprises a deletion of a nuclease domain, a binding site within the nuclease domain, an active site within the nuclease domain, or at least one essential amino acid residue within the nuclease domain. In some embodiments, the mutation is a point mutation in the DR sequence of the gRNA. In one embodiment, the mutated DR sequence is a Cas13d DR sequence comprising a point mutation.

In some embodiments of the methods and/or compositions of the disclosure, the non-guided RNA binding protein does not require multimerization for RNA-binding activity. In some embodiments, the non-guided RNA binding protein is not a monomer of a multimer complex. In some embodiments, a multimer protein complex does not comprise the RNA binding protein.

In some embodiments of the methods and/or compositions of the disclosure, the RNA binding protein selectively binds to a target sequence within the RNA molecule. In some embodiments, the RNA binding protein does not comprise an affinity for a second sequence within the RNA molecule. In some embodiments, the RNA binding protein does not comprise a high affinity for or selectively binds a second sequence within the RNA molecule.

In some embodiments of the methods and/or compositions of the disclosure, an RNA genome or an RNA transcriptome comprises the RNA molecule.

In some embodiments of the methods and/or compositions of the disclosure, the RNA binding protein comprises between 2 and 1300 amino acids, inclusive of the endpoints.

In some embodiments of the methods and/or compositions of the disclosure, the RNA binding fusion protein comprises between 2 and 2000 amino acids, inclusive of the endpoints.

In some embodiments of the methods and/or compositions of the disclosure, the sequence encoding the RNA binding protein further comprises a sequence encoding nuclear localization signal (NLS), a nuclear export signal (NES) or tag. In some embodiments, the sequence encoding a nuclear localization signal (NLS) is positioned at the N-terminus of the sequence encoding the RNA binding protein. In some embodiments, the RNA binding protein comprises an NLS at a C-terminus of the protein. In some embodiments, the sequence encoding the RNA binding protein or system comprises two NLSs or two NESs.

In some embodiments of the methods and/or compositions of the disclosure, the sequence encoding the RNA binding protein further comprises a first sequence encoding a first NLS and a second sequence encoding a second NLS. In some embodiments, the sequence encoding the first NLS or the second NLS is positioned at the N-terminus of the sequence encoding the RNA binding protein. In some embodiments, the RNA binding protein comprises the first NLS or the second NLS at a C-terminus of the protein. In some embodiments, the RNA-binding compositions comprise at least one linker.

In some embodiments of the methods and/or compositions of the disclosure, the composition further comprises a second RNA binding protein. In some embodiments, the second RNA binding protein comprises or consists of a nuclease domain. In some embodiments, the second RNA binding protein binds RNA in a manner in which it associates with RNA. In some embodiments, the second RNA binding protein associates with RNA in a manner in which it cleaves RNA. In some embodiments of the compositions of the disclosure, the sequence encoding the second RNA binding protein comprises or consists of an RNase.

Also disclosed herein are methods of manufacture of the compositions and/or vectors comprising the compositions disclosed herein.

Any of the preceding embodiments, wherein the nucleic acid sequence comprises at least one promoter, wherein the at least one promoter is a constitutive promoter or a tissue-specific promoter. wherein the at least one promoter is an EFS promoter or a tCAG promoter, wherein the tissue-specific promoter is a muscle-specific promoter, wherein the muscle-specific promoter is a MHCK7 promoter, and/or wherein any of the preceding promoters comprise an enhancer and/or an intron.

Any of the preceding embodiments, wherein the level of expression of the toxic target RNA is reduced compared to the reduction in the level of expression of the untreated toxic target RNA, wherein the level of expression of the toxic target RNA is reduced compared to the level of reduction of the toxic target RNA treated with RCas9-based systems, wherein the level of reduction is 1-fold or greater, wherein the level of reduction is 2-fold, 3-fold, 4-fold, 5-fold, 6-fold, 7-fold, 8-fold, 9-fold or 10-fold, wherein the level of reduction is 10-fold or greater, wherein the level of reduction is between 10-fold and 20-fold.

The methods and compositions of any of the preceding embodiments, wherein the nucleic acid sequence comprising a promoter capable of expressing the gRNA in a human cell, wherein the promoter is a human U6 promoter, wherein the promoter is a sequence isolated or derived from a promoter capable of driving expression of a transfer RNA (tRNA), wherein the promoter is selected from the group consisting of an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine tRNA promoter, a methionine tRNA promoter, a phenylalanine tRNA promoter, a proline tRNA promoter, a serine tRNA promoter, a threonine tRNA promoter, a tryptophan tRNA promoter, a tyrosine tRNA promoter, and a valine tRNA promoter. wherein the DR sequence of the gRNA is a Cas13d DR sequence, wherein the nucleic acid sequence further comprises a sequence encoding at least one nuclear localization signal (NLS), nuclear export signal (NES) or tag, wherein the at least one nuclear localization signal (NLS) is positioned at the N-terminus of the sequence encoding the RNA binding protein, wherein the at least one nuclear localization signal (NLS) is positioned at the C-terminus of the sequence encoding the RNA binding protein, wherein the nucleic acid sequence further comprises a sequence encoding two nuclear localization signals (NLSs) or two nuclear export signal (NESs), wherein the RNA-binding protein or fusion protein comprises at least one linker.

### EXAMPLES

### Example 1: Cas13d Systems Destroy DM1 Toxic CUG Repeats

### Materials

Plasmids: CTG960 (960 CTG repeats), Tet transactivator, pcDNA3.1 expressing Cas and sgRNA under human U6 promoter

Transfection reagents: Lipofectamine 3000, opti-MEM, PBS, DMEM

RNA extraction kit: RNEASY PLUS (Qiagen)

qScript™ One-Step SYBR@ Green qRT-PCR Kit, Quantabio

### Methods

Transfection, RNA Extraction, RNA-FISH, and qRT-PCR Analysis

To assess the knockdown of CUG repeat containing RNA, 5×10⁴ COSM6 cells were seeded on 24 well plates and transfected with 50 ng of the DMPK-CTG960 plasmid where CUG-960 expression is driven under the control of the tetracycline regulatable promoter element (TRE), 25 ng of Tet transactivator (tTA) plasmid, and 1 μg of the RBP (Cas+sgRNA). Spacer sequences used in sgRNAs for CUG targeting with Cas13d systems are listed in Table T. Cas proteins and corresponding sgRNAs were expressed from the same plasmid. Cells were incubated with 100 ng/ml doxycycline in Opti-MEM (containing 5% FBS) overnight which is required to drive the expression of the CUG-960 RNA. Cells were washed with PBS and incubated in complete DMEM media supplemented with 100 ng/ml doxycycline for additional 24 hours. Cells were harvested, RNA was extracted, and qRT-PCR was performed. CUG expression ΔΔCT was calculated normalized to housekeeping gene and relative to non-targeting (NT) sgRNA to quantify the CUG-960 knockdown.

TABLE 51
Spacers sequences used in sgRNAs for
CUG targeting with Cas13d systems:

| Spacer | Spacer Sequences |
|---|---|
| 1 | agcagcagcagcagcagcagcagcag |
| | (SEQ ID NO: 457) |
| 2 | gcagcagcagcagcagcagcagcagc |
| | (SEQ ID NO: 458) |
| 3 | cagcagcagcagcagcagcagcagca |
| | (SEQ ID NO:459) |

FIG. 1 demonstrates that CUG-targeting Cas13d systems utilizing spacer sequences SEQ ID NOs 457-459 eliminate >90% of CUG targets compared to control.

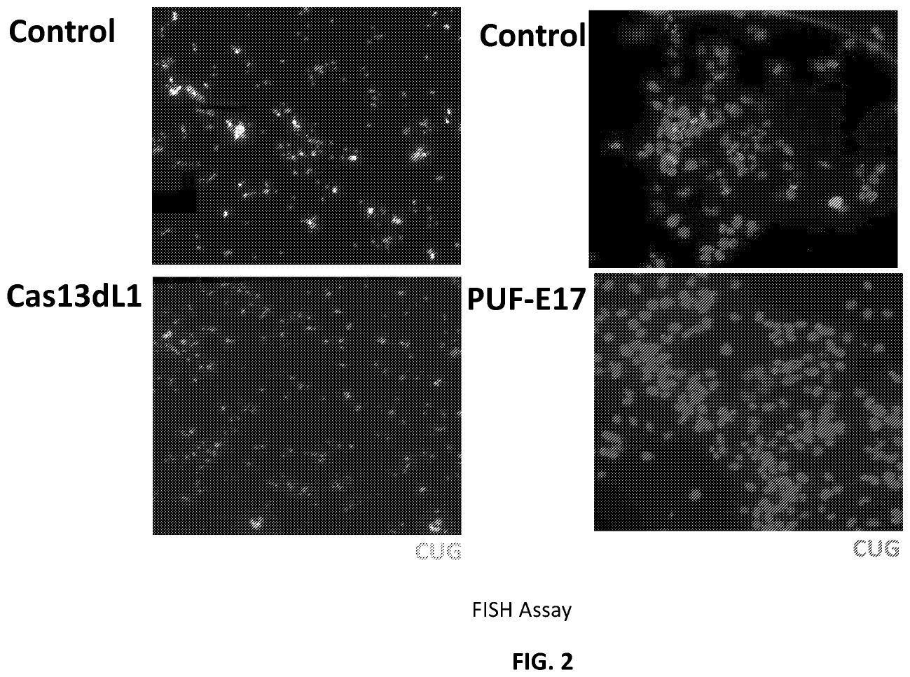

FIG. 2 demonstrates that CUG-targeting Cas13d systems eliminate >90% of CUG-repeat RNA compared to control as analyzed by RNA fluorescence in situ hybridization (RNA-FISH).

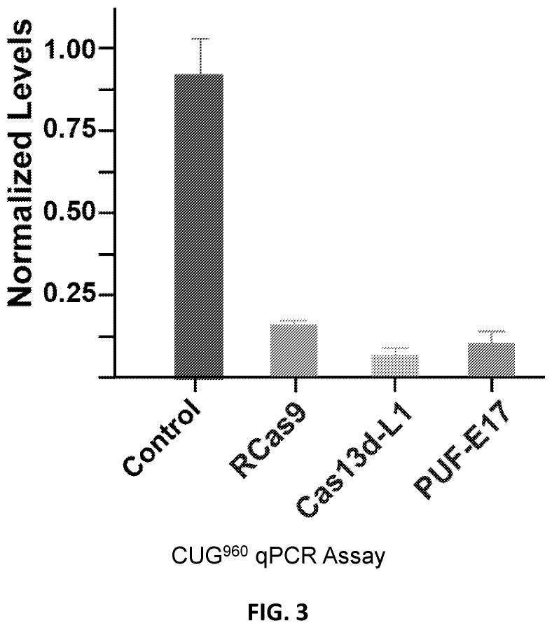

FIG. 3 shows Cas13d systems compared to RCas9 systems. Cas13d systems result in efficient cleavage eliminating >90% of CUG targets compared to control NT sgRNA. Cells treated with RCas9 exhibited about 80% knockdown of the target CUG repeats.

### Example 2: PUF Systems Destroy DM1 Toxic CUG Repeats

### Materials

Plasmids: CTG960 (960 CTG repeats), Tet transactivator, pcDNA3.1 expressing RBP

Transfection reagents: Lipofectamine 3000, opti-MEM, PBS, DMEM

RNA extraction kit: RNEASY PLUS (Qiagen)

qScript™ One-Step SYBR@ Green qRT-PCR Kit, Quantabio Methods Transfection, RNA extraction, FISH, and qRT-PCR Analysis

To assess the knockdown of CUG repeat containing RNA, 5×10⁴ COSM6 cells were seeded on 24 well plates and transfected with 50 ng of the DMPK-CTG960 plasmid where CUG-960 expression is driven under the control of the tetracycline regulatable promoter element (TRE), 25 ng of Tet transactivator (tTA) plasmid, and 1 μg of the RBP (PUFs). Cells were incubated with 100 ng/ml doxycycline in Opti-MEM (containing 5% FBS) overnight. Cells were washed with PBS and incubated in complete DMEM media supplemented with 100 ng/ml doxycycline for additional 24 hours. Cells were harvested, RNA was extracted, and qRT-PCR was performed. CUG expression ΔΔCT was calculated normalized to housekeeping gene and relative to non-targeting (NT) control to quantify CUG960 RNA knockdown.

The PUF constructs (labeled in FIG. 1 as CUG-f1 and CUG-f2) targeted the toxic sequence UGCUGCUG (SEQ ID NO: 453).

FIG. 1 demonstrates that CUG-targeting PUF-E17 fusion systems eliminate >90% of CUG targets compared to control.

FIG. 2 demonstrates that PUF(CUG)-E17 fusion systems eliminate >90% of CUG-repeat RNA compared to control as analyzed by RNA fluorescence in situ hybridization (RNA-FISH).

FIG. 3 shows PUF(CUG) systems compared to RCas9 systems. PUF(CUG) systems result in efficient cleavage eliminating >90% of CUG targets compared to control NT. Cells treated with RCas9 exhibited about 80% knockdown of the target CUG repeats.

### Example 3: Targeting Expanded CUG Repeats at the RNA Level for the Treatment of Myotonic Dystrophy Type 1 (Destruction and Blocking Mechanisms)

RNA-targeting compositions disclosed herein A01215 (Cas13d(CUG) for destruction), A01344 (PUF(CUG)-E17 for destruction), and A01686 (for blocking) were delivered via either systemic or intramuscular routes via AAV-based approaches. For example, the PUF targeting CUG construct for AAV9-based delivery in the below art-recognized animal model for myotonic dystrophy is presented in Table 53.

TABLE 53
PUF targeting CUG construct
for AAV9-based delivery

| Protein | | Target | Amino Acid |
|---|---|---|---|
| Type | Elements | Sequence | Sequence of PUF |
| 8PUF | N-terminal | UGCUGCUG | GRSRLLEDFRNNRYP |
| | SV40 NLS; | (SEQ ID | NLQLREIAGHIMEFS |
| | Linker | NO: 453) | QDQHGSRFIELKLER |
| | between | | ATPAERQLVFNEILQ |
| | PUF and | | AAYQLMVDVFGNYVI |
| | E17 | | QKFFEFGSLEQKLAL |
| | endo- | | AERIRGHVLSLALQM |
| | nuclease | | YGSYVIRKALEFIPS |
| | (VDTANGS); | | DQQNEMVRELDGHVL |
| | C-terminal | | KCVKDQNGSYVVEKC |
| | E17 | | IECVQPQSLQFIIDA |
| | | | FKGQVFALSTHPYGN |
| | | | RVIQRILEHCLPDQT |
| | | | LPILEELHQHTEQL |
| | | | VQDQYGSYVIRHVLE |
| | | | HGRPEDKSKIVAEIR |
| | | | GNVLVLSQHKFASNV |
| | | | VEKCVTHASRTERAV |
| | | | LIDEVCTMNDGPHSA |
| | | | LYTMMKDQYANYVVQ |
| | | | KMIDVAEPGQRKIVM |
| | | | HKIRPHIATLRKYTY |
| | | | GKHILAKLEKYYMKN |
| | | | GVDLG |
| | | | (SEQ ID NO: 444) |

In order to target expanded CUG repeats associated with myotonic dystrophy, vector with DNA encoding CUG-targeting compositions were delivered by an AAV vector. PUF(CUG)-E17, Cas13d(CUG) or PUF(CUG) alone expression was driven by a promoter (FIG. 4A-C and FIG. 12 A-B). In some aspects, a truncated CAG (tCAG) promoter (SEQ ID NO: 385) was used. In some aspects, a short EF1-alpha (EFS) promoter (SEQ ID NO: 520) was used. In some aspects, an EFS promoter with a UBB intron (SEQ ID NO: 609) was used.

AAV-9 preparations were generated according to standard techniques (triple-transfection method) and purified by IDX gradient ultracentrifugation. AAV was titered by qPCR and capsid ELISA. The AAV9 version described above was next injected either intravenously (150 μL total volume, 1*10\hat{ }12 vg) or to the tibialis anterior muscles of HSA-LR myotonic dystrophy type 1 mice (50 μL total volume, 2*10\hat{ }10 vg, 1*10\hat{ }11 vg) and subjected to daily clinical observation subsequently. Mice were sacrificed at 4w and 12w after injection. For each animal, the proximal halves of the tibialis anterior (injection site for IM injection), quadriceps, diaphragm, gastrocnemius muscles, heart, spleen, liver (representative portion, i.e., piece of a lobe), intestines and kidneys were collected, placed individually (except pair organs) into cryovials and flash frozen in liquid nitrogen for RNA/protein assessment and changes in gene expressions. The other halves of all the tissues were embedded in OCT and frozen. The muscle sections were cut in a transverse fashion. The other half of the tissues was used to prepare total RNA.

Electromyography was performed prior to sacrificing mice at 4 and 12 weeks timepoints to assess the reversal of disease-associated myotonia (defect in muscle relaxation) as a measure of efficacy of the RNA targeting compositions in reversing myotonic dystrophy type 1 physiological phenotypes.

RNA isolations from frozen tissue were carried out with RNAeasy columns (Qiagen) according to the manufacturer's protocol. RNA quality and concentrations were estimated using the Nanodrop spectrophotometer. cDNA preparation was done using Superscript III (Thermofisher) with random primers according to the manufacturer's protocol. qPCR and digital droplet PCR (ddPCR) were carried out to assess the levels of the RNA-targeting compositions in tissue among the two mouse groups (RNA-targeting compositions, Vehicle) and to assess durability of transgene expression.

RNA Fluorescence in-situ hybridization (FISH) was performed to measure the disappearance of RNA foci in the muscle sections as a measure of efficacy of the RNA-targeting compositions in eliminating or blocking expanded CUG repeats.

Immunofluorescence with antibodies against the chloride channel Clcn1 and Mbnl1 was performed as a measure of efficacy of the RNA-targeting compositions for the treatment of myotonic dystrophy type 1 molecular pathology. Reconstitution of Clcn1 and Redistribution of nuclear Mbnl1 signified reversal myotonic dystrophy type 1 molecular pathology.

cDNA was prepared from the RNA isolated from muscle tissue. RT-PCR is performed with primers flanking the alternatively spliced exon 22 of Atp2al, exon 7 of Clcn1, exon 11 of BIN1 to show reversal of myotonic dystrophy type 1 associated alternative splicing.

### Example 4: Targeting Expanded CUG Repeats at the RNA Level for the Treatment of Myotonic Dystrophy Type 1 by dCas13d and a CUG-Targeting Guide RNA

A single transgene encoding CUG-targeting guide RNA (gRNA) under the human U6 promoter and a nuclease-dead dCas13d linked to the endonuclease E17 (derived from human ZC3H112A gene) or a nuclease-dead dCas13d (alone) is delivered via either systemic or intramuscular routes via viral or nonviral means. In order to target expanded CUG repeats associated with myotonic dystrophy, vectors with DNA encoding CUG-targeting gRNA and dCas13d(CUG) is delivered by an AAV vector. Cas13d (or dCas13d) expression is driven by a promoter (FIG. 4B, FIG. 4C and FIG. 12B). In some aspects, a truncated CAG (tCAG) promoter (SEQ ID NO: 385) was used. In some aspects, a short EF1-alpha (EFS) promoter (SEQ ID NO: 520) was used. In some aspects, an EFS promoter with a UBB intron (SEQ ID NO: 609) was used.

AAV9 preparations were generated according to standard techniques (triple-transfection method) and purified by IDX gradient ultracentrifugation. AAV was titered by qPCR and capsid ELISA. The AAV9 version described above is next injected into the either intravenously (150 μL total volume, 1*10\hat{ }12 vg) or to the tibialis anterior muscles of HSA-LR myotonic dystrophy type 1 mice (50 μL total volume, 2*10\hat{ }vg, 1*10\hat{ }11 vg vg) and subjected to daily clinical observation subsequently. Mice are sacrificed at 4w and 12w after injection. For each animal, the proximal halves of the tibialis anterior (injection site for IM injection), quadriceps, diaphragm, gastrocnemius muscles, heart, spleen, liver (representative portion, i.e., piece of a lobe), intestines and kidneys are collected, placed individually (except pair organs) into cryovials and flash frozen in liquid nitrogen for RNA/protein assessment and changes in gene expressions. The other halves of all the tissues are embedded in OCT and frozen. The muscle sections are cut in a transverse fashion. The other half of the tissues is used to prepare total RNA.

Electromyography is performed prior to sacrificing mice at 4 and 12 weeks timepoints to assess the reversal of disease-associated myotonia (defect in muscle relaxation) as a measure of efficacy of the dCas13d(CUG)-E17 via destruction or dCas13d(CUG) via blocking in reversing myotonic dystrophy type 1 physiological phenotypes. RNA isolations from frozen tissue is carried out with RNAeasy columns (Qiagen) according to the manufacturer's protocol. RNA quality and concentrations are estimated using the Nanodrop spectrophotometer. cDNA preparation is done using Superscript III (Thermofisher) with random primers according to the manufacturer's protocol. qPCR and digital droplet PCR (ddPCR) are carried out to assess the levels of Cas13d (or dCas13d) and gRNA in tissue among the two mouse groups (CUG targeting RNA along with Cas13d or dCas13d, Vehicle) to assess durability of transgene expression. RNA Fluorescence in-situ hybridization (FISH) is performed to measure the disappearance of RNA foci in the muscle sections as a measure of efficacy of CUG-targeting gRNA+Cas13d (or dCas13d) in eliminating expanded CUG repeats. Immunofluorescence with antibodies against the chloride channel Clcn1 and Mbnl1 is performed as a measure of efficacy of gRNA+Cas13d (or dCas13d) for the treatment of myotonic dystrophy type 1 molecular pathology. Reconstitution of Clcn1 and Redistribution of nuclear Mbnl1 signifies reversal myotonic dystrophy type 1 molecular pathology. cDNA is prepared from the RNA isolated from muscle tissue. RT-PCR is performed with primers flanking the alternatively spliced exon 22 of Atp2al, exon 7 of Clcn1, exon 11 of BIN1 to show reversal of myotonic dystrophy type 1 associated alternative splicing.

### Example 5: Dose-Dependent Reduction in CUG^{exp} RNA Via Destruction or Blocking in Patient Myocytes and HSA DM1 Mice (Described Above)

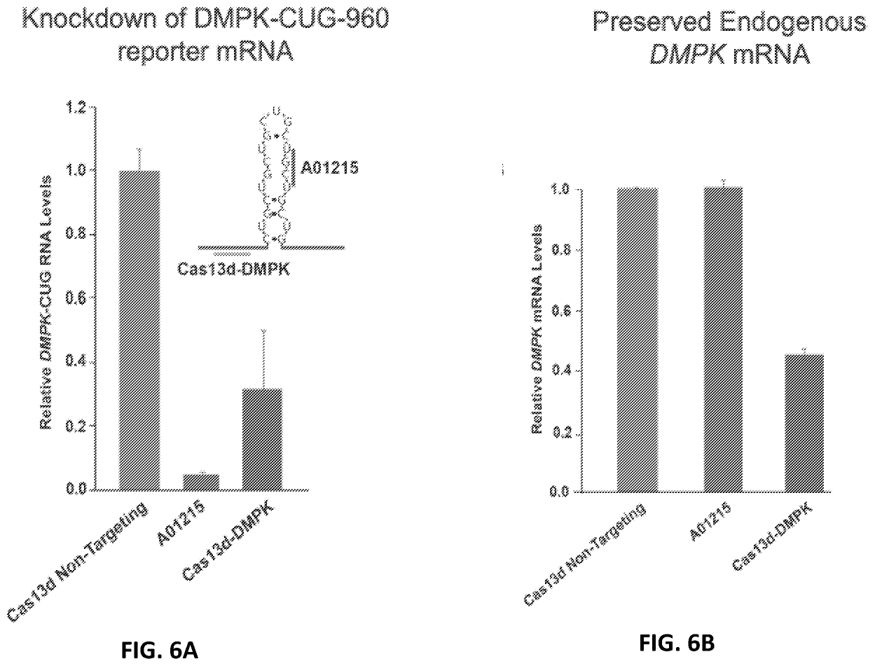

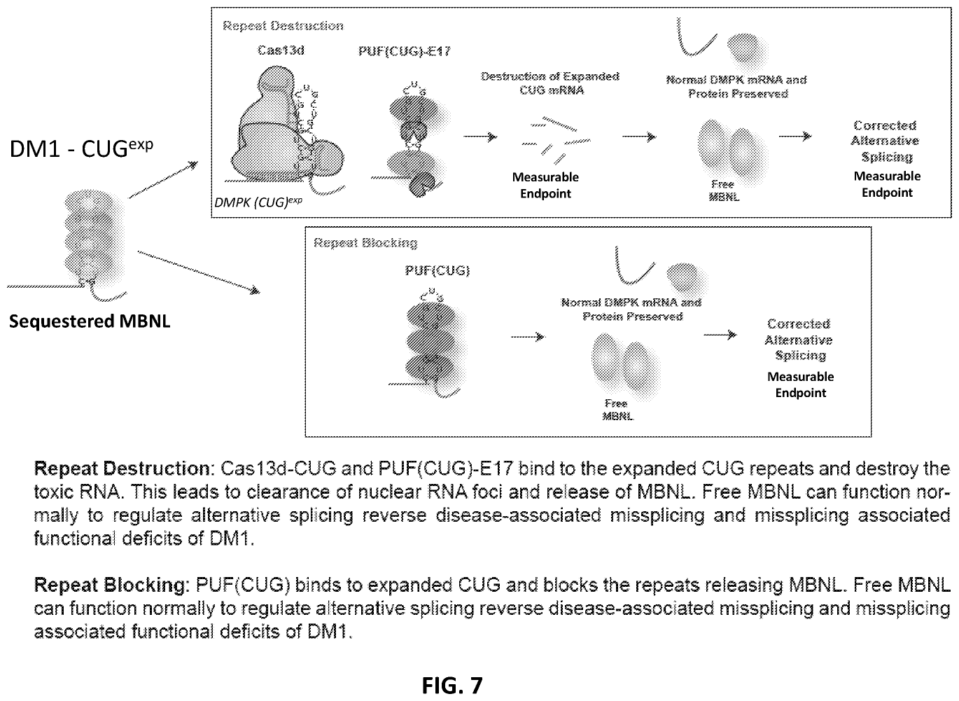

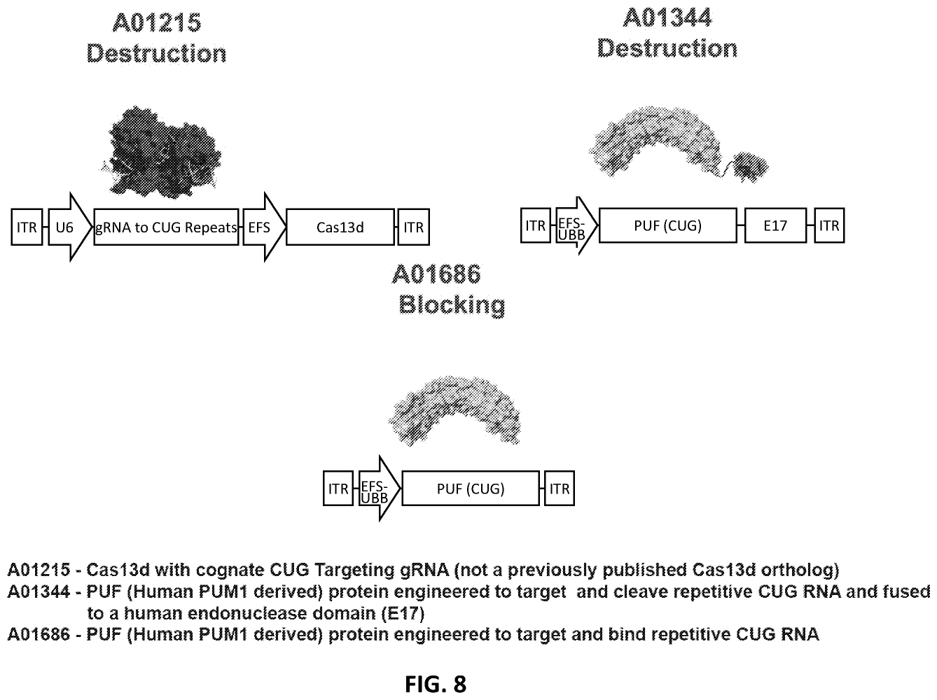

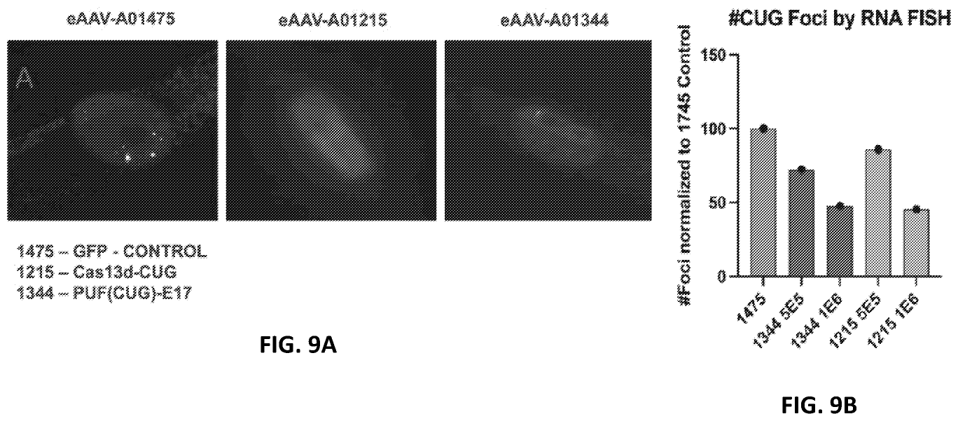

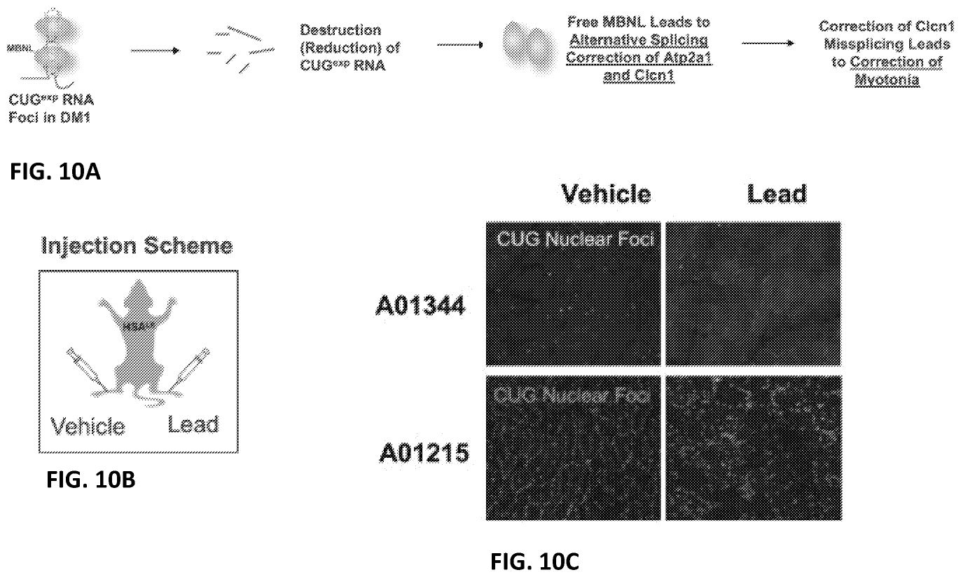

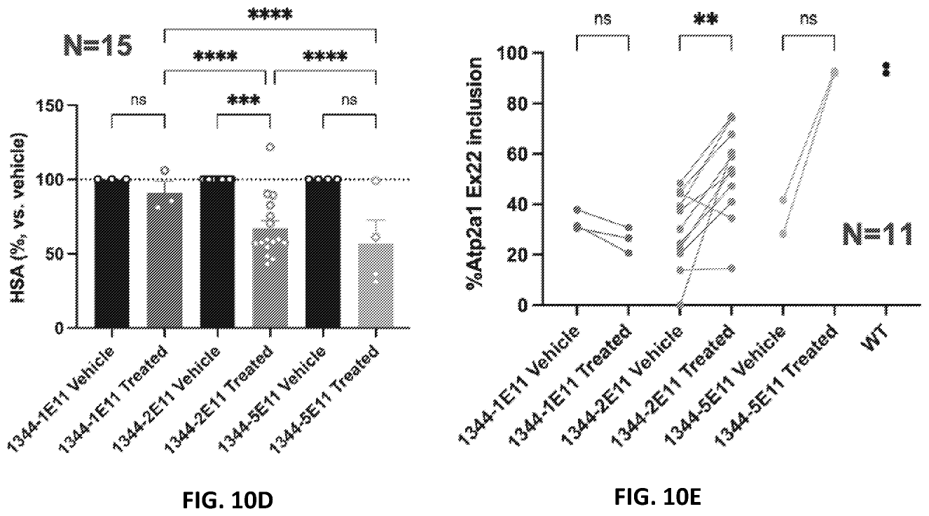

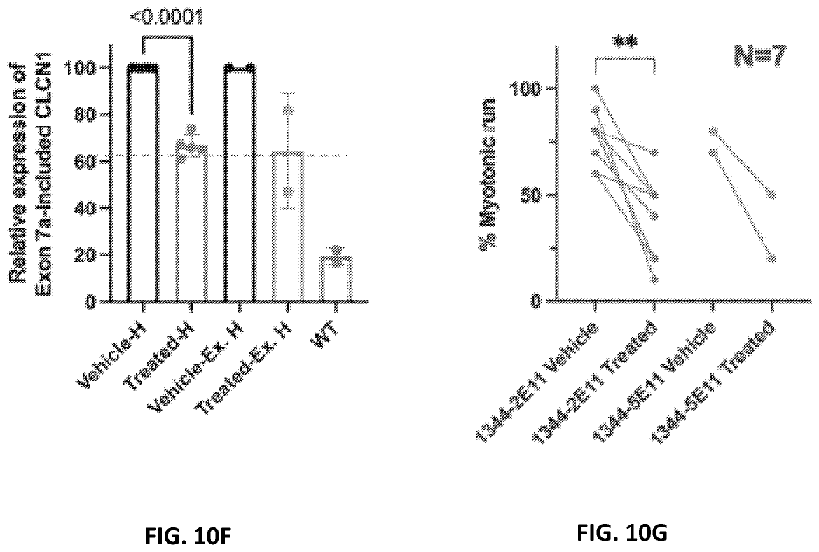

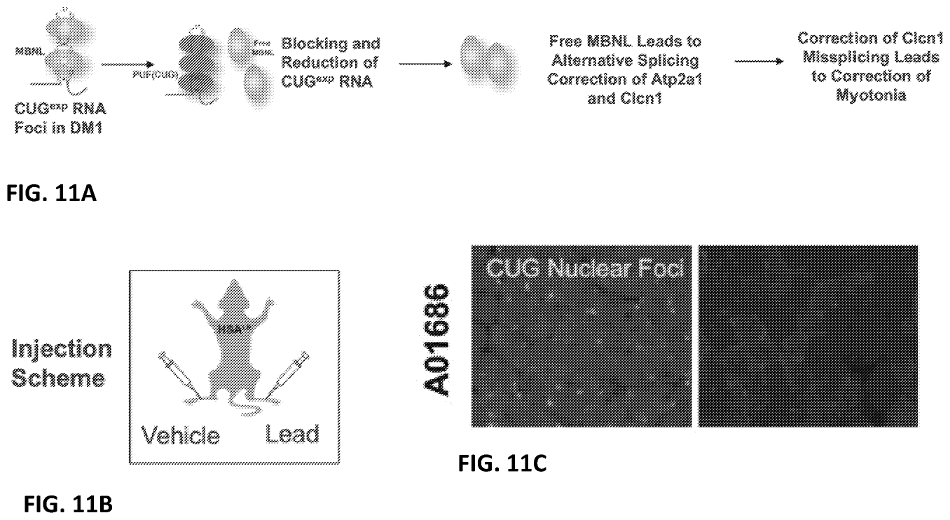

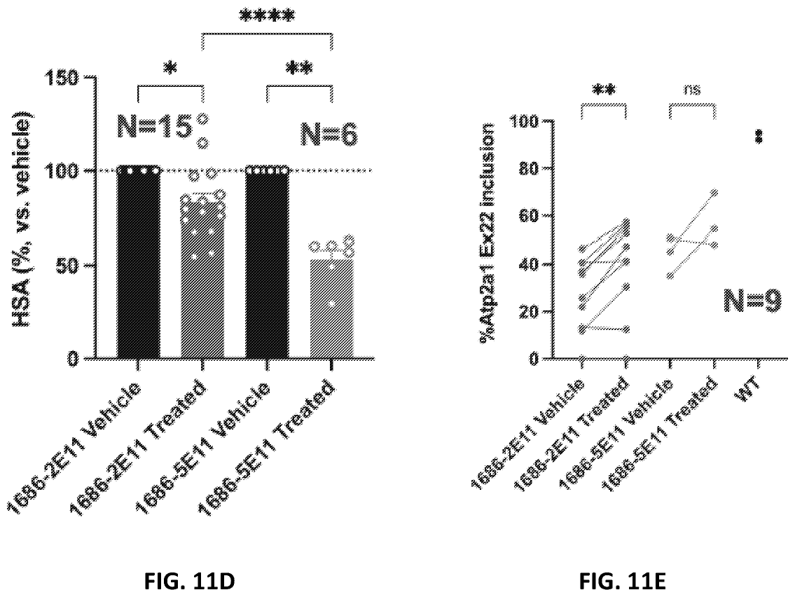

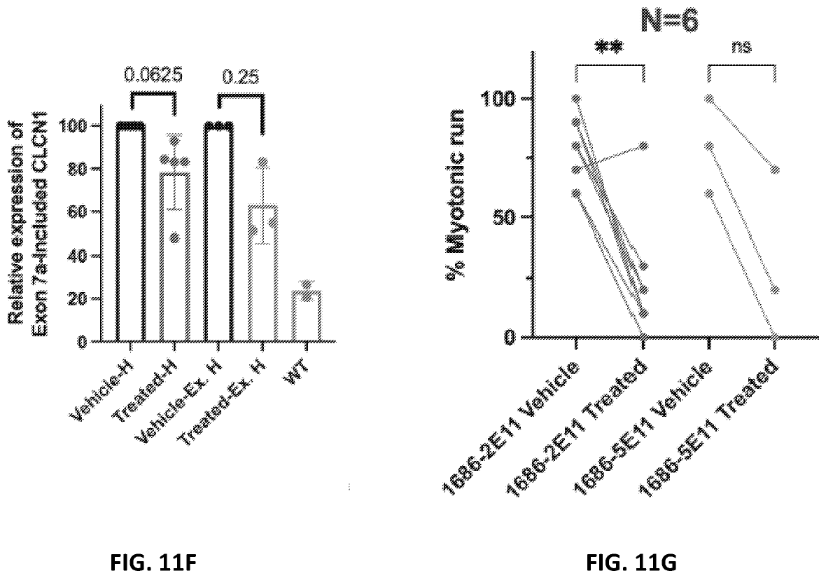

Myotonic dystrophy type I (DM1) is a multisystemic autosomal-dominant inherited disorder caused by CUG microsatellite repeat expansions (MREs) in the 3′ untranslated region (UTR) of the DMPK mRNA. Previously, we showed that a CRISPR/Cas9-based RNA-targeting gene therapy has the potential to eliminate toxic RNAs expressed from repetitive tracts in DM1 in primary patient cells and a DM1 mouse model. To further explore therapeutic MRE targeting strategies which are less wieldy and better suited to lower or eliminate off-target effects, we engineered RNA-targeting systems: 1) a novel CRISPR/Cas13d (A01215), 2) a PUF RNA binding protein system derived from the naturally occurring human PUM1 protein linked with an RNA endonuclease (PUF-E17) derived from human ZC3H112A (A01344), and 3) a PUF RNA binding protein system without an endonuclease (A01686) to target and either cleave (1 & 2) or block expanded DM1-related CUG repeats. Modified AAvrh74-packaged Cas13d and PUF-E17 reduced nuclear CUG RNA foci in DM1 patient myocytes in a dose dependent fashion. Further, unitary AAV9-packaged Cas13d, PUF-E17 and PUF (blocking) were separately delivered via intramuscular (IM) and intravenous (IV) injections to adult (8-12 weeks old) HSA_{LR} DM1 mice. We report dose-dependent transgene expression, correction of DM1-associated alternative splicing and reduction in myotonia with electromyography at 4 weeks post IM injection with all RNA targeting systems. As expected, reduction in RNA foci and improvements in splicing with Cas13d and PUF-E17 were accompanied by reduction in CUG RNA levels (>50%). In summary, we show different mechanistic approaches (destruction and blocking) that can effectively target the MRE in DMPK and improve molecular and clinical features of DM1. FIGS. 6-12.

### Example 6: Tolerability and Transgene Expression in NHP Studies

Non-human primate (NHP) studies with AAV9 encoding the RNA-targeting compositions described above (A01215, A01344, and A01686) under control of a muscle (skeletal, smooth, and cardiac) specific promoter to assess tolerability and transgene expression levels in target tissues (biodistribution) are being carried out. The RNA-targeting compositions. Vector with DNA encoding CUG-targeting compositions were delivered by an AAV9 vector. PUF(CUG)-E17, Cas13d(CUG) or PUF(CUG) alone expression is driven by a promoter (FIG. 4A-C and FIG. 12 A-B). In some aspects, a desmin promoter (full-length desmin SEQ ID NO: 568 or truncated desmin SEQ ID NO: 569) is used.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or embodiment herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

What is claimed is:

1. A composition comprising a nucleic acid sequence encoding an RNA-binding polypeptide comprising a non-guided RNA binding polypeptide or a guided RNA-binding polypeptide capable of binding a toxic target CUG repeat RNA sequence.

2. The composition of claim 1, wherein the RNA-binding polypeptide is a fusion protein.

3. The composition of claim 2, wherein the fusion protein comprises the RNA binding polypeptide fused to an endonuclease capable of cleaving the toxic CUG repeat RNA sequence.

4. The composition of any one of the preceding claims, wherein the non-guided RNA binding polypeptide is a PUF or PUMBY protein.

5. The composition of any one of the preceding claims, wherein the guided RNA-binding polypeptide is a Cas13d protein.

6. The composition of any one of the preceding claims, wherein the cas13d protein is catalytically dead.

7. The composition of any one of the preceding claims, wherein the cas13d protein comprises an amino acid sequence set forth in any one of SEQ ID NOs 583 or 586-589.

8. The composition of any one of the preceding claims, wherein the endonuclease is a nuclease domain of a ZC3H12A zinc-finger endonuclease.

9. The composition of any one of the preceding claims, wherein the PUF RNA binding protein comprises an amino acid sequence set forth in any one of SEQ ID NOs 444-451, 461, 570, or 638-649.

10. The composition of any one of the preceding claims, wherein the PUF RNA binding protein comprises an amino acid sequence set forth in SEQ ID NO: 444.

11. The composition of any one of the preceding claims, wherein the toxic target CUG RNA repeat sequence comprises any one of the nucleic acid sequences set forth in SEQ ID NOs 453-456.

12. The composition of any one of the preceding claims, wherein the toxic target CUG RNA repeat sequence comprises the nucleic acid sequence set forth in SEQ ID NO: 454.

13. The composition of any one of the preceding claims, wherein the CUG-targeting PUF protein is encoded by a nucleic acid sequence as set forth in SEQ ID NO: 452.

14. The composition of any one of the preceding claims, wherein the PUF or PUMBY protein is a human PUF or PUMBY protein.

15. The composition of any one of the preceding claims, wherein the PUF or PUMBY protein is linked to the ZC3H12A endonuclease by a linker sequence.

16. The composition of any one of the preceding claims, wherein the linker comprises the amino acid sequence set forth in SEQ ID NO: 411.

17. The composition of any one of the preceding claims, wherein the fusion protein comprises one or more signal sequences selected from the group consisting of a nuclear localization sequence (NLS), and a nuclear export sequence (NES).

18. The composition of any one of the preceding claims, wherein the ZC3H12A zinc finger nuclease comprises the amino acid sequence set forth in SEQ ID NO: 358 or SEQ ID NO: 359.

19. The composition of any one of the preceding claims, wherein the fusion protein comprises the amino acid sequence set forth in any one of SEQ ID NOs 559-567.

20. The composition of any one of the preceding claims, wherein the fusion protein is encoded by a nucleic acid sequence comprising SEQ ID NO: 460, SEQ ID NO: 516 or SEQ ID NO: 517.

21. The composition of any one of the preceding claims, wherein the nucleic acid molecule encoding the fusion protein comprises a promoter.

22. The composition of claim any one of the preceding claims, wherein the promoter is a tCAG promoter, an EFS/UBB promoter, a desmin promoter, a CK8e promoter, or an EFS promoter.

23. A vector comprising the composition of any one of the preceding claims.

24. The vector of claim 23, wherein the vector is selected from the group consisting of: adeno-associated virus (AAV), retrovirus, lentivirus, adenovirus, nanoparticle, micelle, liposome, lipoplex, polymersome, polyplex, and dendrimer.

25. The vector of claim 23, which is an AAV vector.

26. An AAV vector of any one of the preceding claims, wherein the AAV vector comprises:
a first AAV ITR sequence;
a first promoter sequence;
a polynucleotide sequence encoding for at least one CUG-repeat RNA binding polypeptide; and
a second AAV ITR sequence.

27. The AAV vector of any one of the preceding claims, wherein the CUG-repeat RNA binding polypeptide comprises a PUF or PUMBY protein.

28. The AAV vector of any one of the preceding claims, wherein the polynucleotide sequence encoding the PUF or PUMBY sequence comprises a nucleic acid sequence set forth in SEQ ID NO: 452.

29. The AAV vector of any one of the preceding claims, wherein the CUG-repeat RNA binding polypeptide comprises a Cas13d protein.

30. The AAV vector of any one of the preceding claims, wherein the polynucleotide sequence encoding the Cas13d sequence comprises a nucleic acid sequence set forth in SEQ ID NO: 601, 612, or 615-618.

31. The AAV vector of any one of the preceding claims, wherein the first promoter sequence comprises a nucleic acid sequence set forth in SEQ ID NO:568, 569, 608, 609, 634-637.

32. The AAV vector of any one of the preceding claims, wherein the first AAV ITR sequence comprises a nucleic acid sequence set forth in SEQ ID NO: 599 or 600.

33. The AAV vector of any one of the preceding claims, wherein the second AAV ITR sequence comprises a nucleic acid sequence set forth in SEQ ID NO: 599 or 600.

34. The AAV vector of any one of the preceding claims, wherein the vector further comprises a second promoter sequence.

35. The AAV vector of any one of the preceding claims, wherein the second promoter controls expression of a guide RNA (gRNA) wherein the gRNA comprises (i) a DR sequence and (ii) a spacer sequence.

36. The AAV vector of any one of the preceding claims, wherein the second promoter comprises a nucleic acid sequences set forth in SEQ ID NO: 519.

37. The AAV vector of any one of the preceding claims, wherein the vector further comprises a polyA sequence.

38. The AAV vector of any one of the preceding claims, wherein the vector comprises at least one linker sequence.

39. The AAV vector of any one of the preceding claims, wherein the vector comprises at least one nuclear localization sequence.

40. The AAV vector of any one of the preceding claims, wherein the vector is encoded be a nucleic set forth in any of one of SEQ ID NO: 574-582, 584-585, 590-597.

41. A pharmaceutical composition comprising:
a) the AAV viral vector of any one of claims 26-40; and
b) at least one pharmaceutically acceptable excipient and/or additive.

42. An AAV viral vector comprising:
a) an AAV vector of any one of the preceding claims; and
b) an AAV capsid protein.

43. The AAV viral vector of claim 42, wherein the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein.

44. The AAV viral vector of claim 21, wherein the AAV capsid protein is an AAV9 capsid protein

45. A cell comprising the vector of any one of the preceding claims.

46. A method of treating myotonic dystrophy type 1 (DM1) in a mammal comprising administering a composition or AAV vector according to any one of claims 1-45 to a toxic target CUG microsatellite repeat expansion (MRE) RNA sequence in tissues of the mammal whereby the level of expression of the toxic target RNA is reduced.

47. The method of claim 46, wherein the composition or AAV vector is administered to the subject intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally or intranerve.

48. The method of claim 46, wherein the composition or AAV vector is administered to the subject intravenously.

49. The method of claim 46, wherein the reduced level of expression of the toxic target RNA thereby ameliorates symptoms of DM1 in the mammal.

50. The method of claim 46, wherein the level of expression of the toxic target RNA is reduced compared to the reduction in the level of expression of untreated toxic target CUG RNA.

51. The method of claim 46, wherein the level of reduction is between 1-fold and 20-fold.
